(19) United States
(12) Patent Application Publication
(10) Pub. No.: US 2024/0091278 A1
(43) Pub. Date: Mar. 21, 2024

(54) PHARMACEUTICAL COMPOSITIONS FOR TREATING DISEASES

(71) Applicant: Siolta Therapeutics, Inc., San Carlos, CA (US)

(72) Inventors: Nikole KIMES, San Francisco, CA (US); Ricardo VALLADARES, Brookhaven, GA (US); Neeraja VAJRALA, Burlingame, CA (US)

(21) Appl. No.: 18/477,009

(22) Filed: Sep. 28, 2023

Related U.S. Application Data
(63) Continuation of application No. PCT/US2022/023710, filed on Apr. 6, 2022.
(60) Provisional application No. 63/171,804, filed on Apr. 7, 2021.

(51) Int. Cl. A61K 35/74; A61K 9/19; A61K 9/48; A61K 35/742; C12N 1/20

(52) U.S. Cl. CPC A61K 35/74; A61K 9/19; A61K 9/485; A61K 9/4858; A61K 35/742; C12N 1/20

(57) ABSTRACT

Provided herein are pharmaceutical compositions comprising a bacterial population and one or more pharmaceutically acceptable excipients. The pharmaceutical compositions may be encompassed by containers or packaging forms. Such pharmaceutical compositions can be orally administered to a subject for prevention and/or treatment of dysbiosis, dysbiosis associated conditions, inflammation, and/or metabolic diseases. Also provided herein are methods for manufacturing the pharmaceutical composition.

### CROSS REFERENCE

This application is a continuation of International Application No. PCT/US2022/023710, filed Apr. 6, 2022, which claims the benefit of U.S. Provisional Patent Application No. 63/171,804, filed on Apr. 7, 2021, each of which is hereby incorporated by reference in its entirety.

### BACKGROUND

Recent developments in the areas of microbiome and genome research provide evidence that the microbiome-host relationships fundamentally influence human health, disease onset and progression. For example, they have been implicated in the inflammatory and metabolic diseases, playing key roles in the etiology of these disorders. The rising incidence of these diseases is concerning and represents a major public health challenge.

Restoring the microbiome-host balance can help treating these disorders. Effective treatments comprise administration of live biotherapeutics. Currently available pharmaceutical compositions, however, can be limited due to a lack of effectiveness, scalability, reliability, or stability.

### BRIEF SUMMARY

In an aspect, a pharmaceutical composition comprises: a purified bacterial population comprising at least one strain of Akkermansia sp., at least one strain of Faecalibacterium sp., or at least one strain of Lactobacillus sp., wherein the pharmaceutical composition has a shelf stability subsequent to being stored in anaerobic environment for a period of at least 3 days at a temperature of at least 15° C.

In some embodiments, the shelf stability of the pharmaceutical composition comprises a ratio of a viability of the at least one strain of the purified bacterial population of the pharmaceutical composition relative to a viability of the at least one strain of the purified bacterial population of a comparable pharmaceutical composition prior to or without being stored in the anaerobic environment for the period of at least 3 days at the temperature of at least 15° C. In some embodiments, the viability of the at least one strain of the purified bacterial population of the pharmaceutical composition or the viability of the at least one strain of the purified bacterial population of the comparable pharmaceutical composition is measured in colony forming unit (CFU). In some embodiments, the ratio is at least about 1×10\hat{ }-5%. In some embodiments, the ratio is at least about 1×10\hat{ }-4%. In some embodiments, the ratio is at least about 1×10\hat{ }-2%. In some embodiments, the ratio is at least about 1%. In some embodiments, the ratio is at least about 10%. In some embodiments, the ratio is at least about 20%. In some embodiments, the ratio is at least about 50%. In some embodiments, the pharmaceutical composition is stored in the anaerobic environment for a period of at least 3 days at a temperature of about 25° C. In some embodiments, the pharmaceutical composition is stored in the anaerobic environment for a period of at least 10 days at a temperature of at least 15° C. In some embodiments, the pharmaceutical composition is stored in the anaerobic environment for a period of at least 100 days at a temperature of at least 15° C. In some embodiments, the pharmaceutical composition comprises a cryoprotectant. In some embodiments, the pharmaceutical composition is lyophilized. In some embodiments, the pharmaceutical composition comprises a pharmaceutically acceptable excipient. In some embodiments, the pharmaceutically acceptable excipient comprises maltodextrin, cellulose, methionine, ascorbic acid, magnesium stearate, beta-cyclodextrin, dextrin, 2-Hydroxypropyl-β-cyclodextrin cysteine, riboflavin, starch, Glucidex, mannitol, saccharose, trehalose, anhydrous lactose, or a combination thereof. In some embodiments, the pharmaceutical composition is encapsulated in a capsule. In some embodiments, the capsule is a plant-based capsule. In some embodiments, the pharmaceutical composition is encompassed by a blister pack, a sachet pack, a vial, a bottle, an ampoule, or a combination thereof. In some embodiments, the pharmaceutical composition is encompassed by the blister pack. In some embodiments, the pharmaceutical composition is encompassed by the sachet pack. In some embodiments, the pharmaceutical composition is formulated into a suspension. In some embodiments, the pharmaceutical composition is formulated as an oral dosage form. In some embodiments, the oral dosage form is a capsule, a tablet, an emulsion, a suspension, a syrup, a gel, a gum, a paste, a herbal tea, drops, dissolving granules, powders, tablets, a lyophilizate, a popsicle, a foam, or an ice cream. In some embodiments, the purified bacterial population comprises at least two of: the at least one strain of Akkermansia sp., the at least one strain of Faecalibacterium sp., and the at least one strain of Lactobacillus sp. In some embodiments, the purified bacterial population comprises the at least one strain of Akkermansia sp., the at least one strain of Faecalibacterium sp., and the at least one strain of Lactobacillus sp.

Disclosed herein, in some embodiments, are pharmaceutical compositions. In an aspect, a pharmaceutical composition comprises: i. a purified bacterial population comprising at least one strain of Akkermansia sp., at least one strain of Faecalibacterium sp., or at least one strain of Lactobacillus, and ii. a pharmaceutically acceptable excipient, wherein the pharmaceutically acceptable excipient comprises a moisture absorbent material.

In an aspect, a pharmaceutical composition comprises: i. a purified bacterial population comprising at least one strain of Akkermansia sp., at least one strain of Faecalibacterium sp., or at least one strain of a species of the Lactobacillaceae family, and ii. a pharmaceutically acceptable excipient, wherein the pharmaceutically acceptable excipient comprises a moisture absorbent material.

In some embodiments, the moisture absorbent material is selected from the group consisting of microcrystalline cellulose (MCC), hydroxypropyl methylcellulose, silicon dioxide (SiO₂), polyethylene glycol 8000, lactose, D-Trehalose dihydrate, mannitol, calcium phosphate tribasic, calcium sulfate, corn starch, fructose, xylitol, maltitol, anhydrous lactose and dicalcium phosphate (DCP). In some embodiments, the moisture absorbent material comprises SiO₂. In some embodiments, the SiO₂ is present in an amount of about 0.1% to about 10% by weight. In some embodiments, the SiO₂ is present in an amount of about 1% by weight. In some embodiments, the moisture absorbent material comprises mannitol. In some embodiments, the mannitol is present in an amount of about 1% to about 90% by weight. In some embodiments, the mannitol is present in an amount of about 5% to about 81.5% by weight. In some embodiments, the moisture absorbent material comprises anhydrous lactose. In some embodiments, the anhydrous lactose is present in an amount of about 1% to about 90% by weight. In some embodiments, the anhydrous lactose is present in an amount of about 5% to about 81.5% by weight. In some embodiments, the pharmaceutically acceptable excipient further comprises magnesium stearate. In some embodiments, the magnesium stearate is present in an amount of about 0.1% to about 10% by weight. In some embodiments, the magnesium stearate is present in an amount of about 1.5% by weight. In some embodiments, the pharmaceutically acceptable excipient further comprises microcrystalline cellulose. In some embodiments, the pharmaceutical composition is formulated into a suspension. In some embodiments, the pharmaceutical composition is formulated as an oral dosage form. In some embodiments, the oral dosage form is a capsule, a tablet, an emulsion, a suspension, a syrup, a gel, gum, a paste, a herbal tea, drops, dissolving granules, powders, tablets, a lyophilizate, a popsicle, a foam, or an ice cream. In some embodiments, the purified bacterial population comprises at least two of: the at least one strain of Akkermansia sp., the at least one strain of Faecalibacterium sp., and the at least one strain of Lactobacillus sp. In some embodiments, the purified bacterial population comprises at least two of: the at least one strain of Akkermansia sp., the at least one strain of Faecalibacterium sp., and the at least one strain of a species of the Lactobacillaceae family. In some embodiments, the purified bacterial population comprises the at least one strain of Akkermansia sp., the at least one strain of Faecalibacterium sp., and the at least one strain of Lactobacillus sp. In some embodiments, the purified bacterial population comprises the at least one strains of a species of the Lactobacillaceae family. In some embodiments, the purified bacterial population is lyophilized.

Disclosed herein, in some embodiments, are pharmaceutical compositions. In an aspect, a pharmaceutical composition comprises: i. a purified bacterial population comprising at least one strain of Akkermansia sp., at least one strain of Lactobacillus sp., or at least one strain of Faecalibacterium sp., and ii. a pharmaceutically acceptable excipient, wherein the pharmaceutical composition is encompassed by a plant-based capsule.

In an aspect, a pharmaceutical composition comprises: i. a purified bacterial population comprises at least one strain of Akkermansia sp., at least one strain of a species of the Lactobacillaceae family, or at least one strain of Faecalibacterium sp., and ii. a pharmaceutically acceptable excipient, wherein the pharmaceutical composition is encompassed by a plant-based capsule.

In some embodiments, the plant-based capsule is encompassed by a blister pack. In some embodiments, the plant-based capsule is a hypromellose capsule. In some embodiments, the blister pack is selected from the group consisting of Polyethylene terephthalate (PET), Polyvinyl chloride (PVC), Polyvinylidene chloride (PVDC), Polychlorotrifluoroethylene (PCTFE), Cyclic olefin polymers (COP), oriented polyamide (OPA), aluminum foil, plastic film. In some embodiments, the blister pack is an aluminum blister pack. In some embodiments, the blister pack is filled with nitrogen gas. In some embodiments, the pharmaceutically acceptable excipient comprises a moisture absorbent material. In some embodiments, the moisture absorbent material is selected from a group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose and dicalcium phosphate (DCP). In some embodiments, the moisture absorbent material comprises SiO₂. In some embodiments, the SiO₂ is present in an amount of about 0.1% to about 10% by weight. In some embodiments, the SiO₂ is present in an amount of about 1% by weight. In some embodiments, the moisture absorbent material comprises mannitol. In some embodiments, the mannitol is present in an amount of about 1% to about 90% by weight. In some embodiments, the mannitol is present in an amount of about 5% to about 81.5% by weight. In some embodiments, the moisture absorbent material comprises anhydrous lactose. In some embodiments, the anhydrous lactose is present in an amount of about 1% to about 90% by weight. In some embodiments, the anhydrous lactose is present in an amount of about 5% to about 81.5% by weight. In some embodiments, the pharmaceutically acceptable excipient further comprises magnesium stearate. In some embodiments, the magnesium stearate is present in an amount of about 0.1% to about 10% by weight. In some embodiments, the magnesium stearate is present in an amount of about 1.5% by weight. In some embodiments, the pharmaceutically acceptable excipient further comprises microcrystalline cellulose. In some embodiments, the purified bacterial population comprises at least two of: the at least one strain of Akkermansia sp., the at least one strain of Faecalibacterium sp., and the at least one strain of Lactobacillus sp. In some embodiments, the purified bacterial population comprises at least two of: the at least one strain of Akkermansia sp., the at least one strain of Faecalibacterium sp., and the at least one strain of a species of the Lactobacillaceae family. In some embodiments, the purified bacterial population comprises the at least one strain of Akkermansia sp., the at least one strain of Faecalibacterium sp., and the at least one strain of Lactobacillus sp. In some embodiments, the purified bacterial population comprises the at least one strain of Akkermansia sp., the at least one strain of Faecalibacterium sp., and the at least one strain of a species of the Lactobacillaceae family.

Disclosed herein, in some embodiments, are pharmaceutical compositions. In an aspect, a pharmaceutical composition comprises: i. a purified bacterial population comprising at least one strain of Akkermansia sp., at least one strain of Lactobacillus sp., or at least one strain of Faecalibacterium sp., and ii. a pharmaceutically acceptable excipient, wherein the pharmaceutical composition is encompassed by a blister pack.

In an aspect, a pharmaceutical composition comprises: i. a purified bacterial population comprising at least one strain of Akkermansia sp., at least one strain of a species of the Lactobacillaceae family, or at least one strain of Faecalibacterium sp., and ii. a pharmaceutically acceptable excipient, wherein the pharmaceutical composition is encompassed by a blister pack.

In some embodiments, the blister pack is selected from the group consisting of polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, plastic film. In some embodiments, the blister pack is an aluminum blister pack. In some embodiments, the blister pack is filled with nitrogen gas. In some embodiments, the pharmaceutically acceptable excipient comprises a moisture absorbent material. In some embodiments, the moisture absorbent material is selected from a group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose and dicalcium phosphate (DCP). In some embodiments, the moisture absorbent material comprises SiO₂. In some embodiments, the SiO₂ is present in an amount of about 0.1% to about 10% by weight. In some embodiments, the SiO₂ is present in an amount of about 1% by weight. In some embodiments, the moisture absorbent material comprises mannitol. In some embodiments, the mannitol is present in an amount of about 1% to about 90% by weight. In some embodiments, the mannitol is present in an amount of about 5% to about 81.5% by weight. In some embodiments, the moisture absorbent material comprises anhydrous lactose. In some embodiments, the anhydrous lactose is present in an amount of about 1% to about 90% by weight. In some embodiments, the anhydrous lactose is present in an amount of about 5% to about 81.5% by weight. In some embodiments, the pharmaceutically acceptable excipient further comprises magnesium stearate. In some embodiments, the magnesium stearate is present in an amount of about 0.1% to about 10% by weight. In some embodiments, the magnesium stearate is present in an amount of about 1.5% by weight. In some embodiments, the pharmaceutically acceptable excipient further comprises microcrystalline cellulose.

In some embodiments, the purified bacterial population comprises at least two of: the at least one strain of Akkermansia sp., the at least one strain of Faecalibacterium sp., and the at least one strain of Lactobacillus sp. In some embodiments, the purified bacterial population comprises at least two of: the at least one strain of Akkermansia sp., the at least one strain of Faecalibacterium sp., and the at least one strains of a species of the Lactobacillaceae family. In some embodiments, the purified bacterial population comprises at least one strain of Akkermansia sp., at least one strain of Faecalibacterium sp., and at least one strain of Lactobacillus sp. In some embodiments, the purified bacterial population comprises at least one strain of Akkermansia sp., at least one strain of Faecalibacterium sp., and at least one strain of a species of the Lactobacillaceae family. In some embodiments, the purified bacterial population is lyophilized. In some embodiments, the at least one strain of Akkermansia sp., the at least one strain of Faecalibacterium sp., and the at least one strain of Lactobacillus sp. are selected from the strains listed in Table 1. In some embodiments, the at least one strain of Akkermansia sp., the at least one strain of Faecalibacterium sp., and the at least one strain of a species of the Lactobacillaceae family are selected from the strains listed in Table 1. In some embodiments, the bacterial population comprises A. muciniphila (DSM 33213), F. prausnitzii (DSM 33185), or L. crispatus (DSM 33187). In some embodiments, the bacterial population comprises at least two of the bacterial strains A. muciniphila (DSM 33213), F. prausnitzii (DSM 33185), and L. crispatus (DSM 33187). In some embodiments, the bacterial population comprises the bacterial strains A. muciniphila (DSM 33213), F. prausnitzii (DSM 33185), and L. crispatus (DSM 33187). In some embodiments, each bacterial strain is present in an amount from about 10\hat{ }3 CFU/dose to about 10\hat{ }12 CFU/dose. In some embodiments, each bacterial strain is present in an amount from about 10\hat{ }7 CFU/dose to about 10\hat{ }10 CFU/dose. In some embodiments, the at least one strain of Akkermansia sp. is present in an amount of about 10\hat{ }6 CFU/dose to about 2×10\hat{ }9 CFU/dose. In some embodiments, the at least one strain of Faecalibacterium sp. is present in an amount of about 10\hat{ }6 CFU/dose to about 2×10\hat{ }9 CFU/dose. In some embodiments, the at least one strain of Lactobacillus sp. is present in an amount of about 10\hat{ }7 CFU/dose to about 5×10\hat{ }9 CFU/dose. In some embodiments, the at least one strain of a species of the Lactobacillaceae family is present in an amount of about 10\hat{ }7 CFU/dose to about 5×10\hat{ }9 CFU/dose. In some embodiments, the bacterial population is present in a total amount of about 10\hat{ }3 CFU/dose to about 10\hat{ }12 CFU/dose. In some embodiments, the bacterial population is present in a total amount of about 10\hat{ }7 CFU/dose to about 10\hat{ }10 CFU/dose.

Disclosed herein, in some embodiments, are methods for treating a subject having or suspected of having a disease. In an aspect, a method for treating a subject having or suspected of having a disease comprises administering to the subject a pharmaceutical composition of any pharmaceutical compositions disclosed thereof.

In some embodiments, the disease is an inflammatory disease. In some embodiments, the inflammatory disease is an allergy or dermatitis. In some embodiments, the allergy is allergic asthma, allergic pediatric asthma or food allergy. In some embodiments, the disease is a metabolic disease. In some embodiments, the metabolic disease is obesity, diabetes, or a metabolic syndrome.

Disclosed herein, in some embodiments, are methods for large-scale growth of Lactobacillus sp. In an aspect, a method for a large-scale growth of Lactobacillus sp. comprises performing a plurality of inoculation rounds with an increasing amount of growth media, wherein each inoculation round comprises at least about 0.5% of a total batch material of a preceding inoculation round.

Disclosed herein, in some embodiments, are methods for large-scale growth of species of the Lactobacillaceae family. In an aspect, a method for a large-scale growth of species of the Lactobacillaceae family comprises performing a plurality of inoculation rounds with an increasing amount of growth media, wherein each inoculation round comprises at least about 0.5% of a total batch material of a preceding inoculation round.

Disclosed herein, in some embodiments, are methods for large-scale growth of species of the Lactobacillaceae family. In an aspect, a method for a large-scale growth of species of the Lactobacillaceae family comprises performing a plurality of inoculation rounds with an increasing amount of growth media, wherein an inoculation round comprises at least about 0.5% by volume of a total batch material of a preceding inoculation round, wherein a growth media of the inoculation round is at least about 50 L.

In some embodiments, the Lactobacillus sp. comprises Lactobacillus crispatus. In some embodiments, the species of the Lactobacillaceae family comprises Lactobacillus crispatus. In some embodiments, the Lactobacillus sp. comprises Lactobacillus crispatus (DSM 33187). In some embodiments, the species of the Lactobacillaceae family comprises Lactobacillus crispatus (DSM 33187). In some embodiments, the growth media is from about 100 mL to about 4,000 L. In some embodiments, the growth media is from about 50 L to about 4,000 L. In some embodiments, the method further comprises an initial inoculation round of about 500 mL growth media. In some embodiments, at least one of the inoculation rounds is in a volume of about 3500 L growth media. In some embodiments, at least one of the inoculation rounds is in a volume of about 50 L. In some embodiments, the initial inoculation round comprises a frozen stock of Lactobacillus crispatus of about 0.4% of an initial inoculation round growth media. In some embodiments, the initial inoculation round comprises growing Lactobacillus crispatus in anaerobic condition. In some embodiments, the method further comprises performing a plurality of sterilization and degassing rounds for the growth media. In some embodiments, the method further comprises lyophilizing the batch. In some embodiments, the method further comprises centrifuging the batch before the lyophilizing. In some embodiments, the method further comprises grinding the batch after the lyophilizing.

Disclosed herein, in some embodiments, are methods for producing a pharmaceutical composition. In an aspect, a method for producing a pharmaceutical composition comprises: 1) providing a mixture comprising a purified bacterial population comprising at least one strain of Akkermansia sp., at least one strain of Faecalibacterium sp., or at least one strain of Lactobacillus sp.; 2) filling capsules with the mixture; and 3) packing the capsules into a blister pack; wherein the providing and the packing are under an oxygen-free atmosphere.

In an aspect, a method for producing a pharmaceutical composition comprises: 1) providing a mixture comprising a purified bacterial population comprising at least one strain of Akkermansia sp., at least one strain of Faecalibacterium sp., or at least one strain of a species of the Lactobacillaceae family; 2) filling capsules with the mixture; and 3) packing the capsules into a blister pack; wherein the providing and the packing are under an oxygen-free atmosphere.

In some embodiments, the method further comprises storing the capsules in a container before the packing. In some embodiments, the storing is under an oxygen-free atmosphere. In some embodiments, the oxygen-free atmosphere is accomplished by injecting nitrogen gas or an oxygen scrubber. In some embodiments, the filling is accomplished by a capsule filler. In some embodiments, the filling is accomplished in an anaerobic chamber. In some embodiments, the capsule is a plant-based capsule. In some embodiments, the plant-based capsule is a hypromellose capsule. In some embodiments, the blister pack is selected from polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, plastic film. In some embodiments, the blister pack is an aluminum blister pack. In some embodiments, the blister pack is filled with nitrogen gas. In some embodiments, the pharmaceutical composition further comprises a pharmaceutically acceptable excipient.

In some embodiments, the pharmaceutically acceptable excipient comprises a moisture absorbent material. In some embodiments, the moisture absorbent material is selected from the group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose and dicalcium phosphate (DCP). In some embodiments, the moisture absorbent material comprises SiO₂. In some embodiments, the SiO₂ is present in an amount of about 0.1% to about 10% by weight. In some embodiments, the SiO₂ is present in an amount of about 1% by weight. In some embodiments, the moisture absorbent material comprises mannitol. In some embodiments, the mannitol is present in an amount of about 1% to about 90% by weight. In some embodiments, the mannitol is present in an amount of about 5% to about 81.5% by weight. In some embodiments, the moisture absorbent material comprises anhydrous lactose. In some embodiments, the anhydrous lactose is present in an amount of about 1% to about 90% by weight. In some embodiments, the anhydrous lactose is present in an amount of about 5% to 81.5% by weight. In some embodiments, the pharmaceutically acceptable excipient further comprises magnesium stearate. In some embodiments, the magnesium stearate is present in an amount of about 0.1% to about 10% by weight. In some embodiments, the magnesium stearate is present in an amount of about 1.5% by weight. In some embodiments, the pharmaceutically acceptable excipient further comprises microcrystalline cellulose. In some embodiments, the purified bacterial population comprises at least two of: the at least one strain of Akkermansia sp., the at least one strain of Faecalibacterium sp., or the at least one strain of Lactobacillus sp. In some embodiments, the purified bacterial population comprises at least two of: the at least one strain of Akkermansia sp., the at least one strain of Faecalibacterium sp., or the at least one strain of a species of the Lactobacillaceae family. In some embodiments, the purified bacterial population comprises the at least one strain of Akkermansia sp., the at least one strain of Faecalibacterium sp., and the at least one strain of Lactobacillus sp. In some embodiments, the purified bacterial population comprises the at least one strain of Akkermansia sp., the at least one strain of Faecalibacterium sp., and the at least one strain of a species of the Lactobacillaceae family. In some embodiments, the bacterial population is lyophilized.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The features of the disclosure are set forth with particularity in the appended claims. A better understanding of the features and the disclosure will be obtained by reference to the following detailed description that sets forth illustrative embodiments, in which the principles of the invention are utilized, and the accompanying drawings (also “Figure” and “FIG.” herein), of which:

FIG. 1 shows a schematic flow chart for an optimized and ultra-large-scale growth and manufacturing process for L. crispatus (DSM 33187) in 3500 L culture volume. About 1.84 mL of Working Cell Bank (WCB) L. crispatus (DSM 33187) was thawed in the anaerobic chamber and inoculated in 0.5 L reduced LC100 media (0.4% v/v inoculation rate) in a 2 L flask 101 in an anaerobic chamber. The cultured was stopped when either optical density at 583 nanometer (OD₅₈₃)>4.5 or the culture grew for 20 hours. 0.1 L culture in 101 was used to inoculate in 20 L media (0.5% v/v inoculation rate) in a 20 L fermenter 102. The culture was stopped for one of the following criteria: a) OD₅₈₃>6, b) concentration of glucose <2 g/L, c) total culture time reaches 20 h, or d) a slowing of the growth rate is detected after three subsequent OD₅₈₃ readings. 250 L, defined in TABLE 3, was prepared. 160 L sugar feed was added to a 3500 L fermenter 103. The whole content of 102, or about 20 L culture, was used to inoculate in 3400 L media (0.59% v/v inoculation rate) in a 3500 L fermenter 103. The cultured was stopped when stopped for one of the following criteria: a) OD₅₈₃>8, b) concentration of glucose <2 g/L, c) total culture time reaches 20 h, or d) a slowing of the growth rate is detected after three subsequent OD₅₈₃ readings. The entire culture in 103 was then centrifuged under anaerobic atmosphere and harvested as a biomass. 150 L filter-sterilized degassed cryoprotectant, defined in TABLE 5, was mixed with the biomass in a mixing tank purged with anaerobic gas. The biomass with cryoprotectant was lyophilized (frozen and dried) and ground. For each step, the media used for the bacterial culture were sterilized (autoclaving at 121° C.) and degassed with N₂H₂CO₂ (90:5:5) before use. Sugar components (glucose) was prepared separately from the remaining LC100 media components. Sugar feed was filter sterilized, degassed, and added to the remaining LC100 media components to generate the complete culture media.

FIG. 2 shows a schematic flow chart summarizing the manufacturing steps of the drug product (DP).

FIG. 3 shows a schematic flow chart summarizing the manufacturing steps of the drug product (DP) after the drug substance (DS) batch has been confirmed. The DS comprises A. muciniphila (DSM 33213), L. crispatus (DSM 33187), and F. prausnitzii (DSM 33185).

FIG. 4A shows a schematic flow chart summarizing a potency test of the drug product mixed with different excipients. The excipients tested includes Vivapur 103, Vivapur 112, Vivapur 200 XLM, Vivapur Prosolv SMCC90, and EMDEX. The drug product and the excipient were mixed in a 1:1, 1:2, or 1:3 ratio. The drug product without any excipient was used as a negative control. FIG. 4B shows that Vivapur 103, Vivapur 112, Vivapur 200 XLM, Vivapur Prosolv SMCC90 were not soluble in water. The drug products mixed with different excipients in Eppendorf tubes after 5-minute room temperature incubation are shown.

FIGS. 5A, 5B, & 5C show that F. prausnitzii (DSM 33185) in the drug product (DP) comprising different excipients is stable at 4° C. and −20° C. FIG. 5A shows a schematic flow chart summarizing a potency test of the drug product mixed with different excipients. The drug product comprises drug substances (DS) comprising: A. muciniphila (DSM 33213), L. crispatus (DSM 33187), and F. prausnitzii (DSM 33185). The excipients tested includes EMDEX, mannitol, and anhydrous lactose. The drug product and the excipient were mixed in a 1:2 ratio with or without silicon dioxide (SiO₂). Each drug product mixture contains ˜0.3 g of drug product and excipients and stored in 5/6 glass vials. Desiccant packets were placed in freezer boxes containing glass vials. The drug product without any excipient was used as a negative control. The potency of the drug products was scored on Day 0 (D0), D7, D14, D28, and D60. FIG. 5B shows that F. prausnitzii (DSM 33185) in the drug product comprising various excipients are stable at 4° C. The viability cell count, as the colony-forming unit per gram (CFU/g) of the drug product, of F. prausnitzii (DSM 33185) stored at 4° C. was scored on D0, D7, D14, and D28 and shown on the Y-axis. The X-axis shows the drug product mixed with different excipients and with or without silicon dioxide. FIG. 5C shows that F. prausnitzii (DSM 33185) in the drug product comprising various excipients are stable at −20° C. The viability cell count, as the colony-forming unit per gram (CFU/g) of the drug product, of F. prausnitzii (DSM 33185) stored at −20° C. was scored on D0, D7, D14, and D28 and shown on the Y-axis. The X-axis shows the drug product mixed with different excipients and with or without silicon dioxide.

FIGS. 6A, 6B, & 6C show that F. prausnitzii (DSM 33185) in the drug product (DP) are stable in low humidity, and A. muciniphila (DSM 33213) and L. crispatus (DSM 33187) are stable in low and high humidity. FIG. 6A shows a schematic flow chart summarizing a potency test of the drug product mixed with mannitol and anhydrous lactose, with silicon dioxide (SiO₂). The drug product comprises drug substances (DS) comprising: A. muciniphila (DSM 33213), L. crispatus (DSM 33187), and F. prausnitzii (DSM 33185). About 0.3 g drug product mixtures were filled into 8 capsules per condition in the anaerobic chamber (AS-150; ˜40% humidity) and Biosafety cabinet (BSC; >60% humidity). The capsules were stored in glass vials at 4° C. and −20° C. Desiccant packets were placed in the freezer boxes containing the glass vials. The potency of the mixtures was tested on Day 0 (D0), D7, D14, D21, D28, D60, D90, and D120. FIG. 6B shows that F. prausnitzii (DSM 33185) in the drug product mixtures with anhydrous lactose and SiO² are stable at 4° C. in low humidity but not in high humidity. A. muciniphila (DSM 33213) and L. crispatus (DSM 33187) are stable in low and high humidity. The viability cell count, as the colony-forming unit per gram (CFU/g) of the drug product, of F. prausnitzii (DSM 33185), A. muciniphila (DSM 33213) and L. crispatus (DSM 33187) stored at 4° C. and −20° C. was scored on D0, D7, D14, and D21 and shown on the Y-axis. The X-axis lists the day when the viable cell count was recorded. FIG. 6C shows that F. prausnitzii (DSM 33185) in the drug product mixtures mannitol and SiO² are stable at 4° C. and −20° C. in low humidity but not in high humidity. A. muciniphila (DSM 33213) and L. crispatus (DSM 33187) are stable in low and high humidity. The viability cell count, as the colony-forming unit per gram (CFU/g) of the drug product, of F. prausnitzii (DSM 33185), A. muciniphila (DSM 33213) and L. crispatus (DSM 33187) stored at −20° C. was scored on D0, D7, D14, and D21 and shown on the Y-axis. The X-axis lists the day when the viable cell count was recorded.

FIGS. 7A, 7B, & 7C show that F. prausnitzii (DSM 33185) in the drug product (DP), A. muciniphila (DSM 33213), and L. crispatus (DSM 33187) are stable in the drug product line environment (DP). The drug product line environment (DP) was maintained at 25° C. and <30% humidity. The anaerobic chamber (AC) was maintained at room temperature and >50% humidity. FIG. 7A shows that F. prausnitzii (DSM 33185) is stable in the drug product line environment but not in Aerobic chamber. The viability cell count, as the colony-forming unit per gram (CFU/g) of F. prausnitzii (DSM 33185), was scored and shown on the Y-axis. The X-axis lists the time point when the viability cell count was scored: at the start of experiment (t=0 h), 1 hour (t=1 h), t=2 h, t=4 h, t=8 h, and t=24 h. FIG. 7B shows that A. muciniphila (DSM 33213) is stable in the drug product line environment and Aerobic chamber. The viability cell count, as the colony-forming unit per gram (CFU/g) of F. prausnitzii (DSM 33185), was scored and shown on the Y-axis. The X-axis the time point when the viability cell count was scored: at the start of experiment (t=0 h), 1 hour (t=1 h), t=2 h, t=4 h, t=8 h, and t=24 h. FIG. 7C shows that L. crispatus (DSM 33187) is stable in the drug product line environment and Aerobic chamber. The viability cell count, as the colony-forming unit per gram (CFU/g) of F. prausnitzii (DSM 33185), was scored and shown on the Y-axis. The X-axis lists the time point when the viability cell count was scored: at the start of experiment (t=0 h), 1 hour (t=1 h), t=2 h, t=4 h, t=8 h, and t=24 h.

FIG. 8 shows a schematic distribution of A. muciniphila (DSM 33213), L. crispatus (DSM 33187), and F. prausnitzii (DSM 33185), mannitol, and silicon dioxide in a feasibility batch.

FIGS. 9A & 9B show that the raw materials of the drug product of a feasibility batch form a visually homogenous mixture. FIG. 9A shows the distribution of the raw materials in FIG. 8 before mixing. FIG. 9B shows the distribution of the raw materials in FIG. 8 after mixing.

FIG. 10 shows that the drug product of a feasibility batch has particle of different sizes. The distribution of particle sizes in the drug product of a feasibility batch is plotted. The X-axis shows the sieve size. The Y-axis shows the proportion of particles retain by the sieve.

FIG. 11 shows that the drug product of a feasibility batch has particles of different sizes. The microscopic picture of the drug product under 40× magnification is shown. Particles of different sizes are observed.

FIG. 12 shows that the drug product of a feasibility batch is soluble in water. After 10 seconds of homogenization at 150 rpm, most materials in the drug product dissolved in water.

FIG. 13A shows a schematic flow chart summarizing an example drug product production process. FIG. 13B shows a schematic flow chart summarizing another example drug product production process.

FIG. 14A shows that achieving anaerobic condition inside the capsule is slow inside the anaerobic chamber. The picture of the capsule in different time points inside the anaerobic chamber are shown. The color of the capsule is labeled on top of each image. The V-cap capsule was moved into the anaerobic chamber. The capsule was filled with YFAP broth+0.12% Rezasurin. The capsule was incubated in the anaerobic chamber. Rezasurin is an oxygen indicator: it appears pink in the presence of oxygen, and yellow in the absence of oxygen. When the capsule was filled inside the anaerobic chamber, it took about an hour to remove the oxygen gas inside the capsule.

FIG. 14B shows rapid oxygen movement into the capsule outside the anaerobic chamber. The picture of the capsule in different time points outside the anaerobic chamber are shown. The color of the capsule is labeled on top of each image. The V-cap capsule was incubated the anaerobic chamber overnight. The capsule was filled with YFAP broth+0.12% Rezasurin. The capsule was incubated moved out of the anaerobic chamber. Once moved out of the anaerobic chamber, it took about eight minutes for the oxygen gas to move inside the capsule.

FIGS. 15A & 15B show that the drug product is stable under multiple freeze-thaw cycles. FIG. 15A shows a schematic flow chart summarizing a method to evaluate the drug product stability in freeze-thaw cycles. Drug products comprising F. prausnitzii (DSM 33185), A. muciniphila (DSM 33213), and L. crispatus (DSM 33187) in capsules packed in aluminum blister packs were stored at a −20° C. freezer. The blister packs were removed from the freezer and thawed for 30 minutes at room temperature on day 1 (D1), D2, D3, D4, D5, D6, D7, and D8, representing 1, 2, 3, 4, 5, 6, 7, 8 freeze-thaw cycles. Once thawed, the capsules were opened in the biosafety cabinet and moved into an anaerobic chamber. The drug products were serially diluted with PBS-C and plated on growth plate media. The colony-forming unit per capsule (CFU/capsule) was scored. FIG. 15B shows that F. prausnitzii (DSM 33185), A. muciniphila (DSM 33213), and L. crispatus (DSM 33187) are stable under multiple freeze-thaw cycles. The Y-axis shows the CFU/capsule of the strains. The X-axis shows the number of freeze-thaw cycles the tested drug product had accumulated.

FIGS. 16A & 16B show that the drug product is stable when transported inside a cooler. FIG. 16A shows a schematic flow chart summarizing a method to evaluate the drug product stability in the cooler. Drug products comprising F. prausnitzii (DSM 33185), A. muciniphila (DSM 33213), and L. crispatus (DSM 33187) in capsules packed in aluminum blister packs were stored at a −20° C. freezer. The blister packs were moved to the cooler with ice packs and temperature tale monitor. The temperature inside the cooler was monitored. The blister was removed from the cooler after six hours and thawed for 30 minutes at room temperature. Once thawed, the capsules were opened in the biosafety cabinet and moved into an anaerobic chamber. The drug products were serially diluted with PBS-C and plated on growth plate media. The colony-forming unit per capsule (CFU/capsule) was scored. On different days, different numbers of blisters were tested in the cooler. The DP without being moved in the cooler was used as a control. FIG. 16B shows the cooler temperature on day 1 with 4 blister packs. FIG. 16C shows the cooler temperature on day 2 with 4 blister packs. FIG. 16D shows the cooler temperature on day 3 with 12 blister packs. FIG. 16E shows the cooler temperature on day 4 with 12 blister packs. In FIGS. 16B, 16C, 16D, & 16E, the Y-axis shows the temperature of the cooler, and the X-axis shows the temperature of the time inside the cooler. FIG. 16F shows that F. prausnitzii (DSM 33185), A. muciniphila (DSM 33213), and L. crispatus (DSM 33187) are stable during transportation inside the cooler The Y-axis shows the CFU/capsule of the strains. The X-axis shows the day the drug product was tested, with the number of blister packs tested indicated.

FIG. 17 shows a schematic flow chart summarizing an example method for evaluating the drug product stability.

FIG. 18 shows a schematic flow chart summarizing an example method for evaluating different cryoprotectants.

### DETAILED DESCRIPTION

The present disclosure provides pharmaceutical compositions that may comprise, consist essentially of, or consist of a bacterial population and one or more pharmaceutically acceptable excipients. The pharmaceutical compositions may also be encompassed by plant-based capsules, blister packs, sachet packs, or any combinations thereof. The pharmaceutical may be used to treat inflammatory diseases or metabolic diseases.

Pharmaceutical compositions comprising live bacterial can be prone to degradation or loss of activity (e.g., pharmacological stabilities, pharmaceutical activities, biological/physiological activities elicited by the pharmaceutical compositions when administered by a subject). The loss of activity of the pharmaceutical composition can be related to the loss of viability of the bacteria. The loss of activity of the pharmaceutical compositions can be prevented by using pharmaceutically acceptable excipients, cryoprotectants, containers (including but not limited to capsules), improving packaging forms, or any combinations thereof. The loss of activity of the pharmaceutical compositions can also be prevented by packaging or manufacturing the pharmaceutical compositions in various conditions. The present disclosure provides pharmaceutically acceptable excipients, cryoprotectants, containers, packaging forms, packaging or manufacturing conditions, or any combinations thereof for maintaining the activity of the pharmaceutical compositions or the viability of live bacteria of the pharmaceutical compositions.

Inoculation and growing of bacteria in a large volume in pharmaceutical composition manufacturing (e.g., exceeding routine laboratory scale, such as less than about 50 L) can be challenging due to inability of the bacteria to maintain robust growth in the large volume. Maintenance of the bacterial growth in large volume can be achieved by inoculating and/or growing the bacteria in various conditions or with various substances. The present disclosure provides the methods and compositions for maintaining robust bacterial growth in large volume.

Provided herein are methods for producing pharmaceutical compositions comprising a bacterial population. The methods may comprise blending and packing the pharmaceutical compositions under an oxygen-free atmosphere.

Also disclosed are methods for large-scale growth of Lactobacillus sp. Also disclosed are methods for large-scale growth of species of the Lactobacillaceae family. The methods may comprise performing a plurality of inoculation rounds with an increasing amount of growth media. Each of the plurality of inoculation rounds may comprise at least 0.5% of a total batch material of a preceding round.

### Definitions

The term “purified” or “substantially purified” as used herein when referring to a bacterial strain or a mixture of more than one bacterial strain, refers to the bacterial strain or bacterial strains that are substantially enriched in a sample. A purified or substantially purified bacterial strain(s) in sample may comprise at least about 50%, 60%, 70%, 80%, 85%, 90%, 95%, 99% or greater of the bacterial strain(s) in the sample. A purified or substantially purified bacterial strain(s) in sample may also comprise less than about 40%, 30%, 20%, 15%, 14%, 13%, 12%, 11%, 10%, 9%, 8%, 7%, 6%, 5%, 4%, 3%, 2%, 1% or less of the strains other than the bacterial strain(s) present in the sample. Such strain may comprise a bacterial strain. Such strain may also comprise any non-bacterial strains such as strains from other organisms.

The term “pharmaceutical composition” or “composition”, when referring to a pharmaceutical product, refers to a composition that can elicit or induce at least one physiological effect to a subject when administered by the subject, preferably a human. Such physiological effect may positively contribute to the overall health of a subject. In some cases, such physiological effect may also curb, inhibit, reduce, or decrease a negative physiological phenomenon of the subject.

The term “pharmaceutically acceptable excipient” or “excipient”, when referring to a pharmaceutical composition, refers to an excipient that does not produce an adverse, allergic or other untoward reaction when administered to a subject, preferably a human. A pharmaceutically acceptable excipient may not produce the adverse, allergic or other untoward reaction when administered to the subject when it is administered alone or in combination with the pharmaceutical composition. A pharmaceutically acceptable excipient may include any solvents, dispersion media, coatings, isotonic and absorption delaying agents and the like. A pharmaceutically acceptable excipient may be added to a pharmaceutical composition to stabilize the pharmaceutical composition. A pharmaceutically acceptable excipient may be added to a pharmaceutical composition to prevent the contamination of the pharmaceutical composition. A pharmaceutically acceptable excipient may be an inert substance added to a pharmaceutical composition to facilitate processing, handling, administration, etc. of the pharmaceutical composition.

The term “moisture absorbent material” refers to a material that can absorb water. A moisture absorbent material may decrease the water content or the relative humidity (RH) of an environment, relative to a comparable environment without the moisture absorbent material. When a moisture absorbent material is stored together with another substance (e.g., a pharmaceutical composition), the water content of the substance may be decreased, relative to a comparable substance without the moisture absorbent material.

The term “lyophilization” refers to a water removal process comprising freezing and drying cycles. A lyophilized substance may have less than 10%, 9%, 8%, 7%, 6%, 5%, 4%, 3%, 2%, 1% or less of the water content, by weight, in the substance. Lyophilization may comprise a first freezing cycle, followed by subsequent drying cycles. The freezing cycle may freeze a substance such that a sublimation state and not a melt state of the substance will occur in the subsequent drying cycle. The first drying cycle may comprise lowering pressure and increasing temperature to remove the water content from the substance. The first drying cycle may comprise placing the substance in a vacuum. The first drying cycle may comprise slow heating. Optionally, a second drying cycle may be used following the first drying cycle. A second drying cycle may comprise rapid heating in order to break the bonds between the substance and the water molecules.

The term “oxygen-free” or “anaerobic” as used herein refers to a state with low oxygen or without oxygen. An oxygen-free or anaerobic environment may have 21%, 20%, 19%, 18%, 17%, 16%, 15%, 14%, 13%, 12%, 11%, 10%, 9%, 8%, 7%, 6%, 5%, 4%, 3%, 2%, 1%, 1×10\hat{ }-1%, 1×10\hat{ }-2%, 1×10\hat{ }-3%, 1×10\hat{ }-4%, 1×10\hat{ }-5%, 1×10\hat{ }-6%, 1×10\hat{ }-7%, 1×10\hat{ }-8%, 1×10\hat{ }-9%, 1×10\hat{ }-10%, 1×10\hat{ }-11% or less oxygen, by volume, in the atmosphere of the environment.

The term “oxygen scrubber” as used herein refers to a substance that can absorb or remove oxygen. An oxygen scrubber may sequester the oxygen from contacting other substances. An oxygen scrubber may remove the oxygen by a chemical reaction. An oxygen scrubber may also remove the oxygen from one environment such that other substances in the environment may not contact the oxygen. An oxygen scrubber may be used to create an oxygen-free or anaerobic environment.

The term “capsule” as used herein refers a container comprising a sac covered by a membrane for containing a pharmaceutical composition. In some cases, a capsule may not be administered to a subject. In some cases, a capsule may not be administered to a subject alongside the pharmaceutical composition. In other cases, a capsule may be administered to a subject.

The term “blister pack” as used herein refers to a pre-formed pack for storing and sealing a capsule or a pharmaceutical composition. A blister may comprise a cavity or pocket and a lid sealing the cavity or pocket. A capsule or a pharmaceutical composition may be sealed in the blister such that the capsule or the pharmaceutical composition may not in direct contact with the molecules or substances outside of the sealed cavity or pocket.

The term “capsule filler” as used herein refers to a means to fill a capsule with a pharmaceutical composition. A capsule filler may comprise a mechanical capsule filler. In some cases, a capsule filler may comprise an automated mechanical capsule filler. In other cases, the capsule is manually filled. In other cases, the capsule is manually filled in an anerobic chamber.

The term “plant-based” as used therein refers to a substance or material that is derived from plants. A plant-based substance or material does not comprise any animal or animal-derived meats, products or by-products. A plant-based substance or material does not comprise any meats, products, or by-products that originated in slaughtered animal.

The term “inflammatory disease” as used therein refers to a type of diseases, symptoms, or conditions that when the immune system of a subject attacks the tissues or cells of the subject in which the tissues or cells are not infected, injured, or in a disease state. The tissues or cells may also be in a healthy state.

The term “metabolic disease” as used therein refers to a type of diseases, symptoms, or conditions that disrupt or have a disrupted metabolic process. A disrupted metabolic process may have increased or decreased activity or efficiency relative to a comparable metabolic process that is not disrupted. A disrupted metabolic process may also be different frequency of activity or efficiency relative to a comparable metabolic process that is not disrupted.

The term “inoculation” as used herein refers to a process of introducing a microorganism (e.g., a bacterial strain described herein) into a growth medium substantially pure of any microorganisms. An inoculation may result in an increase of mass, either through an increase in cell numbers through cell division or an increase in cell mass through cell growth, when the microorganism is given time and conditions to undergo cell division or cell growth.

The term “shelf stability” as used herein refers to the stability of the pharmaceutical composition.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20% plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “a” or “an” as used herein refers to one or to more than one (i.e., to at least one) of the grammatical object of the article. By way of example, “an element” means one element or more than one element.

### Pharmaceutical Compositions

### Pharmaceutically Acceptable Excipient

In some instances, a pharmaceutical composition may comprise a pharmaceutically acceptable excipient.

In some cases, a pharmaceutically acceptable excipient may comprise at least a moisture absorbent material, an adjuvant, an antiadherent, a binder, a carrier, a disintegrant, a filler, a flavor, a color, a diluent, a lubricant, a glidant, a preservative, a sorbent, a solvent, a surfactant, a sweetener, or any combination thereof. In some cases, a pharmaceutically acceptable excipient may comprise a moisture absorbent material. In some cases, a pharmaceutically acceptable excipient may comprise an adjuvant. In some cases, a pharmaceutically acceptable excipient may comprise an antiadherent. In some cases, a pharmaceutically acceptable excipient may comprise a binder. In some cases, a pharmaceutically acceptable excipient may comprise a carrier. In some cases, a pharmaceutically acceptable excipient may comprise a disintegrant. In some cases, a pharmaceutically acceptable excipient may comprise a filler. In some cases, a pharmaceutically acceptable excipient may comprise a flavor. In some cases, a pharmaceutically acceptable excipient may comprise a color. In some cases, a pharmaceutically acceptable excipient may comprise a diluent. In some cases, a pharmaceutically acceptable excipient may comprise a lubricant. In some cases, a pharmaceutically acceptable excipient may comprise a glidant. In some cases, a pharmaceutically acceptable excipient may comprise a preservative. In some cases, a pharmaceutically acceptable excipient may comprise a sorbent. In some cases, a pharmaceutically acceptable excipient may comprise a solvent. In some cases, a pharmaceutically acceptable excipient may comprise a surfactant. In some cases, a pharmaceutically acceptable excipient may comprise a sweetener.

In some cases, a moisture absorbent material may comprise microcrystalline cellulose (MCC), hydroxypropyl methylcellulose, silicon dioxide (SiO₂), polyethylene glycol 8000, lactose, d-trehalose dihydrate, mannitol, calcium phosphate tribasic, calcium sulfate, corn starch, fructose, xylitol, maltitol, anhydrous lactose and dicalcium phosphate (DCP). In some cases, a moisture absorbent material may comprise microcrystalline cellulose (MCC), hydroxypropyl methylcellulose, silicon dioxide (SiO₂), polyethylene glycol 8000, lactose, d-trehalose dihydrate, mannitol, calcium phosphate tribasic, calcium sulfate, corn starch, fructose, xylitol, maltitol, anhydrous lactose or dicalcium phosphate (DCP). In some cases, a moisture absorbent material may comprise SiO₂. In some cases, a moisture absorbent material may comprise MCC. In some cases, a moisture absorbent material may comprise anhydrous lactose. In some cases, a moisture absorbent material may comprise hydroxypropyl methylcellulose. In some cases, a moisture absorbent material may comprise polyethylene glycol 8000. In some cases, a moisture absorbent material may comprise lactose. In some cases, a moisture absorbent material may comprise d-trehalose dihydrate. In some cases, a moisture absorbent material may comprise mannitol. In some cases, a moisture absorbent material may comprise calcium phosphate tribasic. In some cases, a moisture absorbent material may comprise calcium sulfate. In some cases, a moisture absorbent material may comprise corn starch. In some cases, a moisture absorbent material may comprise fructose. In some cases, a moisture absorbent material may comprise xylitol. In some cases, a moisture absorbent material may comprise maltitol. In some cases, a moisture absorbent material may comprise DCP. A pharmaceutically acceptable excipient may be present in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. A pharmaceutically acceptable excipient may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. A pharmaceutically acceptable excipient may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the pharmaceutically acceptable excipient may be measured by weight or volume of the pharmaceutical composition. The percentage of the pharmaceutically acceptable excipient may be measured by weight or volume of the pharmaceutical composition. The quantities of any of the multiple components of the pharmaceutical acceptable excipients or multiple pharmaceutical acceptable excipients may be listed as the ratio between each component of the pharmaceutical acceptable excipients or pharmaceutical acceptable excipient. For example, wherein a first pharmaceutical acceptable excipient and second pharmaceutical acceptable excipient are each present in 1% and 1% of the pharmaceutical composition, the first pharmaceutical acceptable excipient may be listed as 1:1 as the second pharmaceutical acceptable excipient. The way the quantities of the pharmaceutical composition listed may depend on the types and amounts of the pharmaceutical acceptable excipient or other components in the pharmaceutical composition. The percentage of the moisture absorbent material may be measured by weight, volume, weight/volume, or volume/weight of the pharmaceutical composition.

In the case that the pharmaceutically acceptable excipient comprises any more of the components described herein, the percentage may be measured as the percentage for each individual component or the mixture of the components relative to the pharmaceutical composition. A moisture absorbent material may be present in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. A moisture absorbent material may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. A moisture absorbent material may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the moisture absorbent material may be measured by weight, volume, weight/volume, or volume/weight of the pharmaceutical composition.

In some instances, SiO₂ may be present in an amount of about 0.1% to about 10% by weight of a pharmaceutical composition. In some cases, SiO₂ may be present in an amount of about 0.2% to about 9% by weight of a pharmaceutical composition. In some cases, SiO₂ may be present in an amount of about 0.3% to about 8% by weight of a pharmaceutical composition. In some cases, SiO₂ may be present in an amount of about 0.4% to about 7% by weight of a pharmaceutical composition. In some cases, SiO₂ may be present in an amount of about 0.5% to about 6% by weight of a pharmaceutical composition. In some cases, SiO₂ may be present in an amount of about 0.6% to about 5% by weight of a pharmaceutical composition. In some cases, SiO₂ may be present in an amount of about 0.7% to about 4% by weight of a pharmaceutical composition. In some cases, SiO₂ may be present in an amount of about 0.8% to about 3% by weight of a pharmaceutical composition. In some cases, SiO₂ may be present in an amount of about 0.9% to about 2% by weight of a pharmaceutical composition. In some cases, SiO₂ may be present in an amount of about 0.91% to about 1.9% by weight of a pharmaceutical composition. In some cases, SiO₂ may be present in an amount of about 0.92% to about 1.8% by weight of a pharmaceutical composition. In some cases, SiO₂ may be present in an amount of about 0.93% to about 1.7% by weight of a pharmaceutical composition. In some cases, SiO₂ may be present in an amount of about 0.94% to about 1.6% by weight of a pharmaceutical composition. In some cases, SiO₂ may be present in an amount of about 0.95% to about 1.5% by weight of a pharmaceutical composition. In some cases, SiO₂ may be present in an amount of about 0.96% to about 1.4% by weight of a pharmaceutical composition. In some cases, SiO₂ may be present in an amount of about 0.97% to about 1.3% by weight of a pharmaceutical composition. In some cases, SiO₂ may be present in an amount of about 0.98% to about 1.2% by weight of a pharmaceutical composition. In some cases, SiO₂ may be present in an amount of about 0.99% to about 1.1% by weight of a pharmaceutical composition. In some cases, SiO₂ may be present in an amount of about 0.08% to about 10%, of about 0.06% to about 10%, of about 0.04% to about 10%, of about 0.02% to about 10%, of about 0.01% to about 10%, of about 0.001% to about 10%, of about 0.0001% to about 10%, of about 0.1% to about 15%, of about 0.1% to about 20%, of about 0.1% to about 25%, of about 0.1% to about 30%, of about 0.1% to about 35%, of about 0.1% to about 40%, of about 0.1% to about 45%, or of about 0.1% to about 50% by weight of a pharmaceutical composition.

In some instances, SiO₂ may be present in an amount of about 1% by weight of a pharmaceutical composition. In some cases, SiO₂ may be present in an amount of about 1.5% by weight of a pharmaceutical composition. In some cases, SiO₂ may be present in an amount of about 2% by weight of a pharmaceutical composition. In some cases, SiO₂ may be present in an amount of about 2.5% by weight of a pharmaceutical composition. In some cases, SiO₂ may be present in an amount of about 3% by weight of a pharmaceutical composition. In some cases, SiO₂ may be present in an amount of about 3.5% by weight of a pharmaceutical composition. In some cases, SiO₂ may be present in an amount of about 4% by weight of a pharmaceutical composition. In some cases, SiO₂ may be present in an amount of about 4.5% by weight of a pharmaceutical composition. In some cases, SiO₂ may be present in an amount of about 5% by weight of a pharmaceutical composition. In some cases, SiO₂ may be present in an amount of about 5.5% by weight of a pharmaceutical composition. In some cases, SiO₂ may be present in an amount of about 6% by weight of a pharmaceutical composition. In some cases, SiO₂ may be present in an amount of about 6.5% by weight of a pharmaceutical composition. In some cases, SiO₂ may be present in an amount of about 7% by weight of a pharmaceutical composition. In some cases, SiO₂ may be present in an amount of about 7.5% by weight of a pharmaceutical composition. In some cases, SiO₂ may be present in an amount of about 8% by weight of a pharmaceutical composition. In some cases, SiO₂ may be present in an amount of about 8.5% by weight of a pharmaceutical composition. In some cases, SiO₂ may be present in an amount of about 9% by weight of a pharmaceutical composition. In some cases, SiO₂ may be present in an amount of about 9.5% by weight of a pharmaceutical composition. In some cases, SiO₂ may be present in an amount of about 10% by weight of a pharmaceutical composition. In some cases, SiO₂ may be present in an amount of about 0.9% by weight of a pharmaceutical composition. In some cases, SiO₂ may be present in an amount of about 0.8% by weight of a pharmaceutical composition. In some cases, SiO₂ may be present in an amount of about 0.7% by weight of a pharmaceutical composition. In some cases, SiO₂ may be present in an amount of about 0.6% by weight of a pharmaceutical composition. In some cases, SiO₂ may be present in an amount of about 0.5% by weight of a pharmaceutical composition. In some cases, SiO₂ may be present in an amount of about 0.4% by weight of a pharmaceutical composition. In some cases, SiO₂ may be present in an amount of about 0.3% by weight of a pharmaceutical composition. In some cases, SiO₂ may be present in an amount of about 0.2% by weight of a pharmaceutical composition. In some cases, SiO₂ may be present in an amount of about 0.1% by weight of a pharmaceutical composition.

In some cases, mannitol may be present in an amount of about 1% to about 90% by weight of a pharmaceutical composition. In some cases, mannitol may be present in an amount of about 1.25% to about 90% by weight of a pharmaceutical composition. In some cases, mannitol may be present in an amount of about 1.5% to about 90% by weight of a pharmaceutical composition. In some cases, mannitol may be present in an amount of about 1.75% to about 90% by weight of a pharmaceutical composition. In some cases, mannitol may be present in an amount of about 2% to about 90% by weight of a pharmaceutical composition. In some cases, mannitol may be present in an amount of about 2.25% to about 90% by weight of a pharmaceutical composition. In some cases, mannitol may be present in an amount of about 2.5% to about 90% by weight of a pharmaceutical composition. In some cases, mannitol may be present in an amount of about 2.75% to about 90% by weight of a pharmaceutical composition. In some cases, mannitol may be present in an amount of about 3% to about 90% by weight of a pharmaceutical composition. In some cases, mannitol may be present in an amount of about 3.25% to about 90% by weight of a pharmaceutical composition. In some cases, mannitol may be present in an amount of about 3.5% to about 90% by weight of a pharmaceutical composition. In some cases, mannitol may be present in an amount of about 3.75% to about 90% by weight of a pharmaceutical composition. In some cases, mannitol may be present in an amount of about 4% to about 90% by weight of a pharmaceutical composition. In some cases, mannitol may be present in an amount of about 4.25% to about 90% by weight of a pharmaceutical composition. In some cases, mannitol may be present in an amount of about 4.5% to about 90% by weight of a pharmaceutical composition. In some cases, mannitol may be present in an amount of about 4.75% to about 90% by weight of a pharmaceutical composition. In some cases, mannitol may be present in an amount of about 5% to about 90% by weight of a pharmaceutical composition. In some cases, mannitol may be present in an amount of about 5% to about 89.5% by weight of a pharmaceutical composition. In some cases, mannitol may be present in an amount of about 5% to about 89% by weight of a pharmaceutical composition. In some cases, mannitol may be present in an amount of about 5% to about 88.5% by weight of a pharmaceutical composition. In some cases, mannitol may be present in an amount of about 5% to about 88% by weight of a pharmaceutical composition. In some cases, mannitol may be present in an amount of about 5% to about 87.5% by weight of a pharmaceutical composition. In some cases, mannitol may be present in an amount of about 5% to about 87% by weight of a pharmaceutical composition. In some cases, mannitol may be present in an amount of about 5% to about 86.5% by weight of a pharmaceutical composition. In some cases, mannitol may be present in an amount of about 5% to about 86% by weight of a pharmaceutical composition. In some cases, mannitol may be present in an amount of about 5% to about 85.5% by weight of a pharmaceutical composition. In some cases, mannitol may be present in an amount of about 5% to about 85% by weight of a pharmaceutical composition. In some cases, mannitol may be present in an amount of about 5% to about 84.5% by weight of a pharmaceutical composition. In some cases, mannitol may be present in an amount of about 5% to about 84% by weight of a pharmaceutical composition. In some cases, mannitol may be present in an amount of about 5% to about 83.5% by weight of a pharmaceutical composition. In some cases, mannitol may be present in an amount of about 5% to about 83% by weight of a pharmaceutical composition. In some cases, mannitol may be present in an amount of about 5% to about 82.5% by weight of a pharmaceutical composition. In some cases, mannitol may be present in an amount of about 5% to about 82% by weight of a pharmaceutical composition. In some cases, mannitol may be present in an amount of about 5% to about 81.5% by weight of a pharmaceutical composition. In some cases, mannitol may be present in an amount of about 1% to about 91%, about 1% to about 92%, about 1% to about 93%, about 1% to about 94%, about 1% to about 95%, about 1% to about 96%, about 1% to about 97%, about 1% to about 98%, about 1% to about 99%, about 0.1% to about 99%, about 0.01% to about 99%, or about 0.001% to about 99% by weight of a pharmaceutical composition.

In some cases, mannitol may be present in an amount of about 1% by weight of a pharmaceutical composition. In some cases, mannitol may be present in an amount of about 5% by weight of a pharmaceutical composition. In some cases, mannitol may be present in an amount of about 10% by weight of a pharmaceutical composition. In some cases, mannitol may be present in an amount of about 15% by weight of a pharmaceutical composition. In some cases, mannitol may be present in an amount of about 20% by weight of a pharmaceutical composition. In some cases, mannitol may be present in an amount of about 25% by weight of a pharmaceutical composition. In some cases, mannitol may be present in an amount of about 30% by weight of a pharmaceutical composition. In some cases, mannitol may be present in an amount of about 35% by weight of a pharmaceutical composition. In some cases, mannitol may be present in an amount of about 40% by weight of a pharmaceutical composition. In some cases, mannitol may be present in an amount of about 45% by weight of a pharmaceutical composition. In some cases, mannitol may be present in an amount of about 50% by weight of a pharmaceutical composition. In some cases, mannitol may be present in an amount of about 55% by weight of a pharmaceutical composition. In some cases, mannitol may be present in an amount of about 60% by weight of a pharmaceutical composition. In some cases, mannitol may be present in an amount of about 65% by weight of a pharmaceutical composition. In some cases, mannitol may be present in an amount of about 70% by weight of a pharmaceutical composition. In some cases, mannitol may be present in an amount of about 75% by weight of a pharmaceutical composition. In some cases, mannitol may be present in an amount of about 80% by weight of a pharmaceutical composition. In some cases, mannitol may be present in an amount of about 85% by weight of a pharmaceutical composition. In some cases, mannitol may be present in an amount of about 90% by weight of a pharmaceutical composition. In some cases, mannitol may be present in an amount of about 95% by weight of a pharmaceutical composition. In some cases, mannitol may be present in an amount of about 96% by weight of a pharmaceutical composition. In some cases, mannitol may be present in an amount of about 97% by weight of a pharmaceutical composition. In some cases, mannitol may be present in an amount of about 98% by weight of a pharmaceutical composition. In some cases, mannitol may be present in an amount of about 99% by weight of a pharmaceutical composition. In some cases, mannitol may be present in a SP. Quantity Sufficient (QS) amount of a pharmaceutical composition. For example, if a pharmaceutical composition is N mg and the other components of the pharmaceutical composition is x mg, then mannitol may be N-x (mg). The percentage of the moisture absorbent material may be measured by weight, volume, weight/volume, or volume/weight of the pharmaceutical composition. Any of these units can substitute the unit of the equation above depending on the unit used.

In some cases, anhydrous lactose may be present in an amount of about 1% to about 90% by weight of a pharmaceutical composition. In some cases, anhydrous lactose may be present in an amount of about 1.25% to about 90% by weight of a pharmaceutical composition. In some cases, anhydrous lactose may be present in an amount of about 1.5% to about 90% by weight of a pharmaceutical composition. In some cases, anhydrous lactose may be present in an amount of about 1.75% to about 90% by weight of a pharmaceutical composition. In some cases, anhydrous lactose may be present in an amount of about 2% to about 90% by weight of a pharmaceutical composition. In some cases, anhydrous lactose may be present in an amount of about 2.25% to about 90% by weight of a pharmaceutical composition. In some cases, anhydrous lactose may be present in an amount of about 2.5% to about 90% by weight of a pharmaceutical composition. In some cases, anhydrous lactose may be present in an amount of about 2.75% to about 90% by weight of a pharmaceutical composition. In some cases, anhydrous lactose may be present in an amount of about 3% to about 90% by weight of a pharmaceutical composition. In some cases, anhydrous lactose may be present in an amount of about 3.25% to about 90% by weight of a pharmaceutical composition. In some cases, anhydrous lactose may be present in an amount of about 3.5% to about 90% by weight of a pharmaceutical composition. In some cases, anhydrous lactose may be present in an amount of about 3.75% to about 90% by weight of a pharmaceutical composition. In some cases, anhydrous lactose may be present in an amount of about 4% to about 90% by weight of a pharmaceutical composition. In some cases, anhydrous lactose may be present in an amount of about 4.25% to about 90% by weight of a pharmaceutical composition. In some cases, anhydrous lactose may be present in an amount of about 4.5% to about 90% by weight of a pharmaceutical composition. In some cases, anhydrous lactose may be present in an amount of about 4.75% to about 90% by weight of a pharmaceutical composition. In some cases, anhydrous lactose may be present in an amount of about 5% to about 90% by weight of a pharmaceutical composition. In some cases, anhydrous lactose may be present in an amount of about 5% to about 89.5% by weight of a pharmaceutical composition. In some cases, anhydrous lactose may be present in an amount of about 5% to about 89% by weight of a pharmaceutical composition. In some cases, anhydrous lactose may be present in an amount of about 5% to about 88.5% by weight of a pharmaceutical composition. In some cases, anhydrous lactose may be present in an amount of about 5% to about 88% by weight of a pharmaceutical composition. In some cases, anhydrous lactose may be present in an amount of about 5% to about 87.5% by weight of a pharmaceutical composition. In some cases, anhydrous lactose may be present in an amount of about 5% to about 87% by weight of a pharmaceutical composition. In some cases, anhydrous lactose may be present in an amount of about 5% to about 86.5% by weight of a pharmaceutical composition. In some cases, anhydrous lactose may be present in an amount of about 5% to about 86% by weight of a pharmaceutical composition. In some cases, anhydrous lactose may be present in an amount of about 5% to about 85.5% by weight of a pharmaceutical composition. In some cases, anhydrous lactose may be present in an amount of about 5% to about 85% by weight of a pharmaceutical composition. In some cases, anhydrous lactose may be present in an amount of about 5% to about 84.5% by weight of a pharmaceutical composition. In some cases, anhydrous lactose may be present in an amount of about 5% to about 84% by weight of a pharmaceutical composition. In some cases, anhydrous lactose may be present in an amount of about 5% to about 83.5% by weight of a pharmaceutical composition. In some cases, anhydrous lactose may be present in an amount of about 5% to about 83% by weight of a pharmaceutical composition. In some cases, anhydrous lactose may be present in an amount of about 5% to about 82.5% by weight of a pharmaceutical composition. In some cases, anhydrous lactose may be present in an amount of about 5% to about 82% by weight of a pharmaceutical composition. In some cases, anhydrous lactose may be present in an amount of about 5% to about 81.5% by weight of a pharmaceutical composition. In some cases, anhydrous lactose may be present in an amount of about 1% to about 91%, about 1% to about 92%, about 1% to about 93%, about 1% to about 94%, about 1% to about 95%, about 1% to about 96%, about 1% to about 97%, about 1% to about 98%, about 1% to about 99%, about 0.1% to about 99%, about 0.01% to about 99%, or about 0.001% to about 99% by weight of a pharmaceutical composition.

In some cases, anhydrous lactose may be present in an amount of about 1% by weight of a pharmaceutical composition. In some cases, anhydrous lactose may be present in an amount of about 5% by weight of a pharmaceutical composition. In some cases, anhydrous lactose may be present in an amount of about 10% by weight of a pharmaceutical composition. In some cases, anhydrous lactose may be present in an amount of about 15% by weight of a pharmaceutical composition. In some cases, anhydrous lactose may be present in an amount of about 20% by weight of a pharmaceutical composition. In some cases, anhydrous lactose may be present in an amount of about 25% by weight of a pharmaceutical composition. In some cases, anhydrous lactose may be present in an amount of about 30% by weight of a pharmaceutical composition. In some cases, anhydrous lactose may be present in an amount of about 35% by weight of a pharmaceutical composition. In some cases, anhydrous lactose may be present in an amount of about 40% by weight of a pharmaceutical composition. In some cases, anhydrous lactose may be present in an amount of about 45% by weight of a pharmaceutical composition. In some cases, anhydrous lactose may be present in an amount of about 50% by weight of a pharmaceutical composition. In some cases, anhydrous lactose may be present in an amount of about 55% by weight of a pharmaceutical composition. In some cases, anhydrous lactose may be present in an amount of about 60% by weight of a pharmaceutical composition. In some cases, anhydrous lactose may be present in an amount of about 65% by weight of a pharmaceutical composition. In some cases, anhydrous lactose may be present in an amount of about 70% by weight of a pharmaceutical composition. In some cases, anhydrous lactose may be present in an amount of about 75% by weight of a pharmaceutical composition. In some cases, anhydrous lactose may be present in an amount of about 80% by weight of a pharmaceutical composition. In some cases, anhydrous lactose may be present in an amount of about 85% by weight of a pharmaceutical composition. In some cases, anhydrous lactose may be present in an amount of about 90% by weight of a pharmaceutical composition. In some cases, anhydrous lactose may be present in an amount of about 95% by weight of a pharmaceutical composition. In some cases, anhydrous lactose may be present in an amount of about 96% by weight of a pharmaceutical composition. In some cases, anhydrous lactose may be present in an amount of about 97% by weight of a pharmaceutical composition. In some cases, anhydrous lactose may be present in an amount of about 98% by weight of a pharmaceutical composition. In some cases, anhydrous lactose may be present in an amount of about 99% by weight of a pharmaceutical composition. In some cases, anhydrous lactose may be present in a Quantity Sufficient (QS) amount of a pharmaceutical composition. For example, if a pharmaceutical composition is N mg and the other components of the pharmaceutical composition is x mg, then anhydrous lactose may be N-x (mg). The percentage of the moisture absorbent material may be measured by weight, volume, weight/volume, or volume/weight of the pharmaceutical composition. Any of these units can substitute the unit of the equation above depending on the unit used.

In some instances, a pharmaceutical composition may comprise mannitol and anhydrous lactose. In some cases, mannitol and anhydrous lactose may be present in an amount of about 1% to about 90% by weight of a pharmaceutical composition. In some cases, mannitol and anhydrous lactose may be present in an amount of about 1.25% to about 90% by weight of a pharmaceutical composition. In some cases, mannitol and anhydrous lactose may be present in an amount of about 1.5% to about 90% by weight of a pharmaceutical composition. In some cases, mannitol and anhydrous lactose may be present in an amount of about 1.75% to about 90% by weight of a pharmaceutical composition. In some cases, mannitol and anhydrous lactose may be present in an amount of about 2% to about 90% by weight of a pharmaceutical composition. In some cases, mannitol and anhydrous lactose may be present in an amount of about 2.25% to about 90% by weight of a pharmaceutical composition. In some cases, mannitol and anhydrous lactose may be present in an amount of about 2.5% to about 90% by weight of a pharmaceutical composition. In some cases, mannitol and anhydrous lactose may be present in an amount of about 2.75% to about 90% by weight of a pharmaceutical composition. In some cases, mannitol and anhydrous lactose may be present in an amount of about 3% to about 90% by weight of a pharmaceutical composition. In some cases, mannitol and anhydrous lactose may be present in an amount of about 3.25% to about 90% by weight of a pharmaceutical composition. In some cases, mannitol and anhydrous lactose may be present in an amount of about 3.5% to about 90% by weight of a pharmaceutical composition. In some cases, mannitol and anhydrous lactose may be present in an amount of about 3.75% to about 90% by weight of a pharmaceutical composition. In some cases, mannitol and anhydrous lactose may be present in an amount of about 4% to about 90% by weight of a pharmaceutical composition. In some cases, mannitol and anhydrous lactose may be present in an amount of about 4.25% to about 90% by weight of a pharmaceutical composition. In some cases, mannitol and anhydrous lactose may be present in an amount of about 4.5% to about 90% by weight of a pharmaceutical composition. In some cases, mannitol and anhydrous lactose may be present in an amount of about 4.75% to about 90% by weight of a pharmaceutical composition. In some cases, mannitol and anhydrous lactose may be present in an amount of about 5% to about 90% by weight of a pharmaceutical composition. In some cases, mannitol and anhydrous lactose may be present in an amount of about 5% to about 89.5% by weight of a pharmaceutical composition. In some cases, mannitol and anhydrous lactose may be present in an amount of about 5% to about 89% by weight of a pharmaceutical composition. In some cases, mannitol and anhydrous lactose may be present in an amount of about 5% to about 88.5% by weight of a pharmaceutical composition. In some cases, mannitol and anhydrous lactose may be present in an amount of about 5% to about 88% by weight of a pharmaceutical composition. In some cases, mannitol and anhydrous lactose may be present in an amount of about 5% to about 87.5% by weight of a pharmaceutical composition. In some cases, mannitol and anhydrous lactose may be present in an amount of about 5% to about 87% by weight of a pharmaceutical composition. In some cases, mannitol and anhydrous lactose may be present in an amount of about 5% to about 86.5% by weight of a pharmaceutical composition. In some cases, mannitol and anhydrous lactose may be present in an amount of about 5% to about 86% by weight of a pharmaceutical composition. In some cases, mannitol and anhydrous lactose may be present in an amount of about 5% to about 85.5% by weight of a pharmaceutical composition. In some cases, mannitol and anhydrous lactose may be present in an amount of about 5% to about 85% by weight of a pharmaceutical composition. In some cases, mannitol and anhydrous lactose may be present in an amount of about 5% to about 84.5% by weight of a pharmaceutical composition. In some cases, mannitol and anhydrous lactose may be present in an amount of about 5% to about 84% by weight of a pharmaceutical composition. In some cases, mannitol and anhydrous lactose may be present in an amount of about 5% to about 83.5% by weight of a pharmaceutical composition. In some cases, mannitol and anhydrous lactose may be present in an amount of about 5% to about 83% by weight of a pharmaceutical composition. In some cases, mannitol and anhydrous lactose may be present in an amount of about 5% to about 82.5% by weight of a pharmaceutical composition. In some cases, mannitol and anhydrous lactose may be present in an amount of about 5% to about 82% by weight of a pharmaceutical composition. In some cases, mannitol and anhydrous lactose may be present in an amount of about 5% to about 81.5% by weight of a pharmaceutical composition. In some cases, mannitol and anhydrous lactose may be present in an amount of about 1% to about 91%, about 1% to about 92%, about 1% to about 93%, about 1% to about 94%, about 1% to about 95%, about 1% to about 96%, about 1% to about 97%, about 1% to about 98%, about 1% to about 99%, about 0.1% to about 99%, about 0.01% to about 99%, or about 0.001% to about 99% by weight of a pharmaceutical composition.

In some cases, mannitol and anhydrous lactose may be present in an amount of about 1% by weight of a pharmaceutical composition. In some cases, mannitol and anhydrous lactose may be present in an amount of about 5% by weight of a pharmaceutical composition. In some cases, mannitol and anhydrous lactose may be present in an amount of about 10% by weight of a pharmaceutical composition. In some cases, mannitol and anhydrous lactose may be present in an amount of about 15% by weight of a pharmaceutical composition. In some cases, mannitol and anhydrous lactose may be present in an amount of about 20% by weight of a pharmaceutical composition. In some cases, mannitol and anhydrous lactose may be present in an amount of about 25% by weight of a pharmaceutical composition. In some cases, mannitol and anhydrous lactose may be present in an amount of about 30% by weight of a pharmaceutical composition. In some cases, mannitol and anhydrous lactose may be present in an amount of about 35% by weight of a pharmaceutical composition. In some cases, mannitol and anhydrous lactose may be present in an amount of about 40% by weight of a pharmaceutical composition. In some cases, mannitol and anhydrous lactose may be present in an amount of about 45% by weight of a pharmaceutical composition. In some cases, mannitol and anhydrous lactose may be present in an amount of about 50% by weight of a pharmaceutical composition. In some cases, mannitol and anhydrous lactose may be present in an amount of about 55% by weight of a pharmaceutical composition. In some cases, mannitol and anhydrous lactose may be present in an amount of about 60% by weight of a pharmaceutical composition. In some cases, mannitol and anhydrous lactose may be present in an amount of about 65% by weight of a pharmaceutical composition. In some cases, mannitol and anhydrous lactose may be present in an amount of about 70% by weight of a pharmaceutical composition. In some cases, mannitol and anhydrous lactose may be present in an amount of about 75% by weight of a pharmaceutical composition. In some cases, mannitol and anhydrous lactose may be present in an amount of about 80% by weight of a pharmaceutical composition. In some cases, mannitol and anhydrous lactose may be present in an amount of about 85% by weight of a pharmaceutical composition. In some cases, mannitol and anhydrous lactose may be present in an amount of about 90% by weight of a pharmaceutical composition. In some cases, mannitol and anhydrous lactose may be present in an amount of about 95% by weight of a pharmaceutical composition. In some cases, mannitol and anhydrous lactose may be present in an amount of about 96% by weight of a pharmaceutical composition. In some cases, mannitol and anhydrous lactose may be present in an amount of about 97% by weight of a pharmaceutical composition. In some cases, mannitol and anhydrous lactose may be present in an amount of about 98% by weight of a pharmaceutical composition. In some cases, mannitol and anhydrous lactose may be present in an amount of about 99% by weight of a pharmaceutical composition. In some cases, mannitol and anhydrous lactose may be present in a Quantity Sufficient (QS) amount of a pharmaceutical composition. For example, if a pharmaceutical composition is N mg and the other components of the pharmaceutical composition is x mg, then mannitol and anhydrous lactose may be N-x (mg). In some cases, when a pharmaceutical composition comprises mannitol and anhydrous lactose, the amount of mannitol and the amount of anhydrous lactose may be interchangeable. In some cases, the amount of mannitol and the amount of anhydrous lactose may vary, wherein the total amount of mannitol and anhydrous lactose may be any amount of mannitol and anhydrous lactose described thereof. The percentage of the moisture absorbent material may be measured by weight, volume, weight/volume, or volume/weight of the pharmaceutical composition. Any of these units can substitute the unit of the equation above depending on the unit used.

In some instances, a pharmaceutical composition may comprise magnesium stearate. In some cases, magnesium stearate may be present in an amount of about 0.1% to about 10% by weight of a pharmaceutical composition. In some cases, magnesium stearate may be present in an amount of about 0.2% to about 9% by weight of a pharmaceutical composition. In some cases, magnesium stearate may be present in an amount of about 0.3% to about 8% by weight of a pharmaceutical composition. In some cases, magnesium stearate may be present in an amount of about 0.4% to about 7% by weight of a pharmaceutical composition. In some cases, magnesium stearate may be present in an amount of about 0.5% to about 6% by weight of a pharmaceutical composition. In some cases, magnesium stearate may be present in an amount of about 0.6% to about 5% by weight of a pharmaceutical composition. In some cases, magnesium stearate may be present in an amount of about 0.7% to about 4% by weight of a pharmaceutical composition. In some cases, magnesium stearate may be present in an amount of about 0.8% to about 3% by weight of a pharmaceutical composition. In some cases, magnesium stearate may be present in an amount of about 0.9% to about 2% by weight of a pharmaceutical composition. In some cases, magnesium stearate may be present in an amount of about 0.91% to about 1.9% by weight of a pharmaceutical composition. In some cases, magnesium stearate may be present in an amount of about 0.92% to about 1.8% by weight of a pharmaceutical composition. In some cases, magnesium stearate may be present in an amount of about 0.93% to about 1.7% by weight of a pharmaceutical composition. In some cases, magnesium stearate may be present in an amount of about 0.94% to about 1.6% by weight of a pharmaceutical composition. In some cases, magnesium stearate may be present in an amount of about 0.95% to about 1.5% by weight of a pharmaceutical composition. In some cases, magnesium stearate may be present in an amount of about 0.96% to about 1.4% by weight of a pharmaceutical composition. In some cases, magnesium stearate may be present in an amount of about 0.97% to about 1.3% by weight of a pharmaceutical composition. In some cases, magnesium stearate may be present in an amount of about 0.98% to about 1.2% by weight of a pharmaceutical composition. In some cases, magnesium stearate may be present in an amount of about 0.99% to about 1.1% by weight of a pharmaceutical composition. In some cases, magnesium stearate may be present in an amount of about 0.08% to about 10%, of about 0.06% to about 10%, of about 0.04% to about 10%, of about 0.02% to about 10%, of about 0.01% to about 10%, of about 0.001% to about 10%, of about 0.0001% to about 10%, of about 0.1% to about 15%, of about 0.1% to about 200%, of about 0.1% to about 25%, of about 0.1% to about 30%, of about 0.1% to about 35%, of about 0.1% to about 40%, of about 0.1% to about 45%, or of about 0.1% to about 50% by weight of a pharmaceutical composition.

In some instances, magnesium stearate may be present in an amount of about 1% by weight of a pharmaceutical composition. In some cases, magnesium stearate may be present in an amount of about 1.5% by weight of a pharmaceutical composition. In some cases, magnesium stearate may be present in an amount of about 2% by weight of a pharmaceutical composition. In some cases, magnesium stearate may be present in an amount of about 2.5% by weight of a pharmaceutical composition. In some cases, magnesium stearate may be present in an amount of about 3% by weight of a pharmaceutical composition. In some cases, magnesium stearate may be present in an amount of about 3.5% by weight of a pharmaceutical composition. In some cases, magnesium stearate may be present in an amount of about 4% by weight of a pharmaceutical composition. In some cases, magnesium stearate may be present in an amount of about 4.5% by weight of a pharmaceutical composition. In some cases, magnesium stearate may be present in an amount of about 5% by weight of a pharmaceutical composition. In some cases, magnesium stearate may be present in an amount of about 5.5% by weight of a pharmaceutical composition. In some cases, magnesium stearate may be present in an amount of about 6% by weight of a pharmaceutical composition. In some cases, magnesium stearate may be present in an amount of about 6.5% by weight of a pharmaceutical composition. In some cases, magnesium stearate may be present in an amount of about 7% by weight of a pharmaceutical composition. In some cases, magnesium stearate may be present in an amount of about 7.5% by weight of a pharmaceutical composition. In some cases, magnesium stearate may be present in an amount of about 8% by weight of a pharmaceutical composition. In some cases, magnesium stearate may be present in an amount of about 8.5% by weight of a pharmaceutical composition. In some cases, magnesium stearate may be present in an amount of about 9% by weight of a pharmaceutical composition. In some cases, magnesium stearate may be present in an amount of about 9.5% by weight of a pharmaceutical composition. In some cases, magnesium stearate may be present in an amount of about 10% by weight of a pharmaceutical composition. In some cases, magnesium stearate may be present in an amount of about 0.9% by weight of a pharmaceutical composition. In some cases, magnesium stearate may be present in an amount of about 0.8% by weight of a pharmaceutical composition. In some cases, magnesium stearate may be present in an amount of about 0.7% by weight of a pharmaceutical composition. In some cases, magnesium stearate may be present in an amount of about 0.6% by weight of a pharmaceutical composition. In some cases, magnesium stearate may be present in an amount of about 0.5% by weight of a pharmaceutical composition. In some cases, magnesium stearate may be present in an amount of about 0.4% by weight of a pharmaceutical composition. In some cases, magnesium stearate may be present in an amount of about 0.3% by weight of a pharmaceutical composition. In some cases, magnesium stearate may be present in an amount of about 0.2% by weight of a pharmaceutical composition. In some cases, magnesium stearate may be present in an amount of about 0.1% by weight of a pharmaceutical composition.

In some cases, a pharmaceutically acceptable excipient may be present in an amount of about 0.0001% to about 10%, about 0.001% to about 10%, about 0.001% to about 99%, about 0.01% to about 10%, about 0.01% to about 99%, about 0.02% to about 10%, about 0.04% to about 10%, about 0.06% to about 10%, about 0.08% to about 10%, about 0.1% to about 15%, about 0.1% to about 20%, about 0.1% to about 25%, about 0.1% to about 30%, about 0.1% to about 35%, about 0.1% to about 40%, about 0.1% to about 45%, about 0.1% to about 50%, about 0.1% to about 99%, about 0.1% to about 10%, about 0.2% to about 9%, about 0.3% to about 8%, about 0.4% to about 7%, about 0.5% to about 6%, about 0.6% to about 5%, about 0.7% to about 4%, about 0.8% to about 3%, about 0.9% to about 2%, about 0.91% to about 1.9%, about 0.92% to about 1.8%, about 0.93% to about 1.7%, about 0.94% to about 1.6%, about 0.95% to about 1.5%, about 0.96% to about 1.4%, about 0.97% to about 1.3%, about 0.98% to about 1.2%, about 0.99% to about 1.1%, about 1% to about 91%, about 1% to about 92%, about 1% to about 93%, about 1% to about 94%, about 1% to about 95%, about 1% to about 96%, about 1% to about 97%, about 1% to about 98%, about 1% to about 99%, about 1.25% to about 90%, about 1.5% to about 90%, about 1.75% to about 90%, about 2% to about 90%, about 2.25% to about 90%, about 2.5% to about 90%, about 2.75% to about 90%, about 3% to about 90%, about 3.25% to about 90%, about 3.5% to about 90%, about 3.75% to about 90%, about 4% to about 90%, about 4.25% to about 90%, about 4.5% to about 90%, about 4.75% to about 90%, about 5% to about 90%, about 5% to about 89.5%, about 5% to about 89%, about 5% to about 88.5%, about 5% to about 88%, about 5% to about 87.5%, about 5% to about 87%, about 5% to about 86.5%, about 5% to about 86%, about 5% to about 85.5%, about 5% to about 85%, about 5% to about 84.5%, about 5% to about 84%, about 5% to about 83.5%, about 5% to about 83%, about 5% to about 82.5%, about 5% to about 82%, or about 5% to about 81.5% of a pharmaceutical composition. In some cases, a pharmaceutical acceptable excipient may comprise about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 0.9%, about 0.8%, about 0.7%, about 0.6%, about 0.5%, about 0.4%, about 0.3%, about 0.2%, about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of a pharmaceutical composition. In some cases, a pharmaceutical acceptable excipient may be present in a QQS. amount of a pharmaceutical composition. For example, if a pharmaceutical composition is N mg and the other components of the pharmaceutical composition is x mg, then mannitol may be N-x (mg). The percentage of the moisture absorbent material may be measured by weight, volume, weight/volume, or volume/weight of the pharmaceutical composition. Any of these units can substitute the unit of the equation above depending on the unit used. The pharmaceutical acceptable excipient. In some cases, the pharmaceutically acceptable excipients may also comprise maltodextrin, cellulose, methionine, ascorbic acid, magnesium stearate, beta-cyclodextrin, dextrin, 2-Hydroxypropyl-β-cyclodextrin, cysteine, riboflavin, starch, Glucidex, mannitol, saccharose, trehalose, anhydrous lactose, or a combination thereof. In some cases, the pharmaceutically acceptable excipients may comprise maltodextrin. In some cases, the pharmaceutically acceptable excipients may comprise cellulose. In some cases, the pharmaceutically acceptable excipients may comprise methionine. In some cases, the pharmaceutically acceptable excipients may comprise ascorbic acid. In some cases, the pharmaceutically acceptable excipients may comprise magnesium stearate. In some cases, the pharmaceutically acceptable excipients may comprise beta-cyclodextrin. In some cases, the pharmaceutically acceptable excipients may comprise dextrin. In some cases, the pharmaceutically acceptable excipients may comprise 2-Hydroxypropyl-β-cyclodextrin. In some cases, the pharmaceutically acceptable excipients may comprise cysteine. In some cases, the pharmaceutically acceptable excipients may comprise riboflavin. In some cases, the pharmaceutically acceptable excipients may comprise starch. In some cases, the pharmaceutically acceptable excipients may comprise Glucidex. In some cases, the pharmaceutically acceptable excipients may comprise mannitol. In some cases, the pharmaceutically acceptable excipients may comprise saccharose. In some cases, the pharmaceutically acceptable excipients may comprise trehalose. In some cases, the pharmaceutically acceptable excipients may comprise anhydrous lactose. The pharmaceutically acceptable excipient may be present in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. The pharmaceutically acceptable excipient may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition.

In some cases, a moisture absorbent material may be present in an amount of about 0.0001% to about 10%, about 0.001% to about 10%, about 0.001% to about 99%, about 0.01% to about 10%, about 0.01% to about 99%, about 0.02% to about 10%, about 0.04% to about 10%, about 0.06% to about 10%, about 0.08% to about 10%, about 0.1% to about 15%, about 0.1% to about 20%, about 0.1% to about 25%, about 0.1% to about 30%, about 0.1% to about 35%, about 0.1% to about 40%, about 0.1% to about 45%, about 0.1% to about 50%, about 0.1% to about 99%, about 0.1% to about 10%, about 0.2% to about 9%, about 0.3% to about 8%, about 0.4% to about 7%, about 0.5% to about 6%, about 0.6% to about 5%, about 0.7% to about 4%, about 0.8% to about 3%, about 0.9% to about 2%, about 0.91% to about 1.9%, about 0.92% to about 1.8%, about 0.93% to about 1.7%, about 0.94% to about 1.6%, about 0.95% to about 1.5%, about 0.96% to about 1.4%, about 0.97% to about 1.3%, about 0.98% to about 1.2%, about 0.99% to about 1.1%, about 1% to about 91%, about 1% to about 92%, about 1% to about 93%, about 1% to about 94%, about 1% to about 95%, about 1% to about 96%, about 1% to about 97%, about 1% to about 98%, about 1% to about 99%, about 1.25% to about 90%, about 1.5% to about 90%, about 1.75% to about 90%, about 2% to about 90%, about 2.25% to about 90%, about 2.5% to about 90%, about 2.75% to about 90%, about 3% to about 90%, about 3.25% to about 90%, about 3.5% to about 90%, about 3.75% to about 90%, about 4% to about 90%, about 4.25% to about 90%, about 4.5% to about 90%, about 4.75% to about 90%, about 5% to about 90%, about 5% to about 89.5%, about 5% to about 89%, about 5% to about 88.5%, about 5% to about 88%, about 5% to about 87.5%, about 5% to about 87%, about 5% to about 86.5%, about 5% to about 86%, about 5% to about 85.5%, about 5% to about 85%, about 5% to about 84.5%, about 5% to about 84%, about 5% to about 83.5%, about 5% to about 83%, about 5% to about 82.5%, about 5% to about 82%, or about 5% to about 81.5% of a pharmaceutical composition. In some cases, a moisture absorbent material may comprise about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 0.9% about 0.8%, about 0.7%, about 0.6%, about 0.5%, about 0.4%, about 0.3%, about 0.2%, about 0.1%, about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of a pharmaceutical composition.

In some instances, a pharmaceutical composition may comprise SiO₂ in an amount of about 0.1% to about 10% by weight of the pharmaceutical composition, mannitol in an amount of about 1% to about 90% of the pharmaceutical composition, and magnesium stearate in an amount of 0.1% to 10% by weight of the pharmaceutical composition. In some cases, a pharmaceutical composition may comprise SiO₂ in an amount of about 0.1% to about 10% by weight of the pharmaceutical composition and mannitol in an amount of about 1% to about 90% of the pharmaceutical composition. In some instances, a pharmaceutical composition may comprise SiO₂ in an amount of about 1% by weight of the pharmaceutical composition, mannitol in an amount of about 5% to about 81.5% of the pharmaceutical composition, and magnesium stearate in an amount of 1.5% by weight of the pharmaceutical composition. In some cases, a pharmaceutical composition may comprise SiO₂ in an amount of about 1% by weight of the pharmaceutical composition and mannitol in an amount of about 5% to about 81.5% of the pharmaceutical composition.

In some instances, a pharmaceutical composition may comprise SiO₂ in an amount of about 0.1% to about 10% by weight of the pharmaceutical composition, anhydrous lactose in an amount of about 1% to about 90% of the pharmaceutical composition, and magnesium stearate in an amount of 0.1% to 10% by weight of the pharmaceutical composition. In some cases, a pharmaceutical composition may comprise SiO₂ in an amount of about 0.1% to about 10% by weight of the pharmaceutical composition and anhydrous lactose in an amount of about 1% to about 90% of the pharmaceutical composition. In some instances, a pharmaceutical composition may comprise SiO₂ in an amount of about 1% by weight of the pharmaceutical composition, anhydrous lactose in an amount of about 5% to about 81.5% of the pharmaceutical composition, and magnesium stearate in an amount of 1.5% by weight of the pharmaceutical composition. In some cases, a pharmaceutical composition may comprise SiO₂ in an amount of about 1% by weight of the pharmaceutical composition and anhydrous lactose in an amount of about 5% to about 81.5% of the pharmaceutical composition.

In some instances, a pharmaceutical composition may comprise SiO₂ in an amount of about 0.1% to about 10% by weight of the pharmaceutical composition, mannitol and anhydrous lactose in an amount of about 1% to about 90% of the pharmaceutical composition, and magnesium stearate in an amount of 0.1% to 10% by weight of the pharmaceutical composition. In some cases, a pharmaceutical composition may comprise SiO₂ in an amount of about 0.1% to about 10% by weight of the pharmaceutical composition and mannitol and anhydrous lactose in an amount of about 1% to about 90% of the pharmaceutical composition. In some instances, a pharmaceutical composition may comprise SiO₂ in an amount of about 1% by weight of the pharmaceutical composition, mannitol and anhydrous lactose in an amount of about 5% to about 81.5% of the pharmaceutical composition, and magnesium stearate in an amount of 1.5% by weight of the pharmaceutical composition. In some cases, a pharmaceutical composition may comprise SiO₂ in an amount of about 1% by weight of the pharmaceutical composition and mannitol and anhydrous lactose in an amount of about 5% to about 81.5% of the pharmaceutical composition.

### Bacterial Populations

In some instances, a bacterial population may comprise at least one strain of Lactobacillus sp., at least one strain of Akkermansia sp., or at least one strain of Faecalibacterium sp. In some instances, a bacterial population may comprise at least one strain of a species of the Lactobacillaceae family, at least one strain of Akkermansia sp., or at least one strain of Faecalibacterium sp. In some instances, a bacterial population may comprise at least two of: at least one strain of Lactobacillus sp., at least one strain of Akkermansia sp., or at least one strain of Faecalibacterium sp. In some instances, a bacterial population may comprise at least two of: at least one strain of a species of the Lactobacillaceae family, at least one strain of Akkermansia sp., or at least one strain of Faecalibacterium sp. In some cases, a bacterial population may comprise at least one strain of Lactobacillus sp., at least one strain of Akkermansia sp., or at least one strain of Faecalibacterium sp. In some cases, a bacterial population may comprise at least one strain of a species of the Lactobacillaceae family, at least one strain of Akkermansia sp., or at least one strain of Faecalibacterium sp. In some cases, a bacterial population may comprise at least one strain of Lactobacillus sp., at least one strain of Akkermansia sp., and at least one strain of Faecalibacterium sp. In some cases, a bacterial population may comprise at least one strain of a species of the Lactobacillaceae family, at least one strain of Akkermansia sp., and at least one strain of Faecalibacterium sp.

In some instances, a bacterial population may comprise at least one strain of Lactobacillus sp. or at least one strain of Akkermansia sp. In some instances, a bacterial population may comprise at least one strain of a species of the Lactobacillaceae family or at least one strain of Akkermansia sp. In some cases, a bacterial population may comprise at least one strain of Lactobacillus sp. or at least one strain of Faecalibacterium sp. In some cases, a bacterial population may comprise at least one strain of a species of the Lactobacillaceae family or at least one strain of Faecalibacterium sp. In some cases, a bacterial population may comprise at least one strain of Akkermansia sp. or at least one strain of Faecalibacterium sp. In some cases, a bacterial population may comprise at least one strain of Lactobacillus sp. and at least one strain of Akkermansia sp. In some cases, a bacterial population may comprise at least one strain of a species of the Lactobacillaceae family and at least one strain of Akkermansia sp. In some cases, a bacterial population may comprise at least one strain of Lactobacillus sp. and at least one strain of Faecalibacterium sp. In some cases, a bacterial population may comprise at least one strain of a species of the Lactobacillaceae family and at least one strain of Faecalibacterium sp. In some cases, a bacterial population may comprise at least one strain of Akkermansia sp. and at least one strain of Faecalibacterium sp.

In some instances, a bacterial population may comprise at least two strains of Lactobacillus sp., at least two strains of Akkermansia sp., or at least two strains of Faecalibacterium sp. In some instances, a bacterial population may comprise at least two strains of species of the Lactobacillaceae family, at least two strains of Akkermansia sp., or at least two strains of Faecalibacterium sp. In some cases, a bacterial population may comprise at least three strains of Lactobacillus sp., at least three strains of Akkermansia sp., or at least three strains of Faecalibacterium sp. In some cases, a bacterial population may comprise at least three strains of species of the Lactobacillaceae family, at least three strains of Akkermansia sp., or at least three strains of Faecalibacterium sp. In some cases, a bacterial population may comprise at least more than three strains of Lactobacillus sp., at least more than three strains of Akkermansia sp., or at least more than three strains of Faecalibacterium sp. In some cases, a bacterial population may comprise at least more than three strains of species of the Lactobacillaceae family, at least more than three strains of Akkermansia sp., or at least more than three strains of Faecalibacterium sp. In some cases, a bacterial population may comprise at least one strain of Lactobacillus sp. In some cases, a bacterial population may comprise at least one strain of a species of the Lactobacillaceae family. In some cases, a bacterial population may comprise at least one strain of Akkermansia sp. In some cases, a bacterial population may comprise at least one strain of Faecalibacterium sp. In some cases, a bacterial population may comprise at least two strains of Lactobacillus sp. In some cases, a bacterial population may comprise at least two strains of species of the Lactobacillaceae family. In some cases, a bacterial population may comprise at least two strains of Akkermansia sp. In some cases, a bacterial population may comprise at least two strains of Faecalibacterium sp. In some cases, a bacterial population may comprise at least three strains of Lactobacillus sp. In some cases, a bacterial population may comprise at least three strains of species of the Lactobacillaceae family. In some cases, a bacterial population may comprise at least three strains of Akkermansia sp. In some cases, a bacterial population may comprise at least three strains of Faecalibacterium sp. In some cases, a bacterial population may comprise at least more than three strains of Lactobacillus sp. In some cases, a bacterial population may comprise at least more than three strains of species of the Lactobacillaceae family. In some cases, a bacterial population may comprise at least more than three strains of Akkermansia sp. In some cases, a bacterial population may comprise at least more than three strains of Faecalibacterium sp.

In some instances, a pharmaceutical composition that can comprise a bacterial population. Such bacterial population can comprise one or more different bacterial species and/or strains. Such bacterial species and/or strains can belong to one or more different bacterial phyla. Such bacterial phyla may include Verrucomicrobia, Firmicutes, or a combination thereof. Such bacterial phyla may also include Verrucomicrobia, Firmicutes, Proteobacteria, Actinobacteria, and/or Bacteroidetes, or a combination thereof.

In some instances, a species of Lactobacillus family may comprise a species of the Lactobacillus genus proposed in 1901, which is described in Zheng, J., et. al. Int. J. Syst. Evol. Microbiol. 2020; 70:2782-2858 and is entirely incorporated herein by reference. The Lactobacillus genus may comprise Gram-positive, fermentative, facultatively anaerobic, and/or non-spore forming microorganisms. In some cases, the number of microorganisms that can be classified as Lactobacillus genus may increase, compared to those classified in 1901, with the definition of the 1901 classification. Lactobacillus genus may comprise about 261 species that comprise distinctive phenotypic, ecological, and/or genotypic characteristics. The number of species in the genus and/or the level of diversity within the Lactobacillus genus may exceed those of other bacterial genera and/or bacterial families. In this case, Lactobacillus can be reclassified. For example, the average nucleotide identity (ANI), average amino acid identity (AAI), core-gene average amino acid identity (cAAI), core genome phylogeny, signature genes, and metabolic, and/or ecological criteria of the bacterial species in the Lactobacillus genus and its sister taxa in the Lactobacillaceae and Leuconostocacae families are used to reclassify the Lactobacillus genus classified using the definition of 1901.

In some cases, under the reclassification system, the species of the Lactobacillaceae family may comprise about 26 different genera (Lactobacillus, Paralactobacillus, Pediococcus, Holzapfelia, Amylolactobacillus, Bombilactobacillus, Companilactobacillus, Lapidilactobacillus, Agrilactobacillus, Schleiferilactobacillus, Loigolactobacillus, Lacticaseibacillus, Latilactobacillus, Dellaglioa, Liquorilactobacillus, Ligilactobacillus, Lactiplantibacillus, Furfurilactobacillus, Paucilactobacillus, Limosilactobacillus, Fructilactobacillus, Acetilactobacillus, Apilactobacillus, Levilactobacillus, Secundilactobacillus, and Lentilactobacillus), as well as merging the Leuconostocacae family into the Lactobacillaceae family. A comparison of the reclassified Lactobacillus species can be found using the Lactotax database, which can be found in the link: http://Lactobacillus.ualberta.ca/ and is entirely incorporated herein by reference. The classification of Lactobacillus described herein, is also provided in Parks, D H et. al. Nat Biotechnol. 2018 November; 36(10):996-1004; Salvetti, E, et. al. Appl Environ Microbio. 2018 Aug. 17; 84(17). Print 2018 Sep. 1 Erratum in: Appl Environ Microbio.2018 Oct. 1; 84(20); Markets and Markets: https://www.marketsandmarkets.com/Market-Reports/probiotic-market-advanced-technologies-and-global-market-69.html); Parker, C T, et. al. Int. J. Syst. Evol. Microbiol. 68:1825-1829; Duar, D M, et. al. FEMS Microbiol Rev. 2017 Aug. 1; 41(Supp_1):S27-S48; or Pane and Vinot 2019: htpps://www.microbiometimes.com/the-Lactobacillus-taxonomy-change-is-coming-why-and-how-to-make-the-most-of-it/, each of which is entirely incorporated herein by reference.

In some instances, a bacterial population described herein may comprise one or more Lactobacillus sp. The one or more Lactobacillus sp. may include Lactobacillus johnsonii, Lactobacillus rhamnosus, Lactobacillus zeae, Lactobacillus acidipiscis, Lactobacillus acidophilus, Lactobacillus agilis, Lactobacillus aviarius, Lactobacillus brevis, Lactobacillus coleohominis, Lactobacillus crispatus, Lactobacillus crustorum, Lactobacillus curvatus, Lactobacillus diolivorans, Lactobacillus farraginis, Lactobacillus fermentum, Lactobacillus fuchuensis, Lactobacillus harbinensis, Lactobacillus helveticus, Lactobacillus hilgardii, Lactobacillus intestinalis, Lactobacillus jensenii, Lactobacillus kefiranofaciens, Lactobacillus kefiri, Lactobacillus lindneri, Lactobacillus mali, Lactobacillus manihotivorans, Lactobacillus mucosae, Lactobacillus oeni, Lactobacillus oligofermentans, Lactobacillus panis, Lactobacillus pantheris, Lactobacillus parabrevis, Lactobacillus paracollinoides, Lactobacillus parakefiri, Lactobacillus paraplantarum, Lactobacillus pentosus, Lactobacillus pontis, Lactobacillus reuteri, Lactobacillus rossiae, Lactobacillus salivarius, Lactobacillus siliginis, Lactobacillus sucicola, Lactobacillus vaccinostercus, Lactobacillus vaginalis, Lactobacillus vini, Laclococcus garvieae, or Lactococcus lactis, or a combination thereof. In some embodiments, the Lactobacillus sp. is Lactobacillus johnsonii or Lactobacillus crispatus. In such instances, a bacterial population herein may comprise one or more Lactobacillus johnsonii or Lactobacillus crispatus strains. Such one or more Lactobacillus crispatus strain(s) may include Lactobacillus crispatus (DSM 33187) (i.e., L. crispatus (DSM 33187)). In various instances, a bacterial population herein comprises Lactobacillus crispatus (DSM 33187).

In some instances, a bacterial population described herein may comprise one or more Lactobacillus sp. The one or more Lactobacillus sp. may comprise Lactobacillus acidophilus, Lactobacillus crispatus, Lactobacillus helveticus, Lactobacillus intestinalis, Lactobacillus jensenii, Lactobacillus johnsonii, or Lactobacillus kefiranofaciens. In some cases, the bacterial population of the pharmaceutical composition may comprise at least two or a combination of Lactobacillus sp. The at least two or the combination of Lactobacillus sp. may comprise any one of Lactobacillus acidophilus, Lactobacillus crispatus, Lactobacillus helveticus, Lactobacillus intestinalis, Lactobacillus jensenii, Lactobacillus johnsonii, or Lactobacillus kefiranofaciens

In some instances, a bacterial population described herein may comprise one or more species of Lactobacillaceae family. The one or more species of the Lactobacillaceae family may include Lactobacillus johnsonii, Lactocaseibacillus rhamnosus, Lactocaseibacillus zeae, Ligilactobacillus acidipiscis, Lactobacillus acidophilus, Ligilactobacillus agilis, Ligilactobacillus aviarius, Levilactobacillus brevis, Limosilactobacillus coleohominis, Lactobacillus crispatus, Companilactobacillus crustorum, Latilactobacillus curvatus, Lentilactobacillus diolivorans, Lentilactobacillus farraginis, Limosilactobacillus fermentum, Latilactobacillus fuchuensis, Schleiferilactobacillus harbinensis, Lactobacillus helveticus, Lentilactobacillus hilgardii, Lactobacillus intestinalis, Lactobacillus jensenii, Lactobacillus kefiranofaciens, Lentilactobacillus kefiri, Fructilactobacillus lindneri, Liquorilactobacillus mali, Lactocaseibacillus manihotivorans, Limosilactobacillus mucosae, Liquorilactobacillus oeni, Paucilactobacillus oligofermentans, Limosilactobacillus panis, Lactocaseibacillus pantheris, Levilactobacillus parabrevis, Secundilactobacillus paracollinoides, Lentilactobacillus parakefiri, Lactoplantibacillus paraplantarum, Lactoplantibacillus pentosus, Limosiactobacillus pontis, Limosilactobacillus reuteri, Furfurilactobacillus rossiae, Ligilactobacillus salivarius, Furfurilactobacillus siliginis, Liquorilactobacillus sucicola, Paucilactobacillus vaccinostercus, Limosilactobacillus vaginalis, Liquorilactobacillus vini, Laclococcus garvieae, or Lactococcus lactis, or a combination thereof. In some embodiments, the species of the Lactobacillaceae family. is Lactobacillus johnsonii or Lactobacillus crispatus. In such instances, a bacterial population herein may comprise one or more Lactobacillus johnsonii or Lactobacillus crispatus strains. Such one or more Lactobacillus crispatus strain(s) may include Lactobacillus crispatus (DSM 33187) (i.e., L. crispatus (DSM 33187)). In various instances, a bacterial population herein comprises Lactobacillus crispatus (DSM 33187).

In some instances, a bacterial population described herein may comprise one or more species of the Lactobacillaceae family. The one or more species of the Lactobacillaceae family may include Lactobacillus acidophilus, Lactobacillus crispatus, Lactobacillus helveticus, Lactobacillus intestinalis, Lactobacillus jensenii, Lactobacillus johnsonii, and Lactobacillus kefiranofaciens. TABLE 39 below shows the names of various Lactobacillus sp. under the 1901 classification and the reclassification.

TABLE 39
Lactobacillus sp. names before and after reclassification

| Name of Lactobacillus | Name of Lactobacillus |
|---|---|
| species classified in 1901 | species under reclassification |
| Lactobacillus acidipiscis | Ligilactobacillus acidipiscis |
| Lactobacillus acidophilus | Lactobacillus acidophilus |
| Lactobacillus agilis | Ligilactobacillus agilis |
| Lactobacillus aviarius | Ligilactobacillus aviarius |
| Lactobacillus brevis | Levilactobacillus brevis |
| Lactobacillus coleohominis | Limosilactobacillus coleohominis |
| Lactobacillus crispatus | Lactobacillus crispatus |
| Lactobacillus crustorum | Companilactobacillus crustorum |
| Lactobacillus curvatus | Latilactobacillus curvatus |
| Lactobacillus diolivorans | Lentilactobacillus diolivorans |
| Lactobacillus farraginis | Lentilactobacillus farraginis |
| Lactobacillus fermentum | Limosilactobacillus fermentum |
| Lactobacillus fuchuensis | Latilactobacillus fuchuensis |
| Lactobacillus harbinensis | Schleiferilactobacillus harbinensis |
| Lactobacillus helveticus | Lactobacillus helveticus |
| Lactobacillus hilgardii | Lentilactobacillus hilgardii |
| Lactobacillus intestinalis | Lactobacillus intestinalis |
| Lactobacillus jensenii | Lactobacillus jensenii |
| Lactobacillus johnsonii | Lactobacillus johnsonii |
| Lactobacillus kefiranofaciens | Lactobacillus kefiranofaciens |
| Lactobacillus kefiri | Lentilactobacillus kefiri |
| Lactobacillus lindneri | Fructilactobacillus lindneri |
| Lactobacillus mali | Liquorilactobacillus mali |
| Lactobacillus manihotivorans | Lacticaseibacillus manihotivorans |
| Lactobacillus mucosae | Limosilactobacillus mucosae |
| Lactobacillus oeni | Liquorilactobacillus oeni |
| Lactobacillus oligofermentans | Paucilactobacillus oligofermentans |
| Lactobacillus panis | Limosilactobacillus panis |
| Lactobacillus pantheris | Lacticaseibacillus pantheris |
| Lactobacillus parabrevis | Levilactobacillus parabrevis |
| Lactobacillus paracollinoides | Secundilactobacillus paracollinoides |
| Lactobacillus parakefiri | Lentilactobacillus parakefiri |
| Lactobacillus paraplantarum | Lactiplantibacillus paraplantarum |
| Lactobacillus pentosus | Lactiplantibacillus pentosus |
| Lactobacillus pontis | Limosilactobacillus pontis |
| Lactobacillus reuteri | Limosilactobacillus reuteri |
| Lactobacillus rhamnosus | Lacticaseibacillus rhamnosus |
| Lactobacillus rossiae | Furfurilactobacillus rossiae |
| Lactobacillus salivarius | Ligilactobacillus salivarius |
| Lactobacillus siliginis | Furfurilactobacillus siliginis |
| Lactobacillus sucicola | Liquorilactobacillus sucicola |
| Lactobacillus vaccinostercus | Paucilactobacillus vaccinostercus |
| Lactobacillus vaginalis | Limosilactobacillus vaginalis |
| Lactobacillus vini | Liquorilactobacillus vini |
| Lactobacillus zeae | Lacticaseibacillus zeae |

In some instances, a bacterial population herein may comprise one or more Akkermansia sp. Such one or more Akkermansia sp. may include Akkermansia muciniphila, Akkermansia glycaniphila, or a combination thereof. In some instances, the one or more Akkermansia sp. can be Akkermansia muciniphila. In such instances, a bacterial population herein may comprise one or more Akkermansia muciniphila strains. Such one or more Akkermansia muciniphila strains may include Akkermansia muciniphila (DSM 33213). In various instances, a bacterial population herein comprises Akkermansia muciniphila (DSM 33213).

In some instances, a bacterial population may comprise one or more Faecalibacterium sp. The one or more Faecalibacterium sp. may include Faecalibacterium prausnitzii. In such instances, a bacterial population herein may comprise one or more Faecalibacterium prausnitzii strains. Such one or more Faecalibacterium prausnitzii strains may include Faecalibacterium prausnitzii (DSM 33185), Faecalibacterium prausnitzii (DSM 33191), Faecalibacterium prausnitzii (DSM 33186), or Faecalibacterium prausnitzii (DSM 33190), or a combination thereof. In various instances, a bacterial population herein comprises Faecalibacterium prausnitzii (DSM 33185).

Further provided herein are bacterial populations that may comprise one or more strains of any one or more of Bacteroides sp., Blautia sp., Bifidobacterium sp., Coprococcus sp., or Dorea sp. In such instances, a bacterial population herein may comprise any one or more of Bacteroides faecis (DSM 22177), Bacteroides thetaiotaomicron (DSM 33178), Blautia producta (DSM 33180), Bifidobacterium longum (DSM 33179), Coprococcus comes (DSM 33176), or Dorea longicatena (DSM 33188). Exemplary strains for inclusion in a bacterial population described herein are listed in TABLE 1.

TABLE 1
Exemplary Bacterial Strains.

| Bacterial Strain | Deposit ID # | Bacterial Strain | Deposit ID # |
|---|---|---|---|
| A. muciniphila | DSM 33213 | F. prausnitzii | DSM 33191 |
| B. longum | DSM 33179 | F. prausnitzii | DSM 33186 |
| B. producta | DSM 33180 | F. prausnitzii | DSM 33190 |
| B. thetaiotaomicron | DSM 33178 | L. crispatus | DSM 33187 |
| C. comes | DSM 33176 | B. faecis | DSM 22177 |
| F. prausnitzii | DSM 33185 | Dorea longicatena | DSM 33188 |

In some instances, a bacterial population may comprise one strain of Lactobacillus sp., one strain of Akkermansia sp., or one strain of Faecalibacterium sp. In some instances, a bacterial population may comprise one strain of a species of the Lactobacillaceae family, one strain of Akkermansia sp., or one strain of Faecalibacterium sp. In some cases, a bacterial population may comprise one strain of Lactobacillus sp., one strain of Akkermansia sp., and one strain of Faecalibacterium sp. In some cases, a bacterial population may comprise one strain of a species of the Lactobacillaceae family, one strain of Akkermansia sp., and one strain of Faecalibacterium sp. In some cases, a bacterial population may comprise one strain of Lactobacillus sp. or one strain of Akkermansia sp. In some cases, a bacterial population may comprise one strain of a species of the Lactobacillaceae family or one strain of Akkermansia sp. In some cases, a bacterial population may comprise one strain of Lactobacillus sp. or one strain of Faecalibacterium sp. In some cases, a bacterial population may comprise one strain of a species of the Lactobacillaceae family or one strain of Faecalibacterium sp. In some cases, a bacterial population may comprise one strain of Akkermansia sp. or one strain of Faecalibacterium sp. In some cases, a bacterial population may comprise one strain of Lactobacillus sp. and one strain of Akkermansia sp. In some cases, a bacterial population may comprise one strain of a species of the Lactobacillaceae family and one strain of Akkermansia sp. In some cases, a bacterial population may comprise one strain of Lactobacillus sp. and one strain of Faecalibacterium sp. In some cases, a bacterial population may comprise one strain of a species of the Lactobacillaceae family and one strain of Faecalibacterium sp. In some cases, a bacterial population may comprise one strain of Akkermansia sp. and one strain of Faecalibacterium sp.

In some instances, a bacterial population may comprise Lactobacillus crispatus (DSM 33187), Akkermansia muciniphila (DSM 33213), or Faecalibacterium prausnitzii (DSM 33185). In some cases, a bacterial population may comprise at least two of Lactobacillus crispatus (DSM 33187), Akkermansia muciniphila (DSM 33213), or Faecalibacterium prausnitzii (DSM 33185). In some cases, a bacterial population may comprise Lactobacillus crispatus (DSM 33187), Akkermansia muciniphila (DSM 33213), and Faecalibacterium prausnitzii (DSM 33185). In some cases, a bacterial population may comprise Lactobacillus crispatus (DSM 33187). In some cases, a bacterial population may comprise Akkermansia sp. In some cases, a bacterial population may comprise Faecalibacterium prausnitzii (DSM 33185). In some cases, a bacterial population may comprise at least two strains of Lactobacillus crispatus (DSM 33187).

In some instances, a bacterial population may comprise, consist essentially of, or consist of 20, 19, 18, 17, 16, 15, 14, 13, 12, 11, 10, 9, 8, 7, 6, 5, 4, 3, 2 or 1 bacterial species and/or strain(s). In some instances, such bacterial populations can comprise at least one bacterial strain selected from TABLE 1. In some embodiments, a bacterial population can consist of up to 3 different bacterial strains. In some embodiments, a bacterial population described herein comprises at least one, at least two, or all three bacterial strains listed in TABLE 2. In some instances, a bacterial population comprises or consists of the bacterial strains L. crispatus (DSM 33187), A. muciniphila (DSM 33213), and F. prausnitzii (DSM 33185).

TABLE 2
A Subset of Bacterial Strains.

| Bacterial strain | Deposit ID # |
|---|---|
| Lactobacillus crispatus | DSM 33187 |
| Akkermansia muciniphila | DSM 33213 |
| Faecalibacterium prausnitzii | DSM 33185 |

In some instances, a bacterial population may comprise a varying number of colony-forming units (CFU/dose) of each of the bacterial species and/or strain it contains. In some cases, a bacterial population may comprise from about 1×10\hat{ }3 CFU/dose to about 1×10\hat{ }12 CFU/dose of a bacterial species or strain. In some cases, a bacterial population may comprise from about 1×10\hat{ }3 CFU/dose to about 1×10\hat{ }11 CFU/dose of a bacterial species or strain. In some cases, a bacterial population may comprise from about 1×10 \hat{ }3 CFU/dose to about 1×10\hat{ }10 CFU/dose of a bacterial species or strain. In some cases, a bacterial population may comprise from about 1×10 \hat{ }3 CFU/dose to about 1×10\hat{ }9 CFU/dose of a bacterial species or strain. In some cases, a bacterial population may comprise from about 1×10 \hat{ }3 CFU/dose to about 1×10\hat{ }8 CFU/dose of a bacterial species or strain. In some cases, a bacterial population may comprise from about 1×10\hat{ }4 CFU/dose to about 1×10\hat{ }12 CFU/dose of a bacterial species or strain. In some cases, a bacterial population may comprise from about 1×10\hat{ }4 CFU/dose to about 1×10\hat{ }11 CFU/dose of a bacterial species or strain. In some cases, a bacterial population may comprise from about 1×10\hat{ }4 CFU/dose to about 1×10\hat{ }10 CFU/dose of a bacterial species or strain. In some cases, a bacterial population may comprise from about 1×10\hat{ }4 CFU/dose to about 1×10\hat{ }9 CFU/dose of a bacterial species or strain. In some cases, a bacterial population may comprise from about 1×10\hat{ }4 CFU/dose to about 1×10\hat{ }8 CFU/dose of a bacterial species or strain. In some cases, a bacterial population may comprise from about 1×10\hat{ }5 CFU/dose to about 1×10\hat{ }12 CFU/dose of a bacterial species or strain. In some cases, a bacterial population may comprise from about 1×10\hat{ }5 CFU/dose to about 1×10\hat{ }11 CFU/dose of a bacterial species or strain. In some cases, a bacterial population may comprise from about 1×10\hat{ }5 CFU/dose to about 1×10\hat{ }10 CFU/dose of a bacterial species or strain. In some cases, a bacterial population may comprise from about 1×10\hat{ }5 CFU/dose to about 1×10\hat{ }9 CFU/dose of a bacterial species or strain. In some cases, a bacterial population may comprise from about 1×10\hat{ }5 CFU/dose to about 1×10\hat{ }8 CFU/dose of a bacterial species or strain. In some cases, a bacterial population may comprise from about 1×10\hat{ }6 CFU/dose to about 1×10\hat{ }12 CFU/dose of a bacterial species or strain. In some cases, a bacterial population may comprise from about 1×10\hat{ }6 CFU/dose to about 1×10\hat{ }11 CFU/dose of a bacterial species or strain. In some cases, a bacterial population may comprise from about 1×10\hat{ }6 CFU/dose to about 1×10\hat{ }10 CFU/dose of a bacterial species or strain. In some cases, a bacterial population may comprise from about 1×10\hat{ }6 CFU/dose to about 1×10\hat{ }9 CFU/dose of a bacterial species or strain. In some cases, a bacterial population may comprise from about 1×10\hat{ }6 CFU/dose to about 1×10\hat{ }8 CFU/dose of a bacterial species or strain. In some cases, a bacterial population may comprise from about 1×10\hat{ }7 CFU/dose to about 1×10\hat{ }12 CFU/dose of a bacterial species or strain. In some cases, a bacterial population may comprise from about 1×10\hat{ }7 CFU/dose to about 1×10\hat{ }11 CFU/dose of a bacterial species or strain. In some cases, a bacterial population may comprise from about 1×10\hat{ }7 CFU/dose to about 1×10\hat{ }10 CFU/dose of a bacterial species or strain. In some cases, a bacterial population may comprise from about 1×10\hat{ }7 CFU/dose to about 1×10\hat{ }9 CFU/dose of a bacterial species or strain. In some cases, a bacterial population may comprise from about 1×10\hat{ }7 CFU/dose to about 1×10\hat{ }8 CFU/dose of a bacterial species or strain. In some cases, a bacterial population may comprise from about 1×10\hat{ }8 CFU/dose to about 1×10\hat{ }12 CFU/dose of a bacterial species or strain. In some cases, a bacterial population may comprise from about 1×10\hat{ }8 CFU/dose to about 1×10\hat{ }11 CFU/dose of a bacterial species or strain. In some cases, a bacterial population may comprise from about 1×10\hat{ }8 CFU/dose to about 1×10\hat{ }10 CFU/dose of a bacterial species or strain. In some cases, a bacterial population may comprise from about 1×10\hat{ }8 CFU/dose to about 1×10\hat{ }9 CFU/dose of a bacterial species or strain. In some cases, a bacterial population may comprise from about 1×10\hat{ }9 CFU/dose to about 1×10\hat{ }12 CFU/dose of a bacterial species or strain. In some cases, a bacterial population may comprise from about 1×10\hat{ }9 CFU/dose to about 1×10\hat{ }11 CFU/dose of a bacterial species or strain. In some cases, a bacterial population may comprise from about 1×10\hat{ }9 CFU/dose to about 1×10\hat{ }10 CFU/dose of a bacterial species or strain. In some cases, such bacterial population may also comprise from about 1×10\hat{ }7 CFU/dose to about 1×10\hat{ }10 CFU/dose of a bacterial species or strain. In some instances, a bacterial population may comprise at least about 1×10 \hat{ }3 CFU/dose, 5×10 \hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of a bacterial species or strain. The bacterial populations may also comprise from about 1×10\hat{ }6 to about 1×10\hat{ }11 CFU/dose per bacterial species or strain. In some cases, the bacterial populations may comprise from about 1×10\hat{ }3 to about 1×10\hat{ }12 CFU/dose per bacterial species or strain. In some instances, the bacterial populations may comprise from about 1×10\hat{ }8 to about 5×10\hat{ }10 CFU/dose per bacterial species or strain. In some instances, the bacterial populations may comprise from about 1×10\hat{ }7 to about 5×10\hat{ }10 CFU/dose per bacterial species or strain. In various cases, a bacterial population may comprise about 5×10\hat{ }8 CFU/dose per bacterial species or strain.

In some instances, a bacterial population may comprise a varying number of colony-forming units (CFU/dose) of bacterial cells. In some cases, a bacterial population may comprise from about 1×10 \hat{ }3 CFU/dose to about 1×10\hat{ }12 CFU/dose of bacterial cells. In some cases, a bacterial population may comprise from about 1×10 \hat{ }3 CFU/dose to about 1×10\hat{ }11 CFU/dose of bacterial cells. In some cases, a bacterial population may comprise from about 1×10 \hat{ }3 CFU/dose to about 1×10\hat{ }10 CFU/dose of bacterial cells. In some cases, a bacterial population may comprise from about 1×10 \hat{ }3 CFU/dose to about 1×10\hat{ }9 CFU/dose of bacterial cells. In some cases, a bacterial population may comprise from about 1×10 \hat{ }3 CFU/dose to about 1×10\hat{ }8 CFU/dose of bacterial cells. In some cases, a bacterial population may comprise from about 1×10\hat{ }4 CFU/dose to about 1×10\hat{ }12 CFU/dose of bacterial cells. In some cases, a bacterial population may comprise from about 1×10\hat{ }4 CFU/dose to about 1×10\hat{ }11 CFU/dose of bacterial cells. In some cases, a bacterial population may comprise from about 1×10\hat{ }4 CFU/dose to about 1×10\hat{ }10 CFU/dose of bacterial cells. In some cases, a bacterial population may comprise from about 1×10\hat{ }4 CFU/dose to about 1×10\hat{ }9 CFU/dose of bacterial cells. In some cases, a bacterial population may comprise from about 1×10\hat{ }4 CFU/dose to about 1×10\hat{ }8 CFU/dose of bacterial cells. In some cases, a bacterial population may comprise from about 1×10\hat{ }5 CFU/dose to about 1×10\hat{ }12 CFU/dose of bacterial cells. In some cases, a bacterial population may comprise from about 1×10\hat{ }5 CFU/dose to about 1×10\hat{ }11 CFU/dose of bacterial cells. In some cases, a bacterial population may comprise from about 1×10\hat{ }5 CFU/dose to about 1×10\hat{ }10 CFU/dose of bacterial cells. In some cases, a bacterial population may comprise from about 1×10\hat{ }5 CFU/dose to about 1×10\hat{ }9 CFU/dose of bacterial cells. In some cases, a bacterial population may comprise from about 1×10\hat{ }5 CFU/dose to about 1×10\hat{ }8 CFU/dose of bacterial cells. In some cases, a bacterial population may comprise from about 1×10\hat{ }6 CFU/dose to about 1×10\hat{ }12 CFU/dose of bacterial cells. In some cases, a bacterial population may comprise from about 1×10\hat{ }6 CFU/dose to about 1×10\hat{ }11 CFU/dose of bacterial cells. In some cases, a bacterial population may comprise from about 1×10\hat{ }6 CFU/dose to about 1×10\hat{ }10 CFU/dose of bacterial cells. In some cases, a bacterial population may comprise from about 1×10\hat{ }6 CFU/dose to about 1×10\hat{ }9 CFU/dose of bacterial cells. In some cases, a bacterial population may comprise from about 1×10\hat{ }6 CFU/dose to about 1×10\hat{ }8 CFU/dose of bacterial cells. In some cases, a bacterial population may comprise from about 1×10\hat{ }7 CFU/dose to about 1×10\hat{ }12 CFU/dose of bacterial cells. In some cases, a bacterial population may comprise from about 1×10\hat{ }7 CFU/dose to about 1×10\hat{ }11 CFU/dose of bacterial cells. In some cases, a bacterial population may comprise from about 1×10\hat{ }7 CFU/dose to about 1×10\hat{ }10 CFU/dose of bacterial cells. In some cases, a bacterial population may comprise from about 1×10\hat{ }7 CFU/dose to about 1×10\hat{ }9 CFU/dose of bacterial cells. In some cases, a bacterial population may comprise from about 1×10\hat{ }7 CFU/dose to about 1×10\hat{ }8 CFU/dose of bacterial cells. In some cases, a bacterial population may comprise from about 1×10\hat{ }8 CFU/dose to about 1×10\hat{ }12 CFU/dose of bacterial cells. In some cases, a bacterial population may comprise from about 1×10\hat{ }8 CFU/dose to about 1×10\hat{ }11 CFU/dose of bacterial cells. In some cases, a bacterial population may comprise from about 1×10\hat{ }8 CFU/dose to about 1×10\hat{ }10 CFU/dose of bacterial cells. In some cases, a bacterial population may comprise from about 1×10\hat{ }8 CFU/dose to about 1×10\hat{ }9 CFU/dose of bacterial cells. In some cases, a bacterial population may comprise from about 1×10\hat{ }9 CFU/dose to about 1×10\hat{ }12 CFU/dose of bacterial cells. In some cases, a bacterial population may comprise from about 1×10\hat{ }9 CFU/dose to about 1×10\hat{ }11 CFU/dose of bacterial cells. In some cases, a bacterial population may comprise from about 1×10\hat{ }9 CFU/dose to about 1×10\hat{ }10 CFU/dose of bacterial cells. In some cases, such bacterial population may also comprise from about 1×10\hat{ }7 CFU/dose to about 1×10\hat{ }10 CFU/dose of bacterial cells. In some instances, a bacterial population may comprise at least about 1×10\hat{ }3 CFU/dose, 5×10 \hat{ }3 CFU/dose, 1×10\hat{ }4 CFU/dose, 5×10\hat{ }4 CFU/dose, 1×10\hat{ }5 CFU/dose, 5×10\hat{ }5 CFU/dose, 1×10\hat{ }6 CFU/dose, 5×10\hat{ }6 CFU/dose, 1×10\hat{ }7 CFU/dose, 5×10\hat{ }7 CFU/dose, 1×10\hat{ }8 CFU/dose, 5×10\hat{ }8 CFU/dose, 1×10\hat{ }9 CFU/dose, 5×10\hat{ }9 CFU/dose, 1×10\hat{ }10 CFU/dose, 5×10\hat{ }10 CFU/dose, 1×10\hat{ }11 CFU/dose, 5×10\hat{ }11 CFU/dose, or 1×10\hat{ }12 CFU/dose, but no more than about 5×10\hat{ }12 CFU/dose of bacterial cells.

In some cases, at least one strain of Akkermansia sp. of a pharmaceutical composition may comprise from about 1×10\hat{ }6 CFU/dose to about 5×10\hat{ }9 CFU/dose. In some cases, at least one strain of Akkermansia sp. of a pharmaceutical composition may comprise from about 1×10\hat{ }6 CFU/dose to about 4×10\hat{ }9 CFU/dose. In some cases, at least one strain of Akkermansia sp. of a pharmaceutical composition may comprise from about 1×10\hat{ }6 CFU/dose to about 3×10\hat{ }9 CFU/dose. In some cases, at least one strain of Akkermansia sp. of a pharmaceutical composition may comprise from about 1×10\hat{ }6 CFU/dose to about 2×10\hat{ }9 CFU/dose. In some cases, at least one strain of Akkermansia sp. of a pharmaceutical composition may comprise from about 1×10\hat{ }6 CFU/dose to about 1×10\hat{ }9 CFU/dose. In some cases, at least one strain of Akkermansia sp. of a pharmaceutical composition may comprise from about 1×10\hat{ }6 CFU/dose to about 9×10\hat{ }8 CFU/dose. In some cases, at least one strain of Akkermansia sp. of a pharmaceutical composition may comprise from about 1×10\hat{ }6 CFU/dose to about 8×10\hat{ }8 CFU/dose. In some cases, at least one strain of Akkermansia sp. of a pharmaceutical composition may comprise from about 1×10\hat{ }6 CFU/dose to about 7×10\hat{ }8 CFU/dose. In some cases, at least one strain of Akkermansia sp. of a pharmaceutical composition may comprise from about 1×10\hat{ }6 CFU/dose to about 6×10\hat{ }8 CFU/dose. In some cases, at least one strain of Akkermansia sp. of a pharmaceutical composition may comprise from about 1×10\hat{ }6 CFU/dose to about 5×10\hat{ }8 CFU/dose. In some cases, at least one strain of Akkermansia sp. of a pharmaceutical composition may comprise from about 1×10\hat{ }6 CFU/dose to about 4×10\hat{ }8 CFU/dose. In some cases, at least one strain of Akkermansia sp. of a pharmaceutical composition may comprise from about 1×10\hat{ }6 CFU/dose to about 3×10\hat{ }8 CFU/dose. In some cases, at least one strain of Akkermansia sp. of a pharmaceutical composition may comprise from about 1×10\hat{ }6 CFU/dose to about 2×10\hat{ }8 CFU/dose. In some cases, at least one strain of Akkermansia sp. of a pharmaceutical composition may comprise from about 1×10\hat{ }6 CFU/dose to about 1×10\hat{ }8 CFU/dose. In some cases, at least one strain of Akkermansia sp. of a pharmaceutical composition may comprise from about 1×10\hat{ }6 CFU/dose to about 9×10\hat{ }7 CFU/dose. In some cases, at least one strain of Akkermansia sp. of a pharmaceutical composition may comprise from about 1×10\hat{ }6 CFU/dose to about 8×10\hat{ }7 CFU/dose. In some cases, at least one strain of Akkermansia sp. of a pharmaceutical composition may comprise from about 1×10\hat{ }6 CFU/dose to about 7×10\hat{ }7 CFU/dose. In some cases, at least one strain of Akkermansia sp. of a pharmaceutical composition may comprise from about 1×10\hat{ }6 CFU/dose to about 6×10\hat{ }7 CFU/dose. In some cases, at least one strain of Akkermansia sp. of a pharmaceutical composition may comprise from about 1×10\hat{ }6 CFU/dose to about 5×10\hat{ }7 CFU/dose. In some cases, at least one strain of Akkermansia sp. of a pharmaceutical composition may comprise from about 1×10\hat{ }6 CFU/dose to about 4×10\hat{ }7 CFU/dose. In some cases, at least one strain of Akkermansia sp. of a pharmaceutical composition may comprise from about 1×10\hat{ }6 CFU/dose to about 3×10\hat{ }7 CFU/dose. In some cases, at least one strain of Akkermansia sp. of a pharmaceutical composition may comprise from about 1×10\hat{ }6 CFU/dose to about 2×10\hat{ }7 CFU/dose. In some cases, at least one strain of Akkermansia sp. of a pharmaceutical composition may comprise from about 1×10\hat{ }6 CFU/dose to about 1×10\hat{ }7 CFU/dose. In some cases, at least one strain of Akkermansia sp. may comprise at least about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, 1×10\hat{ }12 or more CFU/dose. In some cases, at least one strain of Akkermansia sp. may comprise at most about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, or 1×10\hat{ }12 CFU/dose. In some cases, at least one strain of Akkermansia sp. may comprise about 5×10\hat{ }9 CFU/dose. In some cases, at least one strain of Akkermansia sp. may comprise about 4×10\hat{ }9 CFU/dose. In some cases, at least one strain of Akkermansia sp. may comprise about 3×10\hat{ }9 CFU/dose. In some cases, at least one strain of Akkermansia sp. may comprise about 2×10\hat{ }9 CFU/dose. In some cases, at least one strain of Akkermansia sp. may comprise about 1×10\hat{ }9 CFU/dose. In some cases, at least one strain of Akkermansia sp. may comprise about 9×10\hat{ }8 CFU/dose. In some cases, at least one strain of Akkermansia sp. may comprise about 8×10\hat{ }8 CFU/dose. In some cases, at least one strain of Akkermansia sp. may comprise about 7×10\hat{ }8 CFU/dose. In some cases, at least one strain of Akkermansia sp. may comprise about 6×10\hat{ }8 CFU/dose. In some cases, at least one strain of Akkermansia sp. may comprise about 5×10\hat{ }8 CFU/dose. In some cases, at least one strain of Akkermansia sp. may comprise about 4×10\hat{ }8 CFU/dose. In some cases, at least one strain of Akkermansia sp. may comprise about 3×10\hat{ }8 CFU/dose. In some cases, at least one strain of Akkermansia sp. may comprise about 2×10\hat{ }8 CFU/dose. In some cases, at least one strain of Akkermansia sp. may comprise about 1×10\hat{ }8 CFU/dose. In some cases, at least one strain of Akkermansia sp. may comprise about 9×10\hat{ }7 CFU/dose. In some cases, at least one strain of Akkermansia sp. may comprise about 8×10\hat{ }7 CFU/dose. In some cases, at least one strain of Akkermansia sp. may comprise about 7×10\hat{ }7 CFU/dose. In some cases, at least one strain of Akkermansia sp. may comprise about 6×10\hat{ }7 CFU/dose. In some cases, at least one strain of Akkermansia sp. may comprise about 5×10\hat{ }7 CFU/dose. In some cases, at least one strain of Akkermansia sp. may comprise about 4×10\hat{ }7 CFU/dose. In some cases, at least one strain of Akkermansia sp. may comprise about 3×10\hat{ }7 CFU/dose. In some cases, at least one strain of Akkermansia sp. may comprise about 2×10\hat{ }7 CFU/dose. In some cases, at least one strain of Akkermansia sp. may comprise about 1×10\hat{ }7 CFU/dose.

In some cases, A. muciniphila (DSM 33213) of a pharmaceutical composition may comprise from about 1×10\hat{ }6 CFU/dose to about 5×10\hat{ }9 CFU/dose. In some cases, A. muciniphila (DSM 33213) of a pharmaceutical composition may comprise from about 1×10\hat{ }6 CFU/dose to about 4×10\hat{ }9 CFU/dose. In some cases, A. muciniphila (DSM 33213) of a pharmaceutical composition may comprise from about 1×10\hat{ }6 CFU/dose to about 3×10\hat{ }9 CFU/dose. In some cases, A. muciniphila (DSM 33213) of a pharmaceutical composition may comprise from about 1×10\hat{ }6 CFU/dose to about 2×10\hat{ }9 CFU/dose. In some cases, A. muciniphila (DSM 33213) of a pharmaceutical composition may comprise from about 1×10\hat{ }6 CFU/dose to about 1×10\hat{ }9 CFU/dose. In some cases, A. muciniphila (DSM 33213) of a pharmaceutical composition may comprise from about 1×10\hat{ }6 CFU/dose to about 9×10\hat{ }8 CFU/dose. In some cases, A. muciniphila (DSM 33213) of a pharmaceutical composition may comprise from about 1×10\hat{ }6 CFU/dose to about 8×10\hat{ }8 CFU/dose. In some cases, A. muciniphila (DSM 33213) of a pharmaceutical composition may comprise from about 1×10\hat{ }6 CFU/dose to about 7×10\hat{ }8 CFU/dose. In some cases, A. muciniphila (DSM 33213) of a pharmaceutical composition may comprise from about 1×10\hat{ }6 CFU/dose to about 6×10\hat{ }8 CFU/dose. In some cases, A. muciniphila (DSM 33213) of a pharmaceutical composition may comprise from about 1×10\hat{ }6 CFU/dose to about 5×10\hat{ }8 CFU/dose. In some cases, A. muciniphila (DSM 33213) of a pharmaceutical composition may comprise from about 1×10\hat{ }6 CFU/dose to about 4×10\hat{ }8 CFU/dose. In some cases, A. muciniphila (DSM 33213) of a pharmaceutical composition may comprise from about 1×10\hat{ }6 CFU/dose to about 3×10\hat{ }8 CFU/dose. In some cases, A. muciniphila (DSM 33213) of a pharmaceutical composition may comprise from about 1×10\hat{ }6 CFU/dose to about 2×10\hat{ }8 CFU/dose. In some cases, A. muciniphila (DSM 33213) of a pharmaceutical composition may comprise from about 1×10\hat{ }6 CFU/dose to about 1×10\hat{ }8 CFU/dose. In some cases, A. muciniphila (DSM 33213) of a pharmaceutical composition may comprise from about 1×10\hat{ }6 CFU/dose to about 9×10\hat{ }7 CFU/dose. In some cases, A. muciniphila (DSM 33213) of a pharmaceutical composition may comprise from about 1×10\hat{ }6 CFU/dose to about 8×10\hat{ }7 CFU/dose. In some cases, A. muciniphila (DSM 33213) of a pharmaceutical composition may comprise from about 1×10\hat{ }6 CFU/dose to about 7×10\hat{ }7 CFU/dose. In some cases, A. muciniphila (DSM 33213) of a pharmaceutical composition may comprise from about 1×10\hat{ }6 CFU/dose to about 6×10\hat{ }7 CFU/dose. In some cases, A. muciniphila (DSM 33213) of a pharmaceutical composition may comprise from about 1×10\hat{ }6 CFU/dose to about 5×10\hat{ }7 CFU/dose. In some cases, A. muciniphila (DSM 33213) of a pharmaceutical composition may comprise from about 1×10\hat{ }6 CFU/dose to about 4×10\hat{ }7 CFU/dose. In some cases, A. muciniphila (DSM 33213) of a pharmaceutical composition may comprise from about 1×10\hat{ }6 CFU/dose to about 3×10\hat{ }7 CFU/dose. In some cases, A. muciniphila (DSM 33213) of a pharmaceutical composition may comprise from about 1×10\hat{ }6 CFU/dose to about 2×10\hat{ }7 CFU/dose. In some cases, A. muciniphila (DSM 33213) of a pharmaceutical composition may comprise from about 1×10\hat{ }6 CFU/dose to about 1×10\hat{ }7 CFU/dose. In some cases, A. muciniphila (DSM 33213) may comprise about 5×10\hat{ }9 CFU/dose. In some cases, A. muciniphila (DSM 33213) may comprise about 4×10\hat{ }9 CFU/dose. In some cases, A. muciniphila (DSM 33213) may comprise about 3×10\hat{ }9 CFU/dose. In some cases, A. muciniphila (DSM 33213) may comprise about 2×10\hat{ }9 CFU/dose. In some cases, A. muciniphila (DSM 33213) may comprise about 1×10\hat{ }9 CFU/dose. In some cases, A. muciniphila (DSM 33213) may comprise about 9×10\hat{ }8 CFU/dose. In some cases, A. muciniphila (DSM 33213) may comprise about 8×10\hat{ }8 CFU/dose. In some cases, A. muciniphila (DSM 33213) may comprise about 7×10\hat{ }8 CFU/dose. In some cases, A. muciniphila (DSM 33213) may comprise about 6×10\hat{ }8 CFU/dose. In some cases, A. muciniphila (DSM 33213) may comprise about 5×10\hat{ }8 CFU/dose. In some cases, A. muciniphila (DSM 33213) may comprise about 4×10\hat{ }8 CFU/dose. In some cases, A. muciniphila (DSM 33213) may comprise about 3×10\hat{ }8 CFU/dose. In some cases, A. muciniphila (DSM 33213) may comprise about 2×10\hat{ }8 CFU/dose. In some cases, A. muciniphila (DSM 33213) may comprise about 1×10\hat{ }8 CFU/dose. In some cases, A. muciniphila (DSM 33213) may comprise about 9×10\hat{ }7 CFU/dose. In some cases, A. muciniphila (DSM 33213) may comprise about 8×10\hat{ }7 CFU/dose. In some cases, A. muciniphila (DSM 33213) may comprise about 7×10\hat{ }7 CFU/dose. In some cases, A. muciniphila (DSM 33213) may comprise about 6×10\hat{ }7 CFU/dose. In some cases, A. muciniphila (DSM 33213) may comprise about 5×10\hat{ }7 CFU/dose. In some cases, A. muciniphila (DSM 33213) may comprise about 4×10\hat{ }7 CFU/dose. In some cases, A. muciniphila (DSM 33213) may comprise about 3×10\hat{ }7 CFU/dose. In some cases, A. muciniphila (DSM 33213) may comprise about 2×10\hat{ }7 CFU/dose. In some cases, A. muciniphila (DSM 33213) may comprise about 1×10\hat{ }7 CFU/dose.

In some cases, at least one strain of Faecalibacterium sp. of a pharmaceutical composition may comprise from about 1×10\hat{ }6 CFU/dose to about 5×10\hat{ }9 CFU/dose. In some cases, at least one strain of Faecalibacterium sp. of a pharmaceutical composition may comprise from about 1×10\hat{ }6 CFU/dose to about 4×10\hat{ }9 CFU/dose. In some cases, at least one strain of Faecalibacterium sp. of a pharmaceutical composition may comprise from about 1×10\hat{ }6 CFU/dose to about 3×10\hat{ }9 CFU/dose. In some cases, at least one strain of Faecalibacterium sp. of a pharmaceutical composition may comprise from about 1×10\hat{ }6 CFU/dose to about 2×10\hat{ }9 CFU/dose. In some cases, at least one strain of Faecalibacterium sp. of a pharmaceutical composition may comprise from about 1×10\hat{ }6 CFU/dose to about 1×10\hat{ }9 CFU/dose. In some cases, at least one strain of Faecalibacterium sp. of a pharmaceutical composition may comprise from about 1×10\hat{ }6 CFU/dose to about 9×10\hat{ }8 CFU/dose. In some cases, at least one strain of Faecalibacterium sp. of a pharmaceutical composition may comprise from about 1×10\hat{ }6 CFU/dose to about 8×10\hat{ }8 CFU/dose. In some cases, at least one strain of Faecalibacterium sp. of a pharmaceutical composition may comprise from about 1×10\hat{ }6 CFU/dose to about 7×10\hat{ }8 CFU/dose. In some cases, at least one strain of Faecalibacterium sp. of a pharmaceutical composition may comprise from about 1×10\hat{ }6 CFU/dose to about 6×10\hat{ }8 CFU/dose. In some cases, at least one strain of Faecalibacterium sp. of a pharmaceutical composition may comprise from about 1×10\hat{ }6 CFU/dose to about 5×10\hat{ }8 CFU/dose. In some cases, at least one strain of Faecalibacterium sp. of a pharmaceutical composition may comprise from about 1×10\hat{ }6 CFU/dose to about 4×10\hat{ }8 CFU/dose. In some cases, at least one strain of Faecalibacterium sp. of a pharmaceutical composition may comprise from about 1×10\hat{ }6 CFU/dose to about 3×10\hat{ }8 CFU/dose. In some cases, at least one strain of Faecalibacterium sp. of a pharmaceutical composition may comprise from about 1×10\hat{ }6 CFU/dose to about 2×10\hat{ }8 CFU/dose. In some cases, at least one strain of Faecalibacterium sp. of a pharmaceutical composition may comprise from about 1×10\hat{ }6 CFU/dose to about 1×10\hat{ }8 CFU/dose. In some cases, at least one strain of Faecalibacterium sp. of a pharmaceutical composition may comprise from about 1×10\hat{ }6 CFU/dose to about 9×10\hat{ }7 CFU/dose. In some cases, at least one strain of Faecalibacterium sp. of a pharmaceutical composition may comprise from about 1×10\hat{ }6 CFU/dose to about 8×10\hat{ }7 CFU/dose. In some cases, at least one strain of Faecalibacterium sp. of a pharmaceutical composition may comprise from about 1×10\hat{ }6 CFU/dose to about 7×10\hat{ }7 CFU/dose. In some cases, at least one strain of Faecalibacterium sp. of a pharmaceutical composition may comprise from about 1×10\hat{ }6 CFU/dose to about 6×10\hat{ }7 CFU/dose. In some cases, at least one strain of Faecalibacterium sp. of a pharmaceutical composition may comprise from about 1×10\hat{ }6 CFU/dose to about 5×10\hat{ }7 CFU/dose. In some cases, at least one strain of Faecalibacterium sp. of a pharmaceutical composition may comprise from about 1×10\hat{ }6 CFU/dose to about 4×10\hat{ }7 CFU/dose. In some cases, at least one strain of Faecalibacterium sp. of a pharmaceutical composition may comprise from about 1×10\hat{ }6 CFU/dose to about 3×10\hat{ }7 CFU/dose. In some cases, at least one strain of Faecalibacterium sp. of a pharmaceutical composition may comprise from about 1×10\hat{ }6 CFU/dose to about 2×10\hat{ }7 CFU/dose. In some cases, at least one strain of Faecalibacterium sp. of a pharmaceutical composition may comprise from about 1×10\hat{ }6 CFU/dose to about 1×10\hat{ }7 CFU/dose. In some cases, at least one strain of Faecalibacterium sp. may comprise at least about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, 1×10\hat{ }12 or more CFU/dose. In some cases, at least one strain of Faecalibacterium sp. may comprise at most about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, or 1×10\hat{ }12 CFU/dose. In some cases, at least one strain of Faecalibacterium sp. may comprise about 5×10\hat{ }9 CFU/dose. In some cases, at least one strain of Faecalibacterium sp. may comprise about 4×10\hat{ }9 CFU/dose. In some cases, at least one strain of Faecalibacterium sp. may comprise about 3×10\hat{ }9 CFU/dose. In some cases, at least one strain of Faecalibacterium sp. may comprise about 2×10\hat{ }9 CFU/dose. In some cases, at least one strain of Faecalibacterium sp. may comprise about 1×10\hat{ }9 CFU/dose. In some cases, at least one strain of Faecalibacterium sp. may comprise about 9×10\hat{ }8 CFU/dose. In some cases, at least one strain of Faecalibacterium sp. may comprise about 8×10\hat{ }8 CFU/dose. In some cases, at least one strain of Faecalibacterium sp. may comprise about 7×10\hat{ }8 CFU/dose. In some cases, at least one strain of Faecalibacterium sp. may comprise about 6×10\hat{ }8 CFU/dose. In some cases, at least one strain of Faecalibacterium sp. may comprise about 5×10\hat{ }8 CFU/dose. In some cases, at least one strain of Faecalibacterium sp. may comprise about 4×10\hat{ }8 CFU/dose. In some cases, at least one strain of Faecalibacterium sp. may comprise about 3×10\hat{ }8 CFU/dose. In some cases, at least one strain of Faecalibacterium sp. may comprise about 2×10\hat{ }8 CFU/dose. In some cases, at least one strain of Faecalibacterium sp. may comprise about 1×10\hat{ }8 CFU/dose. In some cases, at least one strain of Faecalibacterium sp. may comprise about 9×10\hat{ }7 CFU/dose. In some cases, at least one strain of Faecalibacterium sp. may comprise about 8×10\hat{ }7 CFU/dose. In some cases, at least one strain of Faecalibacterium sp. may comprise about 7×10\hat{ }7 CFU/dose. In some cases, at least one strain of Faecalibacterium sp. may comprise about 6×10\hat{ }7 CFU/dose. In some cases, at least one strain of Faecalibacterium sp. may comprise about 5×10\hat{ }7 CFU/dose. In some cases, at least one strain of Faecalibacterium sp. may comprise about 4×10\hat{ }7 CFU/dose. In some cases, at least one strain of Faecalibacterium sp. may comprise about 3×10\hat{ }7 CFU/dose. In some cases, at least one strain of Faecalibacterium sp. may comprise about 2×10\hat{ }7 CFU/dose. In some cases, at least one strain of Faecalibacterium sp. may comprise about 1×10\hat{ }7 CFU/dose.

In some cases, F. prausnitzii (DSM 33185) of a pharmaceutical composition may comprise from about 1×10\hat{ }6 CFU/dose to about 5×10\hat{ }9 CFU/dose. In some cases, F. prausnitzii (DSM 33185) of a pharmaceutical composition may comprise from about 1×10\hat{ }6 CFU/dose to about 4×10\hat{ }9 CFU/dose. In some cases, F. prausnitzii (DSM 33185) of a pharmaceutical composition may comprise from about 1×10\hat{ }6 CFU/dose to about 3×10\hat{ }9 CFU/dose. In some cases, F. prausnitzii (DSM 33185) of a pharmaceutical composition may comprise from about 1×10\hat{ }6 CFU/dose to about 2×10\hat{ }9 CFU/dose. In some cases, F. prausnitzii (DSM 33185) of a pharmaceutical composition may comprise from about 1×10\hat{ }6 CFU/dose to about 1×10\hat{ }9 CFU/dose. In some cases, F. prausnitzii (DSM 33185) of a pharmaceutical composition may comprise from about 1×10\hat{ }6 CFU/dose to about 9×10\hat{ }8 CFU/dose. In some cases, F. prausnitzii (DSM 33185) of a pharmaceutical composition may comprise from about 1×10\hat{ }6 CFU/dose to about 8×10\hat{ }8 CFU/dose. In some cases, F. prausnitzii (DSM 33185) of a pharmaceutical composition may comprise from about 1×10\hat{ }6 CFU/dose to about 7×10\hat{ }8 CFU/dose. In some cases, F. prausnitzii (DSM 33185) of a pharmaceutical composition may comprise from about 1×10\hat{ }6 CFU/dose to about 6×10\hat{ }8 CFU/dose. In some cases, F. prausnitzii (DSM 33185) of a pharmaceutical composition may comprise from about 1×10\hat{ }6 CFU/dose to about 5×10\hat{ }8 CFU/dose. In some cases, F. prausnitzii (DSM 33185) of a pharmaceutical composition may comprise from about 1×10\hat{ }6 CFU/dose to about 4×10\hat{ }8 CFU/dose. In some cases, F. prausnitzii (DSM 33185) of a pharmaceutical composition may comprise from about 1×10\hat{ }6 CFU/dose to about 3×10\hat{ }8 CFU/dose. In some cases, F. prausnitzii (DSM 33185) of a pharmaceutical composition may comprise from about 1×10\hat{ }6 CFU/dose to about 2×10\hat{ }8 CFU/dose. In some cases, F. prausnitzii (DSM 33185) of a pharmaceutical composition may comprise from about 1×10\hat{ }6 CFU/dose to about 1×10\hat{ }8 CFU/dose. In some cases, F. prausnitzii (DSM 33185) of a pharmaceutical composition may comprise from about 1×10\hat{ }6 CFU/dose to about 9×10\hat{ }7 CFU/dose. In some cases, F. prausnitzii (DSM 33185) of a pharmaceutical composition may comprise from about 1×10\hat{ }6 CFU/dose to about 8×10\hat{ }7 CFU/dose. In some cases, F. prausnitzii (DSM 33185) of a pharmaceutical composition may comprise from about 1×10\hat{ }6 CFU/dose to about 7×10\hat{ }7 CFU/dose. In some cases, F. prausnitzii (DSM 33185) of a pharmaceutical composition may comprise from about 1×10\hat{ }6 CFU/dose to about 6×10\hat{ }7 CFU/dose. In some cases, F. prausnitzii (DSM 33185) of a pharmaceutical composition may comprise from about 1×10\hat{ }6 CFU/dose to about 5×10\hat{ }7 CFU/dose. In some cases, F. prausnitzii (DSM 33185) of a pharmaceutical composition may comprise from about 1×10\hat{ }6 CFU/dose to about 4×10\hat{ }7 CFU/dose. In some cases, F. prausnitzii (DSM 33185) of a pharmaceutical composition may comprise from about 1×10\hat{ }6 CFU/dose to about 3×10\hat{ }7 CFU/dose. In some cases, F. prausnitzii (DSM 33185) of a pharmaceutical composition may comprise from about 1×10\hat{ }6 CFU/dose to about 2×10\hat{ }7 CFU/dose. In some cases, F. prausnitzii (DSM 33185) of a pharmaceutical composition may comprise from about 1×10\hat{ }6 CFU/dose to about 1×10\hat{ }7 CFU/dose. In some cases, F. prausnitzii (DSM 33185) may comprise about 5×10\hat{ }9 CFU/dose. In some cases, F. prausnitzii (DSM 33185) may comprise about 4×10\hat{ }9 CFU/dose. In some cases, F. prausnitzii (DSM 33185) may comprise about 3×10\hat{ }9 CFU/dose. In some cases, F. prausnitzii (DSM 33185) may comprise about 2×10\hat{ }9 CFU/dose. In some cases, F. prausnitzii (DSM 33185) may comprise about 1×10\hat{ }9 CFU/dose. In some cases, F. prausnitzii (DSM 33185) may comprise about 9×10\hat{ }8 CFU/dose. In some cases, F. prausnitzii (DSM 33185) may comprise about 8×10\hat{ }8 CFU/dose. In some cases, F. prausnitzii (DSM 33185) may comprise about 7×10\hat{ }8 CFU/dose. In some cases, F. prausnitzii (DSM 33185) may comprise about 6×10\hat{ }8 CFU/dose. In some cases, F. prausnitzii (DSM 33185) may comprise about 5×10\hat{ }8 CFU/dose. In some cases, F. prausnitzii (DSM 33185) may comprise about 4×10\hat{ }8 CFU/dose. In some cases, F. prausnitzii (DSM 33185) may comprise about 3×10\hat{ }8 CFU/dose. In some cases, F. prausnitzii (DSM 33185) may comprise about 2×10\hat{ }8 CFU/dose. In some cases, F. prausnitzii (DSM 33185) may comprise about 1×10\hat{ }8 CFU/dose. In some cases, F. prausnitzii (DSM 33185) may comprise about 9×10\hat{ }7 CFU/dose. In some cases, F. prausnitzii (DSM 33185) may comprise about 8×10\hat{ }7 CFU/dose. In some cases, F. prausnitzii (DSM 33185) may comprise about 7×10\hat{ }7 CFU/dose. In some cases, F. prausnitzii (DSM 33185) may comprise about 6×10\hat{ }7 CFU/dose. In some cases, F. prausnitzii (DSM 33185) may comprise about 5×10\hat{ }7 CFU/dose. In some cases, F. prausnitzii (DSM 33185) may comprise about 4×10\hat{ }7 CFU/dose. In some cases, F. prausnitzii (DSM 33185) may comprise about 3×10\hat{ }7 CFU/dose. In some cases, F. prausnitzii (DSM 33185) may comprise about 2×10\hat{ }7 CFU/dose. In some cases, F. prausnitzii (DSM 33185) may comprise about 1×10\hat{ }7 CFU/dose.

In some cases, at least one strain of Lactobacillus sp. of a pharmaceutical composition may comprise from about 1×10\hat{ }7 CFU/dose to about 5×10\hat{ }10 CFU/dose. In some cases, at least one strain of species of the Lactobacillaceae family of a pharmaceutical composition may comprise from about 1×10\hat{ }7 CFU/dose to about 5×10\hat{ }10 CFU/dose. In some cases, at least one strain of Lactobacillus sp. of a pharmaceutical composition may comprise from about 1×10\hat{ }7 CFU/dose to about 4×10\hat{ }10 CFU/dose. In some cases, at least one strain of species of the Lactobacillaceae family of a pharmaceutical composition may comprise from about 1×10\hat{ }7 CFU/dose to about 4×10\hat{ }10 CFU/dose. In some cases, at least one strain of Lactobacillus sp. of a pharmaceutical composition may comprise from about 1×10\hat{ }7 CFU/dose to about 3×10\hat{ }10 CFU/dose. In some cases, at least one strain of species of the Lactobacillaceae family of a pharmaceutical composition may comprise from about 1×10\hat{ }7 CFU/dose to about 3×10\hat{ }10 CFU/dose. In some cases, at least one strain of Lactobacillus sp. of a pharmaceutical composition may comprise from about 1×10\hat{ }7 CFU/dose to about 2×10\hat{ }10 CFU/dose. In some cases, at least one strain of species of the Lactobacillaceae family of a pharmaceutical composition may comprise from about 1×10\hat{ }7 CFU/dose to about 2×10\hat{ }10 CFU/dose. In some cases, at least one strain of Lactobacillus sp. of a pharmaceutical composition may comprise from about 1×10\hat{ }7 CFU/dose to about 1×10\hat{ }10 CFU/dose. In some cases, at least one strain of species of the Lactobacillaceae family of a pharmaceutical composition may comprise from about 1×10\hat{ }7 CFU/dose to about 1×10\hat{ }10 CFU/dose. In some cases, at least one strain of Lactobacillus sp. of a pharmaceutical composition may comprise from about 1×10\hat{ }7 CFU/dose to about 9×10\hat{ }9 CFU/dose. In some cases, at least one strain of species of the Lactobacillaceae family of a pharmaceutical composition may comprise from about 1×10\hat{ }7 CFU/dose to about 9×10\hat{ }9 CFU/dose. In some cases, at least one strain of Lactobacillus sp. of a pharmaceutical composition may comprise from about 1×10\hat{ }7 CFU/dose to about 8×10\hat{ }9 CFU/dose. In some cases, at least one strain of species of the Lactobacillaceae family of a pharmaceutical composition may comprise from about 1×10\hat{ }7 CFU/dose to about 8×10\hat{ }9 CFU/dose. In some cases, at least one strain of Lactobacillus sp. of a pharmaceutical composition may comprise from about 1×10\hat{ }7 CFU/dose to about 7×10\hat{ }9 CFU/dose. In some cases, at least one strain of species of the Lactobacillaceae family of a pharmaceutical composition may comprise from about 1×10\hat{ }7 CFU/dose to about 7×10\hat{ }9 CFU/dose. In some cases, at least one strain of Lactobacillus sp. of a pharmaceutical composition may comprise from about 1×10\hat{ }7 CFU/dose to about 6×10\hat{ }9 CFU/dose. In some cases, at least one strain of species of the Lactobacillaceae family of a pharmaceutical composition may comprise from about 1×10\hat{ }7 CFU/dose to about 6×10\hat{ }9 CFU/dose. In some cases, at least one strain of Lactobacillus sp. of a pharmaceutical composition may comprise from about 1×10\hat{ }7 CFU/dose to about 5×10\hat{ }9 CFU/dose. In some cases, at least one strain of species of the Lactobacillaceae family of a pharmaceutical composition may comprise from about 1×10\hat{ }7 CFU/dose to about 5×10\hat{ }9 CFU/dose. In some cases, at least one strain of Lactobacillus sp. of a pharmaceutical composition may comprise from about 1×10\hat{ }7 CFU/dose to about 4×10\hat{ }9 CFU/dose. In some cases, at least one strain of species of the Lactobacillaceae family of a pharmaceutical composition may comprise from about 1×10\hat{ }7 CFU/dose to about 4×10\hat{ }9 CFU/dose. In some cases, at least one strain of Lactobacillus sp. of a pharmaceutical composition may comprise from about 1×10\hat{ }7 CFU/dose to about 3×10\hat{ }9 CFU/dose. In some cases, at least one strain of species of the Lactobacillaceae family of a pharmaceutical composition may comprise from about 1×10\hat{ }7 CFU/dose to about 3×10\hat{ }9 CFU/dose. In some cases, at least one strain of Lactobacillus sp. of a pharmaceutical composition may comprise from about 1×10\hat{ }7 CFU/dose to about 2×10\hat{ }9 CFU/dose. In some cases, at least one strain of species of the Lactobacillaceae family of a pharmaceutical composition may comprise from about 1×10\hat{ }7 CFU/dose to about 2×10\hat{ }9 CFU/dose. In some cases, at least one strain of Lactobacillus sp. of a pharmaceutical composition may comprise from about 1×10\hat{ }7 CFU/dose to about 1×10\hat{ }9 CFU/dose. In some cases, at least one strain of species of the Lactobacillaceae family of a pharmaceutical composition may comprise from about 1×10\hat{ }7 CFU/dose to about 1×10\hat{ }9 CFU/dose. In some cases, at least one strain of Lactobacillus sp. of a pharmaceutical composition may comprise from about 1×10\hat{ }7 CFU/dose to about 9×10\hat{ }8 CFU/dose. In some cases, at least one strain of species of the Lactobacillaceae family of a pharmaceutical composition may comprise from about 1×10\hat{ }7 CFU/dose to about 9×10\hat{ }8 CFU/dose. In some cases, at least one strain of Lactobacillus sp. of a pharmaceutical composition may comprise from about 1×10\hat{ }7 CFU/dose to about 8×10\hat{ }8 CFU/dose. In some cases, at least one strain of species of the Lactobacillaceae family of a pharmaceutical composition may comprise from about 1×10\hat{ }7 CFU/dose to about 8×10\hat{ }8 CFU/dose. In some cases, at least one strain of Lactobacillus sp. of a pharmaceutical composition may comprise from about 1×10\hat{ }7 CFU/dose to about 7×10\hat{ }8 CFU/dose. In some cases, at least one strain of species of the Lactobacillaceae family of a pharmaceutical composition may comprise from about 1×10\hat{ }7 CFU/dose to about 7×10\hat{ }8 CFU/dose. In some cases, at least one strain of Lactobacillus sp. of a pharmaceutical composition may comprise from about 1×10\hat{ }7 CFU/dose to about 6×10\hat{ }8 CFU/dose. In some cases, at least one strain of species of the Lactobacillaceae family of a pharmaceutical composition may comprise from about 1×10\hat{ }7 CFU/dose to about 6×10\hat{ }8 CFU/dose. In some cases, at least one strain of Lactobacillus sp. of a pharmaceutical composition may comprise from about 1×10\hat{ }7 CFU/dose to about 5×10\hat{ }8 CFU/dose. In some cases, at least one strain of species of the Lactobacillaceae family of a pharmaceutical composition may comprise from about 1×10\hat{ }7 CFU/dose to about 5×10\hat{ }8 CFU/dose. In some cases, at least one strain of Lactobacillus sp. of a pharmaceutical composition may comprise from about 1×10\hat{ }7 CFU/dose to about 4×10\hat{ }8 CFU/dose. In some cases, at least one strain of species of the Lactobacillaceae family of a pharmaceutical composition may comprise from about 1×10\hat{ }7 CFU/dose to about 4×10\hat{ }8 CFU/dose. In some cases, at least one strain of Lactobacillus sp. of a pharmaceutical composition may comprise from about 1×10\hat{ }7 CFU/dose to about 3×10\hat{ }8 CFU/dose. In some cases, at least one strain of species of the Lactobacillaceae family of a pharmaceutical composition may comprise from about 1×10\hat{ }7 CFU/dose to about 3×10\hat{ }8 CFU/dose. In some cases, at least one strain of Lactobacillus sp. of a pharmaceutical composition may comprise from about 1×10\hat{ }7 CFU/dose to about 2×10\hat{ }8 CFU/dose. In some cases, at least one strain of species of the Lactobacillaceae family of a pharmaceutical composition may comprise from about 1×10\hat{ }7 CFU/dose to about 2×10\hat{ }8 CFU/dose. In some cases, at least one strain of Lactobacillus sp. of a pharmaceutical composition may comprise from about 1×10\hat{ }7 CFU/dose to about 1×10\hat{ }8 CFU/dose. In some cases, at least one strain of species of the Lactobacillaceae family of a pharmaceutical composition may comprise from about 1×10\hat{ }7 CFU/dose to about 1×10\hat{ }8 CFU/dose. In some cases, at least one strain of Lactobacillus sp. may comprise at least about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, 1×10\hat{ }12 or more CFU/dose. In some cases, at least one strain of Lactobacillus sp. may comprise at most about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, or 1×10\hat{ }12 CFU/dose. In some cases, at least one strain of species of the Lactobacillaceae family may comprise at least about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, 1×10\hat{ }12 or more CFU/dose. In some cases, at least one strain of species of the Lactobacillaceae family may comprise at most about 1×10\hat{ }3, 1×10\hat{ }4, 1×10\hat{ }5, 1×10\hat{ }6, 1×10\hat{ }7, 1×10\hat{ }8, 1×10\hat{ }9, 1×10\hat{ }10, 1×10\hat{ }11, or 1×10\hat{ }12 CFU/dose. In some cases, at least one strain of Lactobacillus sp. may comprise about 5×10\hat{ }9 CFU/dose. In some cases, at least one strain of species of the Lactobacillaceae family may comprise about 5×10\hat{ }9 CFU/dose. In some cases, at least one strain of Lactobacillus sp. may comprise about 4×10\hat{ }9 CFU/dose. In some cases, at least one strain of species of the Lactobacillaceae family may comprise about 4×10\hat{ }9 CFU/dose. In some cases, at least one strain of Lactobacillus sp. may comprise about 3×10\hat{ }9 CFU/dose. In some cases, at least one strain of species of the Lactobacillaceae family may comprise about 3×10\hat{ }9 CFU/dose. In some cases, at least one strain of Lactobacillus sp. may comprise about 2×10\hat{ }9 CFU/dose. In some cases, at least one strain of species of the Lactobacillaceae family may comprise about 2×10\hat{ }9 CFU/dose. In some cases, at least one strain of Lactobacillus sp. may comprise about 1×10\hat{ }9 CFU/dose. In some cases, at least one strain of species of the Lactobacillaceae family may comprise about 1×10\hat{ }9 CFU/dose. In some cases, at least one strain of Lactobacillus sp. may comprise about 9×10\hat{ }8 CFU/dose. In some cases, at least one strain of species of the Lactobacillaceae family may comprise about 9×10\hat{ }8 CFU/dose. In some cases, at least one strain of Lactobacillus sp. may comprise about 8×10\hat{ }8 CFU/dose. In some cases, at least one strain of species of the Lactobacillaceae family may comprise about 8×10\hat{ }8 CFU/dose. In some cases, at least one strain of Lactobacillus sp. may comprise about 7×10\hat{ }8 CFU/dose. In some cases, at least one strain of species of the Lactobacillaceae family may comprise about 7×10\hat{ }8 CFU/dose. In some cases, at least one strain of Lactobacillus sp. may comprise about 6×10\hat{ }8 CFU/dose. In some cases, at least one strain of species of the Lactobacillaceae family may comprise about 6×10\hat{ }8 CFU/dose. In some cases, at least one strain of Lactobacillus sp. may comprise about 5×10\hat{ }8 CFU/dose. In some cases, at least one strain of species of the Lactobacillaceae family may comprise about 5×10\hat{ }8 CFU/dose. In some cases, at least one strain of Lactobacillus sp. may comprise about 4×10\hat{ }8 CFU/dose. In some cases, at least one strain of species of the Lactobacillaceae family may comprise about 4×10\hat{ }8 CFU/dose. In some cases, at least one strain of Lactobacillus sp. may comprise about 3×10\hat{ }8 CFU/dose. In some cases, at least one strain of species of the Lactobacillaceae family may comprise about 3×10\hat{ }8 CFU/dose. In some cases, at least one strain of Lactobacillus sp. may comprise about 2×10\hat{ }8 CFU/dose. In some cases, at least one strain of species of the Lactobacillaceae family may comprise about 2×10\hat{ }8 CFU/dose. In some cases, at least one strain of Lactobacillus sp. may comprise about 1×10\hat{ }8 CFU/dose. In some cases, at least one strain of species of the Lactobacillaceae family may comprise about 1×10\hat{ }8 CFU/dose. In some cases, at least one strain of Lactobacillus sp. may comprise about 9×10\hat{ }7 CFU/dose. In some cases, at least one strain of species of the Lactobacillaceae family may comprise about 9×10\hat{ }7 CFU/dose. In some cases, at least one strain of Lactobacillus sp. may comprise about 8×10\hat{ }7 CFU/dose. In some cases, at least one strain of species of the Lactobacillaceae family may comprise about 8×10\hat{ }7 CFU/dose. In some cases, at least one strain of Lactobacillus sp. may comprise about 7×10\hat{ }7 CFU/dose. In some cases, at least one strain of species of the Lactobacillaceae family may comprise about 7×10\hat{ }7 CFU/dose. In some cases, at least one strain of Lactobacillus sp. may comprise about 6×10\hat{ }7 CFU/dose. In some cases, at least one strain of species of the Lactobacillaceae family may comprise about 6×10\hat{ }7 CFU/dose. In some cases, at least one strain of Lactobacillus sp. may comprise about 5×10\hat{ }7 CFU/dose. In some cases, at least one strain of species of the Lactobacillaceae family may comprise about 5×10\hat{ }7 CFU/dose. In some cases, at least one strain of Lactobacillus sp. may comprise about 4×10\hat{ }7 CFU/dose. In some cases, at least one strain of species of the Lactobacillaceae family may comprise about 4×10\hat{ }7 CFU/dose. In some cases, at least one strain of Lactobacillus sp. may comprise about 3×10\hat{ }7 CFU/dose. In some cases, at least one strain of species of the Lactobacillaceae family may comprise about 3×10\hat{ }7 CFU/dose. In some cases, at least one strain of Lactobacillus sp. may comprise about 2×10\hat{ }7 CFU/dose. In some cases, at least one strain of species of the Lactobacillaceae family may comprise about 2×10\hat{ }7 CFU/dose. In some cases, at least one strain of Lactobacillus sp. may comprise about 1×10\hat{ }7 CFU/dose. In some cases, at least one strain of species of the Lactobacillaceae family may comprise about 1×10\hat{ }7 CFU/dose.

In some cases, L. crispatus (DSM 33187) of a pharmaceutical composition may comprise from about 1×10\hat{ }7 CFU/dose to about 5×10\hat{ }10 CFU/dose. In some cases, L. crispatus (DSM 33187) of a pharmaceutical composition may comprise from about 1×10\hat{ }7 CFU/dose to about 4×10\hat{ }10 CFU/dose. In some cases, L. crispatus (DSM 33187) of a pharmaceutical composition may comprise from about 1×10\hat{ }7 CFU/dose to about 3×10\hat{ }10 CFU/dose. In some cases, L. crispatus (DSM 33187) of a pharmaceutical composition may comprise from about 1×10\hat{ }7 CFU/dose to about 2×10\hat{ }10 CFU/dose. In some cases, L. crispatus (DSM 33187) of a pharmaceutical composition may comprise from about 1×10\hat{ }7 CFU/dose to about 1×10\hat{ }10 CFU/dose. In some cases, L. crispatus (DSM 33187) of a pharmaceutical composition may comprise from about 1×10\hat{ }7 CFU/dose to about 9×10\hat{ }9 CFU/dose. In some cases, L. crispatus (DSM 33187) of a pharmaceutical composition may comprise from about 1×10\hat{ }7 CFU/dose to about 8×10\hat{ }9 CFU/dose. In some cases, L. crispatus (DSM 33187) of a pharmaceutical composition may comprise from about 1×10\hat{ }7 CFU/dose to about 7×10\hat{ }9 CFU/dose. In some cases, L. crispatus (DSM 33187) of a pharmaceutical composition may comprise from about 1×10\hat{ }7 CFU/dose to about 6×10\hat{ }9 CFU/dose. In some cases, L. crispatus (DSM 33187) of a pharmaceutical composition may comprise from about 1×10\hat{ }7 CFU/dose to about 5×10\hat{ }9 CFU/dose. In some cases, L. crispatus (DSM 33187) of a pharmaceutical composition may comprise from about 1×10\hat{ }7 CFU/dose to about 4×10\hat{ }9 CFU/dose. In some cases, L. crispatus (DSM 33187) of a pharmaceutical composition may comprise from about 1×10\hat{ }7 CFU/dose to about 3×10\hat{ }9 CFU/dose. In some cases, L. crispatus (DSM 33187) of a pharmaceutical composition may comprise from about 1×10\hat{ }7 CFU/dose to about 2×10\hat{ }9 CFU/dose. In some cases, L. crispatus (DSM 33187) of a pharmaceutical composition may comprise from about 1×10\hat{ }7 CFU/dose to about 1×10\hat{ }9 CFU/dose. In some cases, L. crispatus (DSM 33187) of a pharmaceutical composition may comprise from about 1×10\hat{ }7 CFU/dose to about 9×10\hat{ }8 CFU/dose. In some cases, L. crispatus (DSM 33187) of a pharmaceutical composition may comprise from about 1×10\hat{ }7 CFU/dose to about 8×10\hat{ }8 CFU/dose. In some cases, L. crispatus (DSM 33187) of a pharmaceutical composition may comprise from about 1×10\hat{ }7 CFU/dose to about 7×10\hat{ }8 CFU/dose. In some cases, L. crispatus (DSM 33187) of a pharmaceutical composition may comprise from about 1×10\hat{ }7 CFU/dose to about 6×10\hat{ }8 CFU/dose. In some cases, L. crispatus (DSM 33187) of a pharmaceutical composition may comprise from about 1×10\hat{ }7 CFU/dose to about 5×10\hat{ }8 CFU/dose. In some cases, L. crispatus (DSM 33187) of a pharmaceutical composition may comprise from about 1×10\hat{ }7 CFU/dose to about 4×10\hat{ }8 CFU/dose. In some cases, L. crispatus (DSM 33187) of a pharmaceutical composition may comprise from about 1×10\hat{ }7 CFU/dose to about 3×10\hat{ }8 CFU/dose. In some cases, L. crispatus (DSM 33187) of a pharmaceutical composition may comprise from about 1×10\hat{ }7 CFU/dose to about 2×10\hat{ }8 CFU/dose. In some cases, L. crispatus (DSM 33187) of a pharmaceutical composition may comprise from about 1×10\hat{ }7 CFU/dose to about 1×10\hat{ }8 CFU/dose. In some cases, L. crispatus (DSM 33187) may comprise about 5×10\hat{ }9 CFU/dose. In some cases, L. crispatus (DSM 33187) may comprise about 4×10\hat{ }9 CFU/dose. In some cases, L. crispatus (DSM 33187) may comprise about 3×10\hat{ }9 CFU/dose. In some cases, L. crispatus (DSM 33187) may comprise about 2×10\hat{ }9 CFU/dose. In some cases, L. crispatus (DSM 33187) may comprise about 1×10\hat{ }9 CFU/dose. In some cases, L. crispatus (DSM 33187) may comprise about 9×10\hat{ }8 CFU/dose. In some cases, L. crispatus (DSM 33187) may comprise about 8×10\hat{ }8 CFU/dose. In some cases, L. crispatus (DSM 33187) may comprise about 7×10\hat{ }8 CFU/dose. In some cases, L. crispatus (DSM 33187) may comprise about 6×10\hat{ }8 CFU/dose. In some cases, L. crispatus (DSM 33187) may comprise about 5×10\hat{ }8 CFU/dose. In some cases, L. crispatus (DSM 33187) may comprise about 4×10\hat{ }8 CFU/dose. In some cases, L. crispatus (DSM 33187) may comprise about 3×10\hat{ }8 CFU/dose. In some cases, L. crispatus (DSM 33187) may comprise about 2×10\hat{ }8 CFU/dose. In some cases, L. crispatus (DSM 33187) may comprise about 1×10\hat{ }8 CFU/dose. In some cases, L. crispatus (DSM 33187) may comprise about 9×10\hat{ }7 CFU/dose. In some cases, L. crispatus (DSM 33187) may comprise about 8×10\hat{ }7 CFU/dose. In some cases, L. crispatus (DSM 33187) may comprise about 7×10\hat{ }7 CFU/dose. In some cases, L. crispatus (DSM 33187) may comprise about 6×10\hat{ }7 CFU/dose. In some cases, L. crispatus (DSM 33187) may comprise about 5×10\hat{ }7 CFU/dose. In some cases, L. crispatus (DSM 33187) may comprise about 4×10\hat{ }7 CFU/dose. In some cases, L. crispatus (DSM 33187) may comprise about 3×10\hat{ }7 CFU/dose. In some cases, L. crispatus (DSM 33187) may comprise about 2×10\hat{ }7 CFU/dose. In some cases, L. crispatus (DSM 33187) may comprise about 1×10\hat{ }7 CFU/dose.

In some cases herein, a bacterial population for use in a pharmaceutical composition of this disclosure may comprise or consist of about 5×10\hat{ }8 CFU/mL of any of the bacterial strains L. crispatus (DSM 33187), A. muciniphila (DSM 33213), and/or F. prausnitzii (DSM 33185). In such instances, the bacterial population may consist of about 5×10\hat{ }8 CFU/mL of the bacterial strains L. crispatus (DSM 33187), A. muciniphila (DSM 33213), and F. prausnitzii (DSM 33185).

In instances where a pharmaceutical composition is formulated into a unit dose for administration, such CFU/dose values may be per mass unit (e.g., 5×10\hat{ }8 CFU/dose/g) or volume unit (e.g., 5×10\hat{ }8 CFU/dose/mL) of such dosage form. In other cases, the CFU/dose value may be equivalent to CFU/capsule if one dose is administered as one capsule, or if one capsule contains a dose. In some cases, a dose may be in other forms described elsewhere in this disclosure.

In some instance, a bacterial population for use in a pharmaceutical composition described herein may comprise at least about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, 5×10\hat{ }15 CFU/gram of bacterial cells or more. In some instance, a bacterial population for use in a pharmaceutical composition described herein may comprise at most about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, or 5×10\hat{ }15 CFU/gram of bacterial cells.

In some instance, at least one strain of Akkermansia sp. in a pharmaceutical composition described herein may comprise at least about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, 5×10\hat{ }15 CFU/gram of bacterial cells or more. In some instance, at least one strain of Akkermansia sp. in a pharmaceutical composition described herein may comprise at most about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, or 5×10\hat{ }15 CFU/gram of bacterial cells.

In some instance, at least one strain of Faecalibacterium sp. in a pharmaceutical composition described herein may comprise at least about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, 5×10\hat{ }15 CFU/gram of bacterial cells or more. In some instance, at least one strain of Faecalibacterium sp. in a pharmaceutical composition described herein may comprise at most about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, or 5×10\hat{ }15 CFU/gram of bacterial cells.

In some instance, at least one strain of Lactobacillus sp. in a pharmaceutical composition described herein may comprise at least about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, 5×10\hat{ }15 CFU/gram of bacterial cells or more. In some instance, at least one strain of Lactobacillus sp. in a pharmaceutical composition described herein may comprise at most about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, or 5×10\hat{ }15 CFU/gram of bacterial cells.

In some instance, at least one strain of a species of the Lactobacillaceae family in a pharmaceutical composition described herein may comprise at least about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, 5×10\hat{ }15 CFU/gram of bacterial cells or more. In some instance, at least one strain of a species of the Lactobacillaceae family in a pharmaceutical composition described herein may comprise at most about 1×10\hat{ }3 CFU/gram of bacterial cells, 5×10\hat{ }3 CFU/gram of bacterial cells, 1×10\hat{ }4 CFU/gram of bacterial cells, 5×10\hat{ }4 CFU/gram of bacterial cells, 1×10\hat{ }5 CFU/gram of bacterial cells, 5×10\hat{ }5 CFU/gram of bacterial cells, 1×10\hat{ }6 CFU/gram of bacterial cells, 5×10\hat{ }6 CFU/gram of bacterial cells, 1×10\hat{ }7 CFU/gram of bacterial cells, 5×10\hat{ }7 CFU/gram of bacterial cells, 1×10\hat{ }8 CFU/gram of bacterial cells, 5×10\hat{ }8 CFU/gram of bacterial cells, 1×10\hat{ }9 CFU/gram of bacterial cells, 5×10\hat{ }9 CFU/gram of bacterial cells, 1×10\hat{ }10 CFU/gram of bacterial cells, 5×10\hat{ }10 CFU/gram of bacterial cells, 1×10\hat{ }11 CFU/gram of bacterial cells, 5×10\hat{ }11 CFU/gram of bacterial cells, 1×10\hat{ }12 CFU/gram of bacterial cells, 5×10\hat{ }12 CFU/gram of bacterial cells, 1×10\hat{ }13 CFU/gram of bacterial cells, 5×10\hat{ }13 CFU/gram of bacterial cells, 1×10\hat{ }14 CFU/gram of bacterial cells, 5×10\hat{ }14 CFU/gram of bacterial cells, 1×10\hat{ }15 CFU/gram of bacterial cells, or 5×10\hat{ }15 CFU/gram of bacterial cells.

### Pharmaceutical Composition Forms and Administrations

In some instances, a pharmaceutical composition may be formulated into a suspension. In some cases, a pharmaceutical composition may be formulated into an oral dosage form. An oral dosage form of a pharmaceutical composition, in some cases, may comprise a capsule, tablet, emulsion, suspension, syrup, gel, gum, paste, herbal tea, drops, dissolving granules, powders, tablets, lyophilizate, a popsicle, foams, or ice cream. In some cases, an oral dosage form of a pharmaceutical composition may comprise a capsule. In some cases, an oral dosage form of a pharmaceutical composition may comprise a dissolving granule. In some cases, an oral dosage form of a pharmaceutical composition may comprise a drop. In some cases, an oral dosage form of a pharmaceutical composition may comprise an emulsion. In some cases, an oral dosage form of a pharmaceutical composition may comprise a foam. In some cases, an oral dosage form of a pharmaceutical composition may comprise a gel. In some cases, an oral dosage form of a pharmaceutical composition may comprise a gum. In some cases, an oral dosage form of a pharmaceutical composition may comprise a herbal tea. In some cases, an oral dosage form of a pharmaceutical composition may comprise an ice cream. In some cases, an oral dosage form of a pharmaceutical composition may comprise a lyophilizate. In some cases, an oral dosage form of a pharmaceutical composition may comprise a paste. In some cases, an oral dosage form of a pharmaceutical composition may comprise a popsicle. In some cases, an oral dosage form of a pharmaceutical composition may comprise a powder. In some cases, an oral dosage form of a pharmaceutical composition may comprise a suspension. In some cases, an oral dosage form of a pharmaceutical composition may comprise a syrup. In some cases, an oral dosage form of a pharmaceutical composition may comprise a tablet. In some cases, an oral dosage form of a pharmaceutical composition may comprise a pill, geltab, sachet, a lozenge, or any other suitable oral dosage form. In some cases, a pharmaceutical composition in an oral dosage or suspension form may be administered alone. In other cases, a pharmaceutical composition in an oral dosage or suspension form may be mixed with a food product for administration to a subject. Such a food product may comprise baby formula, milk, or any derivatives thereof. In some cases, a pharmaceutical composition may be formulated into a parenteral administration form. A parenteral administration form, in some cases, may comprise various non-oral routes, e.g., in the form of a suppository.

In some cases, a pharmaceutical composition may be encompassed by a primary container. In some instances, a pharmaceutical composition may be encompassed by a capsule. In some cases, a capsule encompassing a pharmaceutical composition may comprise a plant-based capsule. In some cases, a capsule encompassing a pharmaceutical composition may comprise a vegan capsule. In some cases, a plant-based capsule may comprise a plant-derived material. The plant-derived material, in some cases, may comprise a cellulose-based polymer. In some cases, a plant-based capsule may comprise a hypromellose capsule. In some cases, a plant-based capsule may comprise a hydroxypropyl methylcellulose (HPMC) capsule. In some cases, a plant-based capsule may comprise a starch capsule. In some cases, a plant-based capsule may comprise a hydrolyzed plant-based collagen capsule. In some cases, a plant-based capsule may comprise a pullulan capsule. In some cases, a plant-based capsule may comprise a tapioca capsule. In some cases, a plant-based capsule may comprise the combinations of any plant-based materials described thereof. A primary container, in some cases, may comprise any capsules described herein and thereof and derivatives herein and thereof.

In some cases, a capsule may not be administered to a subject. In some cases, a capsule may not be administered to a subject alongside the pharmaceutical composition. In other cases, a capsule may be administered to a subject.

A capsule may be enteric-coated. An enteric-coated capsule may comprise fatty acids, waxes, shellac, plastics, plant fibers, or any combination thereof. A capsule may have a size of 000, 00, 0, 1, 2, 3, 4, or 5 Empty Pill Capsule Size. In some cases, a capsule may comprise gelatin.

A capsule may be starch-free, gluten-free, and preservative-free. >90% of a capsule dissolves in water, pH=1.2 solution, sodium acetate buffer USP. (pH=4.5), or sodium phosphate buffer (pH=7.2) within 60 minutes, when measured by the dissolution of acetaminophen when the capsule is filled with unformulated acetaminophen. A capsule may have a disintegration endpoint of about 1.6 minutes, as measured at 37° C. with de-ionized water. A capsule may have a disintegration endpoint of about 0.1, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 3.4, 2.5, 2.6, 2.7, 2.8, 2.9, 3, 3.1, 3.2, 3.3, 3.4, 3.5, 3.6, 3.7, 3.8, 3.9, or 4 minutes, as measured at 37° C. with de-ionized water. A capsule may have a disintegration endpoint of 0.1 to 0.5 minutes, 0.51 to 0.6 minutes, 0.61 to 0.7 minutes, 0.71 to 0.8 minutes, 0.81 to 0.9 minutes, 0.91 to 1 minutes, 1.01 to 1.1 minutes, 1.11 to 1.2 minutes, 1.21 to 1.3 minutes, 1.31 to 1.4 minutes, 1.41 to 1.5 minutes, 1.51 to 1.6 minutes, 1.61 to 1.7 minutes, 1.71 to 1.8 minutes, 1.81 to 1.9 minutes, 1.91 to 2 minutes, 2.01 to 2.1 minutes, 2.11 to 2.2 minutes, 2.21 to 2.3 minutes, 2.31 to 2.4 minutes, 2.41 to 2.5 minutes, 2.51 to 2.6 minutes, 2.61 to 2.7 minutes, 2.71 to 2.8 minutes, 2.81 to 2.9 minutes, 2.91 to 3 minutes, 3.01 to 3.1 minutes, 3.11 to 3.2 minutes, 3.21 to 3.3 minutes, 3.31 to 3.4 minutes, 3.41 to 3.5 minutes, 3.51 to 3.6 minutes, 3.61 to 3.7 minutes, 3.71 to 3.8 minutes, 3.81 to 3.9 minutes, or 3.91 to 4 minutes, as measured at 37° C. with de-ionized water. A capsule may have an oxygen permeability (cm³/m²/day) of <0.5, as measured by a gas composition in the capsule. A capsule may have an oxygen permeability (cm³/m²/day) of <0.0001, <0.0005, <0.001, <0.005, <0.01, <0.05, <0.1, <0.5, <1, <1.5, <2, <5, or <10, as measured by a gas composition in the capsule.

In some cases, a pharmaceutical composition may be lyophilized. In some cases, a pharmaceutical composition may be frozen. Such frozen or lyophilized formulations may be administered in a frozen or lyophilized state to a subject. In some instances, such frozen formulation may be a popsicle, an ice cream, or other frozen formulations.

In some cases, a liquid suspension may be aliquoted into certain volumes to provide a unit dose of such oral dosage form. Such unit dose may have a volume of about 0.25, 0.5, 1, 2, 3, 5, or 10 mL. In some instances, the unit dose of a pharmaceutical composition herein has a volume of about 1 mL. Such pharmaceutical composition may comprise a bacterial population, a cryoprotectant, an antioxidant, an aqueous buffer solution that may from a liquid cell suspension. Such cell suspension may be tested for quality control to ensure it contains a certain number of metabolically active cells per bacterial strain as described herein.

In some instances, pharmaceutical compositions described herein are lyophilized or frozen. Bacterial cells in the lyophilized or frozen pharmaceutical compositions can be stored at −70° C. In some embodiments, the bacterial cells can be stored at 10° C., 4° C., 0° C., −5° C., −10° C., −15° C., −20° C., −25° C., −30° C., −35° C., −40° C., −45° C., −50° C., −55° C., −60° C., −65° C., −70° C., −75° C., or −80° C. In other cases, the bacterial cells can also be stored from −80° C. to −70° C., from −70° C. to −60° C., from −60° C. to −50° C., from −50° C. to −40° C., from −40° C. to −30° C., from −30° C. to −20° C., from −20° C. to −10° C., from −10° C. to 0° C., or from 0° C. to 10° C. In some embodiments, at least 70% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some cases, at least 75% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In other cases, at least 80% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some embodiments, at least 85% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In other embodiments, at least 90% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. At least 95% of the stored lyophilized or frozen bacterial cells can also remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some embodiments, at least 99% of the stored lyophilized or frozen bacterial cells can remain viable after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months.

In some instances, the pharmaceutical composition has a shelf stability. The shelf stability of the pharmaceutical composition may comprise the maintenance of the stability of the pharmaceutical composition. The shelf stability of the pharmaceutical composition can comprise maintenance of the pharmaceutical activity or pharmacological stability of the pharmaceutical composition. The shelf stability of the pharmaceutical composition can also comprise maintenance of the biological or physiological activity the pharmaceutical composition it elicits in a subject. (e.g., the ability to treat or prevent a risk of a disease in the subject). In some cases, shelf stability can comprise a measurable variable that indicates or correlates with the stability of the pharmaceutical composition, pharmaceutical activity of the pharmaceutical composition, pharmacological stability of the pharmaceutical composition, biological or physiological activity the pharmaceutical composition it elicits in a subject, or any combination thereof. In some cases, the shelf stability of the pharmaceutical composition can comprise the viability of a strain(s) of the pharmaceutical composition.

In some instances, shelf stability can be assessed subsequent to the pharmaceutical composition is stored in an environment. In some instances, shelf stability can be assessed during the pharmaceutical composition is stored in an environment. In some instances, shelf stability can be assessed prior to the pharmaceutical composition is stored in an environment. Shelf stability, in some cases, can be assessed at any point after production or manufacturing of the pharmaceutical composition. The shelf stability can be assessed for the entirety of the pharmaceutical composition. The shelf stability can be assessed for the individual bacterial strains in the bacterial population. The shelf stability can be assessed for the combinations of bacterial strains in the bacterial population. In some instances, the shelf stability is assessed at a time point subsequent to the pharmaceutical composition is manufactured. In some instances, the shelf stability is assessed at a time point prior to the pharmaceutical composition is manufactured. In some instances, the shelf stability is assessed at a time point during the pharmaceutical composition is being manufactured. To attain to the shelf stability, a pharmaceutical composition may comprise a cryoprotectant, a pharmaceutically acceptable excipient, be stored in a capsule, or a combination thereof.

In some instances, shelf stability of a pharmaceutical composition can be assessed by measuring the viability of the bacterial population of the pharmaceutical composition. The viability of the bacterial population of the pharmaceutical can be measured as the number of bacterial cells within the pharmaceutical composition. The viability of the bacterial populations can be measured in CFU. The viability of the bacterial population can also be measured by other means, including but not limited to optical density, that can measure the number of bacterial cells.

In some instances, shelf stability of a pharmaceutical composition can be expressed by a ratio of the viability of the number of the bacterial cells of the bacterial population of the pharmaceutical composition subsequent to the pharmaceutical composition is stored in an environment relative to the number of the bacterial cells of the bacterial population of a comparable pharmaceutical composition prior to the comparable pharmaceutical composition is stored in an environment. In some instances, shelf stability of a pharmaceutical composition can be expressed by a ratio of the viability of the number of the bacterial cells of the bacterial population of the pharmaceutical composition subsequent to the pharmaceutical composition is stored in an environment relative to the number of the bacterial cells of the bacterial population of a comparable pharmaceutical composition without being stored in an environment. The environment can be an anaerobic environment or aerobic environment. The environment can be an anaerobic environment. The environment can be an aerobic environment. A comparable pharmaceutical composition of a pharmaceutical composition can comprise a pharmaceutical composition with the same or substantially the same make-up of the pharmaceutical composition. The make-up of the pharmaceutical composition (or a comparable one) can comprise any one strain or all strains of the bacterial population, the numbers or proportions of the bacterial cells of the bacterial strains, the amount of the pharmaceutically acceptable excipients or cryoprotectant, the methods of manufacturing used, or any combination thereof.

For example, shelf stability of a pharmaceutical composition can be calculated by comparing the viability of the bacterial population of the pharmaceutical composition at a time point subsequent to the pharmaceutical composition is stored in an environment. In some cases, the shelf stability can be measured between about 0 to about 500 days after being stored in an environment. In some cases, the shelf stability can be measured about 3 days after being stored in an environment. In some cases, the shelf stability can be measured about 10 days after being stored in an environment. In some cases, the shelf stability can be measured about 100 days after being stored in an environment.

In some instances, the pharmaceutical composition can have a shelf stability of between about 1×10\hat{ }-8% and about 100% subsequent to being stored in an environment. In some cases, the pharmaceutical composition can have a shelf stability of at least about 1×10\hat{ }-8%, at least about 1×10\hat{ }-7%, at least about 1×10\hat{ }-6%, at least about 1×10\hat{ }-5%, at least about 1×10\hat{ }-4%, at least about 1×10\hat{ }-3%, at least about 1×10\hat{ }-2%, at least about 0.1%, at least about 1%, at least about 5%, at least about 10%, at least about 20%, at least about 25%, at least about 30%, at least about 40%, at least about 50%, at least about 60%, at least about 70%, at least about 80%, at least about 90%, at least about 100%, or more subsequent to being stored in an environment. In some cases, the pharmaceutical composition can have a shelf stability of at most about 100%, at most about 90%, at most about 80%, at most about 70%, at most about 60%, at most about 50%, at most about 40%, at most about 30%, at most about 25%, at most about 20%, at most about 10%, at most about 5%, at most about 1%, at most about 0.1%, at most about 1×10\hat{ }-2%, at most about 1×10\hat{ }-3%, at most about 1×10\hat{ }-4%, at most about 1×10\hat{ }-5%, at most about 1×10\hat{ }-6%, at most about 1×10\hat{ }-7%, at most about 1×10\hat{ }-8%, or less subsequent to being stored in an environment. In some cases, the pharmaceutical composition can have a shelf stability of at least about 1×10\hat{ }-6%, at least about 1×10\hat{ }-5%, at least about 1×10\hat{ }-4%, at least about 1×10\hat{ }-2%, at least about 1%, at least about 10%, at least about 25%, or at least about 50%.

In some instances, the pharmaceutical composition can have a shelf stability of between about 1×10\hat{ }-8% and about 100% subsequent to being stored in an anaerobic environment. In some cases, the pharmaceutical composition can have a shelf stability of at least about 1×10\hat{ }-8%, at least about 1×10\hat{ }-7%, at least about 1×10\hat{ }-6%, at least about 1×10\hat{ }-5%, at least about 1×10\hat{ }-4%, at least about 1×10\hat{ }-3%, at least about 1×10\hat{ }-2%, at least about 0.1%, at least about 1%, at least about 5%, at least about 10%, at least about 20%, at least about 25%, at least about 30%, at least about 40%, at least about 50%, at least about 60%, at least about 70%, at least about 80%, at least about 90%, at least about 100%, or more subsequent to being stored in an anaerobic environment. In some cases, the pharmaceutical composition can have a shelf stability of at most about 100%, at most about 90%, at most about 80%, at most about 70%, at most about 60%, at most about 50%, at most about 40%, at most about 30%, at most about 25%, at most about 20%, at most about 10%, at most about 5%, at most about 1%, at most about 0.1%, at most about 1×10\hat{ }-2%, at most about 1×10\hat{ }-3%, at most about 1×10\hat{ }-4%, at most about 1×10\hat{ }-5%, at most about 1×10\hat{ }-6%, at most about 1×10\hat{ }-7%, at most about 1×10\hat{ }-8%, or less subsequent to being stored in an anaerobic environment. In some cases, the pharmaceutical composition can have a shelf stability of at least about 1×10\hat{ }-6%, at least about 1×10\hat{ }-5%, at least about 1×10\hat{ }-4%, at least about 1×10\hat{ }-2%, at least about 1%, at least about 10%, at least about 25%, or at least about 50%.

In some instances, the pharmaceutical composition can have a shelf stability after being stored in an environment for a period of between about 1 day to about 500 days. In some cases, the pharmaceutical compositions can have a shelf stability after being stored in an environment for a period of at least about 1 day, at least about 2 days, at least about 3 days, at least about 4 days, at least about 5 days, at least about 6 days, at least about 7 days, at least about 8 days, at least about 9 days, at least about 10 days, at least about 20 days, at least about 30 days, at least about 40 days, at least about 50 days, at least about 60 days, at least about 70 days, at least about 80 days, at least about 90 days, at least about 100 days, at least about 110 days, at least about 120 days, at least about 130 days, at least about 140 days, at least about 150 days, at least about 200 days, at least about 250 days, at least about 300 days, at least about 350 days, at least about 400 days, at least about 450 days, at least about 500 days, or more. In some cases, the pharmaceutical composition can have a shelf stability after being stored in an environment for a period of at most about 500 days, at most about 450 days, at most about 400 days, at most about 350 days, at most about 300 days, at most about 250 days, at most about 200 days, at most about 150 days, at most about 140 days, at most about 130 days, at most about 120 days, at most about 110 days, at most about 100 days, at most about 90 days, at most about 80 days, at most about 70 days, at most about 60 days, at most about 50 days, at most about 40 days, at most about 30 days, at most about 20 days, at most about 10 days, at most about 9 days, at most about 8 days, at most about 7 days, at most about 6 days, at most about 5 days, at most about 4 days, at most about 3 days, at most about 2 days, at most about 1 day, or less.

In some instances, the pharmaceutical composition can have a shelf stability after being stored in an anaerobic environment for a period of between about 1 day to about 500 days. In some cases, the pharmaceutical compositions can have a shelf stability after being stored in an anaerobic environment for a period of at least about 1 day, at least about 2 days, at least about 3 days, at least about 4 days, at least about 5 days, at least about 6 days, at least about 7 days, at least about 8 days, at least about 9 days, at least about 10 days, at least about 20 days, at least about 30 days, at least about 40 days, at least about 50 days, at least about 60 days, at least about 70 days, at least about 80 days, at least about 90 days, at least about 100 days, at least about 110 days, at least about 120 days, at least about 130 days, at least about 140 days, at least about 150 days, at least about 200 days, at least about 250 days, at least about 300 days, at least about 350 days, at least about 400 days, at least about 450 days, at least about 500 days, or more. In some cases, the pharmaceutical composition can have a shelf stability after being stored in an anaerobic environment for a period of at most about 500 days, at most about 450 days, at most about 400 days, at most about 350 days, at most about 300 days, at most about 250 days, at most about 200 days, at most about 150 days, at most about 140 days, at most about 130 days, at most about 120 days, at most about 110 days, at most about 100 days, at most about 90 days, at most about 80 days, at most about 70 days, at most about 60 days, at most about 50 days, at most about 40 days, at most about 30 days, at most about 20 days, at most about 10 days, at most about 9 days, at most about 8 days, at most about 7 days, at most about 6 days, at most about 5 days, at most about 4 days, at most about 3 days, at most about 2 days, at most about 1 day, or less.

In some instances, the pharmaceutical composition can have a shelf stability after being stored in an environment at a temperature of between about −80° C. and about 42° C. In some cases, the pharmaceutical composition can have a shelf stability after being stored in an environment at a temperature of at least about −80° C., at least about −20° C., at least about −10° C., at least about −4° C., at least about 0° C., at least about 5° C., at least about 10° C., at least about 15° C., at least about 20° C., at least about 25° C., at least about 30° C., at least about 35° C., at least about 40° C., at least about 42° C., or more. In some cases, the pharmaceutical composition can have a shelf stability after being stored in an environment at a temperature of at most about 42° C., at most about 40° C., at most about 35° C., at most about 30° C., at most about 25° C., at most about 20° C., at most about 15° C., at most about 10° C., at most about 5° C., at most about 0° C., at most about −4° C., at most about −10° C., at most about −20° C., at most about −80° C., or less.

In some instances, the pharmaceutical composition can have a shelf stability after being stored in an anaerobic environment at a temperature of between about −80° C. and about 42° C. In some cases, the pharmaceutical composition can have a shelf stability after being stored in an anaerobic environment at a temperature of at least about −80° C., at least about −20° C., at least about −10° C., at least about −4° C., at least about 0° C., at least about 5° C., at least about 10° C., at least about 15° C., at least about 20° C., at least about 25° C., at least about 30° C., at least about 35° C., at least about 40° C., at least about 42° C., or more. In some cases, the pharmaceutical composition can have a shelf stability after being stored in an anaerobic environment at a temperature of at most about 42° C., at most about 40° C., at most about 35° C., at most about 30° C., at most about 25° C., at most about 20° C., at most about 15° C., at most about 10° C., at most about 5° C., at most about 0° C., at most about −4° C., at most about −10° C., at most about −20° C., at most about −80° C., or less.

### Pharmaceutical Composition Packs

In some instances, a plant-based capsule encompassing a pharmaceutical composition may be encompassed by a blister pack. In some instances, a plant-based capsule encompassing a pharmaceutical composition may be encompassed by a sachet pack. In some cases, a plant-based capsule encompassing a pharmaceutical composition may be encompassed by a secondary container. The secondary container may be a blister pack, a vial, a bottle, an ampoule, or a combination thereof. The secondary container may be a blister pack. The secondary container may be a vial. The secondary container may be a bottle. The secondary container may be an ampoule. In some cases, a blister pack encompassing a plant-based capsule that encompasses a pharmaceutical composition may comprise an aluminum blister pack. In some cases, a blister pack may comprise polyethylene terephthalate (PET), polyvinyl chloride (PVC), polyvinylidene chloride (PVDC), polychlorotrifluoroethylene (PCTFE), cyclic olefin polymers (COP), oriented poly amide (OPA), aluminum foil, or plastic film. In some cases, a sachet pack may comprise metal foils, cloths, plastics, papers, polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), or a combination thereof. In some cases, a blister pack encompassing a plant-based capsule that encompasses a pharmaceutical composition may comprise polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, or plastic film. In some cases, a blister pack encompassing a plant-based capsule that encompasses a pharmaceutical composition may comprise the derivatives or combinations of the material described thereof. In some cases, a blister pack encompassing a plant-based capsule that encompasses a pharmaceutical composition may comprise PET. In some cases, a blister pack encompassing a plant-based capsule that encompasses a pharmaceutical composition may comprise PVC. In some cases, a blister pack encompassing a plant-based capsule that encompasses a pharmaceutical composition may comprise PVDC. In some cases, a blister pack encompassing a plant-based capsule that encompasses a pharmaceutical composition may comprise PCTFE. In some cases, a blister pack encompassing a plant-based capsule that encompasses a pharmaceutical composition may comprise COP. In some cases, a blister pack encompassing a plant-based capsule that encompasses a pharmaceutical composition may comprise OPA. In some cases, a blister pack encompassing a plant-based capsule that encompasses a pharmaceutical composition may comprise aluminum foil. In some cases, a blister pack encompassing a plant-based capsule that encompasses a pharmaceutical composition may comprise plastic film. In some cases, a secondary container encompassing a plant-based capsule that encompasses a pharmaceutical composition may comprise any materials described for a blister pack. In some cases, a pharmaceutical composition may be encompassed by a blister pack, a vial, a bottle, an ampoule, or a secondary container without being encompassed by a capsule. In some cases, a pharmaceutical composition may be encompassed by a blister pack, a vial, a bottle, an ampoule, or a secondary container without being encompassed by a primary container.

In some cases, a blister pack may be filled with nitrogen gas. In some cases, a blister pack may be filled with pure nitrogen gas. In some cases, a blister pack may be filled with about 100% nitrogen gas by volume of the atmosphere. In some cases, a blister pack that encompasses a pharmaceutical composition may be filled with 99% nitrogen gas by volume of the atmosphere. In some cases, a blister pack that encompasses a pharmaceutical composition may be filled with 98% nitrogen gas by volume of the atmosphere. In some cases, a blister pack that encompasses a pharmaceutical composition may be filled with 97% nitrogen gas by volume of the atmosphere. In some cases, a blister pack that encompasses a pharmaceutical composition may be filled with 96% nitrogen gas by volume of the atmosphere. In some cases, a blister pack that encompasses a pharmaceutical composition may be filled with 95% nitrogen gas by volume of the atmosphere. In some cases, a blister pack that encompasses a pharmaceutical composition may be filled with 94% nitrogen gas by volume of the atmosphere. In some cases, a blister pack that encompasses a pharmaceutical composition may be filled with 93% nitrogen gas by volume of the atmosphere. In some cases, a blister pack that encompasses a pharmaceutical composition may be filled with 92% nitrogen gas by volume of the atmosphere. In some cases, a blister pack that encompasses a pharmaceutical composition may be filled with 91% nitrogen gas by volume of the atmosphere. In some cases, a blister pack that encompasses a pharmaceutical composition may be filled with 90% nitrogen gas by volume of the atmosphere. In some cases, a blister pack that encompasses a pharmaceutical composition may be filled with 89% nitrogen gas by volume of the atmosphere. In some cases, a blister pack that encompasses a pharmaceutical composition may be filled with 88% nitrogen gas by volume of the atmosphere. In some cases, a blister pack that encompasses a pharmaceutical composition may be filled with 87% nitrogen gas by volume of the atmosphere. In some cases, a blister pack that encompasses a pharmaceutical composition may be filled with 86% nitrogen gas by volume of the atmosphere. In some cases, a blister pack that encompasses a pharmaceutical composition may be filled with 85% nitrogen gas by volume of the atmosphere. In some cases, a blister pack that encompasses a pharmaceutical composition may be filled with 84% nitrogen gas by volume of the atmosphere. In some cases, a blister pack that encompasses a pharmaceutical composition may be filled with 83% nitrogen gas by volume of the atmosphere. In some cases, a blister pack that encompasses a pharmaceutical composition may be filled with 82% nitrogen gas by volume of the atmosphere. In some cases, a blister pack that encompasses a pharmaceutical composition may be filled with 81% nitrogen gas by volume of the atmosphere. In some cases, a blister pack that encompasses a pharmaceutical composition may be filled with 80% nitrogen gas by volume of the atmosphere. In some cases, a blister pack that encompasses a pharmaceutical composition may be filled with 80% to about 99% nitrogen gas by volume of the atmosphere. In some cases, a blister pack that encompasses a pharmaceutical composition may be filled with 85% to about 99% nitrogen gas by volume of the atmosphere. In some cases, a blister pack that encompasses a pharmaceutical composition may be filled with 90% to about 99% nitrogen gas by volume of the atmosphere. In some cases, a blister pack that encompasses a pharmaceutical composition may be filled with 95% to about 99% nitrogen gas by volume of the atmosphere. In some cases, a blister pack that encompasses a pharmaceutical composition may be filled with 96% to about 99% nitrogen gas by volume of the atmosphere. In some cases, a blister pack that encompasses a pharmaceutical composition may be filled with 97% to about 99% nitrogen gas by volume of the atmosphere. In some cases, a blister pack that encompasses a pharmaceutical composition may be filled with 98% to about 99% nitrogen gas by volume of the atmosphere.

In some cases, a blister pack encompassing a plant-based capsule that encompasses a pharmaceutical composition may comprise about 100% nitrogen gas by volume of the atmosphere. In some cases, a blister pack encompassing a plant-based capsule that encompasses a pharmaceutical composition may comprise about 99% nitrogen gas by volume of the atmosphere. In some cases, a blister pack encompassing a plant-based capsule that encompasses a pharmaceutical composition may comprise about 98% nitrogen gas by volume of the atmosphere. In some cases, a blister pack encompassing a plant-based capsule that encompasses a pharmaceutical composition may comprise about 97% nitrogen gas by volume of the atmosphere. In some cases, a blister pack encompassing a plant-based capsule that encompasses a pharmaceutical composition may comprise about 96% nitrogen gas by volume of the atmosphere. In some cases, a blister pack encompassing a plant-based capsule that encompasses a pharmaceutical composition may comprise about 95% nitrogen gas by volume of the atmosphere. In some cases, a blister pack encompassing a plant-based capsule that encompasses a pharmaceutical composition may comprise about 94% nitrogen gas by volume of the atmosphere. In some cases, a blister pack encompassing a plant-based capsule that encompasses a pharmaceutical composition may comprise about 93% nitrogen gas by volume of the atmosphere. In some cases, a blister pack encompassing a plant-based capsule that encompasses a pharmaceutical composition may comprise about 92% nitrogen gas by volume of the atmosphere. In some cases, a blister pack encompassing a plant-based capsule that encompasses a pharmaceutical composition may comprise about 91% nitrogen gas by volume of the atmosphere. In some cases, a blister pack encompassing a plant-based capsule that encompasses a pharmaceutical composition may comprise about 90% nitrogen gas by volume of the atmosphere. In some cases, a blister pack encompassing a plant-based capsule that encompasses a pharmaceutical composition may comprise about 89% nitrogen gas by volume of the atmosphere. In some cases, a blister pack encompassing a plant-based capsule that encompasses a pharmaceutical composition may comprise about 88% nitrogen gas by volume of the atmosphere. In some cases, a blister pack encompassing a plant-based capsule that encompasses a pharmaceutical composition may comprise about 87% nitrogen gas by volume of the atmosphere. In some cases, a blister pack encompassing a plant-based capsule that encompasses a pharmaceutical composition may comprise about 86% nitrogen gas by volume of the atmosphere. In some cases, a blister pack encompassing a plant-based capsule that encompasses a pharmaceutical composition may comprise about 85% nitrogen gas by volume of the atmosphere. In some cases, a blister pack encompassing a plant-based capsule that encompasses a pharmaceutical composition may comprise about 84% nitrogen gas by volume of the atmosphere. In some cases, a blister pack encompassing a plant-based capsule that encompasses a pharmaceutical composition may comprise about 83% nitrogen gas by volume of the atmosphere. In some cases, a blister pack encompassing a plant-based capsule that encompasses a pharmaceutical composition may comprise about 82% nitrogen gas by volume of the atmosphere. In some cases, a blister pack encompassing a plant-based capsule that encompasses a pharmaceutical composition may comprise about 81% nitrogen gas by volume of the atmosphere. In some cases, a blister pack encompassing a plant-based capsule that encompasses a pharmaceutical composition may comprise about 80% nitrogen gas by volume of the atmosphere. In some cases, a blister pack encompassing a plant-based capsule that encompasses a pharmaceutical composition may comprise about 80% to about 99% nitrogen gas by volume of the atmosphere. In some cases, a blister pack encompassing a plant-based capsule that encompasses a pharmaceutical composition may comprise about 85% to about 99% nitrogen gas by volume of the atmosphere. In some cases, a blister pack encompassing a plant-based capsule that encompasses a pharmaceutical composition may comprise about 90% to about 99% nitrogen gas by volume of the atmosphere. In some cases, a blister pack encompassing a plant-based capsule that encompasses a pharmaceutical composition may comprise about 95% to about 99% nitrogen gas by volume of the atmosphere. In some cases, a blister pack encompassing a plant-based capsule that encompasses a pharmaceutical composition may comprise about 96% to about 99% nitrogen gas by volume of the atmosphere. In some cases, a blister pack encompassing a plant-based capsule that encompasses a pharmaceutical composition may comprise about 97% to about 99% nitrogen gas by volume of the atmosphere. In some cases, a blister pack encompassing a plant-based capsule that encompasses a pharmaceutical composition may comprise about 98% to about 99% nitrogen gas by volume of the atmosphere.

In some cases, a sachet pack may be filled with nitrogen gas. In some cases, a sachet pack may be filled with pure nitrogen gas. In some cases, a sachet pack may be filled with about 100% nitrogen gas by volume of the atmosphere. In some cases, a sachet pack that encompasses a pharmaceutical composition may be filled with 99% nitrogen gas by volume of the atmosphere. In some cases, a sachet pack that encompasses a pharmaceutical composition may be filled with 98% nitrogen gas by volume of the atmosphere. In some cases, a sachet pack that encompasses a pharmaceutical composition may be filled with 97% nitrogen gas by volume of the atmosphere. In some cases, a sachet pack that encompasses a pharmaceutical composition may be filled with 96% nitrogen gas by volume of the atmosphere. In some cases, a sachet pack that encompasses a pharmaceutical composition may be filled with 95% nitrogen gas by volume of the atmosphere. In some cases, a sachet pack that encompasses a pharmaceutical composition may be filled with 94% nitrogen gas by volume of the atmosphere. In some cases, a sachet pack that encompasses a pharmaceutical composition may be filled with 93% nitrogen gas by volume of the atmosphere. In some cases, a sachet pack that encompasses a pharmaceutical composition may be filled with 92% nitrogen gas by volume of the atmosphere. In some cases, a sachet pack that encompasses a pharmaceutical composition may be filled with 91% nitrogen gas by volume of the atmosphere. In some cases, a sachet pack that encompasses a pharmaceutical composition may be filled with 90% nitrogen gas by volume of the atmosphere. In some cases, a sachet pack that encompasses a pharmaceutical composition may be filled with 89% nitrogen gas by volume of the atmosphere. In some cases, a sachet pack that encompasses a pharmaceutical composition may be filled with 88% nitrogen gas by volume of the atmosphere. In some cases, a sachet pack that encompasses a pharmaceutical composition may be filled with 87% nitrogen gas by volume of the atmosphere. In some cases, a sachet pack that encompasses a pharmaceutical composition may be filled with 86% nitrogen gas by volume of the atmosphere. In some cases, a sachet pack that encompasses a pharmaceutical composition may be filled with 85% nitrogen gas by volume of the atmosphere. In some cases, a sachet pack that encompasses a pharmaceutical composition may be filled with 84% nitrogen gas by volume of the atmosphere. In some cases, a sachet pack that encompasses a pharmaceutical composition may be filled with 83% nitrogen gas by volume of the atmosphere. In some cases, a sachet pack that encompasses a pharmaceutical composition may be filled with 82% nitrogen gas by volume of the atmosphere. In some cases, a sachet pack that encompasses a pharmaceutical composition may be filled with 81% nitrogen gas by volume of the atmosphere. In some cases, a sachet pack that encompasses a pharmaceutical composition may be filled with 80% nitrogen gas by volume of the atmosphere. In some cases, a sachet pack that encompasses a pharmaceutical composition may be filled with 80% to about 99% nitrogen gas by volume of the atmosphere. In some cases, a sachet pack that encompasses a pharmaceutical composition may be filled with 85% to about 99% nitrogen gas by volume of the atmosphere. In some cases, a sachet pack that encompasses a pharmaceutical composition may be filled with 90% to about 99% nitrogen gas by volume of the atmosphere. In some cases, a sachet pack that encompasses a pharmaceutical composition may be filled with 95% to about 99% nitrogen gas by volume of the atmosphere. In some cases, a sachet pack that encompasses a pharmaceutical composition may be filled with 96% to about 99% nitrogen gas by volume of the atmosphere. In some cases, a sachet pack that encompasses a pharmaceutical composition may be filled with 97% to about 99% nitrogen gas by volume of the atmosphere. In some cases, a sachet pack that encompasses a pharmaceutical composition may be filled with 98% to about 99% nitrogen gas by volume of the atmosphere.

In some cases, a sachet pack encompassing a plant-based capsule that encompasses a pharmaceutical composition may comprise about 100% nitrogen gas by volume of the atmosphere. In some cases, a sachet pack encompassing a plant-based capsule that encompasses a pharmaceutical composition may comprise about 99% nitrogen gas by volume of the atmosphere. In some cases, a sachet pack encompassing a plant-based capsule that encompasses a pharmaceutical composition may comprise about 98% nitrogen gas by volume of the atmosphere. In some cases, a sachet pack encompassing a plant-based capsule that encompasses a pharmaceutical composition may comprise about 97% nitrogen gas by volume of the atmosphere. In some cases, a sachet pack encompassing a plant-based capsule that encompasses a pharmaceutical composition may comprise about 96% nitrogen gas by volume of the atmosphere. In some cases, a sachet pack encompassing a plant-based capsule that encompasses a pharmaceutical composition may comprise about 95% nitrogen gas by volume of the atmosphere. In some cases, a sachet pack encompassing a plant-based capsule that encompasses a pharmaceutical composition may comprise about 94% nitrogen gas by volume of the atmosphere. In some cases, a sachet pack encompassing a plant-based capsule that encompasses a pharmaceutical composition may comprise about 93% nitrogen gas by volume of the atmosphere. In some cases, a sachet pack encompassing a plant-based capsule that encompasses a pharmaceutical composition may comprise about 92% nitrogen gas by volume of the atmosphere. In some cases, a sachet pack encompassing a plant-based capsule that encompasses a pharmaceutical composition may comprise about 91% nitrogen gas by volume of the atmosphere. In some cases, a sachet pack encompassing a plant-based capsule that encompasses a pharmaceutical composition may comprise about 90% nitrogen gas by volume of the atmosphere. In some cases, a sachet pack encompassing a plant-based capsule that encompasses a pharmaceutical composition may comprise about 89% nitrogen gas by volume of the atmosphere. In some cases, a sachet pack encompassing a plant-based capsule that encompasses a pharmaceutical composition may comprise about 88% nitrogen gas by volume of the atmosphere. In some cases, a sachet pack encompassing a plant-based capsule that encompasses a pharmaceutical composition may comprise about 87% nitrogen gas by volume of the atmosphere. In some cases, a sachet pack encompassing a plant-based capsule that encompasses a pharmaceutical composition may comprise about 86% nitrogen gas by volume of the atmosphere. In some cases, a sachet pack encompassing a plant-based capsule that encompasses a pharmaceutical composition may comprise about 85% nitrogen gas by volume of the atmosphere. In some cases, a sachet pack encompassing a plant-based capsule that encompasses a pharmaceutical composition may comprise about 84% nitrogen gas by volume of the atmosphere. In some cases, a sachet pack encompassing a plant-based capsule that encompasses a pharmaceutical composition may comprise about 83% nitrogen gas by volume of the atmosphere. In some cases, a sachet pack encompassing a plant-based capsule that encompasses a pharmaceutical composition may comprise about 82% nitrogen gas by volume of the atmosphere. In some cases, a sachet pack encompassing a plant-based capsule that encompasses a pharmaceutical composition may comprise about 81% nitrogen gas by volume of the atmosphere. In some cases, a sachet pack encompassing a plant-based capsule that encompasses a pharmaceutical composition may comprise about 80% nitrogen gas by volume of the atmosphere. In some cases, a sachet pack encompassing a plant-based capsule that encompasses a pharmaceutical composition may comprise about 80% to about 99% nitrogen gas by volume of the atmosphere. In some cases, a sachet pack encompassing a plant-based capsule that encompasses a pharmaceutical composition may comprise about 85% to about 99% nitrogen gas by volume of the atmosphere. In some cases, a sachet pack encompassing a plant-based capsule that encompasses a pharmaceutical composition may comprise about 90% to about 99% nitrogen gas by volume of the atmosphere. In some cases, a sachet pack encompassing a plant-based capsule that encompasses a pharmaceutical composition may comprise about 95% to about 99% nitrogen gas by volume of the atmosphere. In some cases, a sachet pack encompassing a plant-based capsule that encompasses a pharmaceutical composition may comprise about 96% to about 99% nitrogen gas by volume of the atmosphere. In some cases, a sachet pack encompassing a plant-based capsule that encompasses a pharmaceutical composition may comprise about 97% to about 99% nitrogen gas by volume of the atmosphere. In some cases, a sachet pack encompassing a plant-based capsule that encompasses a pharmaceutical composition may comprise about 98% to about 99% nitrogen gas by volume of the atmosphere.

### Plant-Based Pharmaceutical Compositions

In some instances, the pharmaceutical composition described in this disclosure may be plant-based. In some cases, the pharmaceutical composition described in this disclosure may be vegan. In some cases, a pharmaceutical composition may be plant-based if it does not comprise a substance or product that is derived from a slaughter. In some cases, a pharmaceutical composition may be vegan if it does not comprise a substance or product that is derived from an animal. A substance may be derived from an animal if it is synthesized by or isolated from an animal. A substance may also be derived from an animal if it is derived directly from a product synthesized by or isolated from an animal. A substance may be synthesized by or isolated from an animal if a significant portion of the population of the molecules of the substance is synthesized by the animal. A product may be synthesized by or isolated from an animal if a significant portion of the population of the molecules of the product is synthesized by the animal. The significant portion may comprise an amount of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

### Cryoprotectant and Antioxidant

Provided herein are pharmaceutical compositions that can comprise one or more cryoprotectant. A cryoprotectant may have the composition described in TABLE 5 or EXAMPLE 1. Such cryoprotectant can be used to maintain viability of the bacterial cells in a pharmaceutical composition when such composition is frozen or lyophilized, for example, during transport and/or storage prior to use. In some instances, the one or more cryoprotectant can be glycerol, dimethyl sulfoxide (DMSO), ethylene glycol, propylene glycol, 2-methyl-2,4-pentanediol, trehalose, sucrose, diethyl glycol, triethylene glycol, polyvinylpyrrolidone (PVP), polyethylene glycol (PEG), saccharose, formamide, glycerol 3-phosphate, proline, methyl alcohol, glucose, bovine serum albumin, polyvinyl alcohol, hydroxyethyl starch, sorbitol, or a combination thereof. Cryoprotectant can comprise an ice blocker. An ice blocker can comprise polyglycerol, polyvinyl alcohol, X-1000 and Z-1000. Such cryoprotectant can be used in a pharmaceutical composition in an amount of about 5, 10, 15, 20, 25, or 30 volume percent (% v/v) or weight percent (% w/w), e.g., depending on whether the pharmaceutical composition is a solid dosage from (e.g., a capsule or tablet) or a liquid dosage from (e.g., a suspension or a gel). Cryoprotectant can also comprise a carbohydrate or an antioxidant. A carbohydrate can comprise trehalose, sucrose, sorbitol, glucose, fructose, saccharose, or a combination thereof.

In some embodiments, pharmaceutical compositions described herein further comprise an antioxidant. A cryoprotectant may comprise an antioxidant. A cryoprotectant may be present in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. A cryoprotectant may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. A cryoprotectant may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the cryoprotectant may be measured by weight or volume of the pharmaceutical composition.

In some embodiments, the antioxidant is L-cysteine. In some embodiments, the L-cysteine is present in an amount of about 0.001%, 0.005%, 0.01%, 0.05%, 0.1%, 0.5%, 1%, 1.5%, 2%, 5%, 10%, 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10%. Saccharose can be present in an amount of about 0.1%, 0.5%, 1%, 5%, 10%, 15%, 20%, 25%, 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 0.1% to 1%, 1% to 5%, 5% to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 51 to 61%, 52 to 62%, 53 to 63%, 54 to 64%, 55 to 65%, 56 to 66%, 57 to 67%, 58 to 68%, or 59 to 69% of the pharmaceutical composition. Trehalose can be present in an amount of about 0.01%, 0.05%, 0.1%0, 0.5%, 1%, 1.%, 2%, 2.5%, 3%, 3.5%, 4%, 4.5%5, 5% 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 0.01% to 15%, 0.1% to 20%, 0.01% to 0.1%, 0.11% to 10%, 1 to 11%, 2 to 12%, 3 to 13%, 4 to 14%, 5 to 15%, 6 to 16%, 7 to 17%, 8 to 18%, 9 to 19%, 10 to 20%, 11 to 21%, 12 to 22%, 13 to 23%, 14 to 24%, or 15 to 25% of the pharmaceutical composition. Glycerol can be present in an amount of about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1 to 21%, 2 to 22%, 3 to 23%, 4 to 24%, 5 to 25%, 6 to 26%, 7 to 27%, 8 to 28%, 9 to 29%, 10 to 30%, 11 to 31%, 12 to 32%, 13 to 33%, 14 to 34%, 15 to 35%, 16 to 36%, 17 to 37%, 18 to 38%, 19 to 39%, or 20 to 40% of the pharmaceutical composition. The percentages thereof may be measured by weight or volume of the pharmaceutical composition.

In some embodiments herein, the cryoprotectant of a pharmaceutical composition herein is glycerol. Such glycerol can be used in an amount of about 20% v/v in a pharmaceutical composition that can comprise a bacterial consortium of one or more, two or more, or three or more bacterial strains selected from TABLE 1. In some embodiments, the bacteria populations can be lyophilized. A lyophilization process can comprise a low temperature dehydration of the bacterial population. In some embodiments, the lyophilization process can comprise subjecting the bacterial population at low temperature and low pressure.

Provided herein are pharmaceutical compositions that can comprise one or more antioxidant. In some instances, such antioxidant can be used to protect anaerobic bacterial species and/or strain(s) that may be present in the pharmaceutical composition. In such instances, the one or more antioxidant can be used to provide anaerobic conditions during storage and/or transport, and/or to protect the bacterial cells from reactive oxygen species. In some embodiments herein, the antioxidant can be ascorbic acid, dithiothreitol, glutathione, phenolic acids (e.g., gallic, protochatechuic, caffeic, and rosmarinic acids), phenolic diterpenes (e.g., camosol and carnosic acid), flavonoids (e.g., quercetin and catechin), volatile oils (e.g., eugenol, carvacrol, thymol, and menthol), α-Tocopherol (e.g., vitamin E), Trolox, ascorbic acid, vitamin A, vitamin C, coenzyme Q10, manganese, iodide, melatonin, alpha-carotene, astaxanthin, beta-carotene, canthaxanthin, cryptoxanthin, lutein, lycopene, zeaxanthin, flavonoids (e.g., flavones such as apigentin), luteolin, tangeithin, flavonols, isorhamnetin, kaempferol, myricetin, proanthocyanidins, quercetin, eriodictyol, hesperetin, naringenin, catechin, gallocatechin, epicatechin, epigallocatechin, theaflavin, thearubigins, isoflavone phytoestrogens, daidzein, genistein, glycitein, stilbenoids such as resveratrol, pterostilbene, anthocyanins, cyanidin, delphinidin, malvidin, pelargonidin, peonidin, petunidin, chicoric acid, chlorogenic acid, cinnamic acid, ellagic acid, ellagitannins, gallic acid, gallotannins, rosmarinic acid, curcumin, xanthones, capsaicin, bilirubin, citric acid, oxalic acid, phytic acid, N-acetylcysteine, L-cysteine, L-glutamate, L-proline, R-α-lipoic acid, anthocyanins, copper, cryptoxanthins, flavonoids, indoles, isoflavonoids, lignans, selenium, zinc, or a combination thereof. Such one or more antioxidant(s) can be present in a pharmaceutical composition in an amount of at least about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, 99% or more of the pharmaceutical composition. The antioxidant may be present in an amount of at most about 0.001%, 0.005%, 0.01%, 0.05%, 0.1%, 0.5%, 1%, 1.5%, 2%, 5%, 10%, 15%, 20%, 25%, 30%, 35%, 40%, 45%, 50%, 55%, 60%, 65%, 70%, 75%, 80%, 85%, 90%, 95%, or 99% of the pharmaceutical composition. The antioxidant may be present in an amount of about 0.001% to 0.005%, 0.0051% to 0.01%, 0.011% to 0.05%, 0.05% to 0.1%, 0.051% to 0.1%, 0.11% to 0.5%, 0.51% to 1%, 1.1% to 1.5%, 1.5% to 2%, 2.1% to 5%, or 5.1% to 10% of the pharmaceutical composition. The percentage of the antioxidant may be measured by weight or volume of the pharmaceutical composition. L-glutamate can be present in an amount of about 0.01%, 0.05%, 0.1%, 0.5%, 1%, 1.5%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 8%, 9%, 10%, 1 to 5%, 1.1 to 5.1%, 1.2 to 5.2%, 1.3 to 5.3%, 1.4 to 5.4%, 1.5 to 5.5%, 1.6 to 5.6%, 1.7 to 5.7%, 1.8 to 5.8%, 1.9 to 5.9%, 2 to 6%, 2.1 to 6.1%, 2.2 to 6.2%, 2.3 to 6.3%, 2.4 to 6.4%, 2.5 to 6.5%, 2.6 to 6.6%, 2.7 to 6.7%, 2.8 to 6.8%, 2.9 to 6.9%, 3 to 7%, 3.1 to 7.1%, 3.2 to 7.2%, 3.3 to 7.3%, 3.4 to 7.4%, 3.5 to 7.5%, 3.6 to 7.6%, 3.7 to 7.7%, 3.8 to 7.8%, 3.9 to 7.9%, or 4 to 8% of the pharmaceutical composition. The percentage of the L-glutamate may be measured by weight or volume of the pharmaceutical composition. In some case, the cryoprotectant can comprise, by weight, about 60% saccharose, about 10% trehalose, about 1% L-cysteine, and about 4% L-glutamate of the pharmaceutical composition.

Provided herein are pharmaceutical compositions that can comprise an aqueous buffer solution. Such aqueous medium can be used as the main storage and transport medium for the bacterial cells. As such, the buffer can contain any one or more of bacterial consortium, cryoprotectant, and antioxidant, either dissolved or suspended, to form a pharmaceutical composition as described herein. In some instances, the aqueous buffer solution can be phosphate buffered saline (PBS), HEPES, or Tris buffer, any other suitable buffer, or any combination thereof. In some embodiments, the buffer is PBS and comprises 137 mM NaCl, 2.7 mM KCl, 10 mM Na₂HPO₄, and 1.8 mM KH₂PO₄. In other cases, the buffer can be PBS and can have a pH of about 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, or 9.

Thus, in some embodiments herein, a pharmaceutical composition comprises a bacterial consortium consisting of about 5×10\hat{ }8 CFU of each of the bacterial strains A. muciniphila (DSM 33213), F. prausnitzii (DSM 33185), and L. crispatus (DSM 33187), about 20% v/v glycerol as cryoprotectant, 0.1% w/w L-cysteine as antioxidant, and PBS buffer containing 137 mM NaCl, 2.7 mM KCl, 10 mM Na₂HPO₄, and 1.8 mM KH₂PO₄. Such pharmaceutical composition can be manufactured and formulated into an orally administrable dosage form using the methods and compositions described herein.

Provided herein are pharmaceutical compositions that can be designed and manufactured to allow storage and/or transport of the pharmaceutical compositions. In some instances, a pharmaceutical composition herein comprising a bacterial consortium can be designed such that the viability of the bacterial cells in the pharmaceutical composition is not or only minimally affected by storage and/or transport. In such instances, the viability of at least about 80%, 85%, 90%, 95%, 97%, or 99% of bacterial cells in the pharmaceutical composition is maintained during storage and/or transport.

In some instances, a pharmaceutical composition herein comprises a cryoprotectant to allow storage at low temperatures at about −70° C. or −80° C. to preserve viability of the bacterial cells. In such instances, the pharmaceutical composition can comprise about 20% v/v glycerol as a cryoprotectant. A pharmaceutical composition herein can further comprise an antioxidant that can preserve an anaerobic environment in the storage or transport vial and can protect the bacterial cells from reactive oxygen species.

In one example, in a pharmaceutical composition herein, live, vegetative bacteria can be preserved frozen in phosphate buffered saline (PBS) with 20% v/v glycerol and 0.1% w/w cysteine to preserve their viability. In such instances, the live bacteria can belong to any one or more of the strains shown in TABLE 1.

In some instances, the cryoprotectants or antioxidants described herein and thereof may be used with the bacterial populations, the pharmaceutical compositions, the methods for producing pharmaceutical compositions, or the methods for large-scale growth of Lactobacillus sp. described in this disclosure. In some instances, the cryoprotectants or antioxidants described herein and thereof may be used with the bacterial populations, the pharmaceutical compositions, the methods for producing pharmaceutical compositions, or the methods for large-scale growth of species of the Lactobacillaceae family described in this disclosure. In some cases, the cryoprotectants or antioxidants described herein and thereof may be used with the growth media or excipients described in this disclosure. In other cases, the cryoprotectants or antioxidants described herein and thereof may be used in any embodiments or examples described in this disclosure.

### Growth Media for Bacterial Populations

Provided herein are methods for manufacturing L. crispatus (DSM 33187) cell batches that may be used in a pharmaceutical composition described herein. Such methods may comprise preparing a L. crispatus (DSM 33187) cell culture medium. Such culture medium may be a vMRS medium or Boullion vMRS broth. In some cases, such medium may comprise the media described elsewhere in this disclosure. In some cases, a medium for growing L. crispatus (DSM 33187) may be described in TABLES 3 & 4 or EXAMPLE 1. The media for growing L. crispatus (DSM 33187) may also be used for growing any bacterial strains or species of Lactobacillus sp. The media for growing L. crispatus (DSM 33187) may also be used for growing any bacterial strains or species of species of the Lactobacillaceae family.

In some instances, such culture medium is not HiMedia vMRS broth. Such culture medium may be specific for a L. crispatus (DSM 33187) strain and may comprise vMRS powder and dipotassium phosphate (K₂HPO₄). In such instances, a medium for growing and culturing L. crispatus (DSM 33187) cells may comprise about 250-300 g of vMRS powder and dipotassium phosphate (K₂HPO₄). In certain instances, such medium may comprise about 273 g vMRS powder and about 12.5 g dipotassium phosphate (K₂HPO₄) and about 4.9 L of water. The pH of such vMRS media may be adjusted to about 6.5±0.1 using, e.g., 5 M hydrochloride solution or glacial acetic acid. The media may then be filtered, reduced to an anaerobic state, and transferred to, e.g., a starter culture tube containing stock L. crispatus (DSM 33187) solution, and incubated at 37° C. for about 16-20 hours. Following incubation and expansion, the absorbance of the cell culture at 600 nm may be determined and repeated in triplicates to ensure absorbance of the cell suspension falls within the range from about 0.8 to about 1.6, preferably of about 1.0-1.4. The contents of the culture flasks may be centrifuged, the residual cell pellets re-suspended in 25 mL of sterile PBS containing an antioxidant and cryoprotectant such as 20% v/v glycerol, and then combined to yield a homogenous cell suspension.

Further provided herein are methods for manufacturing A. muciniphila (DSM 33213) cell batches that may be used in a pharmaceutical composition described herein. Such methods may comprise preparing an A. muciniphila (DSM 33213) cell culture medium. The media for growing A. muciniphila (DSM 33213) may also be used for growing any bacterial strains or species of Akkermansia sp.

In some instances, such A. muciniphila (DSM 33213) culture medium may be a modified NAGT medium. Such modified NAGT medium may contain soytone or N-acetyl glucosamine (NAG), or both soytone and NAG. In some cases, such modified NAGT medium may not contain magnesium, calcium, glucose, or a combination thereof. In some instances, a modified NAGT medium may provide improved cell growth. Such improved cell growth may be about 30%, 35%, 40%, 45%, or 50% higher compared to cell growth in unmodified NAGT medium.

Thus, in some instance, such NAGT culture medium may be specific for a A. muciniphila strain (DSM 33213) and may comprise any one or more of the ingredients: soytone, pea peptone, yeast extract, sodium bicarbonate (NaHCO₃), dibasic potassium phosphate (K₂HPO₄), sodium chloride (NaCl), magnesium sulfate (e.g., MgSO₄×7H₂O), calcium chloride (CaCl₂)), glucose, N-acetylglucosamine, L-threonine, and/or L-cysteine. In such instances, a volume of about 5 L of a modified NAGT medium for growing and culturing A. muciniphila (DSM 33213) cells may comprise from about 75 g to 100 g of SOLABIA Pea Peptone, from about 75 g to about 85 g of Difcom Select Soytone, from about 10 g to about 15 g of Bactom Yeast Extract, from about 2 g to about 8 g of sodium bicarbonate (NaHCO₃), from about 10 g to about 15 g of dibasic potassium phosphate (K₂HPO₄), from about 0.5 g to about 5 g of sodium chloride (NaCl), from about 0.5 g to about 5 g of magnesium sulfate heptahydrate (MgSO₄×7H₂O), from about 0.5 g to about 5 g of calcium chloride (CaCl₂)), from about 20 g to about 25 g of glucose (dextrose), from about 25 g to about 30 g of N-acetylglucosamine, from about 15 g to about 25 g of L-threonine, and/or from about 2 g to about 8 g of L-cysteine. In one example, a volume of about 5 L of a modified NAGT medium for growing and culturing A. muciniphila (DSM 33213) cells may comprise about 82.5 g SOLABIA Pea Peptone, 82.5 g of Difcom Select Soytone, about 12.5 g of Bactom Yeast Extract, about 5 g of sodium bicarbonate (NaHCO₃), about 12.5 g of dibasic potassium phosphate (K₂HPO₄), about 1.5 g of sodium chloride (NaCl), about 0.5 g of magnesium sulfate heptahydrate (MgSO₄×7H₂O), about 0.5 g of calcium chloride (CaCl₂)), about 22.6 g of glucose (dextrose), about 27.7 g of N-acetylglucosamine, about 20 g of L-threonine, and/or about 5 g of L-cysteine.

The pH of such NAGT media may be adjusted, e.g., to about 6.5±0.1 using, e.g., 5 M hydrochloride solution. The pH of such NAGT media may also be adjusted to about 7. A. muciniphila (DSM 33213) bacterial cells may be added into prepared vials containing such NAGT growth medium. Following incubation for a time period that may be specific for the A. muciniphila (DSM 33213) strain, the absorbance of the cell culture at 600 nm may be measured and recorded to achieve an absorbance value of about 0.5 to about 1.2, preferably about 0.7-1.1. The contents of the culture flasks may then be centrifuged, the supernatants removed, and the residual cell pellets re-suspended in sterile PBS containing an antioxidant and cryoprotectant such as 20% v/v glycerol.

Further provided herein are methods for manufacturing F. prausnitzii (DSM 33185) cell batches that may be used in a bacterial consortium of a pharmaceutical composition described herein. The media for growing F. prausnitzii (DSM 33185) may also be used for growing any bacterial strains or species of Faecalibacterium sp.

Such methods may comprise preparing a complete vitamin mix solution (e.g., YFAP vitamin mix) and a F. prausnitzii (DSM 33185) cell culture medium. The YFAP vitamin mix may be specific for the F. prausnitzii (DSM 33185) strain and may comprise any one or more of biotin, cobalamin, p-aminobenzoic acid, folic acid, pyridoxamine, thiamine, and/or riboflavin. In such instances, a 1 L volume of the YFAP vitamin mix may comprise about 10 mg of biotin, about 10 mg of cobalamin, about 30 mg of p-aminobenzoic acid, about 50 mg of folic acid, about 150 mg of pyridoxamine, about 50 mg of thiamine, and about 50 mg of riboflavin. All media components may be dissolved, resulting in a solution that is clear and free of solids and precipitates. The YFAP Vitamin mix medium may be filtered and sterilized for use in a F. prausnitzii (DSM 33185) culture medium as described below.

Such F. prausnitzii (DSM 33185) culture medium may be prepared to comprise any one or more of BBL™ Phytone Peptone, SOLABIA Pea Peptone, Difco™ Select Soytone, Bactom Yeast Extract, sodium bicarbonate (NaHCO₃), dibasic potassium phosphate (K₂HPO₄), sodium chloride (NaCl), magnesium sulfate heptahydrate (MgSO₄×7H₂O), sodium acetate (NaOAc), glucose (dextrose), sodium propionate, L-cysteine, and/or YFAP Vitamin Mix solution, e.g., prepared as described above. In such instances, a volume of about 5 L of the F. prausnitzii (DSM 33185) culture medium may comprise from about 75 g to 100 g of SOLABIA Pea Peptone, from about 45 g to about 55 g of BBL™ Phytone Peptone, from about 45 g to about 55 g of Difco™ Select Soytone, from about 20 g to about 30 g of Bactom Yeast Extract, from about 2 g to about 8 g of sodium bicarbonate (NaHCO₃), from about 10 g to about 15 g of dibasic potassium phosphate (K₂HPO₄), from about 2 g to about 8 g of sodium chloride, from about 0.5 g to about 2 g of magnesium sulfate heptahydrate (MgSO₄×7H₂O), from about 20 g to about 30 g of sodium acetate (NaOAc), from about 40 g to about 60 g of glucose (dextrose), from about 2 g to about 8 g of sodium propionate, from about 2 g to about 8 g of L-cysteine, and about 0.5 to about 3 mL of YFAP Vitamin Mix solution, e.g., prepared as described above. Thus, in an example, a volume of about 5 L of the F. prausnitzii (DSM 33185) culture medium may comprise about 100 g SOLABIA Pea Peptone, 50 g of BBL™ Phytone Peptone, about 50 g of Difco™ Select Soytone, about 25 g of Bactom Yeast Extract, about 5 g of sodium bicarbonate (NaHCO₃), about 12.5 g of dibasic potassium phosphate (K₂HPO₄), about 5 g of sodium chloride, about 1 g of magnesium sulfate heptahydrate (MgSO₄×7H₂O), about 25 g of sodium acetate (NaOAc), about 50 g of glucose (dextrose), about 5 g of sodium propionate, about 5 g of L-cysteine, and about 1 mL of YFAP Vitamin Mix solution, e.g., prepared as described above.

A F. prausnitzii (DSM 33185) culture medium, YFAP-NU, may also be prepared to comprise any one or more of Pea Peptone, NuCel® 783 Yeast Extract, sodium bicarbonate (NaHCO₃), dibasic potassium phosphate (K₂HPO₄), sodium chloride (NaCl), magnesium sulfate heptahydrate (MgSO₄×7H₂O), sodium acetate (NaOAc), glucose (dextrose), L-cysteine, and/or cobalamin. In such instances, a volume of about 5 L of the F. prausnitzii (DSM 33185) culture medium may comprise from about 75 g to 100 g pea peptone, about 50 g NuCel® 783 Yeast Extract, 5 g sodium bicarbonate (NaHCO₃), about 12.5 g dibasic potassium phosphate (K₂HPO₄), about 5 g sodium chloride (NaCl), about 1 g magnesium sulfate heptahydrate (MgSO₄×7H₂O), about 25 g sodium acetate (NaOAc), about 50 g glucose (dextrose), about 5 g L-cysteine, and about 5 g cobalamin.

The pH of such cell media may be adjusted to about 6.5±0.1, e.g., using glacial acetic acid. Such pH may vary from about 6.2 to about 6.8, depending on the bacterial strain used. In some cases, the pH of the cell media may not be regulated. Such pH may vary from about 4.5 to about 7.5. In some embodiment, the pH of such media may be about 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5. After reduction to an anaerobic state, starter cultures for F. prausnitzii (DSM 33185) may be prepared by adding a certain volume of stock F. prausnitzii (DSM 33185) solution, e.g., approximately 500 μL of a cell bank stock solution, to starter culture tubes containing reduced culture medium, followed by incubation at 37° C. for about 12-16 hours. After expansion of the starter culture (e.g., after about 12-24 additional hours of incubation), the absorbance of the cell culture at 600 nm may be determined and repeated in triplicates to ensure absorbance is in a specific range. Such absorbance range may be from about 1.2 to about 2.0, preferably from about 1.4 to about 1.8. The culture flasks may then be centrifuged, the supernatants removed, and the residual cell pellets re-suspended in sterile PBS to yield a homogenous solution.

### Methods for Pharmaceutical Composition Manufacturing

### Methods for Producing Pharmaceutical Compositions.

Disclosed herein, are methods for producing the pharmaceutical compositions described herein and thereof. In some instances, the method comprises: 1) providing a mixture comprising a purified bacterial population; 2) filling the blended pharmaceutical composition into capsules; and 3) packing the capsules into a blister pack. In some cases, the purified bacterial population may comprise any purified bacterial population described in this disclosure. In some cases, the providing and packing may be under an oxygen-free atmosphere. In some cases, the providing may be under an oxygen-free atmosphere. In some cases, the packing may be under an oxygen-free atmosphere. In some cases, the providing or packing may be under an oxygen-free atmosphere. In some cases, an oxygen-free atmosphere may not have oxygen in the atmosphere. In some cases, an oxygen-free atmosphere may have less than about 10% oxygen, by volume, in the atmosphere. In some cases, an oxygen-free atmosphere may have less than about 9% oxygen, by volume, in the atmosphere. In some cases, an oxygen-free atmosphere may have less than about 8% oxygen, by volume, in the atmosphere. In some cases, an oxygen-free atmosphere may have less than about 7% oxygen, by volume, in the atmosphere. In some cases, an oxygen-free atmosphere may have less than about 6% oxygen, by volume, in the atmosphere. In some cases, an oxygen-free atmosphere may have less than about 5% oxygen, by volume, in the atmosphere. In some cases, an oxygen-free atmosphere may have less than about 4% oxygen, by volume, in the atmosphere. In some cases, an oxygen-free atmosphere may have less than about 3% oxygen, by volume, in the atmosphere. In some cases, an oxygen-free atmosphere may have less than about 2% oxygen, by volume, in the atmosphere. In some cases, an oxygen-free atmosphere may have less than about 1% oxygen, by volume, in the atmosphere. In some cases, an oxygen-free atmosphere may have less than about 1×10\hat{ }-1% oxygen, by volume, in the atmosphere. In some cases, an oxygen-free atmosphere may have less than about 1×10\hat{ }-2% oxygen, by volume, in the atmosphere. In some cases, an oxygen-free atmosphere may have less than about 1×10\hat{ }-3% oxygen, by volume, in the atmosphere. In some cases, an oxygen-free atmosphere may have less than about 1×10\hat{ }-4% oxygen, by volume, in the atmosphere. In some cases, an oxygen-free atmosphere may have less than about 1×10\hat{ }-5% oxygen, by volume, in the atmosphere. In some cases, an oxygen-free atmosphere may have less than about 1×10\hat{ }-6% oxygen, by volume, in the atmosphere. In some cases, an oxygen-free atmosphere may have less than about 1×10\hat{ }-7% oxygen, by volume, in the atmosphere. In some cases, an oxygen-free atmosphere may have less than about 1×10\hat{ }-8% oxygen, by volume, in the atmosphere. In some cases, an oxygen-free atmosphere may have less than about 1×10\hat{ }-9% oxygen, by volume, in the atmosphere. In some cases, an oxygen-free atmosphere may have less than about 1×10\hat{ }-10% oxygen, by volume, in the atmosphere. In some cases, an oxygen-free atmosphere may have less than about 1×10\hat{ }-11% oxygen, by volume, in the atmosphere. In some cases, an oxygen-free atmosphere may have less than about 21%, 20%, 19%, 18%, 17%, 16%, 15%, 14%, 13%, 12%, or 11% oxygen, by volume, in the atmosphere. In some instances, the pharmaceutical compositions may comprise any bacterial populations described herein and thereof.

In some instances, a method for producing a pharmaceutical composition may comprise storing the capsules in a bag before packing. In some cases, a method for producing a pharmaceutical composition may comprise storing the capsules in a bag in an oxygen-free atmosphere before packing. In some cases, the oxygen-free atmosphere is accomplished by injecting nitrogen gas or an oxygen scrubber into an atmosphere. In some cases, the nitrogen gas injected may comprise pure nitrogen gas. In some cases, the nitrogen gas injected may comprise 100%, 99%, 98%, 97%, 96%, 95%, 94%, 93%, 92%, 91%, 90%, 89%, 88%, 87%, 86%, 85%, 84%, 83%, 82%, 81%, or 80% nitrogen gas by volume of the atmosphere. In some cases, the nitrogen gas injected may comprise pure nitrogen gas. In some cases, the nitrogen gas injected may comprise 80% to about 99%, 85% to about 99%, 90% to about 99%, 95% to about 99%, 96% to about 99%, 97% to about 99%, 98% to about 99% nitrogen gas by volume of the atmosphere. In some cases, the oxygen scrubber injected may comprise pure nitrogen gas. In some cases, the oxygen scrubber injected may comprise 100%, 99%, 98%, 97%, 96%, 95%, 94%, 93%, 92%, 91%, 900%, 89%, 88%, 87%, 860%, 85%, 84%, 83%, 82%, 81%, or 80% oxygen scrubber by volume of the atmosphere. In some cases, the oxygen scrubber injected may comprise pure nitrogen gas. In some cases, the oxygen scrubber injected may comprise 80% to about 99%, 85% to about 99%, 90% to about 99%, 95% to about 99%, 96% to about 99%, 97% to about 99%, 98% to about 99% oxygen scrubber by volume of the atmosphere.

In some instances, an oxygen scrubber may comprise an inert gas. In some cases, an oxygen scrubber may comprise carbon dioxide. In other cases, an oxygen scrubber may comprise a noble gas. A noble gas may comprise helium, neon, argon, krypton, xenon, radon, or oganesson. In some cases, an oxygen scrubber may comprise N₂H₂CO₂ (90:5:5) gas mix. In some cases, the filling may be accomplished by a capsule filler. In other cases, the filling may be accomplished manually. In some cases, the filling may be accomplished in an anaerobic chamber. In some instances, the capsule may comprise any capsule described herein and thereof. In some cases, the filling may also fill a pharmaceutical composition in any primary container described herein and thereof. In some instances, the pack may comprise any blister pack described herein and thereof. In some cases, the pack may comprise any secondary container described herein and thereof.

### Methods for Large-Scale Growth of Lactobacillus sp.

Disclosed herein, are methods for large-scale growth of Lactobacillus sp. The method may comprise performing a plurality of inoculation rounds with an increasing amount of growth media. Lactobacillus sp., in some instances, may comprise Lactobacillus crispatus. In some cases, Lactobacillus sp. may comprise Lactobacillus crispatus (DSM 33187). In some cases, Lactobacillus sp. may comprise any bacterial species or strains of Lactobacillus sp. described herein and thereof.

In some cases, an inoculation round of a large-scale growth of Lactobacillus sp. may comprise at least about 0.001%, 0.002%, 0.005%, 0.01%, 0.02%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 20%, 50% or more by volume of a total batch material of a preceding inoculation. In some cases, an inoculation round of a large-scale growth of Lactobacillus sp. may comprise at most about 0.001%, 0.002%, 0.005%, 0.01%, 0.02%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.50%, 8%, 8.5%, 9%, 9.5% 10%, 20%, or 50% by volume of a total batch material of a preceding inoculation. In some cases, an inoculation round of a large-scale growth of Lactobacillus sp. may comprise at least about 5% by volume of a total batch material of a preceding inoculation. In some cases, an inoculation round of a large-scale growth of Lactobacillus sp. may comprise at least about 0.5% by volume of a total batch material of a preceding inoculation.

In some cases, an inoculation round of a large-scale growth of Lactobacillus sp. may comprise at least about 0.1% by volume of a total batch material of a preceding inoculation. In some cases, an inoculation round of the method may comprise at least about 0.2% by volume of a total batch material of a preceding inoculation. In some cases, an inoculation round of the method may comprise at least about 0.3% by volume of a total batch material of a preceding inoculation. In some cases, an inoculation round of the method may comprise at least about 0.4% by volume of a total batch material of a preceding inoculation. In some cases, an inoculation round of the method may comprise at least about 0.5% by volume of a total batch material of a preceding inoculation. In some cases, an inoculation round of the method may comprise at least about 0.6% by volume of a total batch material of a preceding inoculation. In some cases, an inoculation round of the method may comprise at least about 0.7% by volume of a total batch material of a preceding inoculation. In some cases, an inoculation round of the method may comprise at least about 0.8% by volume of a total batch material of a preceding inoculation. In some cases, an inoculation round of the method may comprise at least about 0.9% by volume of a total batch material of a preceding inoculation. In some cases, an inoculation round of the method may comprise at least about 1% by volume of a total batch material of a preceding inoculation. In some cases, an inoculation round of the method may comprise at least about 1.5% by volume of a total batch material of a preceding inoculation. In some cases, an inoculation round of the method may comprise at least about 2% by volume of a total batch material of a preceding inoculation. In some cases, an inoculation round of the method may comprise at least about 2.5% by volume of a total batch material of a preceding inoculation. In some cases, an inoculation round of the method may comprise at least about 3% by volume of a total batch material of a preceding inoculation. In some cases, an inoculation round of the method may comprise at least about 3.5% by volume of a total batch material of a preceding inoculation. In some cases, an inoculation round of the method may comprise at least about 4% by volume of a total batch material of a preceding inoculation. In some cases, an inoculation round of the method may comprise at least about 4.5% by volume of a total batch material of a preceding inoculation. In some cases, an inoculation round of the method may comprise at least about 5% by volume of a total batch material of a preceding inoculation. In some cases, an inoculation round of the method may comprise at least about 5.5% by volume of a total batch material of a preceding inoculation. In some cases, an inoculation round of the method may comprise at least about 6% by volume of a total batch material of a preceding inoculation. In some cases, an inoculation round of the method may comprise at least about 6.5% by volume of a total batch material of a preceding inoculation. In some cases, an inoculation round of the method may comprise at least about 7% by volume of a total batch material of a preceding inoculation. In some cases, an inoculation round of the method may comprise at least about 7.5% by volume of a total batch material of a preceding inoculation. In some cases, an inoculation round of the method may comprise at least about 8% by volume of a total batch material of a preceding inoculation. In some cases, an inoculation round of the method may comprise at least about 8.5% by volume of a total batch material of a preceding inoculation. In some cases, an inoculation round of the method may comprise at least about 9% by volume of a total batch material of a preceding inoculation. In some cases, an inoculation round of the method may comprise at least about 9.5% by volume of a total batch material of a preceding inoculation. In some cases, an inoculation round of the method may comprise at least about 10% by volume of a total batch material of a preceding inoculation.

In some cases, an inoculation round of a large-scale growth of Lactobacillus sp. may comprise less than about 0.1% by volume of a total batch material of a preceding inoculation. In some cases, an inoculation round of the method may comprise less than about 0.2% by volume of a total batch material of a preceding inoculation. In some cases, an inoculation round of the method may comprise less than about 0.3% by volume of a total batch material of a preceding inoculation. In some cases, an inoculation round of the method may comprise less than about 0.4% by volume of a total batch material of a preceding inoculation. In some cases, an inoculation round of the method may comprise less than about 0.5% by volume of a total batch material of a preceding inoculation. In some cases, an inoculation round of the method may comprise less than about 0.6% by volume of a total batch material of a preceding inoculation. In some cases, an inoculation round of the method may comprise less than about 0.7% by volume of a total batch material of a preceding inoculation. In some cases, an inoculation round of the method may comprise less than about 0.8% by volume of a total batch material of a preceding inoculation. In some cases, an inoculation round of the method may comprise less than about 0.9% by volume of a total batch material of a preceding inoculation. In some cases, an inoculation round of the method may comprise less than about 1% by volume of a total batch material of a preceding inoculation. In some cases, an inoculation round of the method may comprise less than about 1.5% by volume of a total batch material of a preceding inoculation. In some cases, an inoculation round of the method may comprise less than about 2% by volume of a total batch material of a preceding inoculation. In some cases, an inoculation round of the method may comprise less than about 2.5% by volume of a total batch material of a preceding inoculation. In some cases, an inoculation round of the method may comprise less than about 3% by volume of a total batch material of a preceding inoculation. In some cases, an inoculation round of the method may comprise less than about 3.5% by volume of a total batch material of a preceding inoculation. In some cases, an inoculation round of the method may comprise less than about 4% by volume of a total batch material of a preceding inoculation. In some cases, an inoculation round of the method may comprise less than about 4.5% by volume of a total batch material of a preceding inoculation. In some cases, an inoculation round of the method may comprise less than about 5% by volume of a total batch material of a preceding inoculation. In some cases, an inoculation round of the method may comprise less than about 5.5% by volume of a total batch material of a preceding inoculation. In some cases, an inoculation round of the method may comprise less than about 6% by volume of a total batch material of a preceding inoculation. In some cases, an inoculation round of the method may comprise less than about 6.5% by volume of a total batch material of a preceding inoculation. In some cases, an inoculation round of the method may comprise less than about 7% by volume of a total batch material of a preceding inoculation. In some cases, an inoculation round of the method may comprise less than about 7.5% by volume of a total batch material of a preceding inoculation. In some cases, an inoculation round of the method may comprise less than about 8% by volume of a total batch material of a preceding inoculation. In some cases, an inoculation round of the method may comprise less than about 8.5% by volume of a total batch material of a preceding inoculation. In some cases, an inoculation round of the method may comprise less than about 9% by volume of a total batch material of a preceding inoculation. In some cases, an inoculation round of the method may comprise less than about 9.5% by volume of a total batch material of a preceding inoculation. In some cases, an inoculation round of the method may comprise less than about 10% by volume of a total batch material of a preceding inoculation.

In some cases, an inoculation round of a large-scale growth of Lactobacillus sp. may comprise at least about 0.1% by volume of a total batch material of a preceding inoculation. In some cases, an inoculation round of the method may comprise at least about 0.2% by volume of a total batch material of a preceding inoculation. In some cases, an inoculation round of the method may comprise at least about 0.3% by volume of a total batch material of a preceding inoculation. In some cases, an inoculation round of the method may comprise at least about 0.4% by volume of a total batch material of a preceding inoculation. In some cases, an inoculation round of the method may comprise at least about 0.5% by volume of a total batch material of a preceding inoculation. In some cases, an inoculation round of the method may comprise at least about 0.6% by volume of a total batch material of a preceding inoculation. In some cases, an inoculation round of the method may comprise at least about 0.7% by volume of a total batch material of a preceding inoculation. In some cases, an inoculation round of the method may comprise at least about 0.8% by volume of a total batch material of a preceding inoculation. In some cases, an inoculation round of the method may comprise at least about 0.9% by volume of a total batch material of a preceding inoculation. In some cases, an inoculation round of the method may comprise at least about 1% by volume of a total batch material of a preceding inoculation. In some cases, an inoculation round of the method may comprise at least about 1.5% by volume of a total batch material of a preceding inoculation. In some cases, an inoculation round of the method may comprise at least about 2% by volume of a total batch material of a preceding inoculation. In some cases, an inoculation round of the method may comprise at least about 2.5% by volume of a total batch material of a preceding inoculation. In some cases, an inoculation round of the method may comprise at least about 3% by volume of a total batch material of a preceding inoculation. In some cases, an inoculation round of the method may comprise at least about 3.5% by volume of a total batch material of a preceding inoculation. In some cases, an inoculation round of the method may comprise at least about 4% by volume of a total batch material of a preceding inoculation. In some cases, an inoculation round of the method may comprise at least about 4.5% by volume of a total batch material of a preceding inoculation. In some cases, an inoculation round of the method may comprise at least about 5% by volume of a total batch material of a preceding inoculation. In some cases, an inoculation round of the method may comprise at least about 5.5% by volume of a total batch material of a preceding inoculation. In some cases, an inoculation round of the method may comprise at least about 6% by volume of a total batch material of a preceding inoculation. In some cases, an inoculation round of the method may comprise at least about 6.5% by volume of a total batch material of a preceding inoculation. In some cases, an inoculation round of the method may comprise at least about 7% by volume of a total batch material of a preceding inoculation. In some cases, an inoculation round of the method may comprise at least about 7.5% by volume of a total batch material of a preceding inoculation. In some cases, an inoculation round of the method may comprise at least about 8% by volume of a total batch material of a preceding inoculation. In some cases, an inoculation round of the method may comprise at least about 8.5% by volume of a total batch material of a preceding inoculation. In some cases, an inoculation round of the method may comprise at least about 9% by volume of a total batch material of a preceding inoculation. In some cases, an inoculation round of the method may comprise at least about 9.5% by volume of a total batch material of a preceding inoculation. In some cases, an inoculation round of the method may comprise at least about 10% by volume of a total batch material of a preceding inoculation.

In some cases, an inoculation round of a large-scale growth of Lactobacillus sp. may comprise less than about 0.1% by volume of a total batch material of a preceding inoculation. In some cases, an inoculation round of the method may comprise less than about 0.2% by volume of a total batch material of a preceding inoculation. In some cases, an inoculation round of the method may comprise less than about 0.3% by volume of a total batch material of a preceding inoculation. In some cases, an inoculation round of the method may comprise less than about 0.4% by volume of a total batch material of a preceding inoculation. In some cases, an inoculation round of the method may comprise less than about 0.5% by volume of a total batch material of a preceding inoculation. In some cases, an inoculation round of the method may comprise less than about 0.6% by volume of a total batch material of a preceding inoculation. In some cases, an inoculation round of the method may comprise less than about 0.7% by volume of a total batch material of a preceding inoculation. In some cases, an inoculation round of the method may comprise less than about 0.8% by volume of a total batch material of a preceding inoculation. In some cases, an inoculation round of the method may comprise less than about 0.9% by volume of a total batch material of a preceding inoculation. In some cases, an inoculation round of the method may comprise less than about 1% by volume of a total batch material of a preceding inoculation. In some cases, an inoculation round of the method may comprise less than about 1.5% by volume of a total batch material of a preceding inoculation. In some cases, an inoculation round of the method may comprise less than about 2% by volume of a total batch material of a preceding inoculation. In some cases, an inoculation round of the method may comprise less than about 2.5% by volume of a total batch material of a preceding inoculation. In some cases, an inoculation round of the method may comprise less than about 3% by volume of a total batch material of a preceding inoculation. In some cases, an inoculation round of the method may comprise less than about 3.5% by volume of a total batch material of a preceding inoculation. In some cases, an inoculation round of the method may comprise less than about 4% by volume of a total batch material of a preceding inoculation. In some cases, an inoculation round of the method may comprise less than about 4.5% by volume of a total batch material of a preceding inoculation. In some cases, an inoculation round of the method may comprise less than about 5% by volume of a total batch material of a preceding inoculation. In some cases, an inoculation round of the method may comprise less than about 5.5% by volume of a total batch material of a preceding inoculation. In some cases, an inoculation round of the method may comprise less than about 6% by volume of a total batch material of a preceding inoculation. In some cases, an inoculation round of the method may comprise less than about 6.5% by volume of a total batch material of a preceding inoculation. In some cases, an inoculation round of the method may comprise less than about 7% by volume of a total batch material of a preceding inoculation. In some cases, an inoculation round of the method may comprise less than about 7.5% by volume of a total batch material of a preceding inoculation. In some cases, an inoculation round of the method may comprise less than about 8% by volume of a total batch material of a preceding inoculation. In some cases, an inoculation round of the method may comprise less than about 8.5% by volume of a total batch material of a preceding inoculation. In some cases, an inoculation round of the method may comprise less than about 9% by volume of a total batch material of a preceding inoculation. In some cases, an inoculation round of the method may comprise less than about 9.5% by volume of a total batch material of a preceding inoculation. In some cases, an inoculation round of the method may comprise less than about 10% by volume of a total batch material of a preceding inoculation. In some cases, the percentage of the inoculation round described herein may also be measured by weight or by weight/volume. For example, for an inoculation round of the method that may comprise at least about 0.5% by volume of a total batch material of a preceding inoculation, inoculation round of the method that may also comprise at least about 0.5% by weight and/or at least about 0.5% weight/volume of the total batch material of a preceding inoculation.

In some instances, at least one of the inoculation rounds of the method may comprise of at least about 1 L, 5 L, 10 L, 20 L, 30 L, 40 L, 50 L, 60 L, 70 L, 80 L, 90 L, 100 L, 150 L, 200 L, 250 L, 300 L, 350 L, 400 L, 450 L, 500 L, 1000 L, 1500 L, 2000 L, 2500 L, 3000 L, 3500 L, 4000 L, 4500 L, 5000 L, 7500 L, 10000 L, 50000 L or more growth media. In some cases, at least one of the inoculation rounds of the method may comprise of at most about 1 L, 5 L, 10 L, 20 L, 30 L, 40 L, 50 L, 60 L, 70 L, 80 L, 90 L, 100 L, 150 L, 200 L, 250 L, 300 L, 350 L, 400 L, 450 L, 500 L, 1000 L, 1500 L, 2000 L, 2500 L, 3000 L, 3500 L, 4000 L, 4500 L, 5000 L, 7500 L, 10000 L, or 50000 L growth media. In some cases, the growth media of a large-scale growth of Lactobacillus sp. may be from about 100 ml to about 4000 L. In some cases, the growth media may be about 500 mL. In some cases, at least one of the inoculation rounds of the method may comprise of about 4000 L growth media. In some cases, at least one of the inoculation rounds of the method may comprise of about 3900 L growth media. In some cases, at least one of the inoculation rounds of the method may comprise of about 3800 L growth media. In some cases, at least one of the inoculation rounds of the method may comprise of about 3700 L growth media. In some cases, at least one of the inoculation rounds of the method may comprise of about 3600 L growth media. In some cases, at least one of the inoculation rounds of the method may comprise of about 3500 L growth media. In some cases, at least one of the inoculation rounds of the method may comprise of about 3400 L growth media. In some cases, at least one of the inoculation rounds of the method may comprise of about 3300 L growth media. In some cases, at least one of the inoculation rounds of the method may comprise of about 3200 L growth media. In some cases, at least one of the inoculation rounds of the method may comprise of about 3100 L growth media. In some cases, at least one of the inoculation rounds of the method may comprise of about 3000 L growth media. In some cases, at least one of the inoculation rounds of the method may comprise of about 2500 L growth media. In some cases, at least one of the inoculation rounds of the method may comprise of about 2000 L growth media. In some cases, at least one of the inoculation rounds of the method may comprise of about 1500 L growth media. In some cases, at least one of the inoculation rounds of the method may comprise of about 1000 L growth media. In some cases, at least one of the inoculation rounds of the method may comprise of about 500 L growth media. In some cases, at least one of the inoculation rounds of the method may comprise of about 500 L growth media. In some cases, at least one of the inoculation rounds of the method may comprise of about 450 L growth media. In some cases, at least one of the inoculation rounds of the method may comprise of about 400 L growth media. In some cases, at least one of the inoculation rounds of the method may comprise of about 350 L growth media. In some cases, at least one of the inoculation rounds of the method may comprise of about 300 L growth media. In some cases, at least one of the inoculation rounds of the method may comprise of about 250 L growth media. In some cases, at least one of the inoculation rounds of the method may comprise of about 200 L growth media. In some cases, at least one of the inoculation rounds of the method may comprise of about 150 L growth media. In some cases, at least one of the inoculation rounds of the method may comprise of about 100 L growth media. In some cases, at least one of the inoculation rounds of the method may comprise of about 90 L growth media. In some cases, at least one of the inoculation rounds of the method may comprise of about 80 L growth media. In some cases, at least one of the inoculation rounds of the method may comprise of about 70 L growth media. In some cases, at least one of the inoculation rounds of the method may comprise of about 60 L growth media. In some cases, at least one of the inoculation rounds of the method may comprise of about 50 L growth media. In some cases, at least one of the inoculation rounds of the method may comprise of about 40 L growth media. In some cases, at least one of the inoculation rounds of the method may comprise of about 30 L growth media. In some cases, at least one of the inoculation rounds of the method may comprise of about 20 L growth media. In some cases, at least one of the inoculation rounds of the method may comprise of about 10 L growth media. In some cases, at least one of the inoculation rounds of the method may comprise of about 5 L growth media. In some cases, at least one of the inoculation rounds of the method may comprise of about 1 L growth media.

Methods of this disclosure for manufacturing bacterial populations can include media preparation, which can involve dissolving various dry media components such as salts, vitamins, antioxidants, etc., in USP. grade water for injection. After complete dissolution, the pH of the media can be adjusted to ensure optimal growth of the respective bacterial cells. The pH adjusted media can then be transferred to a biosafety cabinet and sterilized. In various instances, a microbial population herein can comprise one or more anaerobic bacterial strains. In such instances, media can be transferred to an anaerobic chamber containing an atmosphere of about N₂H₂CO₂ (90:5:5) to reduce prior to inoculation of anaerobic bacteria.

The manufacturing methods herein can comprise generating a starter culture of the bacterial strains/species. Such methods can include generating a starter culture for the bacterial strains to be included in a pharmaceutical composition by using a certain volume from each flask containing filtered media and transfer such volume to a sterile, pre-reduced screw cap tube, followed by a transfer of thawed bacterial cells using a stock solution from a cell bank that contains the respective bacterial cells. In instances where anaerobic bacterial cells are used, the starter cultures can be grown at about 37° C. for about 12-16 hours under anaerobic conditions. After a certain incubation time period, the starter cultures can be visibly inspected for growth (turbidity) and, upon confirmation of bacterial growth, transferred into additional, larger culture flasks for cell expansion. After incubation for about 12, 18, 24, 30 hours, the cell density and absorbance of the cell culture medium can be measured. Such measurements can be performed by measuring the absorbance at of the cell suspension at around 583 or 600 nanometers to ensure the absorbance falls within a specified optical density range.

In some cases, the initial inoculation round may comprise a frozen stock of Lactobacillus crispatus of about 0.1% of an initial inoculation round growth media. In some cases, the initial inoculation round may comprise a frozen stock of Lactobacillus crispatus of about 0.2% of an initial inoculation round growth media. In some cases, the initial inoculation round may comprise a frozen stock of Lactobacillus crispatus of about 0.3% of an initial inoculation round growth media. In some cases, the initial inoculation round may comprise a frozen stock of Lactobacillus crispatus of about 0.4% of an initial inoculation round growth media. In some cases, the initial inoculation round may comprise a frozen stock of Lactobacillus crispatus of about 0.5% of an initial inoculation round growth media. In some cases, the initial inoculation round may comprise a frozen stock of Lactobacillus crispatus of about 0.6% of an initial inoculation round growth media. In some cases, the initial inoculation round may comprise a frozen stock of Lactobacillus crispatus of about 0.7% of an initial inoculation round growth media. In some cases, the initial inoculation round may comprise a frozen stock of Lactobacillus crispatus of about 0.8% of an initial inoculation round growth media. In some cases, the initial inoculation round may comprise a frozen stock of Lactobacillus crispatus of about 0.9% of an initial inoculation round growth media. In some cases, the initial inoculation round may comprise a frozen stock of Lactobacillus crispatus of about 1% of an initial inoculation round growth media. In some cases, a frozen stock of Lactobacillus crispatus may comprise Lactobacillus crispatus (DSM 33187). In some cases, the method may comprise performing a plurality of sterilization and degassing rounds for the growth media. Sterilization may comprise filter-sterilization. In some cases, sterilization may comprise autoclaving at 121° C. for 20 mins. In some cases, degassing may comprise backfilling the media or the environment with N₂H₂CO₂ (90:5:5) gas mix. In some cases, degassing may comprise backfilling the media or the environment with nitrogen gas or the oxygen scrubbers described herein and thereof. In some cases, the method may comprise lyophilizing the batch. In other cases, the method may comprise centrifuging the batch before the lyophilizing. In one case, the method may comprise grinding the batch after the lyophilizing.

### Methods for Large-Scale Growth of Species of the Lactobacillaceae Family

Disclosed herein, are methods for large-scale growth of species of the Lactobacillaceae family. Disclosed herein, are methods for large-scale growth of species of the Lactobacillaceae family. The method may comprise performing a plurality of inoculation rounds with an increasing amount of growth media. Species of the Lactobacillaceae family, in some instances, may comprise Lactobacillus crispatus. In some cases, species of the Lactobacillaceae family may comprise Lactobacillus crispatus (DSM 33187). In some cases, species of the Lactobacillaceae family may comprise any bacterial species or strains of the Lactobacillaceae family described herein and thereof.

In some cases, an inoculation round of a large-scale growth of species of the Lactobacillaceae family may comprise at least about 0.001%, 0.002%, 0.005%, 0.01%, 0.02%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 20%, 50% or more by volume of a total batch material of a preceding inoculation. In some cases, an inoculation round of a large-scale growth of species of the Lactobacillaceae family may comprise at most about 0.001%, 0.002%, 0.005%, 0.01%, 0.02%, 0.05%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 20%, or 50% by volume of a total batch material of a preceding inoculation. In some cases, an inoculation round of a large-scale growth of species of the Lactobacillaceae family may comprise at least about 5% by volume of a total batch material of a preceding inoculation. In some cases, an inoculation round of a large-scale growth of species of the Lactobacillaceae family may comprise at least about 0.5% by volume of a total batch material of a preceding inoculation.

In some cases, an inoculation round of a large-scale growth of species of the Lactobacillaceae family may comprise at least about 0.1% by volume of a total batch material of a preceding inoculation. In some cases, an inoculation round of the method may comprise at least about 0.2% by volume of a total batch material of a preceding inoculation. In some cases, an inoculation round of the method may comprise at least about 0.3% by volume of a total batch material of a preceding inoculation. In some cases, an inoculation round of the method may comprise at least about 0.4% by volume of a total batch material of a preceding inoculation. In some cases, an inoculation round of the method may comprise at least about 0.5% by volume of a total batch material of a preceding inoculation. In some cases, an inoculation round of the method may comprise at least about 0.6% by volume of a total batch material of a preceding inoculation. In some cases, an inoculation round of the method may comprise at least about 0.7% by volume of a total batch material of a preceding inoculation. In some cases, an inoculation round of the method may comprise at least about 0.8% by volume of a total batch material of a preceding inoculation. In some cases, an inoculation round of the method may comprise at least about 0.9% by volume of a total batch material of a preceding inoculation. In some cases, an inoculation round of the method may comprise at least about 1% by volume of a total batch material of a preceding inoculation. In some cases, an inoculation round of the method may comprise at least about 1.5% by volume of a total batch material of a preceding inoculation. In some cases, an inoculation round of the method may comprise at least about 2% by volume of a total batch material of a preceding inoculation. In some cases, an inoculation round of the method may comprise at least about 2.5% by volume of a total batch material of a preceding inoculation. In some cases, an inoculation round of the method may comprise at least about 3% by volume of a total batch material of a preceding inoculation. In some cases, an inoculation round of the method may comprise at least about 3.5% by volume of a total batch material of a preceding inoculation. In some cases, an inoculation round of the method may comprise at least about 4% by volume of a total batch material of a preceding inoculation. In some cases, an inoculation round of the method may comprise at least about 4.5% by volume of a total batch material of a preceding inoculation. In some cases, an inoculation round of the method may comprise at least about 5% by volume of a total batch material of a preceding inoculation. In some cases, an inoculation round of the method may comprise at least about 5.5% by volume of a total batch material of a preceding inoculation. In some cases, an inoculation round of the method may comprise at least about 6% by volume of a total batch material of a preceding inoculation. In some cases, an inoculation round of the method may comprise at least about 6.5% by volume of a total batch material of a preceding inoculation. In some cases, an inoculation round of the method may comprise at least about 7% by volume of a total batch material of a preceding inoculation. In some cases, an inoculation round of the method may comprise at least about 7.5% by volume of a total batch material of a preceding inoculation. In some cases, an inoculation round of the method may comprise at least about 8% by volume of a total batch material of a preceding inoculation. In some cases, an inoculation round of the method may comprise at least about 8.5% by volume of a total batch material of a preceding inoculation. In some cases, an inoculation round of the method may comprise at least about 9% by volume of a total batch material of a preceding inoculation. In some cases, an inoculation round of the method may comprise at least about 9.5% by volume of a total batch material of a preceding inoculation. In some cases, an inoculation round of the method may comprise at least about 10% by volume of a total batch material of a preceding inoculation.

In some cases, an inoculation round of a large-scale growth of species of the Lactobacillaceae family may comprise less than about 0.1% by volume of a total batch material of a preceding inoculation. In some cases, an inoculation round of the method may comprise less than about 0.2% by volume of a total batch material of a preceding inoculation. In some cases, an inoculation round of the method may comprise less than about 0.3% by volume of a total batch material of a preceding inoculation. In some cases, an inoculation round of the method may comprise less than about 0.4% by volume of a total batch material of a preceding inoculation. In some cases, an inoculation round of the method may comprise less than about 0.5% by volume of a total batch material of a preceding inoculation. In some cases, an inoculation round of the method may comprise less than about 0.6% by volume of a total batch material of a preceding inoculation. In some cases, an inoculation round of the method may comprise less than about 0.7% by volume of a total batch material of a preceding inoculation. In some cases, an inoculation round of the method may comprise less than about 0.8% by volume of a total batch material of a preceding inoculation. In some cases, an inoculation round of the method may comprise less than about 0.9% by volume of a total batch material of a preceding inoculation. In some cases, an inoculation round of the method may comprise less than about 1% by volume of a total batch material of a preceding inoculation. In some cases, an inoculation round of the method may comprise less than about 1.5% by volume of a total batch material of a preceding inoculation. In some cases, an inoculation round of the method may comprise less than about 2% by volume of a total batch material of a preceding inoculation. In some cases, an inoculation round of the method may comprise less than about 2.5% by volume of a total batch material of a preceding inoculation. In some cases, an inoculation round of the method may comprise less than about 3% by volume of a total batch material of a preceding inoculation. In some cases, an inoculation round of the method may comprise less than about 3.5% by volume of a total batch material of a preceding inoculation. In some cases, an inoculation round of the method may comprise less than about 4% by volume of a total batch material of a preceding inoculation. In some cases, an inoculation round of the method may comprise less than about 4.5% by volume of a total batch material of a preceding inoculation. In some cases, an inoculation round of the method may comprise less than about 5% by volume of a total batch material of a preceding inoculation. In some cases, an inoculation round of the method may comprise less than about 5.5% by volume of a total batch material of a preceding inoculation. In some cases, an inoculation round of the method may comprise less than about 6% by volume of a total batch material of a preceding inoculation. In some cases, an inoculation round of the method may comprise less than about 6.5% by volume of a total batch material of a preceding inoculation. In some cases, an inoculation round of the method may comprise less than about 7% by volume of a total batch material of a preceding inoculation. In some cases, an inoculation round of the method may comprise less than about 7.5% by volume of a total batch material of a preceding inoculation. In some cases, an inoculation round of the method may comprise less than about 8% by volume of a total batch material of a preceding inoculation. In some cases, an inoculation round of the method may comprise less than about 8.5% by volume of a total batch material of a preceding inoculation. In some cases, an inoculation round of the method may comprise less than about 9% by volume of a total batch material of a preceding inoculation. In some cases, an inoculation round of the method may comprise less than about 9.5% by volume of a total batch material of a preceding inoculation. In some cases, an inoculation round of the method may comprise less than about 10% by volume of a total batch material of a preceding inoculation.

In some cases, an inoculation round of a large-scale growth of species of the Lactobacillaceae family may comprise at least about 0.1% by volume of a total batch material of a preceding inoculation. In some cases, an inoculation round of the method may comprise at least about 0.2% by volume of a total batch material of a preceding inoculation. In some cases, an inoculation round of the method may comprise at least about 0.3% by volume of a total batch material of a preceding inoculation. In some cases, an inoculation round of the method may comprise at least about 0.4% by volume of a total batch material of a preceding inoculation. In some cases, an inoculation round of the method may comprise at least about 0.5% by volume of a total batch material of a preceding inoculation. In some cases, an inoculation round of the method may comprise at least about 0.6% by volume of a total batch material of a preceding inoculation. In some cases, an inoculation round of the method may comprise at least about 0.7% by volume of a total batch material of a preceding inoculation. In some cases, an inoculation round of the method may comprise at least about 0.8% by volume of a total batch material of a preceding inoculation. In some cases, an inoculation round of the method may comprise at least about 0.9% by volume of a total batch material of a preceding inoculation. In some cases, an inoculation round of the method may comprise at least about 1% by volume of a total batch material of a preceding inoculation. In some cases, an inoculation round of the method may comprise at least about 1.5% by volume of a total batch material of a preceding inoculation. In some cases, an inoculation round of the method may comprise at least about 2% by volume of a total batch material of a preceding inoculation. In some cases, an inoculation round of the method may comprise at least about 2.5% by volume of a total batch material of a preceding inoculation. In some cases, an inoculation round of the method may comprise at least about 3% by volume of a total batch material of a preceding inoculation. In some cases, an inoculation round of the method may comprise at least about 3.5% by volume of a total batch material of a preceding inoculation. In some cases, an inoculation round of the method may comprise at least about 4% by volume of a total batch material of a preceding inoculation. In some cases, an inoculation round of the method may comprise at least about 4.5% by volume of a total batch material of a preceding inoculation. In some cases, an inoculation round of the method may comprise at least about 5% by volume of a total batch material of a preceding inoculation. In some cases, an inoculation round of the method may comprise at least about 5.5% by volume of a total batch material of a preceding inoculation. In some cases, an inoculation round of the method may comprise at least about 6% by volume of a total batch material of a preceding inoculation. In some cases, an inoculation round of the method may comprise at least about 6.5% by volume of a total batch material of a preceding inoculation. In some cases, an inoculation round of the method may comprise at least about 7% by volume of a total batch material of a preceding inoculation. In some cases, an inoculation round of the method may comprise at least about 7.5% by volume of a total batch material of a preceding inoculation. In some cases, an inoculation round of the method may comprise at least about 8% by volume of a total batch material of a preceding inoculation. In some cases, an inoculation round of the method may comprise at least about 8.5% by volume of a total batch material of a preceding inoculation. In some cases, an inoculation round of the method may comprise at least about 9% by volume of a total batch material of a preceding inoculation. In some cases, an inoculation round of the method may comprise at least about 9.5% by volume of a total batch material of a preceding inoculation. In some cases, an inoculation round of the method may comprise at least about 10% by volume of a total batch material of a preceding inoculation.

In some cases, an inoculation round of a large-scale growth of species of the Lactobacillaceae family may comprise less than about 0.1% by volume of a total batch material of a preceding inoculation. In some cases, an inoculation round of the method may comprise less than about 0.2% by volume of a total batch material of a preceding inoculation. In some cases, an inoculation round of the method may comprise less than about 0.3% by volume of a total batch material of a preceding inoculation. In some cases, an inoculation round of the method may comprise less than about 0.4% by volume of a total batch material of a preceding inoculation. In some cases, an inoculation round of the method may comprise less than about 0.5% by volume of a total batch material of a preceding inoculation. In some cases, an inoculation round of the method may comprise less than about 0.6% by volume of a total batch material of a preceding inoculation. In some cases, an inoculation round of the method may comprise less than about 0.7% by volume of a total batch material of a preceding inoculation. In some cases, an inoculation round of the method may comprise less than about 0.8% by volume of a total batch material of a preceding inoculation. In some cases, an inoculation round of the method may comprise less than about 0.9% by volume of a total batch material of a preceding inoculation. In some cases, an inoculation round of the method may comprise less than about 1% by volume of a total batch material of a preceding inoculation. In some cases, an inoculation round of the method may comprise less than about 1.5% by volume of a total batch material of a preceding inoculation. In some cases, an inoculation round of the method may comprise less than about 2% by volume of a total batch material of a preceding inoculation. In some cases, an inoculation round of the method may comprise less than about 2.5% by volume of a total batch material of a preceding inoculation. In some cases, an inoculation round of the method may comprise less than about 3% by volume of a total batch material of a preceding inoculation. In some cases, an inoculation round of the method may comprise less than about 3.5% by volume of a total batch material of a preceding inoculation. In some cases, an inoculation round of the method may comprise less than about 4% by volume of a total batch material of a preceding inoculation. In some cases, an inoculation round of the method may comprise less than about 4.5% by volume of a total batch material of a preceding inoculation. In some cases, an inoculation round of the method may comprise less than about 5% by volume of a total batch material of a preceding inoculation. In some cases, an inoculation round of the method may comprise less than about 5.5% by volume of a total batch material of a preceding inoculation. In some cases, an inoculation round of the method may comprise less than about 6% by volume of a total batch material of a preceding inoculation. In some cases, an inoculation round of the method may comprise less than about 6.5% by volume of a total batch material of a preceding inoculation. In some cases, an inoculation round of the method may comprise less than about 7% by volume of a total batch material of a preceding inoculation. In some cases, an inoculation round of the method may comprise less than about 7.5% by volume of a total batch material of a preceding inoculation. In some cases, an inoculation round of the method may comprise less than about 8% by volume of a total batch material of a preceding inoculation. In some cases, an inoculation round of the method may comprise less than about 8.5% by volume of a total batch material of a preceding inoculation. In some cases, an inoculation round of the method may comprise less than about 9% by volume of a total batch material of a preceding inoculation. In some cases, an inoculation round of the method may comprise less than about 9.5% by volume of a total batch material of a preceding inoculation. In some cases, an inoculation round of the method may comprise less than about 10% by volume of a total batch material of a preceding inoculation. In some cases, the percentage of the inoculation round described herein may also be measured by weight or by weight/volume. For example, for an inoculation round of the method that may comprise at least about 0.5% by volume of a total batch material of a preceding inoculation, inoculation round of the method that may also comprise at least about 0.5% by weight and/or at least about 0.5% weight/volume of the total batch material of a preceding inoculation.

In some instances, at least one of the inoculation rounds of the method may comprise of at least about 1 L, 5 L, 10 L, 20 L, 30 L, 40 L, 50 L, 60 L, 70 L, 80 L, 90 L, 100 L, 150 L, 200 L, 250 L, 300 L, 350 L, 400 L, 450 L, 500 L, 1000 L, 1500 L, 2000 L, 2500 L, 3000 L, 3500 L, 4000 L, 4500 L, 5000 L, 7500 L, 10000 L, 50000 L or more growth media. In some cases, at least one of the inoculation rounds of the method may comprise of at most about 1 L, 5 L, 10 L, 20 L, 30 L, 40 L, 50 L, 60 L, 70 L, 80 L, 90 L, 100 L, 150 L, 200 L, 250 L, 300 L, 350 L, 400 L, 450 L, 500 L, 1000 L, 1500 L, 2000 L, 2500 L, 3000 L, 3500 L, 4000 L, 4500 L, 5000 L, 7500 L, 10000 L, or 50000 L growth media. In some cases, the growth media of a large-scale growth of species of the Lactobacillaceae family may be from about 100 ml to about 4000 L. In some cases, the growth media may be about 500 mL. In some cases, at least one of the inoculation rounds of the method may comprise of about 4000 L growth media. In some cases, at least one of the inoculation rounds of the method may comprise of about 3900 L growth media. In some cases, at least one of the inoculation rounds of the method may comprise of about 3800 L growth media. In some cases, at least one of the inoculation rounds of the method may comprise of about 3700 L growth media. In some cases, at least one of the inoculation rounds of the method may comprise of about 3600 L growth media. In some cases, at least one of the inoculation rounds of the method may comprise of about 3500 L growth media. In some cases, at least one of the inoculation rounds of the method may comprise of about 3400 L growth media. In some cases, at least one of the inoculation rounds of the method may comprise of about 3300 L growth media. In some cases, at least one of the inoculation rounds of the method may comprise of about 3200 L growth media. In some cases, at least one of the inoculation rounds of the method may comprise of about 3100 L growth media. In some cases, at least one of the inoculation rounds of the method may comprise of about 3000 L growth media. In some cases, at least one of the inoculation rounds of the method may comprise of about 2500 L growth media. In some cases, at least one of the inoculation rounds of the method may comprise of about 2000 L growth media. In some cases, at least one of the inoculation rounds of the method may comprise of about 1500 L growth media. In some cases, at least one of the inoculation rounds of the method may comprise of about 1000 L growth media. In some cases, at least one of the inoculation rounds of the method may comprise of about 500 L growth media. In some cases, at least one of the inoculation rounds of the method may comprise of about 500 L growth media. In some cases, at least one of the inoculation rounds of the method may comprise of about 450 L growth media. In some cases, at least one of the inoculation rounds of the method may comprise of about 400 L growth media. In some cases, at least one of the inoculation rounds of the method may comprise of about 350 L growth media. In some cases, at least one of the inoculation rounds of the method may comprise of about 300 L growth media. In some cases, at least one of the inoculation rounds of the method may comprise of about 250 L growth media. In some cases, at least one of the inoculation rounds of the method may comprise of about 200 L growth media. In some cases, at least one of the inoculation rounds of the method may comprise of about 150 L growth media. In some cases, at least one of the inoculation rounds of the method may comprise of about 100 L growth media. In some cases, at least one of the inoculation rounds of the method may comprise of about 90 L growth media. In some cases, at least one of the inoculation rounds of the method may comprise of about 80 L growth media. In some cases, at least one of the inoculation rounds of the method may comprise of about 70 L growth media. In some cases, at least one of the inoculation rounds of the method may comprise of about 60 L growth media. In some cases, at least one of the inoculation rounds of the method may comprise of about 50 L growth media. In some cases, at least one of the inoculation rounds of the method may comprise of about 40 L growth media. In some cases, at least one of the inoculation rounds of the method may comprise of about 30 L growth media. In some cases, at least one of the inoculation rounds of the method may comprise of about 20 L growth media. In some cases, at least one of the inoculation rounds of the method may comprise of about 10 L growth media. In some cases, at least one of the inoculation rounds of the method may comprise of about 5 L growth media. In some cases, at least one of the inoculation rounds of the method may comprise of about 1 L growth media.

Methods of this disclosure for manufacturing bacterial populations can include media preparation, which can involve dissolving various dry media components such as salts, vitamins, antioxidants, etc., in USP. grade water for injection. After complete dissolution, the pH of the media can be adjusted to ensure optimal growth of the respective bacterial cells. The pH adjusted media can then be transferred to a biosafety cabinet and sterilized. In various instances, a microbial population herein can comprise one or more anaerobic bacterial strains. In such instances, media can be transferred to an anaerobic chamber containing an atmosphere of about N₂H₂CO₂ (90:5:5) to reduce prior to inoculation of anaerobic bacteria.

The manufacturing methods herein can comprise generating a starter culture of the bacterial strains/species. Such methods can include generating a starter culture for the bacterial strains to be included in a pharmaceutical composition by using a certain volume from each flask containing filtered media and transfer such volume to a sterile, pre-reduced screw cap tube, followed by a transfer of thawed bacterial cells using a stock solution from a cell bank that contains the respective bacterial cells. In instances where anaerobic bacterial cells are used, the starter cultures can be grown at about 37° C. for about 12-16 hours under anaerobic conditions. After a certain incubation time period, the starter cultures can be visibly inspected for growth (turbidity) and, upon confirmation of bacterial growth, transferred into additional, larger culture flasks for cell expansion. After incubation for about 12, 18, 24, 30 hours, the cell density and absorbance of the cell culture medium can be measured. Such measurements can be performed by measuring the absorbance at of the cell suspension at around 583 or 600 nanometers to ensure the absorbance falls within a specified optical density range.

In some cases, the initial inoculation round may comprise a frozen stock of Lactobacillus crispatus of about 0.1% of an initial inoculation round growth media. In some cases, the initial inoculation round may comprise a frozen stock of Lactobacillus crispatus of about 0.2% of an initial inoculation round growth media. In some cases, the initial inoculation round may comprise a frozen stock of Lactobacillus crispatus of about 0.3% of an initial inoculation round growth media. In some cases, the initial inoculation round may comprise a frozen stock of Lactobacillus crispatus of about 0.4% of an initial inoculation round growth media. In some cases, the initial inoculation round may comprise a frozen stock of Lactobacillus crispatus of about 0.5% of an initial inoculation round growth media. In some cases, the initial inoculation round may comprise a frozen stock of Lactobacillus crispatus of about 0.6% of an initial inoculation round growth media. In some cases, the initial inoculation round may comprise a frozen stock of Lactobacillus crispatus of about 0.7% of an initial inoculation round growth media. In some cases, the initial inoculation round may comprise a frozen stock of Lactobacillus crispatus of about 0.8% of an initial inoculation round growth media. In some cases, the initial inoculation round may comprise a frozen stock of Lactobacillus crispatus of about 0.9% of an initial inoculation round growth media. In some cases, the initial inoculation round may comprise a frozen stock of Lactobacillus crispatus of about 1% of an initial inoculation round growth media. In some cases, a frozen stock of Lactobacillus crispatus may comprise Lactobacillus crispatus (DSM 33187). In some cases, the method may comprise performing a plurality of sterilization and degassing rounds for the growth media. Sterilization may comprise filter-sterilization. In some cases, sterilization may comprise autoclaving at 121° C. for 20 mins. In some cases, degassing may comprise backfilling the media or the environment with N₂H₂CO₂ (90:5:5) gas mix. In some cases, degassing may comprise backfilling the media or the environment with nitrogen gas or the oxygen scrubbers described herein and thereof. In some cases, the method may comprise lyophilizing the batch. In other cases, the method may comprise centrifuging the batch before the lyophilizing. In one case, the method may comprise grinding the batch after the lyophilizing.

Methods of Treatment with Pharmaceutical Compositions

### Methods of Treatment

In some instances, a pharmaceutical composition may be administered to a subject having or suspected of having a disease. In some cases, a pharmaceutical composition may be administered to a subject having a disease. In other cases, a pharmaceutical composition may be administered to a subject suspected of having a disease. In some cases, a pharmaceutical composition may be administered to a subject to treat a disease in the subject. In some cases, a pharmaceutical composition may be administered to a subject to prevent a disease in the subject. In some cases, when used to prevent a disease, the subject may not have developed the disease before being administered with the pharmaceutical composition.

In some instances, a disease treated by a pharmaceutical composition may comprise an inflammatory disease. In some cases, an inflammatory disease treated by a pharmaceutical composition allergy or dermatitis. In some cases, a disease treated by a pharmaceutical composition may comprise an allergy. In some cases, an inflammatory disease treated by a pharmaceutical composition may comprise dermatitis. In some instances, an inflammatory disease treated by a pharmaceutical composition may comprise atopy, asthma, an autoimmune disease, an autoinflammatory disease, a hypersensitivity, pediatric allergic asthma, allergic asthma, inflammatory bowel disease, Celiac disease, Crohn's disease, colitis, ulcerative colitis, collagenous colitis, lymphocytic colitis, diverticulitis, irritable bowel syndrome, short bowel syndrome, stagnant loop syndrome, chronic persistent diarrhea, intractable diarrhea of infancy, Traveler's diarrhea, immunoproliferative small intestinal disease, chronic prostatitis, postenteritis syndrome, tropical sprue, Whipple's disease, Wolman disease, arthritis, rheumatoid arthritis, Behcet's disease, uveitis, pyoderma gangrenosum, erythema nodosum, traumatic brain injury, psoriatic arthritis, juvenile idiopathic arthritis, multiple sclerosis, systemic lupus erythematosus (SLE), mayasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, bullous pemphigoid, sarcoidosis, ichthyosis, Graves ophthalmopathy, Addison's disease, Vitiligo, acne vulgaris, pelvic inflammatory disease, reperfusion injury, sarcoidosis, transplant rejection, interstitial cystitis, atherosclerosis, food allergy or atopic dermatitis atopic dermatitis. In some instances, an inflammatory disease treated by a pharmaceutical composition may comprise pediatric allergic asthma or inflammatory bowel disease. In some instances, an allergy treated by a pharmaceutical composition may comprise allergic asthma, food allergy, or allergic pediatric asthma. In some cases, an allergy treated by a pharmaceutical composition may comprise allergic asthma. In some cases, an allergy treated by a pharmaceutical composition may comprise food allergy. In some cases, an allergy treated by a pharmaceutical composition may comprise allergic pediatric asthma.

In some instances, a disease treated by a pharmaceutical composition may comprise a metabolic disease. In some cases, a metabolic disease treated by a pharmaceutical composition may comprise obesity, diabetes, or a metabolic syndrome. In some cases, a metabolic disease treated by a pharmaceutical composition may comprise obesity. In some cases, a metabolic disease treated by a pharmaceutical composition may comprise diabetes. In some cases, a metabolic disease treated by a pharmaceutical composition may comprise a metabolic syndrome. In some cases, a metabolic disease treated by a pharmaceutical composition may also comprise prediabetes, type 1 diabetes, type 2 diabetes, diabetes complications, prediabetes, non-alcoholic fatty liver disease (NAFLD), weight loss, insulin-deficiency or insulin-resistance related disorders, glucose intolerance, abnormal lipid metabolism, atherosclerosis, hypertension, cardiac pathology, stroke, hyperglycemia, hepatic steatosis, dyslipidemia, dysfunction of the immune system associated with overweight and obesity, cardiovascular diseases, high cholesterol, elevated triglycerides, asthma, sleep apnoea, osteoarthritis, neuro-degeneration, gallbladder disease, syndrome X, inflammatory and immune disorders, atherogenic dyslipidemia or cancer.

In some cases, a subject administered with the pharmaceutical compositions described herein and thereof may comprise a human subject. A human subject may be an infant of an adult. An infant that is administered with the pharmaceutical compositions described herein and thereof, in some cases, have an age of at least about 1 day old, 2 days old, 3 days old, 4 days old, 5 days old, 6 days old, 1 week old, 2 weeks old, 3 weeks old, 4 weeks old, 1 month old, 2 months old, 3 months old, 4 months old, 5 months old, 6 months old, 7 months old, 8 months old, 9 months old, 10 months old, 11 months old, 1 year old, 2 years old, 3 years old, 4 years old, 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, 23 years old, 24 years old, 25 years old, 26 years old, 27 years old, 28 years old, 29 years old, 30 years old, 31 years old, 32 years old, 33 years old, 34 years old, 35 years old, 36 years old, 37 years old, 38 years old, 39 years old, 40 years old, 41 years old, 42 years old, 43 years old, 44 years old, 45 years old, 46 years old, 47 years old, 48 years old, 49 years old, 50 years old, 51 years old, 52 years old, 53 years old, 54 years old, 55 years old, 56 years old, 57 years old, 58 years old, 59 years old, 60 years old, 61 years old, 62 years old, 63 years old, 64 years old, 65 years old, 66 years old, 67 years old, 68 years old, 69 years old, 70 years old, 71 years old, 72 years old, 73 years old, 74 years old, 75 years old, 76 years old, 77 years old, 78 years old, 79 years old, 80 years old, 81 years old, 82 years old, 83 years old, 84 years old, 85 years old, 86 years old, 87 years old, 88 years old, 89 years old, 90 years old, 91 years old, 92 years old, 93 years old, 94 years old, 95 years old, 96 years old, 97 years old, 98 years old, 99 years old, or 100 years old.

A pharmaceutical composition herein can be administered for various periods of time according to different administration schedules. A treatment period may vary between subjects and individuals and can depend on various factors as described herein, e.g., disease state, age, etc. In some instances, a subject can be treated for one day to at least about one week, for about a week to about one month, or for about one month to about one year. In such instances, the subject can be treated for about one month, two months, or three months. In some cases, treatment can be performed on consecutive days, consecutive weeks, and/or consecutive months. In some embodiments, a pharmaceutical composition is administered for about 28, 29, or 30 consecutive days.

Methods of treatment herein can include administering a pharmaceutical composition of this disclosure once, two, three, four, five, six, seven, eight, nine, ten, eleven, or twelve times daily. In various instances, a pharmaceutical composition of this disclosure is administered twice daily. Such twice daily administration can be performed in the morning and in the evening. In such cases, there can be a period of about 8, 12, or 16 hours between the first and the second administration of a given day.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

These examples are provided for illustrative purposes only and not to limit the scope of the claims provided herein.

### Example 1—Optimized and Ultra-Large-Scale Growth and Manufacturing Process for Lactobacillus crispatus (DSM 33187)

Provided herein are optimized and ultra-large-scale growth (˜3000 L) and manufacturing process for Lactobacillus crispatus (DSM 33187). The strain may be used as part of a pharmaceutical composition described herein and thereof.

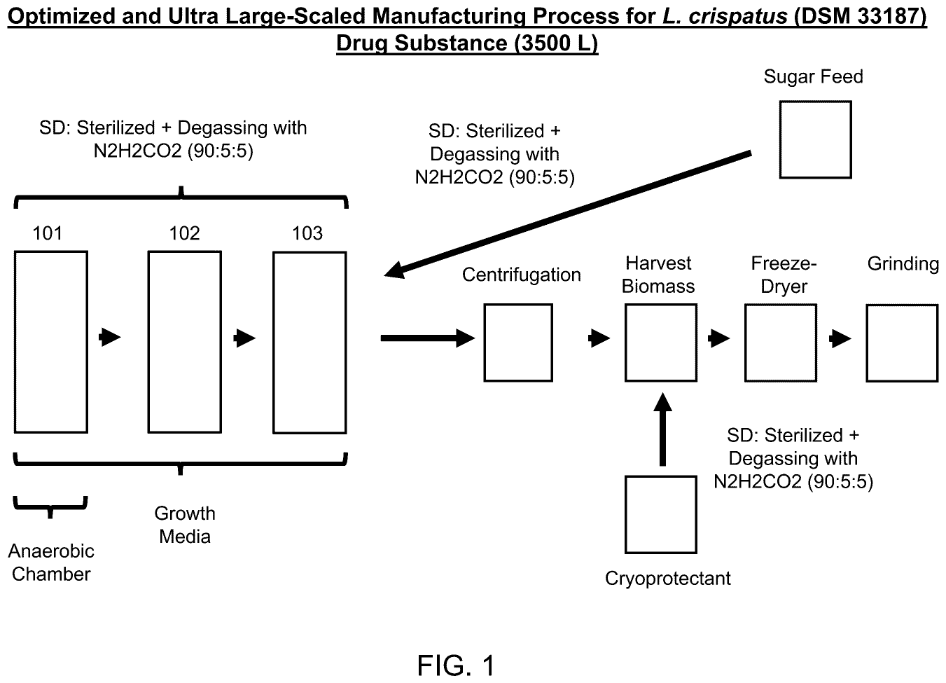

Manufacturing conditions and procedures for growing Lactobacillus crispatus (DSM 33187) in 3500 L volume of culture, as outlined in FIG. 1, were generated to increase yield and growth rate of bacterial strains described herein.

### Media Preparation

The components for the 250 L sugar feed (TABLE 3), 3160 L of LC100 media (TABLE 4), and cryoprotectant mix (TABLE 5) were weighted.

TABLE 3
Recipe for the 250 L sugar feed for L. crispatus (DSM 33187).

| Components | Weight (kg) for 250 L feed |
|---|---|
| Dextrose | 39.8 |

TABLE 4
Recipe for the 3160 L LC100 Media.

| | Weight (kg) for 3400 |
|---|---|
| Components | L LC100 media |
| Peptone | 68 |
| Yeast extract | 34 |
| dipotassium phosphate (K₂HPO₄) | 8.5 |
| Ammonium citrate | 1.02 |
| Magnesium sulfate heptahydrate | 0.34 |
| Magnesium sulfate | 0.34 |
| Sodium acetate | 18.8 |
| Tween 80 | 14.7 |
| Acid | 2 |
| Antimousse | 1 |

TABLE 5
Recipe for 150 L Cryoprotectant Mix.

| Components | Weight (kg) for 150 L cryoprotectant mix |
|---|---|
| Saccharose | 12 |
| Trehalose | 1.995 |
| Sodium Glutamate | 0.825 |
| L-Cysteine HCI | 0.195 |

### Sugar Fraction & Feed Preparation and Decontamination

A 300 L container and mixing tank clean in place (CIP) was completed. 125 L of hot softened water was added to the sterile mixing tank using a 0.22 μm filter. One third of the sugar feed components, by weight, was added to the mixing tank and stirred at 150 rpm for 10 minutes until they were completely dissolved. Subsequently, the additional two thirds of sugar feed components and 125 L of hot softened water were added to the mixing tank and completely dissolved. The sugar feed was filtered sterilized using a 0.2 μm filter and stored in the sterile 300 L container.

### 3500 L Culture Media Preparation and Decontamination

To generate the LC100 media, a 3,500 L stirred fermenter is sterilized by CIP and fitted with calibrated pH and redox sensing probes. A total of 400 L of 0.22 μm filtered water was added to a presterilized mixing tank and combined with LC100 culture media components. The mixture was homogenized for 10 min at 150 rpm. The concentrated media was transferred to the 3500 L bioreactor and 3000 L of 0.22 μm filtered softened water was added to the fermenter. The pH of the culture media was adjusted to pH=6.5. The media was sterilized in place at 121° C. for 20 mins. Using a steam sterilized connection, 100 L of the sugar feed was added to the sterilized LC100 culture medium. The completed LC100 culture medium was then degassed using a sparger adding a N₂H₂CO₂ (90:5:5) gas mix at a rate of 0.1 vvm while stirring at 100 rpm and maintaining a headspace pressure of 0.2 bar. The redox value of the LC100 media was monitored from the start of degassing. The degassing continued until the redox value dropped and maintained at a steady value for 1 hour. The LC100 was then stored at 10° C.±2° C. with 90 rpm stirring and sparging 0.01 vvm of gas mix until use.

### 20 L Fermenter Preparation and Decontamination and Media Transfer

A 20 L clean in place (CIP) was completed. 20.5 L of sterile culture media was transferred to the 20 L fermenter using a sterile connector from the 3500 L fermenter.

### Initial Inoculation Preparation

500 ml of sterile LC100 media was transferred from the 20 L fermenter to a sterile 1 bottle. The sterile bottle was then transferred to an anaerobic chamber. 1.84 mL of WCB L. crispatus (DSM 33187) was thawed in the anaerobic chamber and inoculated in the 500 mL of reduced LC100 media (˜0.4% v/v inoculation rate) using a sterile pipette inoculate. The cell and media mixture were homogenized by gently swirling and incubated at 37° C. with periodic optical density measurements at 583 nm (OD₅₈₃). The culture was stopped when a) OD₅₈₃>4.5 or b) the culture grew for 20 hours.

### 20 L Inoculation

20 L culture media was warmed to 37° C. The media was degassed using the parameters listed in TABLE 6.

TABLE 6
Parameters for degassing inoculation.

| Parameter | Setting |
|---|---|
| Stirring | 100 rpm |
| Temperature | 37 = 2° C. |
| pH | 6.5 |
| Gas | N₂H₂CO₂ (90:5:5) |
| Gas flow rate | 0.01 vvm |
| Overlay | 0.2 Bar |

The degassing continued until the redox value stabilized for 1 hour, as measured by the standard redox sensor. A sterilized 3-way valve was connected to the 20 L fermenter. The 0.1 L inoculum culture of L. crispatus (DSM 33187) (0.5% v/v inoculum) was added to the 20 L fermenter via the 3-way valve. The optical density of the 20 L culture was monitored using OD₅₈₅. The culture was stopped when any one of the following criteria was reached: The culture was stopped for one of the following criteria: a) OD₅₈₃>6, b) concentration of glucose <2 g/L, c) total culture time reaches 20 h, or d) a slowing of the growth rate is detected after three subsequent OD₅₈₃ readings.

### 3500 L Inoculation

3400 L culture media was warmed to 37° C. The media was degassed using the parameters listed in TABLE 6. The degassing continued until the redox value stabilized for 1 hour, as measured by the standard redox sensor. The 20 L fermenter was connected to the 3500 L fermenter. The entire inoculum culture of L. crispatus (DSM 33187) (˜0.6% v/v inoculum) from the 20 L fermenter was added to the 3500 L fermenter via the 3-way valve. 160 L sugar feed was added to in the 3500 L fermenter. The optical density of the 3500 L culture was monitored using OD₅₈₅. The culture was stopped when any one of the following criteria are reached: a) OD₅₈₃>8, b) concentration of glucose <2 g/L, c) total culture time reaches 20 h, or d) a slowing of the growth rate is detected after three subsequent OD₅₈₃ readings. When one of these parameters was met, the fermenter was set to 4° C.+/−3° C. to start cooling the culture.

### Centrifugation

The GEA centrifuge and mixing tank clean in place (CIP) was completed. The mix gas line (N₂H₂CO₂, 90:5:5) was connected to and degas the GEA centrifuge and mix tank for 30 mins. The 3500 L culture was centrifuged using the Sharples parameter listed in TABLE 7.

TABLE 7
Sharples Parameter for the Centrifugation of the 3500 L culture.

| Parameter | Setting |
|---|---|
| Feed flow rate | 600 L/h |
| Counter Pressure | 1.5 bar |

The concentrated bacteria fraction (biomass) was collected in the degassed mix tank. The weight of the concentrated biomass collected was weighted.

### Cryoprotectant Solution Preparation and Addition to Concentrated Biomass

The mixing tank clean in place (CIP) was completed. 75 L of 0.22 μm filtered hot softened water was added to the mix tank. One third of the cryoprotectant mix components was added to the mixing tank until they were completely dissolved. An additional 20 L of softened, filtered water was added. The mixture was homogenized for 10 min at 150 rpm. The cryoprotectant solution was transferred to a sterile bioreactor. The baseline redox value of the solution was recorded. The cryoprotectant solution was degassed using the parameters listed in TABLE 8.

TABLE 8
Parameters for degassing the cryoprotectant solution.

| Parameter | Setting |
|---|---|
| Stirring | 100 rpm |
| Gas | N₂H₂CO₂ (90:5:5) |
| Gas flow rate | 0.1 vvm |
| Overlay | 0.3 Bar |

The degassed cryoprotectant solution was added to the anaerobic concentrated biomass in the mix tank in a 1:1 (w/w) ratio. The total mass and volume of biomass and cryoprotectant available for lyophilization were recorded.

### Lyophilization & Grinding

The sterile plastic freeze dry trays were loaded so that the total thickness does not exceed 1 cm, corresponding to 1.5 L of cell and cryoprotectant mix per tray. The trays were moved into the pre-frozen lyophilizer shelves as they were filled to expedite the freezing process. The lyophilization cycle was initiated according to the parameters listed in TABLE 9.

TABLE 9
Parameters for the lyophilization cycle of the concentrated biomass.

| | | Shelf | Ramp | Hold | |
|---|---|---|---|---|---|
| | | temperature | time | time | Vacuum |
| Procedure | Step | (° C.) | (hour) | (hour) | (μBar) |
| Freezing | 1 | −45 | 0 | 3 | None |
| Vacuum pull- | 2 | −45 | 0 | 1 | 400 |
| down | | | | | |
| Primary | 3 | −5 | 26 | Wait until the | 400 |
| drying | | | | cell mixture | |
| | | | | temperature | |
| | | | | was > −8° C. | |
| Secondary | 4 | 25 | 4 | ≥11.5 | 27 |
| Drying | | | | | |

The lyophilized material was ground on speed 1, using the spacer 1 and a 1 mm grid. After grinding, the lyophilized cell material was immediately sealed in polyethylene (PE) bags, each containing 1.5 kg or less material. The lyophilized cell material was stored at <−18° C. and be used to manufacture drug products, as defined in EXAMPLE 4, 5, 6, or 9.

### Example 2—Drug Product Manufacturing

Provided herein are methods for manufacturing a drug product using the bacterial strains and other materials described herein and thereof, comprising the excipients. The drug product may be the pharmaceutical compositions described in this disclosure. The bacterial strains or drug substances may comprise the bacterial populations described in this disclosure.

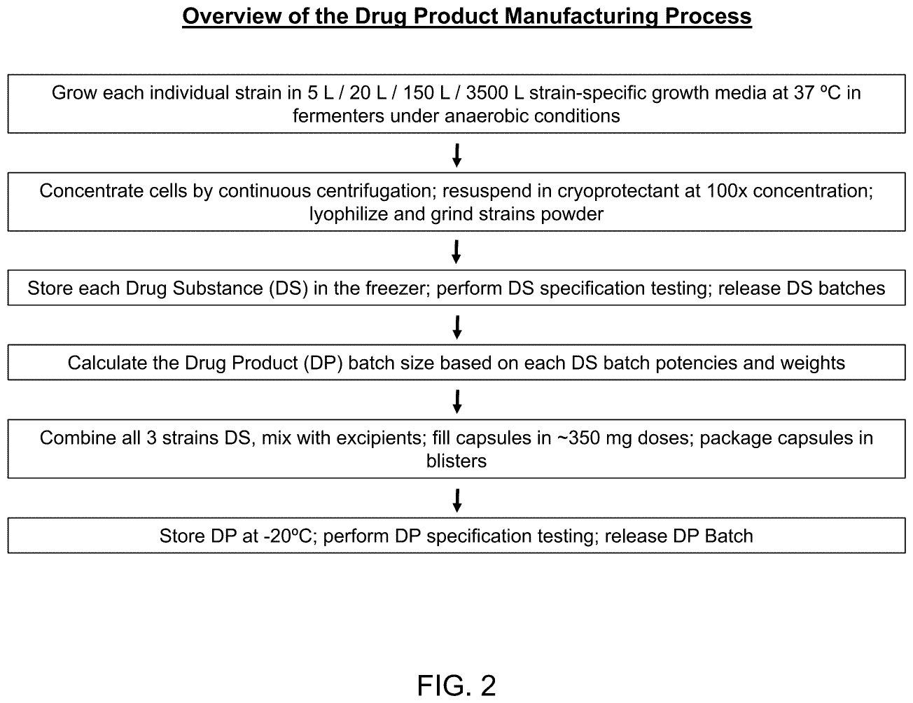

As depicted in FIG. 2, generally, to produce a drug product or pharmaceutical composition comprising A. muciniphila (DSM 33213), L. crispatus (DSM 33187), and F. prausnitzii (DSM 33185), each strain is grown in various (e.g., 1 L, 5 L, 20 L, 150 L, 300 L, or 3500 L) in strain-specific media at 37° C. in fermenters under anaerobic conditions. The cells are then concentrated by continuous centrifugation in cryoprotectant (e.g., at 100× concentration). The concentrated cells are then lyophilized and grinded into powder forms. Each strain is stored in freezer (e.g., any temperature from −80° C. or −20° C.) as a drug substance with its own excipients, comprising glycerol or antioxidants. A specification testing was performed on each batch of drug substance. One specification standard is described herein or in any one of EXAMPLES 4, 5, 6 or 9. The drug substance can be synthesized as a feasibility batch, an engineering batch, or a good manufacturing practice (GMP) batch.

Upon passing the specification, the drug substance batch is released for drug product manufacturing. A drug product batch is calculated based on the potency and weight of each drug substance. Three drug substance batches, each containing one of A. muciniphila (DSM 33213), L. crispatus (DSM 33187), and F. prausnitzii (DSM 33185), are mixed with additional excipients and materials to form the drug product. ˜350 mg of the drug product is filled into each capsule. The filled capsules are packed in aluminum blisters. The drug product is stored at freezer (e.g., any temperature from −80° C. or −20° C.). Each batch of drug product undergoes a specification testing. One specification standard is described in one of EXAMPLES 4, 5, 6 or 9. The drug product can then be released. The drug product can be synthesized as a feasibility batch, an engineering batch, or a good manufacturing practice (GMP) batch.

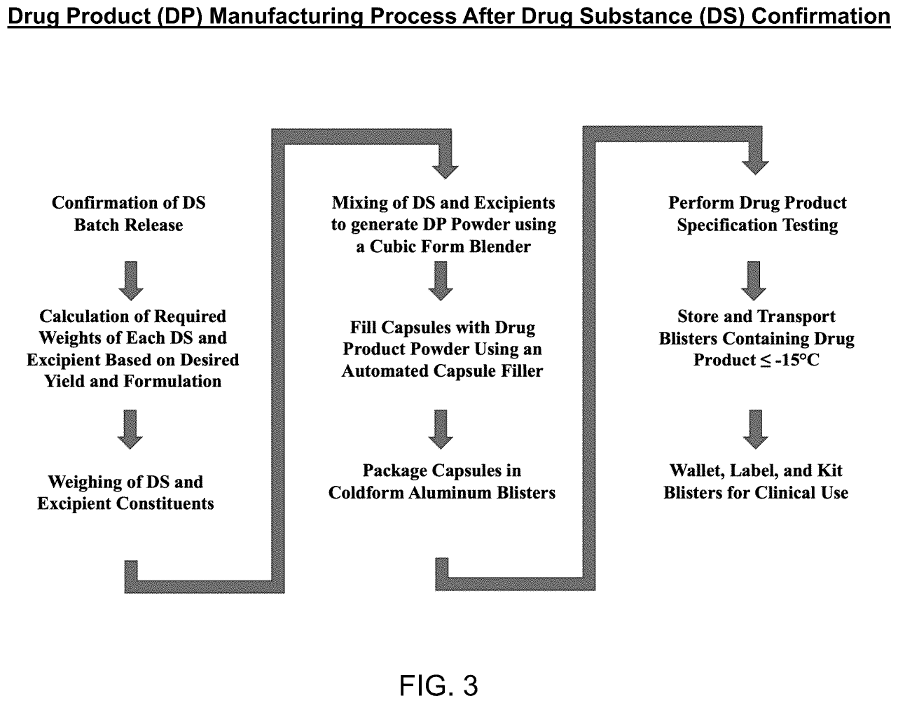

FIG. 3 depicts manufacturing steps after the drug substance batches are released. The required weights of each drug substance and excipient in the drug product are calculated based on the potency of the specific constituent drug substance lots, the batch formula, and the desired batch yield. The required weight of each drug substance is calculated based on the desired yield and formation, such as that described in one of EXAMPLES 4, 5, 6 or 9. The drug product production takes place in a manufacturing suite with temperature (20° C.±2° C.) and humidity (<30% RH) environmental controls and positive pressure ventilation. Using a dedicated weighing area and calibrated balances, raw materials are weighed and distributed into pre-labeled polyethylene bags. The raw material weighing process takes place sequentially, such that no raw materials are processed simultaneously. The constituent powders contain: A. muciniphila (DSM 33213), L. crispatus (DSM 33187), and F. prausnitzii (DSM 33185), mannitol, magnesium stearate, and silicon dioxide. The drug substances and the excipient components are then weighted. They are mixed to generate the drug product powder using a cubic form blender. Other blenders can also be used. The pre-measured raw material powders are blended to homogeneity using a cubic form blender. Stratified blend samples are removed for assessment of uniformity and moisture. The bulk drug product powder blend is dispensed into pre-labeled PE bags and sealed. Bulk drug product powder blend is added to the hopper of an automatic capsule filler and size 00 hypromellose capsules (primary container closure, not for consumption) are filled with 325-350 mg of the drug product powder. The capsule can be filled automatically using a mechanical filler. Alternatively, the capsule can be filled manually. Closed capsules not meeting the weight specification disclosed in EXAMPLES 4, 5, 6 or 9 for uniformity are automatically discarded as part of the filling process. Filled capsules are collected in PE bags, which are purged of air using nitrogen gas and sealed using a link. A second PE bag containing two oxygen absorbers is placed over the first bag, purged with nitrogen, and closed with a seal link. Filled capsules are stored at 2-8° C. until blister packing. Filled capsules are fed into an automated cold form foil blister packing machine for secondary packing. Blisters are generated to contain 8 capsules, each sealed in individual wells with a nitrogen gas headspace. Bulk blisters are boxed, enclosed in a PE bag, sealed with link and stored at <−15° C. prior to shipping. A pre-defined number of blisters are removed for quality control analysis. Bulk blisters are shipped to the packager and labeler (Caligor Coghlan Pharma Services, TX, USA) on dry ice. Blisters are walleted, labeled, and kitted into labeled monthly dosing cartons containing four walleted blisters in each kit.

To provide control of target capsule fill weight, in-line weight checking is utilized during automated encapsulation, with rejection of capsules outside of the specified weight tolerance. Additional in process controls can be developed and implemented as applicable in later phases of development.

### Example 3—Drug Substance Specification

Provided herein are specifications of the drug substances. Other specifications, such as those modified from the ones disclosed in this EXAMPLE, can also be used depending on the application of the drug products. The drug substances may comprise the bacterial populations described in this disclosure.

The specification of the drug substances—A. muciniphila (DSM 33213), L. crispatus (DSM 33187), and F. prausnitzii (DSM 33185)—is summarized in TABLE 10.

TABLE 10
The specification of the drug substances

| | Target | | |
|---|---|---|---|
| | specification | Initial viable cell | Theoretical |
| | per capsule | counts for the drug | weight |
| Drug substance | (CFU/capsule) | substance (CFU/g) | per capsule (mg) |
| L. crispatus | 5 × 10^{∧}8 | 5 × 10^{∧}10 | 10.0 |
| (DSM 33187) | | | |
| F. prausnitzii | 2 × 10^{∧}8 | 2.5 × 10^{∧}9 | 120.0* |
| (DSM 33185) | | | |
| A. muciniphila | 2 × 10^{∧}8 | 1.9 × 10^{∧}11 | 1.58* |
| (DSM 33213) | | | |

*over-formulated drug substance by 1 × 10^{∧}8 CFU/capsule

### Example 4—Testing Excipients for a Drug Product

Provided herein are methods for testing the excipients for the drug products. Also provided are the excipients of the drug products. The drug product may be the pharmaceutical compositions described in this disclosure. The bacterial strains or drug substances may comprise the bacterial populations described in this disclosure.

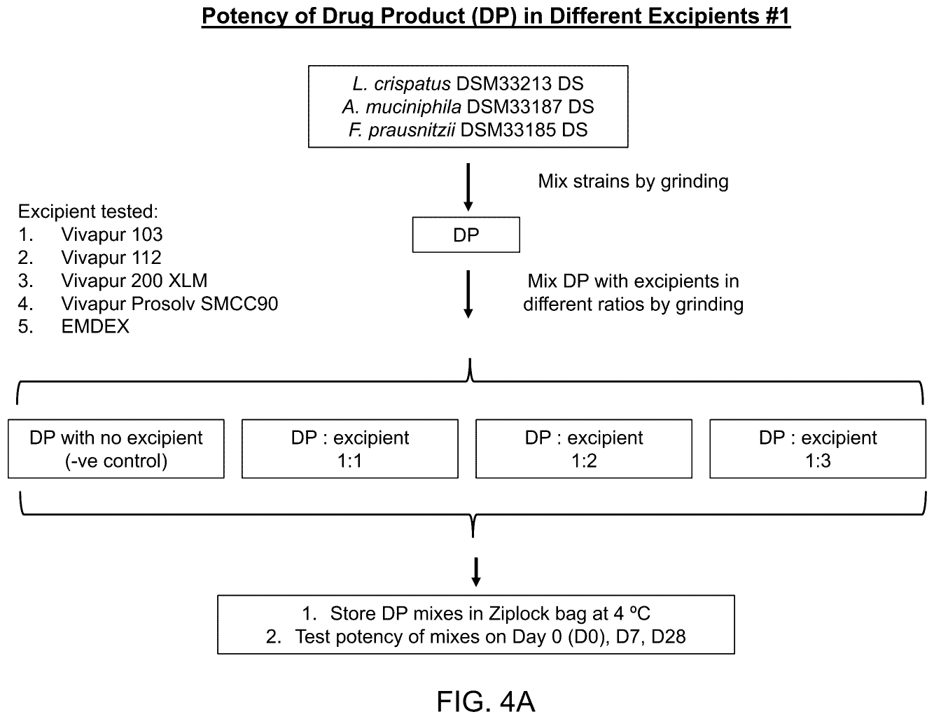

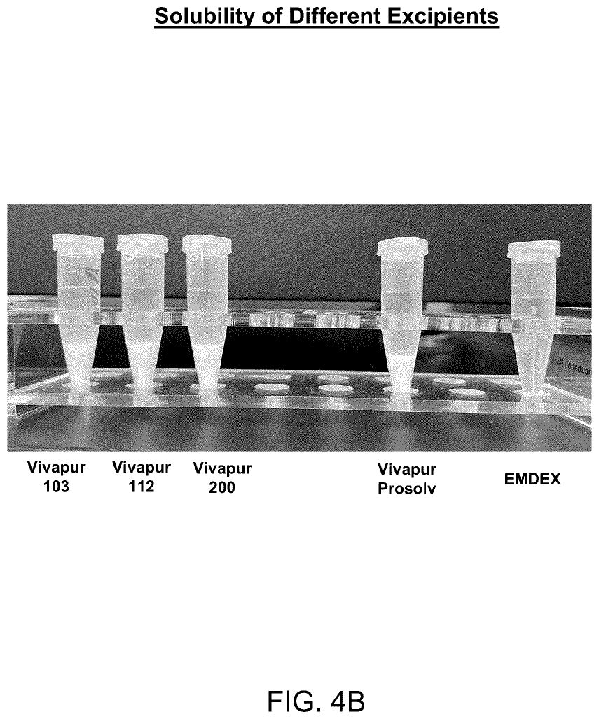

As depicted in FIG. 4A, the drug substances A. muciniphila (DSM 33213), L. crispatus (DSM 33187), and F. prausnitzii (DSM 33185) were mixed with Vivapur 103, Vivapur 112, Vivapur 200 XLM, Vivapur Prosolv SMCC90, and EMDEX in a 1:1, 1:2, or 1:3 ratio. The drug product (DP) without any excipient was used as a negative control. Each mixture was stored in a Ziplock bag at 4° C. The potency of F. prausnitzii (DSM 33185) was tested on day 0 (D0), D7, and D28 after the mixing and storing. The experiment was performed in Biosafety Cabinet (BSC) with 68° F. and >60% RH. The potency of F. prausnitzii (DSM 33185) at these time points are summarized in TABLE 11.

TABLE 11
The amount of bacterial cells (CFU) of F. prausnitzii (DSM 33185)
at various time points after mixing with different
Vivapur or EMDEX as excipients.

| | | DP: Vivapur | DP: Vivapur | DP: Vivapur |
|---|---|---|---|---|
| Time point | DP control | 103 (1:1) | 103 (1:2) | 103 (1:3) |
| D0 | 6.75 × 10^{∧}9 | 6.75 × 10^{∧}9 | 5.90 × 10^{∧}9 | 5.00 × 10^{∧}9 |
| D7 | 4.60 × 10^{∧}9 | 3.18 × 10^{∧}9 | 4.50 × 10^{∧}9 | 4.50 × 10^{∧}9 |
| D28 | 1.02 × 10^{∧}9 | 2.22 × 10^{∧}9 | 3.89 × 10^{∧}9 | 3.89 × 10^{∧}9 |
| | | DP: Vivapur | DP: Vivapur | DP: Vivapur |
| | DP control | 112 (1:1) | 112 (1:2) | 112 (1:3) |
| D0 | 6.75 × 10^{∧}9 | 3.50 × 10^{∧}9 | 9.00 × 10^{∧}9 | 2.60 × 10^{∧}9 |
| D7 | 4.60 × 10^{∧}9 | 9.26 × 10^{∧}8 | 7.04 × 10^{∧}9 | 1.13 × 10^{∧}9 |
| D28 | 1.02 × 10^{∧}9 | 8.32 × 10^{∧}8 | 6.23 × 10^{∧}9 | 1.19 × 10^{∧}9 |
| | | | | DP: Vivapur |
| | DP control | / | / | 200 (1:3) |
| D0 | 6.75 × 10^{∧}9 | / | / | 5.00 × 10^{∧}9 |
| D7 | 4.60 × 10^{∧}9 | / | / | 4.66 × 10^{∧}9 |
| D28 | 1.02 × 10^{∧}9 | / | / | 3.37 × 10^{∧}9 |
| | | | DP: Prosolv | DP: Prosolv |
| | | DP: Prosolv | 112 | 112 |
| | DP control | (1:1) | (1:2) | (1:3) |
| D0 | 1.33 × 10^{∧}9 | 1.50 × 10^{∧}9 | 6.39 × 10^{∧}9 | 4.28 × 10^{∧}9 |
| D7 | 9.81 × 10^{∧}8 | 9.87 × 10^{∧}8 | 2.45 × 10^{∧}9 | 2.35 × 10^{∧}9 |
| D28 | 5.23 × 10^{∧}8 | 7.58 × 10^{∧}8 | 1.89 × 10^{∧}9 | 2.12 × 10^{∧}9 |
| | | DP: EMDEX | DP: EMDEX | DP: EMDEX |
| | DP control | (1:1) | (1:2) | (1:3) |
| D0 | 2.33 × 10^{∧}9 | / | 1.33 × 10^{∧}9 | 2.90 × 10^{∧}9 |
| D7 | 2.77 × 10^{∧}9 | 3.94 × 10^{∧}7 | 4.81 × 10^{∧}8 | 2.89 × 10^{∧}8 |
| D28 | 8.67 × 10^{∧}8 | 4.14 × 10^{∧}6 | 5.67 × 10^{∧}8 | 6.76 × 10^{∧}7 |

Compared to the DP control, the potency of F. prausnitzii (DSM 33185) at these time points were relatively stable when mixed with all grades of Vivapur microcrystalline cellulose tested. The potency of F. prausnitzii (DSM 33185) dropped when mixed with EMDEX, possibly due to the hygroscopicity of EMDEX. The drug product should avoid exposure to oxygen or humidity; or storage at 4′° C. As depicted in FIG. 4B, various Vivapur microcrystalline celluloses were not soluble in water. Vivapur microcrystalline celluloses could still be a useful excipients depending on the administration methods. For example, they could be used as excipients if the drug product did not need to dissolve in water for administration.

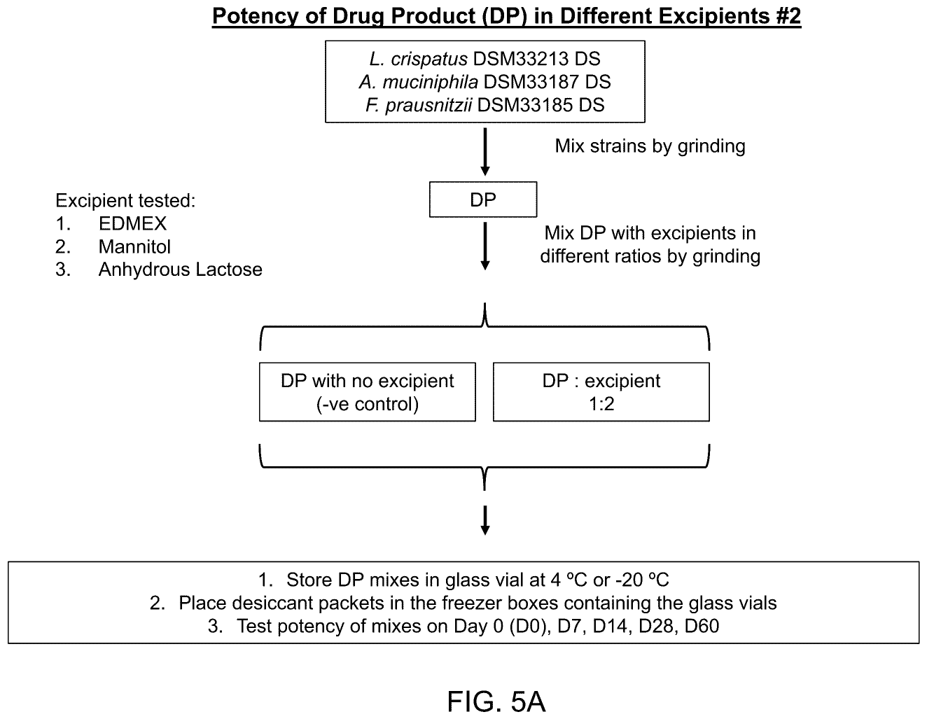

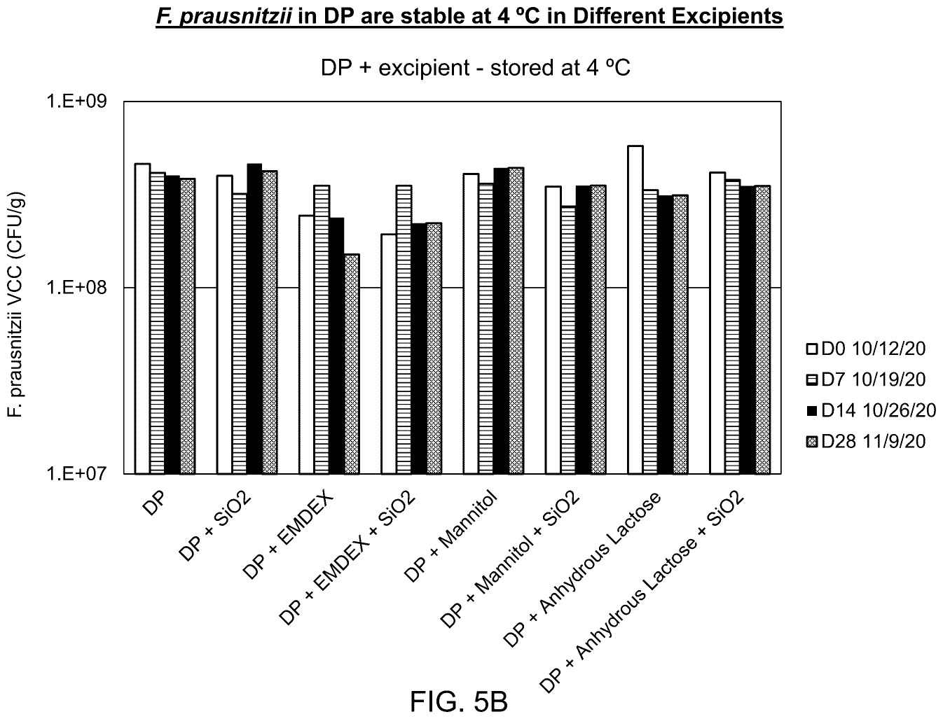

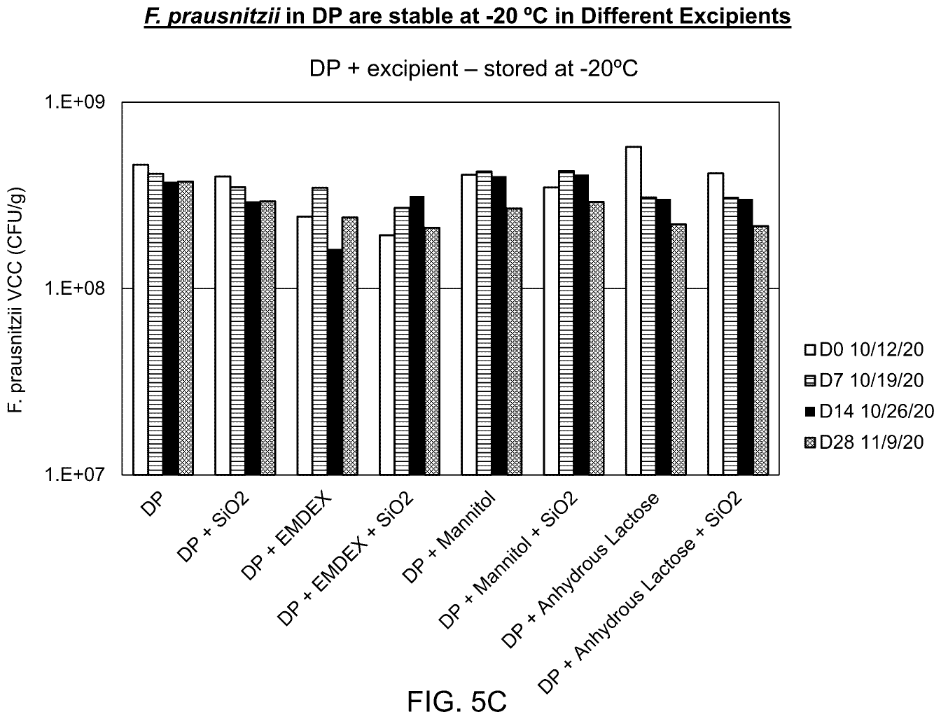

As depicted in FIG. 5A, the drug substances A. muciniphila (DSM 33213), L. crispatus (DSM 33187), and F. prausnitzii (DSM 33185) were tested with more excipients. The drug substances were mixed with EMDEX, mannitol, or anhydrous lactose in a 1:2 ratio with or without silicon dioxide (S102). Each drug product mixture contained ˜0.3 g of drug product and excipients and stored in 5/6 glass vials at 4° C. or −20° C. Desiccant packets were placed in freezer boxes containing glass vials. The drug product without any excipient was used as a negative control. The experiment was performed in anaerobic chamber with 68° F. and ˜40% RH. The potency of F. prausnitzii (DSM 33185) was scored on Day 0 (D0), D7, D14, D28, and D60 after the mixing and storing. The potency of F. prausnitzii (DSM 33185) at some of these time points are summarized in TABLE 12.

TABLE 12
The potency of F. prausnitzii (DSM 33185) at various time points after mixing
with mannitol, anhydrous lactose, or EMDEX at 4° C. or −20° C.

| Conditions | Excipients | D0 | D7 | D14 | D28 |
|---|---|---|---|---|---|
| −20° C. | DP | 4.62 × 10^{∧}8 | 4.13 × 10^{∧}8 | 3.75 × 10^{∧}8 | 3.75 × 10^{∧}8 |
| −20° C. | DP + SiO₂ | 3.99 × 10^{∧}8 | 3.50 × 10^{∧}8 | 2.94 × 10^{∧}8 | 2.94 × 10^{∧}8 |
| −20° C. | DP + EMDEX | 2.44 × 10^{∧}8 | 3.48 × 10^{∧}8 | 1.63 × 10^{∧}8 | 2.41 × 10^{∧}8 |
| −20° C. | DP + EMDEX + SiO₂ | 1.93 × 10^{∧}8 | 2.71 × 10^{∧}8 | 3.14 × 10^{∧}8 | 2.12 × 10^{∧}8 |
| −20° C. | DP + mannitol | 4.08 × 10^{∧}8 | 4.25 × 10^{∧}8 | 4.02 × 10^{∧}8 | 2.69 × 10^{∧}8 |
| −20° C. | DP + mannitol + SiO₂ | 3.49 × 10^{∧}8 | 4.27 × 10^{∧}8 | 4.10 × 10^{∧}8 | 2.92 × 10^{∧}8 |
| −20° C. | DP + anhydrous lactose | 5.76 × 10^{∧}8 | 3.08 × 10^{∧}8 | 3.03 × 10^{∧}8 | 2.21 × 10^{∧}8 |
| −20° C. | DP + anhydrous lactose + SiO₂ | 4.15 × 10^{∧}8 | 3.07 × 10^{∧}8 | 3.03 × 10^{∧}8 | 2.17 × 10^{∧}8 |
| 4° C. | DP | 4.62 × 10^{∧}8 | 4.14 × 10^{∧}8 | 4.01 × 10^{∧}8 | 3.84 × 10^{∧}8 |
| 4° C. | DP + SiO₂ | 3.99 × 10^{∧}8 | 3.19 × 10^{∧}8 | 4.64 × 10^{∧}8 | 4.22 × 10^{∧}8 |
| 4° C. | DP + EMDEX | 2.44 × 10^{∧}8 | 3.53 × 10^{∧}8 | 2.38 × 10^{∧}8 | 1.51 × 10^{∧}8 |
| 4° C. | DP + EMDEX + SiO₂ | 1.93 × 10^{∧}8 | 3.53 × 10^{∧}8 | 2.22 × 10^{∧}8 | 2.22 × 10^{∧}8 |
| 4° C. | DP + mannitol | 4.08 × 10^{∧}8 | 3.62 × 10^{∧}8 | 4.40 × 10^{∧}8 | 4.40 × 10^{∧}8 |
| 4° C. | DP + mannitol + SiO₂ | 3.49 × 10^{∧}8 | 2.73 × 10^{∧}8 | 3.53 × 10^{∧}8 | 3.53 × 10^{∧}8 |
| 4° C. | DP + anhydrous lactose | 5.76 × 10^{∧}8 | 3.34 × 10^{∧}8 | 3.13 × 10^{∧}8 | 3.13 × 10^{∧}8 |
| 4° C. | DP + anhydrous lactose + SiO₂ | 4.15 × 10^{∧}8 | 3.79 × 10^{∧}8 | 3.52 × 10^{∧}8 | 3.52 × 10^{∧}8 |

As depicted in TABLE 12, FIG. 5B and FIG. 5C, F. prausnitzii (DSM 33185) cells were relatively stable with all the excipients tested when DP was manufactured under anaerobic conditions at low humidity and stored at 4° C. or −20° C.

When mixing with mannitol or anhydrous lactose and silicon dioxide, F. prausnitzii (DSM 33185) in the drug product (DP) was stable in low humidity, and A. muciniphila (DSM 33213) and L. crispatus (DSM 33187) were stable in low and high humidity. The drug product comprises drug substances (DS) comprising: A. muciniphila (DSM 33213), L. crispatus (DSM 33187), and F. prausnitzii (DSM 33185). About 0.3 g drug product mixtures were filled into 8 capsules per condition in the anaerobic chamber (AS-150; ˜40% humidity) and Biosafety cabinet (BSC; >60% humidity). The capsules were stored in glass vials at 4° C. and −20° C. Desiccant packets were placed in the freezer boxes containing the glass vials. The potency of the mixtures was tested on Day 0 (D0), D7, D14, D21, D28, D60, D90, and D120. The potency of F. prausnitzii (DSM 33185) at some of these time points are summarized in TABLE 13.

TABLE 13
The potency of F. prausnitzii (DSM 33185) at various time points after mixing
with mannitol, anhydrous lactose, or EMDEX at 4° C. or -20° C.

| | | Drug | | | |
|---|---|---|---|---|---|
| Mixture | Conditions | substance | D0 | D7 | D14 |
| DP + mannitol + SiO₂ | AS-150 at −20° C. | L. crispatus | 3.9 × 10^{∧}10 | 3.86 × 10^{∧}10 | 3.9 × 10^{∧}10 |
| | | (DSM 33187) | | | |
| DP + mannitol + SiO₂ | AS-150 at 4° C. | L. crispatus | 3.9 × 10^{∧}10 | 3.48 × 10^{∧}10 | 3.6 × 10^{∧}10 |
| | | (DSM 33187) | | | |
| DP + mannitol + SiO₂ | BSC at −20° C. | L. crispatus | 3.9 × 10^{∧}10 | 3.16 × 10^{∧}10 | 3.2 × 10^{∧}10 |
| | | (DSM 33187) | | | |
| DP + mannitol + SiO₂ | BSC at 4° C. | L. crispatus | 3.9 × 10^{∧}10 | 3.16 × 10^{∧}10 | 2.8 × 10^{∧}10 |
| | | (DSM 33187) | | | |
| DP + | AS-150 at −20° C. | L. crispatus | 3.9 × 10^{∧}10 | 3.31 × 10^{∧}10 | 3.7 × 10^{∧}10 |
| anhydrous | | (DSM 33187) | | | |
| lactose + SiO₂ | | | | | |
| DP + | AS-150 at 4° C. | L. crispatus | 3.9 × 10^{∧}10 | 3.55 × 10^{∧}10 | 3.2 × 10^{∧}10 |
| anhydrous | | (DSM 33187) | | | |
| lactose + SiO₂ | | | | | |
| DP + | BSC at −20° C. | L. crispatus | 3.3 × 10^{∧}10 | 2.43 × 10^{∧}10 | 2.7 × 10^{∧}10 |
| anhydrous | | (DSM 33187) | | | |
| lactose + SiO₂ | | | | | |
| DP + | BSC at 4° C. | L. crispatus | 3.3 × 10^{∧}10 | 2.96 × 10^{∧}10 | 2.6 × 10^{∧}10 |
| anhydrous | | (DSM 33187) | | | |
| lactose + SiO₂ | | | | | |
| DP + mannitol | AS-150 at −20° C. | A. | 5.62 × 10^{∧}9 | 5.60 × 10^{∧}9 | 5.18 × 10^{∧}9 |
| + SiO₂ | | muciniphila | | | |
| | | (DSM 33213) | | | |
| DP + mannitol | AS-150 at 4° C. | A. | 5.62 × 10^{∧}9 | 5.40 × 10^{∧}9 | 5.50 × 10^{∧}9 |
| + SiO₂ | | muciniphila | | | |
| | | (DSM 33213) | | | |
| DP + mannitol + | BSC at −20° C. | A. | 5.86 × 10^{∧}9 | 5.10 × 10^{∧}9 | 5.49 × 10^{∧}9 |
| SiO₂ | | muciniphila | | | |
| | | (DSM 33213) | | | |
| DP + mannitol + | BSC at 4° C. | A. | 5.86 × 10^{∧}9 | 4.60 × 10^{∧}9 | 4.25 × 10^{∧}9 |
| SiO₂ | | muciniphila | | | |
| | | (DSM 33213) | | | |
| DP + | AS-150 at −20° C. | A. | 5.40 × 10^{∧}9 | 5.30 × 10^{∧}9 | 4.99 × 10^{∧}9 |
| anhydrous | | muciniphila | | | |
| lactose + SiO₂ | | (DSM 33213) | | | |
| DP + | AS-150 at 4° C. | A. | 5.40 × 10^{∧}9 | 5.20 × 10^{∧}9 | 5.01 × 10^{∧}9 |
| anhydrous | | muciniphila | | | |
| lactose + SiO₂ | | (DSM 33213) | | | |
| DP + | BSC at −20° C. | A. | 4.76 × 10^{∧}9 | 4.60 × 10^{∧}9 | 4.04 × 10^{∧}9 |
| anhydrous | | muciniphila | | | |
| lactose + SiO₂ | | (DSM 33213) | | | |
| DP + | BSC at 4° C. | A. | 4.76 × 10^{∧}9 | 4.50 × 10^{∧}9 | 4.67 × 10^{∧}9 |
| anhydrous | | muciniphila | | | |
| lactose + SiO₂ | | (DSM 33213) | | | |
| DP + mannitol + | AS-150 at −20° C. | F. prausnitzii | 1.91 × 10^{∧}9 | 1.88 × 10^{∧}9 | 1.80 × 10^{∧}9 |
| SiO₂ | | (DSM 33185) | | | |
| DP + mannitol + | AS-150 at 4° C. | F. prausnitzii | 1.91 × 10^{∧}9 | 1.86 × 10^{∧}9 | 1.90 × 10^{∧}9 |
| SiO₂ | | (DSM 33185) | | | |
| DP + mannitol + | BSC at −20° C. | F. prausnitzii | 6.24 × 10^{∧}8 | 2.34 × 10^{∧}8 | 3.50 × 10^{∧}8 |
| SiO₂ | | (DSM 33185) | | | |
| DP + mannitol + | BSC at 4° C. | F. prausnitzii | 6.24 × 10^{∧}8 | 1.35 × 10^{∧}7 | 1.50 × 10^{∧}7 |
| SiO₂ | | (DSM 33185) | | | |
| DP + | AS-150 at −20° C. | F. prausnitzii | 1.24 × 10^{∧}9 | 1.46 × 10^{∧}9 | 1.30 × 10^{∧}9 |
| anhydrous | | (DSM 33185) | | | |
| lactose + SiO₂ | | | | | |
| DP + | AS-150 at 4° C. | F. prausnitzii | 1.24 × 10^{∧}9 | 1.13 × 10^{∧}9 | 1.20 × 10^{∧}9 |
| anhydrous | | (DSM 33185) | | | |
| lactose + SiO₂ | | | | | |
| DP + | BSC at −20° C. | F. prausnitzii | 1.01 × 10^{∧}8 | 6.71 × 10^{∧}7 | 1.20 × 10^{∧}8 |
| anhydrous | | (DSM 33185) | | | |
| lactose + SiO₂ | | | | | |
| DP + | BSC at 4° C. | F. prausnitzii | 1.01 × 10^{∧}8 | 3.40 × 10^{∧}7 | 5.60 × 10^{∧}7 |
| anhydrous | | (DSM 33185) | | | |
| lactose + SiO₂ | | | | | |

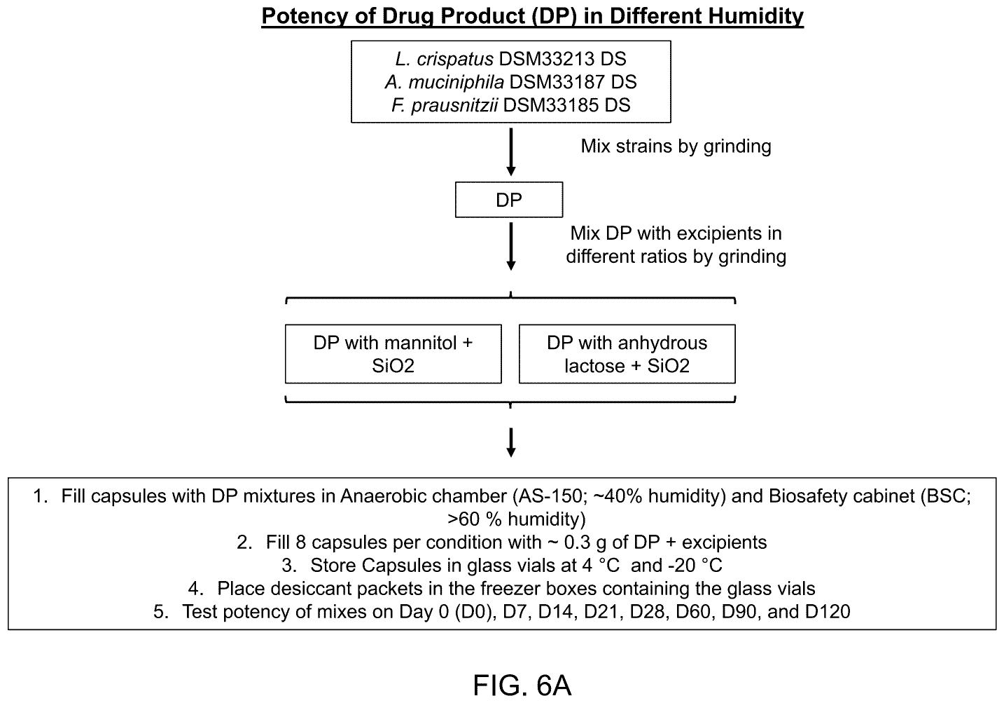

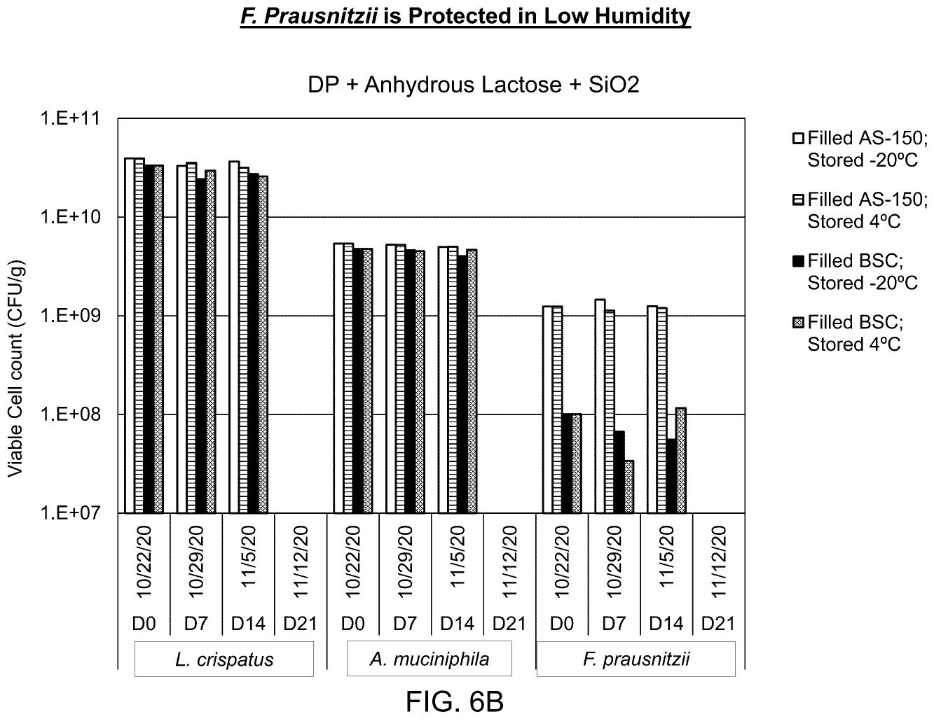

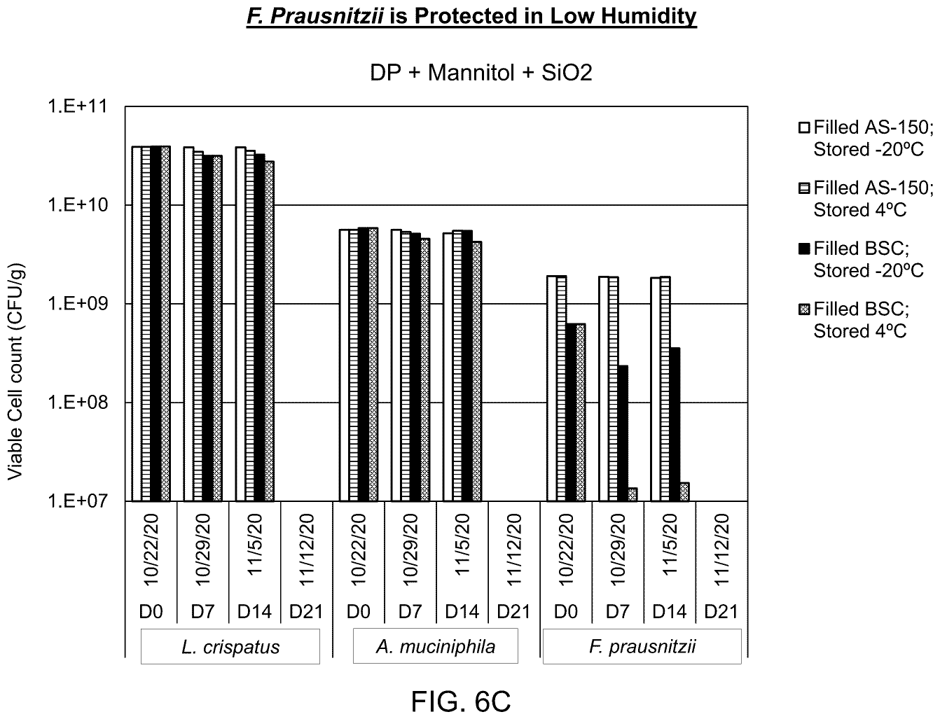

As depicted in TABLE 13, FIG. 6B and FIG. 6C, A. muciniphila (DSM 33213) and L. crispatus (DSM 33187) were stable when capsules were filled with the drug product, silicon dioxide and mannitol or anhydrous lactose, in either AS-150 or BSC in low and high humidity. F. prausnitzii (DSM 33185) was when capsules were filled with the drug product, silicon dioxide and mannitol or anhydrous lactose, in either AS-150 or BSC in low humidity.

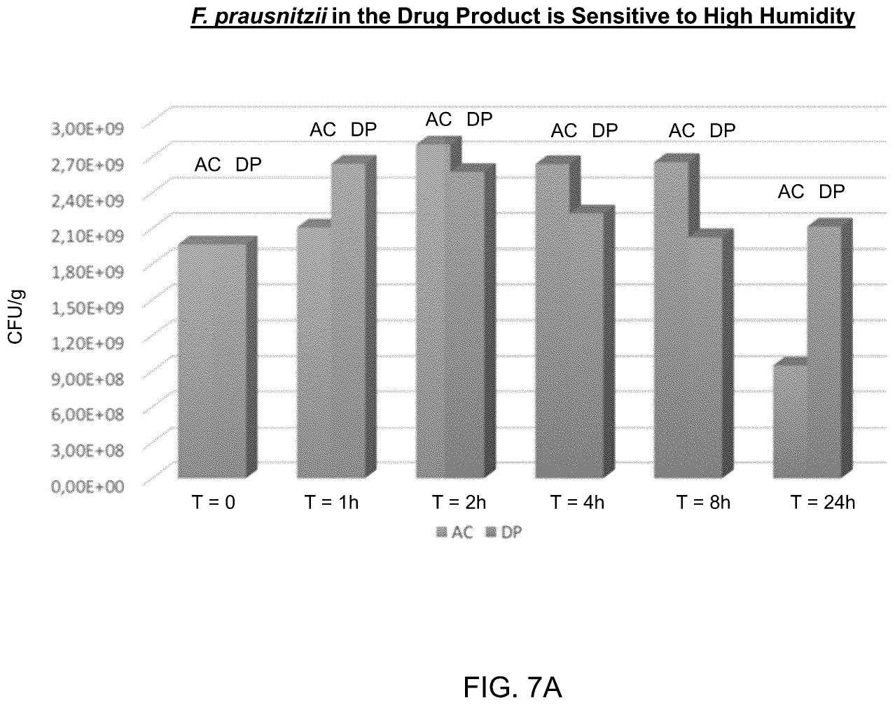

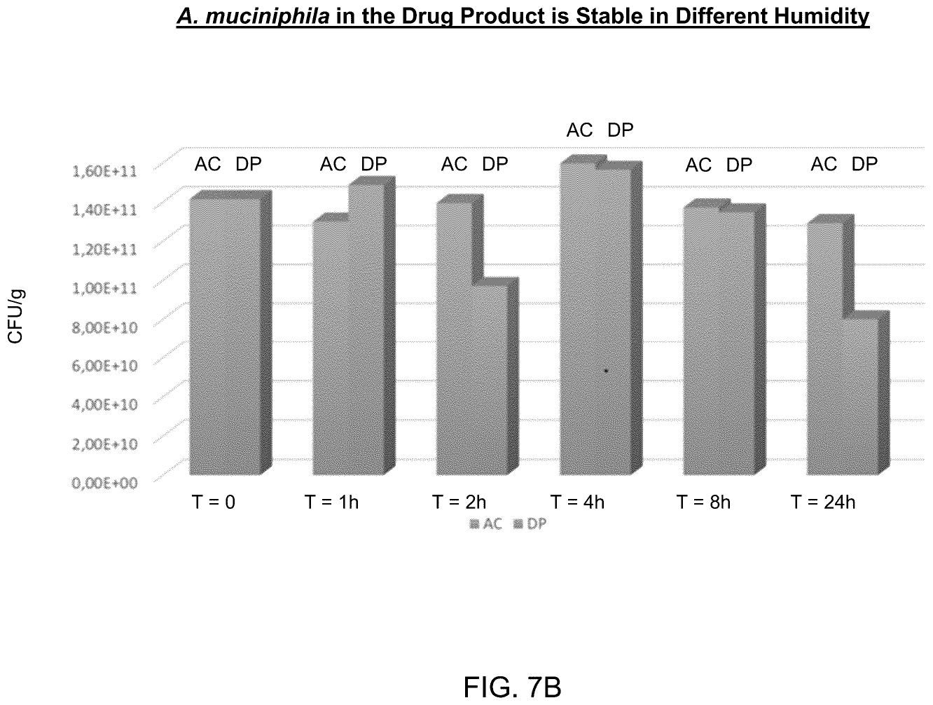

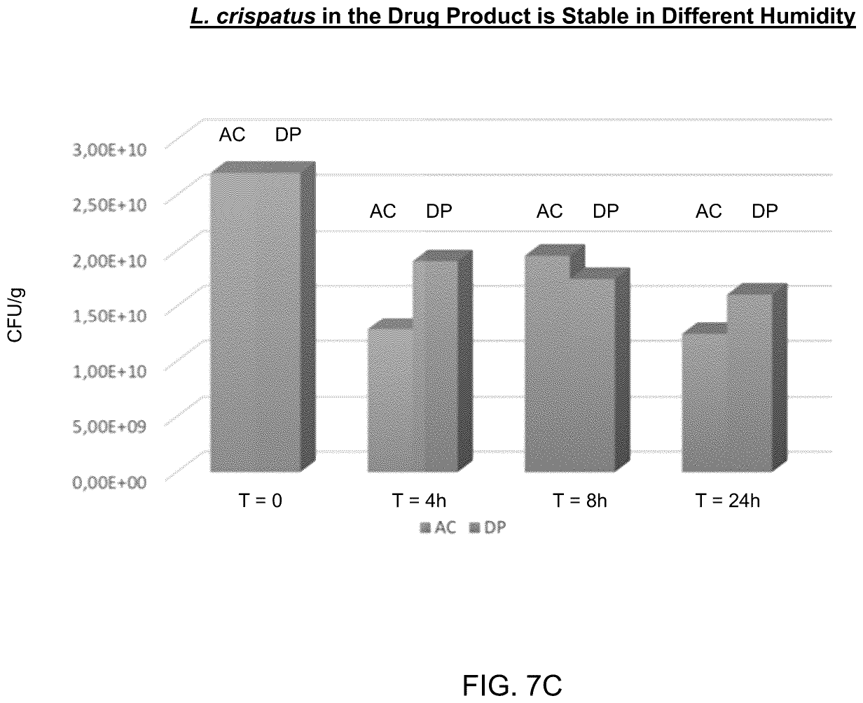

As further depicted in FIGS. 7A, 7B, and 7C, F. prausnitzii (DSM 33185) was stable when the drug product environment was maintained at 25° C. and <30% humidity (RH) but not in the anaerobic chamber (AC) maintained at room temperature and >50%˜ humidity. Both A. muciniphila (DSM 33213), and L. crispatus (DSM 33187) were stable both environments.

### Example 5—Drug Product Composition and Specification

Provided herein are specifications of the drug product. Other specifications, such as those modified from the ones disclosed in this EXAMPLE, can also be used depending on the application of the drug products. The drug product may be the pharmaceutical compositions described in this disclosure. The bacterial strains or drug substances may comprise the bacterial populations described in this disclosure.

The theoretical weight or mass of a drug product should be 325 mg. A theoretical drug product batch should produce 35000 capsules. The theoretical composition of the drug product is summarized in TABLE 14.

TABLE 14
The theoretical composition of the drug product

| | Target | | |
|---|---|---|---|
| | specification | Theoretical | Theoretical |
| | per capsule | weight | weight |
| Materials | (CFU/capsule) | per capsule (mg) | per batch (g) |
| L. crispatus (DSM | 5 × 10^{∧}8 | 10.0 | 350.0 |
| 33187) | | | |
| F. prausnitzii (DSM | 2 × 10^{∧}8 | 120.0* | 4200.0 |
| 33185) | | | |
| A. muciniphila (DSM | 2 × 10^{∧}8 | 1.58* | 55.3 |
| 33213) | | | |
| Silicon Dioxide | 1% (w:w) | 3.25 | 113.8 |
| Mannitol | QS | 190.17 | 6656.0 |

*over-formulated drug substance by 1 × 10^{∧}8 CFU/capsule

The physical and chemical specification of the drug product is summarized in TABLE 15.

TABLE 15
The physical and chemical specification of the drug product.

| Physical and | Acceptance | | |
|---|---|---|---|
| Chemical Tests | Criteria | Method | Justification |
| Appearance | Size 00 yellow | Visual | To confirm the |
| | capsules containing a | analysis | final dosage form |
| | white to off-white to | | |
| | beige powder | | |
| Uniformity of | 325 mg +/− 10% | USP <905> | USP <905>, |
| Dosage Units | | | Weight variation |
| | | | for hard capsule |
| Water | ≤7% | USP <921> | Higher water |
| Determination | | | content |
| | | | in lyophilized |
| | | | formulations |
| | | | likely contributes |
| | | | to loss of viability |

The bacteriological specification of the drug product is summarized in TABLE 16.

TABLE 16
The bacteriological specification of the drug product.

| Bacteriological test | Acceptance Criteria | Method | Justification |
|---|---|---|---|
| Identity by PCR for | Positive | PCR amplification | To confirm identity |
| the presence of A. | | | of the strains |
| muciniphila (DSM | | | |
| 33213), L. crispatus | | | |
| (DSM 33187), and F. | | | |
| prausnitzii (DSM | | | |
| 33185) | | | |
| Viable Cell Count | 1 × 10^{∧}6 − 2 × 10^{∧}9 | Dilution plating on | Set to deliver the |
| (VCC) for A. | CFU/capsule | solid growth media | required number of |
| muciniphila (DSM | | | cells per dose for the clinical study |
| 33213) | | | |
| Viable Cell Count | 1 × 10^{∧}7 − 5 × 10^{∧}9 | Dilution plating on | Set to deliver the |
| (VCC) for L. | CFU/capsule | solid growth media | required number of |
| crispatus (DSM | | | cells per dose for the |
| 33187) | | | clinical study |
| Viable Cell Count | 1 × 10^{∧}6 − 2 × 10^{∧}9 | Dilution plating on | Set to deliver the |
| (VCC) for F. | CFU/capsule | solid growth media | required number of |
| prausnitzii (DSM | | | cells per dose for the |
| 33185) | | | clinical study |
| Total Cell Count | Report Result | Coulter counter | Additional |
| (TCC) | | | information and |
| | | | monitoring of the |
| | | | drug product |
| Bioburden (TAMC) | NMT 200 CFU/g | USP <61> | Increased stringency |
| | | | (lower limit) from |
| | | | USP <1111> |
| | | | requirements for oral |
| | | | solid dosage forms |
| Bioburden (TYMC) | NMT 20 CFU/g. If | USP <61> | Increased stringency |
| | different than 0 | | (lower limit) from |
| | colony: Identify | | USP <1111> |
| | colonies for | | requirements for oral |
| | investigation | | solid dosage forms, |
| | | | with risk-based |
| | | | assessment if any |
| | | | yeast or mold |
| | | | colonies are present |
| Specified | Absent (in 1 g) | USP <62> | Expanded objectionable |
| Microorganisms: | | | organism testing for infant formulation |
| Bile-tolerant Gram- | | | |
| negatives | | | |
| Specified | Absent (in 10 g) | USP <62> | Expanded objectionable |
| Microorganisms: | | | organism testing for infant formulation |
| Salmonella | | | |
| Specified | Absent (in 1 g) | USP <62> | Expanded objectionable |
| Microorganisms: | | | organism testing for infant formulation |
| Staphylococcus | | | |
| aureus | | | |
| Specified | Absent (in 1 g) | USP <62> | Expanded objectionable |
| Microorganisms: | | | organism testing for infant formulation |
| Escherichia coli | | | |
| Specified | Absent (in 1 g) | USP <62> | Expanded objectionable |
| Microorganisms: | | | organism testing for infant formulation |
| Pseudomonas | | | |
| aeruginosa | | | |

### Example 6—Drug Product Feasibility Study

Provided herein are methods for testing the drug product produced by methods and composition described in this disclosure. The drug product may be the pharmaceutical compositions described in this disclosure. The bacterial strains or drug substances may comprise the bacterial populations described in this disclosure.

### Drug Production Formation Protocol

In order to perform the tests exposed in this report, 100 g of drug product was produced according to the drug product specification described in TABLE 17.

TABLE 17
Specification of the drug product used in the drug feasibility test

| | | Specifi- | | Quantity for |
|---|---|---|---|---|
| | Initial VCC | cations | Theoretical | 100 g of |
| Materials | (CFU/g) | (CFU/g) | quantity (%) | drug product |
| L. crispatus | 2.75 × 10^{∧}10 | 5 × 10^{∧}8 | 5.59 | 5.59 |
| (DSM 33187) | | | | |
| A. muciniphila | 2.00 × 10^{∧}11 | 5 × 10^{∧}8 | 0.77 | 0.77 |
| (DSM 33213) | | | | |
| F. prausnitzii | 2.20 × 10^{∧}9 | 5 × 10^{∧}8 | 69.93 | 69.93 |
| (DSM 33185) | | | | |
| Mannitol | / | / | 22.71 | 22.71 |
| Silicon dioxide | / | / | 1.00 | 1.00 |

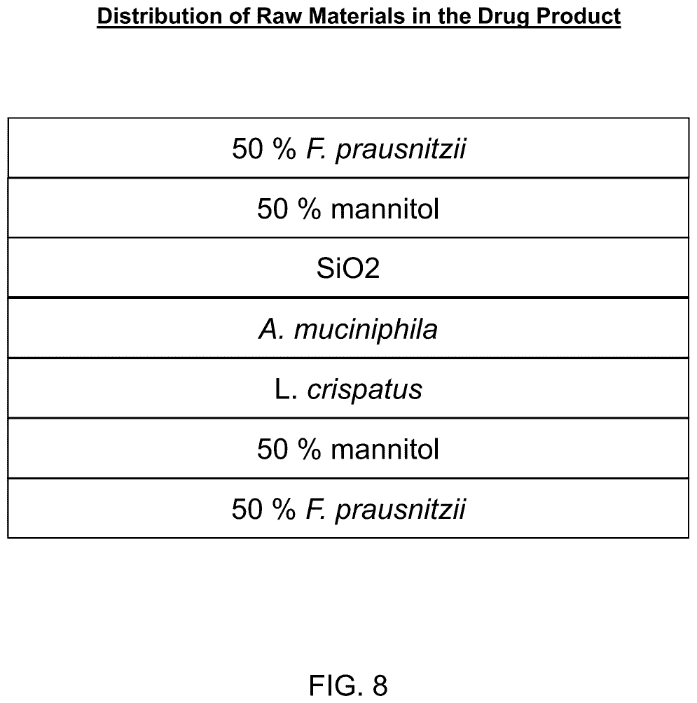

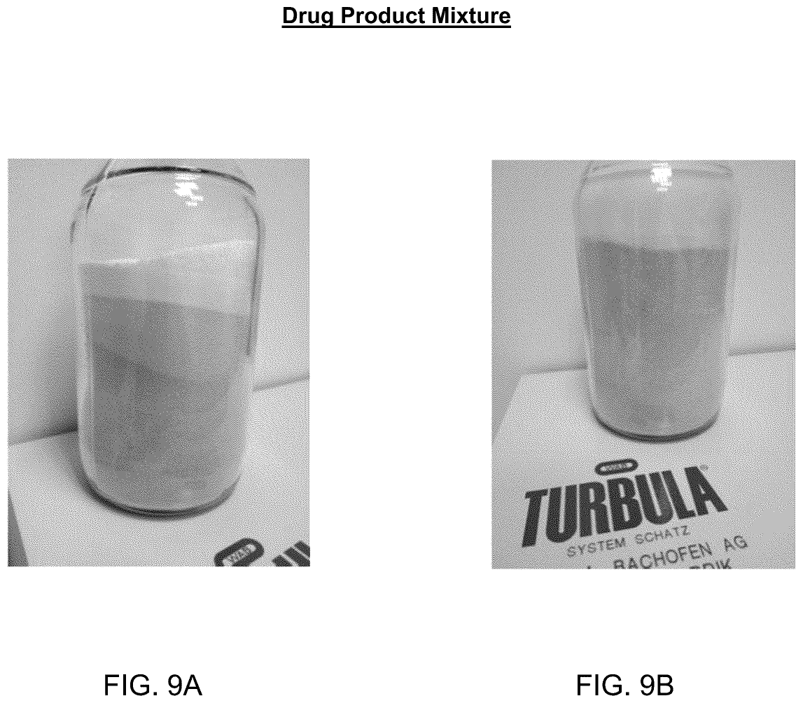

Each of the raw material was weighted in one container (Balance (E01459), temperature=21.9° C., humidity=26.36% RH). The materials were distributed according to FIG. 8 and shown in FIG. 9A. The gray color was provided by the F. prausnitzii (DSM 33185) powder. The materials were mixed in a blending machine (Turbulat E01106) for 5 minutes at 49 rpm. As depicted in FIG. 9B, the mixture was visually homogenous. There were agglomerates of silicon dioxide, which were difficult to disperse in the mix. The color of the mixed powders appeared white to off white to beige.

### Compressibility

Compressibility was assessed by measuring the bulk and tapped densities of the drug product. The bulk density was measured according to method 2 of the 9.6 supplement of European Pharmacopoeia (Ph. Eur. 9.6) (2.9.34.) and USP (616) using a graduated cylinder. A sample of 100 g of the drug product described thereof (weighed to the nearest 0.1%) was poured using a funnel into a 250 mL graduated cylinder allowing the reading to the nearest 2 mL, taking care not to disturb or compact the bed of powder that had passed. The bulk density reading was then taken at the nearest graduation. The measurement of the tapped density was made according to method 2 of the Ph. Eur. 9.6 (2.9.34.), using a volumetric meter (STAV 2003, J Engelsmann AG®) (E00868). The apparatus consisted of a graduated cylinder as well as a tamping system capable of producing 250±15 drops from a height of 3±0.1 millimeter (mm). After determining the aerated volume, the test tube filled with powder was subjected to 10, 500 and then 1250 drops. The tapped volume was recorded at the nearest graduation between each series of drops. If the difference between the volume recorded after 500 and 1250 drops was greater than 2 mL, a new series of 1250 drops was performed. The tapped volume was recorded when the last two volumes reading differed by 2 mL or less. The Carr and Hausner indexes were then calculated for each measurement repeated on the drug product. The average of the three values obtained was defined as the result of the characterization. The sample volumes are summarized in TABLE 18.

TABLE 18
Sample volumes according to the number of settlements.

| | Mass of | | | | | | Mass of |
|---|---|---|---|---|---|---|---|
| | the bulk | V₀ | V₁₀ | V₅₀₀ | V₁₂₅₀ | V₂₅₀₀ | the tapped |
| Test | sample (g) | (ml) | (ml) | (ml) | (ml) | (ml) | sample |
| 1 | 32.75 | 100 | 90 | 71 | 68 | 67 | 32.73 |
| 2 | 32.68 | 100 | 91 | 71 | 69 | 67 | 32.67 |
| 3 | 32.82 | 100 | 90 | 71 | 68 | 67 | 32.80 |

The Hausner ratio is the ratio of tapped density and bulk density and is calculated using Formula 1.

$$HR = \frac{{ρ}_{tassée}}{{ρ}_{aérée}} = \frac{{V}_{aéré}}{{V}_{tassé}} \qquad Formula 1$$

wherein HR is the Haunser ratio; ρ_{tassée} is tapped density; ρ_{aérée} is bulk density; V_{tassé} is tapped volume; and V_{aéré} is bulk volume.

The Carr index is the ratio of the difference between the tapped and bulk density and the tapped density and is calculated using Formula 2.

$$CI = \frac{{ρ}_{tassée} - {ρ}_{aérée}}{{ρ}_{tassée}} \times 100 = \frac{{V}_{aéré} - {V}_{tassé}}{{V}_{aéré}} \times 100 \qquad Formula 2$$

wherein CI is the Carr index; ρ_{tassée} is tapped density; ρ_{aérée} is bulk density; V_{tassé} is tapped volume; and V_{aéré} is bulk volume.

The bulk density was 328 kg/m³, the tapped density was 489 kg/m³, the Carr Index was 33%, and the Hausner ratio was 1.49.

The scale of the flowability according methods described in Ph. Eur. 9.6 (2.9.36), USP. 1174 was summarized in TABLE 19.

TABLE 19
The scale of the flowability (Ph. Eur. 9.6 (2.9.36), USP. 1174)

| Compressibility index (%) | Flow character | Hausner ratio |
|---|---|---|
| 1-10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| >38 | Very, very poor | >1.60 |

The drug product exhibited very poor flowability due to the cohesive behavior of the powder. The powder particles showed significant interparticle interactions.

### Particle Size Analysis

The drug product particle size distribution was measured using a sieve (E01536) a 50 g drug product sample. The data is summarized in TABLE 20.

TABLE 20
Particle size distribution of a drug product.

| | Sieve | Powder + sieve | Powder | Particle size |
|---|---|---|---|---|
| Sieve (mm) | mass (g) | mass (g) | mass (g) | distribution (%) |
| >0.800 | 555.7 | 555.88 | 0.18 | 0.36 |
| 0.500 | 383.77 | 385.75 | 1.98 | 3.93 |
| 0.250 | 333.94 | 349.15 | 15.21 | 30.21 |
| 0.200 | 353.8 | 360.86 | 7.06 | 14.02 |
| 0.100 | 326.76 | 345.19 | 18.43 | 36.6 |
| 0.050 | 324.26 | 331.61 | 7.35 | 14.6 |
| <0.050 | 446.65 | 446.79 | 0.14 | 0.28 |

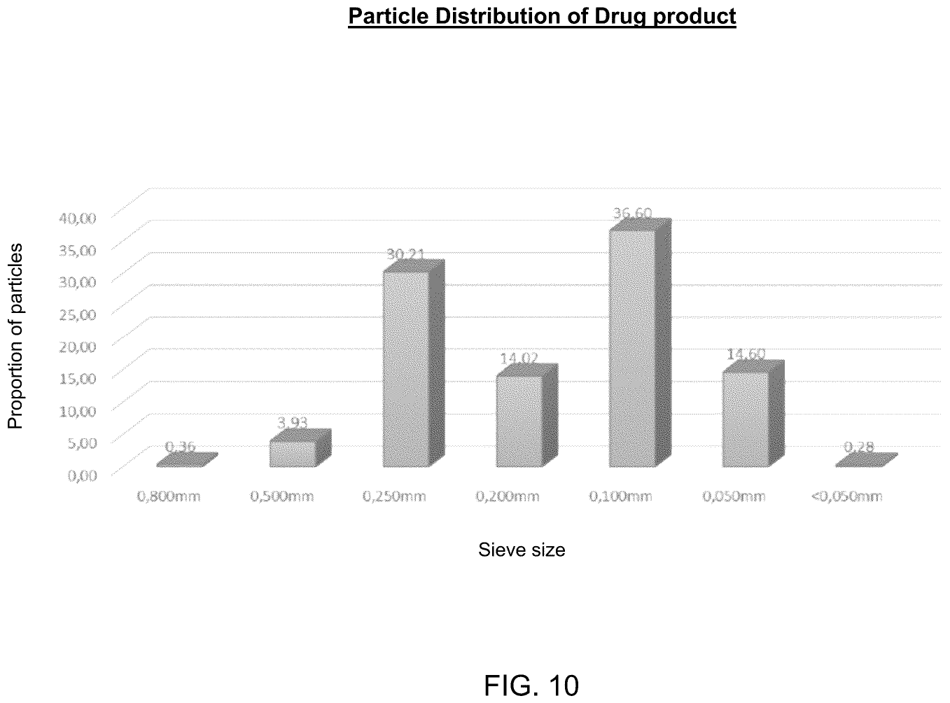

As depicted in FIG. 10, the particle size distribution of the drug product was heterogeneous and consisted of two dominant populations between 0.500 mm-0.250 mm and 0.200 mm-0.100 mm as well as two populations present in smaller quantities between 0.250 mm-0.200 mm and 0.100 mm-0.050 mm.

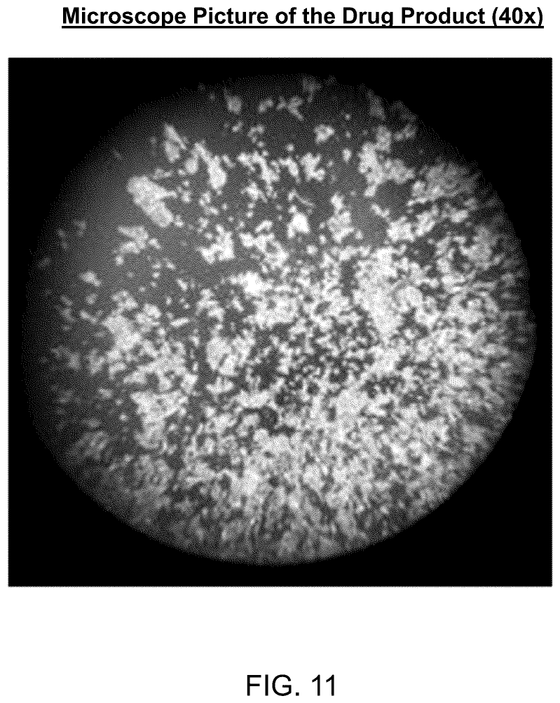

As depicted in FIG. 11, the presence of fine particles was confirmed by the study of the particle size distribution. The drug product under the microscope showed different particles sizes. Some of them were on the order of less than 50 μm. Agglomerates as well as numerous fines that could adsorb to the surface of larger particles were present within the powder. Due to the presence of the agglomerates, the drug product powder tended to be cohesive and therefore had difficulty in flowing.

### Capsule Filling

The Vcaps Plus size 00 white with a low humidity (less than 9%) and a low gas permeability was used for drug product filling. A plate of 100 Vcaps Plus size 00 capsules per manipulator was made in order to test the repeatability of the manipulation. A quantity of 32.1 g of the drug product mix was used. The empty capsules were weighted and recorded. The capsules were placed one by one on the plates of the capsule filler. The capsules were opened filled by leveling. The filled capsules were then closed. The filled capsules were weighted and recorded. The order in which the capsules were placed ensure the same empty and filled capsule were weighted properly. The theoretical mass is calculated as Formula 3.

Theoretical mass=Measurement of bulk density (following the European Pharmacopoeia 2.9.36)×Volume of a capsule. Formula 3:

Since the measurement of bulk density (following the European Pharmacopoeia 2.9.36) was 0.328 g/ml and the volume of a Vcaps Plus size 00 cap was 0.95 mL, the theoretical mass of the drug product should be 313 mg per capsule.

The distribution of the masses on a plate of 100 capsules is summarized in TABLE 21.

TABLE 21
The distribution of the masses (g) on of each of 100 capsules on a plate.

| | | | | |
|---|---|---|---|---|
| 304.9 | 306.9 | 308.2 | 310 | 311.2 |
| 314.1 | 310.7 | 313 | 306.3 | 314.3 |
| 313.5 | 312.9 | 314.6 | 314.1 | 306.3 |
| 312.9 | 304.5 | 323.7 | 307 | 302 |
| 327.8 | 315.5 | 317.8 | 305.3 | 313.1 |
| 320.6 | 308.9 | 322.6 | 306.28 | 313 |
| 315.5 | 301.4 | 319.5 | 320.3 | 312.6 |
| 312.7 | 305.9 | 309.8 | 315.2 | 313.9 |
| 304.5 | 310 | 313.2 | 320.8 | 319.2 |
| 305.9 | 315.1 | 311.2 | 319.8 | 323.7 |
| 307.6 | 310.6 | 311.6 | 323.4 | 319.4 |
| 311 | 306.2 | 317 | 325.5 | 314 |
| 314.6 | 313.3 | 313.5 | 317.8 | 310.5 |
| 316 | 308 | 308.1 | 317 | 308.5 |
| 308.6 | 312 | 306.9 | 318.8 | 303.9 |
| 302.6 | 309.7 | 312.5 | 318.2 | 311.3 |
| 304.5 | 314.2 | 309.8 | 312.2 | 315.9 |
| 309.3 | 307.7 | 315.9 | 313.5 | 308.8 |
| 313.8 | 312.7 | 308.2 | 307.8 | 304 |
| 306.7 | 312.4 | 301.7 | 307.5 | 302.4 |

The parameters of the 100 capsules are summarize in TABLE 22.

TABLE 22
The parameters of the 100 capsules.

| Weight parameter | | Deviation from the | | | |
|---|---|---|---|---|---|
| of 100 Capsules (g) | | theoretical weight | | Deviation from the mean | |
| Mean | 311.99 | Spec (%0 | 5.00 | Spec (%) | 10.00 |
| Min | 301.4 | Min (g) | 297.35 | Min (g) | 280.79 |
| Max | 327.8 | Max (g) | 328.65 | Max (g) | 343.19 |

The distribution of capsules according to the theoretical weight and Ph. 2.9.5. is summarized in TABLE 23.

TABLE 23
The distribution of capsules according
to the theoretical weight and Ph. 2.9.5.

| | Characteristics | Number of | Percentage |
|---|---|---|---|
| | conditions | capsules | (%) |
| Below the mean | Loss | 0 | 0 |
| weight | Between −10% | 0 | 0 |
| | and −20% | | |
| | Between −10% | 100 | 100 |
| | and 10% | | |
| Above the mean | Between 10% | 0 | 0 |
| weight | and 20% | | |

The loss corresponded to capsules that couldn't be used because they did not comply with the theoretical weight. They might had been broken, cracked or damaged during manufacturing The average mass obtained was 311.99 mg and is within ±5% of the theoretical weight, i.e., between 297.35 mg and 328.65 mg. Regarding mass uniformity, the results obtained for the 100 capsules were within ±10% of the average obtained, between 280.79 mg and 343.19 mg.

Mechanical filling allowed a slightly larger amount of powder in the capsules, compared to that of manual filling.

### Solubilization in Water

The capsule of the drug product could be opened, and the powder was poured into a glass of liquid before consumption.

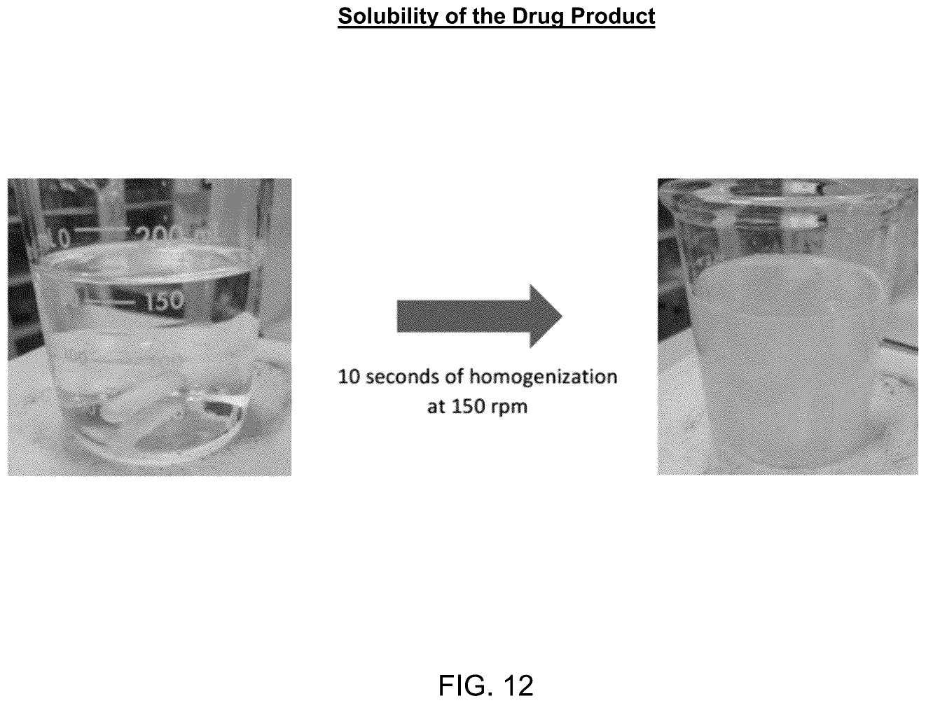

As depicted in FIG. 12, the drug product solubilizes in water without large silicon agglomeration or precipitate formation.

### Shelf Stability of the Pharmaceutical Composition at Room Temperature

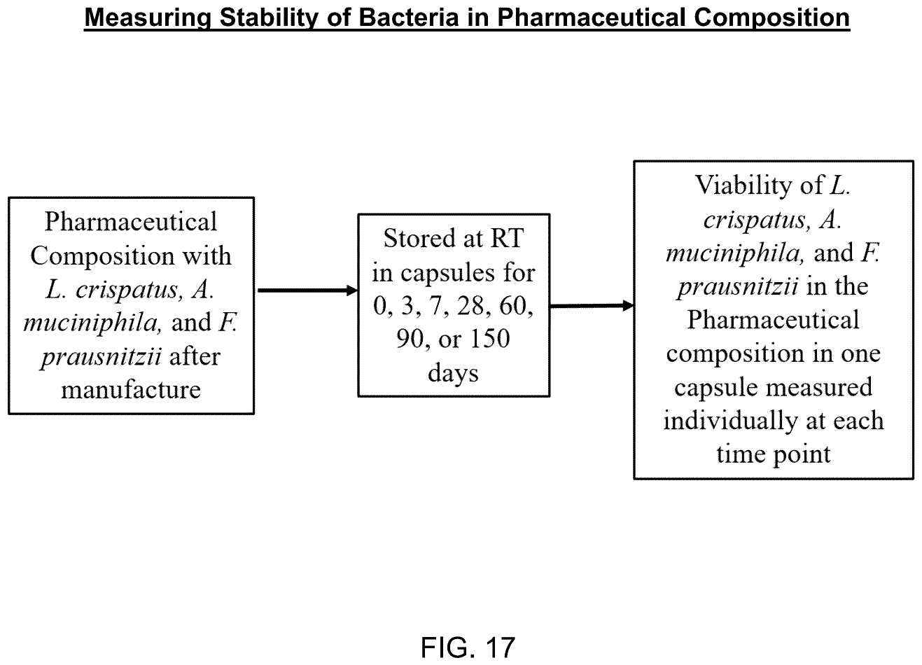

The stability of the drug product was tested by storing the drug product at room temperature (25° C.) for 150 days. The drug product was manufactured according to methods described in EXAMPLES 2-9. The drug product comprised the pharmaceutically acceptable excipient, cryoprotectant, capsule, and packaging described in this disclosure The viability of the drug substances in the drug product was measured via colony-forming units per capsule (CFU/capsule) and the drug product was tested on days 0, 3, 7, 28, 60, 90, and 150. The stability of the drug substances L. crispatus, A. muciniphila, and F. prausnitzii was measured individually, as depicted in FIG. 17. The stability data is summarized in TABLE 38.

TABLE 38
Stability of drug substances L. crispatus, A. muciniphilia, and
F. prausnitzii in drug product at room temperature (25° C.).
CFU/capsule

| RT storage (days) | L. crispatus | A. muciniphila | F. prausnitzii |
|---|---|---|---|
| 0 | 2.23E+09 | 1.11E+09 | 4.96E+08 |
| 3 | 1.57E+09 | 4.98E+08 | 1.80E+08 |
| 7 | 4.87E+09 | 1.15E+09 | 4.02E+08 |
| 28 | 2.07E+09 | 4.62E+08 | 1.20E+08 |
| 60 | 1.93E+09 | 5.88E+08 | 2.67E+07 |
| 90 | 6.60E+08 | 2.82E+08 | 2.50E+07 |
| 150 | 4.60E+09 | 3.15E+08 | 2.28E+07 |

The above disclosed method can be used to test the stability of any drug product at room temperature for any bacterial strain, species, genus, or family disclosed in the present disclosure.

In conclusion, the drug product contained a major fraction of thin particles. The particles were the source of a cohesive powder having a poor Carr index or Hausner index. Silicon dioxide would facilitate the flow of the drug product when it was encapsulated. Ideally, sieving upstream of the mixture would be required in order to limit the appearance of agglomerates

### Example 7—Manufacturing of the Drug Substances in Static Cultures

Provided herein are methods for manufacturing the drug substances in static cultures. The drug product may be the pharmaceutical compositions described in this disclosure. The bacterial strains or drug substances may comprise the bacterial populations described in this disclosure.

To produce a drug product in a 5-L GMP scale, each of A. muciniphila (DSM 33213), L. crispatus (DSM 33187), and F. prausnitzii (DSM 33185) was grown in 5 L strain-specific vegan growth media at 37° C. under anaerobic conditions in static cultures in four batches. For each batch, the cells were concentrated by centrifugation in sealed bottle. and resuspended manually in the buffered glycerol solution at 40× concentration into a drug substance in an anaerobic chamber. The buffered glycerol solution was composed of standard phosphate buffered saline (PBS, 137 mM NaCl, 2.7 mM KCl, 10 mM Na₂HPO₄, and 1.8 mM KH₂PO₄), 20% v/v glycerol, and 0.1% w/w cysteine as an antioxidant. Each drug substance was aliquoted into 1-ml doses. Each strain was stored at −80° C. A specification testing was performed on each batch of drug substance. One specification standard is described in Examples 3.

Upon passing the specification standard, the drug substance batch was released for drug product manufacturing. A drug product batch size was calculated based on the potencies and volumes of the drug substance batches, as shown in TABLES 24, 25 and 26.

TABLE 24
Specification of drug substance L. crispatus
(DSM 33187) manufactured in static cultures.

| | Batch | Viable cell | Total # |
|---|---|---|---|
| Batch # | volume (L) | count (CFU/ml) | of doses |
| 1 | 5 | 4.89 × 10\hat{ }9 | 743 |
| 2 | 5 | 3.82 × 10\hat{ }9 | 993 |
| 3 | 5 | 3.68 × 10\hat{ }9 | 1104 |
| 4 | 5 | 3.46 × 10\hat{ }9 | 1038 |

TABLE 25
Specification of drug substance A. muciniphila
(DSM 33213) manufactured in static cultures.

| | Batch | Viable cell | Total # |
|---|---|---|---|
| Batch # | volume (L) | count (CFU/ml) | of doses |
| 1 | 5 | 1.13 × 10\hat{ }10 | 2599 |
| 2 | 5 | 2.2 × 10\hat{ }10 | 5720 |
| 3 | 5 | 1.13 × 10\hat{ }10 | 3390 |
| 4 | 5 | 2.09 × 10\hat{ }10 | 6270 |

TABLE 26
Specification of drug substance F. prausnitzii
(DSM 33185) manufactured in static cultures.

| | Batch | Viable cell | Total # |
|---|---|---|---|
| Batch # | volume (L) | count (CFU/ml) | of doses |
| 1 | 5 | 5.01 × 10\hat{ }9 | 932 |
| 2 | 5 | 2.60 × 10\hat{ }9 | 676 |
| 3 | 5 | 8.80 × 10\hat{ }9 | 2605 |
| 4 | 5 | 3.00 × 10\hat{ }10 | 9000 |

The three drug substances were then combined according to the calculated drug product batch size, diluted in the PBS-CG buffer, and aliquoted in 1-ml doses. The doses were then stored at −80° C.

### Example 8—Manufacturing of the Drug Product in Anaerobic Fermenters

Provided herein are methods for manufacturing the drug substances in anaerobic fermenters.

Each of A. muciniphila (DSM 33213), L. crispatus (DSM 33187), and F. prausnitzii (DSM 33185) was grown in batches of 5 L, 20 L, or 150 L strain-specific vegan growth media at 37° C. under anaerobic conditions in different fermenters. For F. prausnitzii (DSM 33185), it was further grown in batches of 2 L strain-specific vegan growth media at 37° C. under anaerobic conditions in different fermenters. For F. prausnitzii (DSM 33185), it was further grown in batches of 2 L strain-specific vegan growth media at 37° C. under anaerobic conditions in different fermenters. For each batch, the cells were concentrated by centrifugation and resuspended in the cryoprotectant, as described in TABLE 5 and EXAMPLE 1, at 100× concentration into a drug substance automatically in a stirred-tank mixing vessel under anaerobic atmosphere. Each drug substance mixture was lyophilized and grinded into powder forms. Each strain was stored at −20° C. A specification testing was performed on each batch of drug substance. One specification standard is described in EXAMPLE 3.

Upon passing the specification standard, the drug substance batches were released for drug product manufacturing. A drug product batch size was calculated based on the potencies and volumes of the drug substance batches, as shown in TABLES 27, 28 and 29.

TABLE 27
Specification of drug substance L. crispatus
(DSM 33187) in anaerobic fermenter.

| | | | VCC on | |
|---|---|---|---|---|
| Batch volume | | Lyophilized | lyophilized | Total |
| (L)/number | End of culture | powder | powder | # of |
| of batches | VCC (CFU/ml) | weight (g) | (CFU/g) | doses |
| 5/4 | 1.39 × 10\hat{ }9 | 17.77 | 6.51 × 10\hat{ }10 | 2320 |
| 20/1 | 3.15 × 10\hat{ }9 | 49 | 6.7 × 10\hat{ }10 | 6566 |
| 150/1 | 3.03 × 10\hat{ }9 | 1200 | 2.75 × 10\hat{ }10 | 66000 |

TABLE 28
Specification of drug substance A. muciniphila
(DSM 33213) in anaerobic fermenter.

| | | | VCC on | |
|---|---|---|---|---|
| Batch volume | | Lyophilized | lyophilized | Total |
| (L)/number | End of culture | powder | powder | # of |
| of batches | VCC (CFU/ml) | weight (g) | (CFU/g) | doses |
| 5/3 | 2.50 × 10\hat{ }8 | 13.04 | 1.61 × 10\hat{ }10 | 420 |
| 20/1 | 3.45 × 10\hat{ }9 | 35.6 | 1.10 × 10\hat{ }10 | 783 |
| 150/1 | 4.50 × 10\hat{ }8 | 1200 | 4.51 × 10\hat{ }9 | 3879 |

TABLE 29
Specification of drug substance F. prausnitzii
(DSM 33185) in anaerobic fermenter.

| | | | VCC on | |
|---|---|---|---|---|
| Batch volume | | Lyophilized | lyophilized | Total |
| (L)/number | End of culture | powder | powder | # of |
| of batches | VCC (CFU/ml) | weight (g) | (CFU/g) | doses |
| 2/5 | 1.00 × 10\hat{ }6 | 2 | 3.70 × 10\hat{ }9 | ~15 |
| 5/5 | 1.10 × 10\hat{ }6 | N/A | 6.40 × 10\hat{ }6 | N/A |
| 20/1 | N/A | N/A | 8.00 × 10\hat{ }7 | N/A |
| 150/1 | 5.00 × 10\hat{ }5 | 130 | 5.00 × 10\hat{ }5 | >1 |

N/A: not available

To produce the drug product, the three drug substances were then combined with excipients blends (bulking agents, glidants, adsorbents) according to the calculated drug product batch size in a Turbular blender. The excipient blend contained 1% silicon dioxide, 1.5% magnesium stearate, and a variable percentage of mannitol bulking agent based on drug substance potency. Because total drug substance mass per drug product batch varied based on potency of the specific constituent drug substance lots, mannitol concentration was adjusted accordingly (5-81.5%) to achieve a 325-350 mg total capsule fill weight. The drug product was manually or mechanically filled into size 00 hypromellose capsules.

### Example 9—Scaled-Up Manufacturing of the Drug Product in Anaerobic Fermenters

Provided herein are methods for manufacturing the drug substances in anaerobic fermenters in ultra-large scale. The drug product may be the pharmaceutical compositions described in this disclosure. The bacterial strains or drug substances may comprise the bacterial populations described in this disclosure.

Each of A. muciniphila (DSM 33213), L. crispatus (DSM 33187), and F. prausnitzii (DSM 33185) was grown in strain-specific vegan growth media at 37° C. under anaerobic conditions in fermenters. The inoculation scheme involved continuous culturing in subsequently larger culture volume. For L. crispatus (DSM 33187), and F. prausnitzii (DSM 33185), a WCB stock was used to inoculate a 1-L culture. The 1-L culture was used to inoculate a 20-L culture, and the 20-L culture was used to inoculate a 3500-L culture. For A. muciniphila (DSM 33213), the 1-L culture inoculated from a WCB stock was used to inoculate a 20-L culture, and the 20-L culture was used to inoculate a 300-L culture. Finally, the 300-L culture was used to inoculate a 3000-L media.

The cells were concentrated by centrifugation and resuspended in the cryoprotectant, as described in TABLE 5 and EXAMPLE 1, at 100× concentration into a drug substance automatically in a stirred-tank mixing vessel under anaerobic atmosphere. Each drug substance mixture was lyophilized and grinded into powder forms. Each strain was stored at −20° C. A specification testing was performed on each batch of drug substance. One specification standard is described in EXAMPLE 3.

Upon passing the specification standard, the drug substance batches were released for drug product manufacturing. A drug product batch size was calculated based on the potencies and volumes of the drug substance batches, as shown in TABLES 30, 31 and 32.

TABLE 30
Specification of drug substance L. crispatus
(DSM 33187) in anaerobic fermenter.

| | | | VCC on | |
|---|---|---|---|---|
| | | Lyophilized | lyophilized | Total |
| Batch | End of culture | powder | powder | # of |
| volume (L) | VCC (CFU/ml) | weight (g) | (CFU/g) | doses |
| 1 | 2.70 × 10\hat{ }8 | N/A | N/A | N/A |
| 20 | 2.48 × 10\hat{ }9 | N/A | N/A | N/A |
| 3500 | 1.30 × 10\hat{ }9 | 18000 | 7.00 × 10\hat{ }10 | 2520000 |

N/A: not available

TABLE 31
Specification of drug substance A. muciniphila
(DSM 33213) in anaerobic fermenter.

| | | | VCC on | |
|---|---|---|---|---|
| Batch volume | | Lyophilized | lyophilized | Total |
| (L)/number | End of culture | powder | powder | # of |
| of batches | VCC (CFU/ml) | weight (g) | (CFU/g) | doses |
| 1 | 3.30 × 10\hat{ }8 | N/A | N/A | N/A |
| 20 | 3.10 × 10\hat{ }8 | N/A | N/A | N/A |
| 300 | 3.60 × 10\hat{ }8 | N/A | N/A | N/A |
| 3000 | 5.10 × 10\hat{ }8 | 2950 | 2.10 × 10\hat{ }11 | 1239000 |

N/A: not available

TABLE 32
Specification of drug substance F. prausnitzi
(DSM 33185) in anaerobic fermenter.

| | | | VCC on | |
|---|---|---|---|---|
| Batch volume | | Lyophilized | lyophilized | Total |
| (L)/number | End of culture | powder | powder | # of |
| of batches | VCC (CFU/ml) | weight (g) | (CFU/g) | doses |
| 1 | 3.30 × 10\hat{ }9 | N/A | N/A | N/A |
| 20 | 1.40 × 10\hat{ }9 | N/A | N/A | N/A |
| 3500 | 5.40 × 10\hat{ }8 | 13600 | 2.20 × 10\hat{ }9 | 59840 |

N/A: not available

To produce the drug product, the mass of each drug substance and excipient blend powder was calculated based on the desired total yield and capsule fill weight (approximately 10,000 capsules with a 325 mg fill weight). The excipient blend powder contained 1% silicon dioxide, 1.5% magnesium stearate, and a variable percentage of mannitol bulking agent based on drug substance potency. Because total drug substance mass per drug product batch varied based on potency of the specific constituent drug substance lots, mannitol concentration was adjusted accordingly (5-81.5%) to achieve the 325 mg total capsule fill weight. Once all excipient powder powders (mannitol, magnesium stearate, and silicon dioxide) and drug substance powders were dispensed, the powders were mixed to homogeneity using a cubic form blender. The uniformity of the blend was analyzed using a pre-defined, stratified sampling approach, in which discrete samples were removed in triplicate from mapped locations within the cubic form blender using a sample thief, and each sample was analyzed for concentration each of the three drug substances as well as moisture. Analysis of these stratified samples demonstrated a homogeneous blend, with potency results for all three drug substances consistently within the target potency for the drug product, and there was no significant increase in moisture.

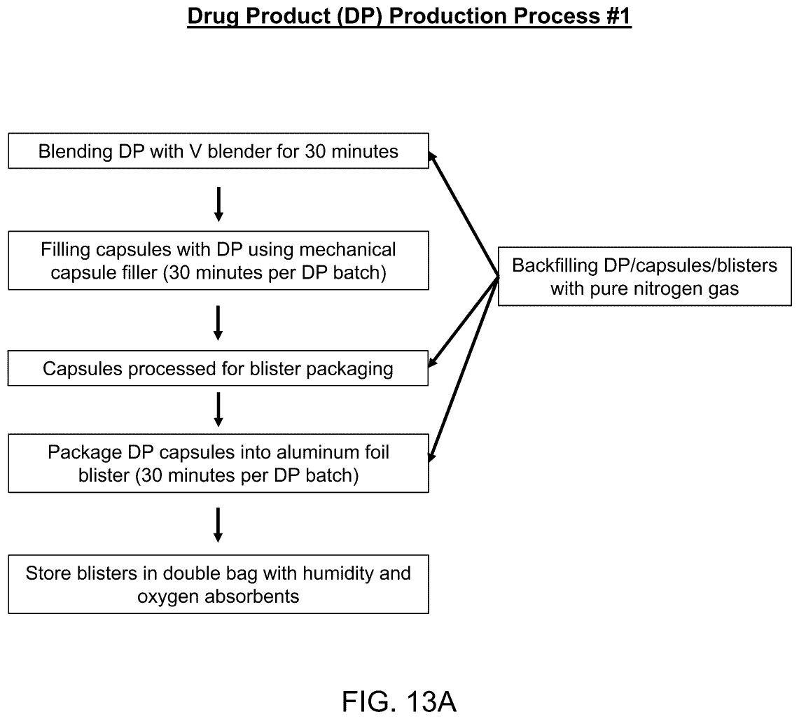

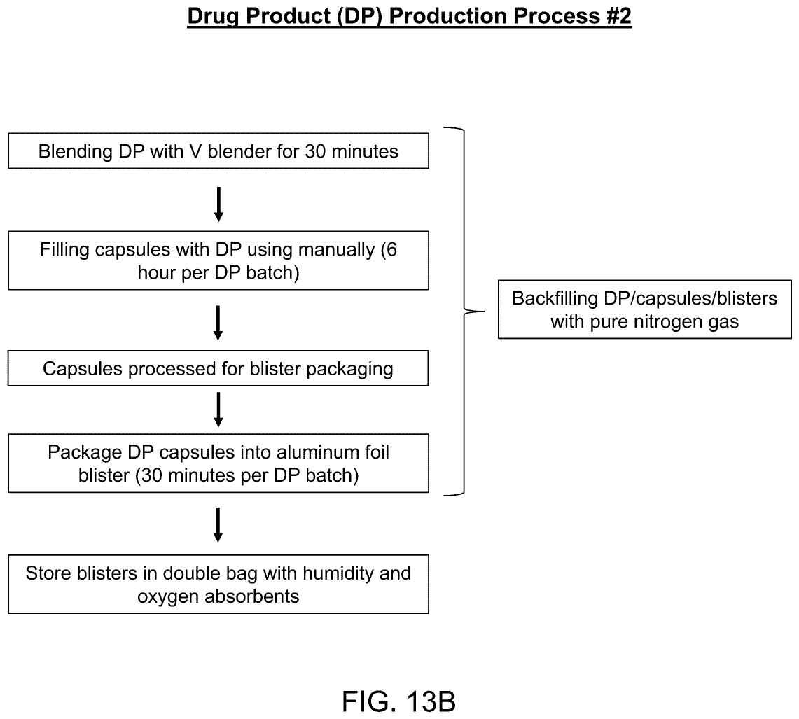

The homogenized bulk powder was subsequently filled into size 00 hypromellose capsules (primary container closure; capsule shell is not for consumption) using an automated mechanical capsule filler to achieve the target 325 mg fill weight per dose: as depicted in FIG. 13A, the drug product powders were blended with a V blender for 30 minutes. The capsule was filled with the drug product powder using the mechanical filler. The capsule was then processed for aluminum blister packing. The packing process took about 30 minutes per drug product batch. The blistered were then stored in double bag with humidity and oxygen absorbents. During the blending, the processing of the capsule blister packing, and the blister packing, the drug product, the capsule, and the blister pack were backfilled with pure nitrogen.

The specification of the drug product in the capsule filled by the automated capsule filler is shown in TABLE 33.

TABLE 33
The drug product specification of the capsule filled by automated mechanical
capsule filler.

| Physical | | Drug | |
|---|---|---|---|
| and Chemical Tests | Acceptance Criteria | product result | Method |
| Appearance | Size 00 yellow capsules | Compiles | Visual analysis |
| | containing a white to off- | | |
| | white to beige powder | | |
| Uniformity of Dosage | 325 mg +/− 10% | 319 mg/ | USP <905> |
| Units | | Uniformity of | |
| | | mass compiles | |
| Water Determination | ≤7% | 2% | USP <921> |
| | | Drug | |
| Bacteriological Tests | Acceptance Criteria | product result | Method |
| Identity by PCR for the | Positive | N/A | PCR amplification |
| presence of A. muciniphila | | | |
| (DSM 33213), L. crispatus | | | |
| (DSM 33187), and F. | | | |
| prausnitzii (DSM 33185) | | | |
| Viable Cell Count (VCC) for | 1 × 10\hat{ }6-2 × 10\hat{ }9 | 2 × 10\hat{ }8 | Dilution plating on |
| A. muciniphila (DSM 33213) | CFU/capsule | CFU/capsule | solid growth media |
| Viable Cell Count (VCC) for | 1 × 10\hat{ }7-5 × 10\hat{ }9 | 4 × 10\hat{ }8 | Dilution plating on |
| L. crispatus (DSM 33187) | CFU/capsule | CFU/capsule | solid growth media |
| Viable Cell Count (VCC) for | 1 × 10\hat{ }6-2 × 10\hat{ }9 | 8 × 10\hat{ }7 | Dilution plating on |
| F. prausnitzii (DSM 33185) | CFU/capsule | CFU/capsule | solid growth media |
| Total Cell Count (TCC) | Report Result | 3.9 × 10\hat{ }11 | Coulter counter |
| | | CFU/capsule | |
| Bioburden (TAMC) | NMT 200 CFU/g | 20 CFU/g | USP <61> |
| Bioburden (TYMC) | NMT 20 CFU/g. If | <5 | USP <61> |
| | different than 0 colony: | CFU/capsule | |
| | Identify colonies for | | |
| | investigation | | |
| Specified Microorganisms: | Absent (in 1 g) | Absent | USP <62> |
| Bile-tolerant Gram-negatives | | | |
| Specified Microorganisms: | Absent (in 10 g) | Absent | USP <62> |
| Salmonella | | | |
| Specified Microorganisms: | Absent (in 1 g) | Absent | USP <62> |
| Staphylococcus aureus | | | |
| Specified Microorganisms: | Absent (in 1 g) | Absent | USP <62> |
| Escherichia coli | | | |
| Specified Microorganisms: | Absent (in 1 g) | Absent | USP <62> |
| Pseudomonas aeruginosa | | | |

N/A: Not available

The homogenized bulk powder was also filled into size 00 hypromellose capsules (primary container closure; capsule shell is not for consumption) manually to achieve the target 325 mg fill weight per dose: as depicted in FIG. 13B, the drug product powders were blended with a V blender for 30 minutes. The capsule was filled with the drug product powder manually. This process took about 6 hours per drug product batch. The capsule was then processed for aluminum blister packing. The packing process took about 30 minutes per drug product batch. The blistered were then stored in double bag with humidity and oxygen absorbents. In every process pre-storage, the drug product, the capsule, and the blister were backfilled with pure nitrogen. The specification of the drug product in the capsule filled manually is shown in TABLE 34.

TABLE 34
The drug product specification of the capsule filled manually.

| | | Drug | |
|---|---|---|---|
| Physical and Chemical Tests | Acceptance Criteria | product result | Method |
| Appearance | Size 00 yellow | Compiles | Visual analysis |
| | capsules containing a | | |
| | white to off-white to | | |
| | beige powder | | |
| Uniformity of Dosage Units | 325 mg +/− 10% | 362 mg/ | USP <905> |
| | | Uniformity of | |
| | | mass compiles | |
| Water Determination | ≤7% | 2% | USP <921> |
| | | Drug | |
| Bacteriological Tests | Acceptance Criteria | product result | Method |
| Identity by PCR for the | Positive | N/A | PCR amplification |
| presence of A. muciniphila | | | |
| (DSM 33213), L. crispatus | | | |
| (DSM 33187), and F. | | | |
| prausnitzii (DSM 33185) | | | |
| Viable Cell Count (VCC) for | 1 × 10\hat{ }6-2 × 10\hat{ }9 | 2 × 10\hat{ }8 | Dilution plating on |
| A. muciniphila (DSM 33213) | CFU/capsule | CFU/capsule | solid growth media |
| Viable Cell Count (VCC) for | 1 × 10\hat{ }7-5 × 10\hat{ }9 | 3 × 10\hat{ }8 | Dilution plating on |
| L. crispatus (DSM 33187) | CFU/capsule | CFU/capsule | solid growth media |
| Viable Cell Count (VCC) for | 1 × 10\hat{ }6-2 × 10\hat{ }9 | 8 × 10\hat{ }7 | Dilution plating on |
| F. prausnitzii (DSM 33185) | CFU/capsule | CFU/capsule | solid growth media |
| Total Cell Count (TCC) | Report Result | 2.8 × 10\hat{ }11 | Coulter counter |
| | | CFU/capsule | |
| Bioburden (TAMC) | NMT 200 CFU/g | <20 CFU/g | USP <61> |
| Bioburden (TYMC) | NMT 20 CFU/g. If | <5 | USP <61> |
| | different than 0 | CFU/capsule | |
| | colony: Identify | | |
| | colonies for | | |
| | investigation | | |
| Specified Microorganisms: | Absent (in 1 g) | Absent | USP <62> |
| Bile-tolerant Gram-negatives | | | |
| Specified Microorganisms: | Absent (in 10 g) | Absent | USP <62> |
| Salmonella | | | |
| Specified Microorganisms: | Absent (in 1 g) | Absent | USP <62> |
| Staphylococcus aureus | | | |
| Specified Microorganisms: | Absent (in 1 g) | Absent | USP <62> |
| Escherichia coli | | | |
| Specified Microorganisms: | Absent (in 1 g) | Absent | USP <62> |
| Pseudomonas aeruginosa | | | |

N/A: Not available

Filled capsules were secondary packed in cold form aluminum blisters (8 capsules per blister) at low humidity using a nitrogen gas filled headspace. Blisters were stored in HDPE bags inside cartons at <−15° C., with a pre-defined number of blisters removed for QC analysis and for placement on formal, supportive stability.

### Example 10—Testing the Gas Permeability of a Capsule Storing a Drug Product

Provided herein are methods for assaying the gas permeability of the capsule containing the drug product. The drug product may be the pharmaceutical compositions described in this disclosure. The bacterial strains or drug substances may comprise the bacterial populations described in this disclosure.

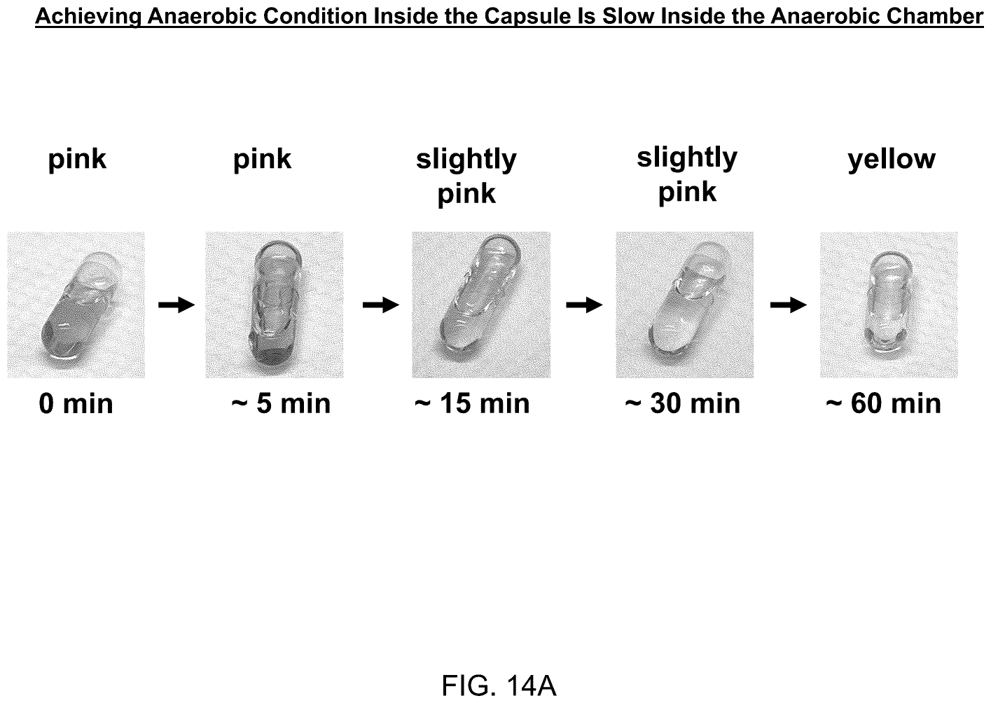

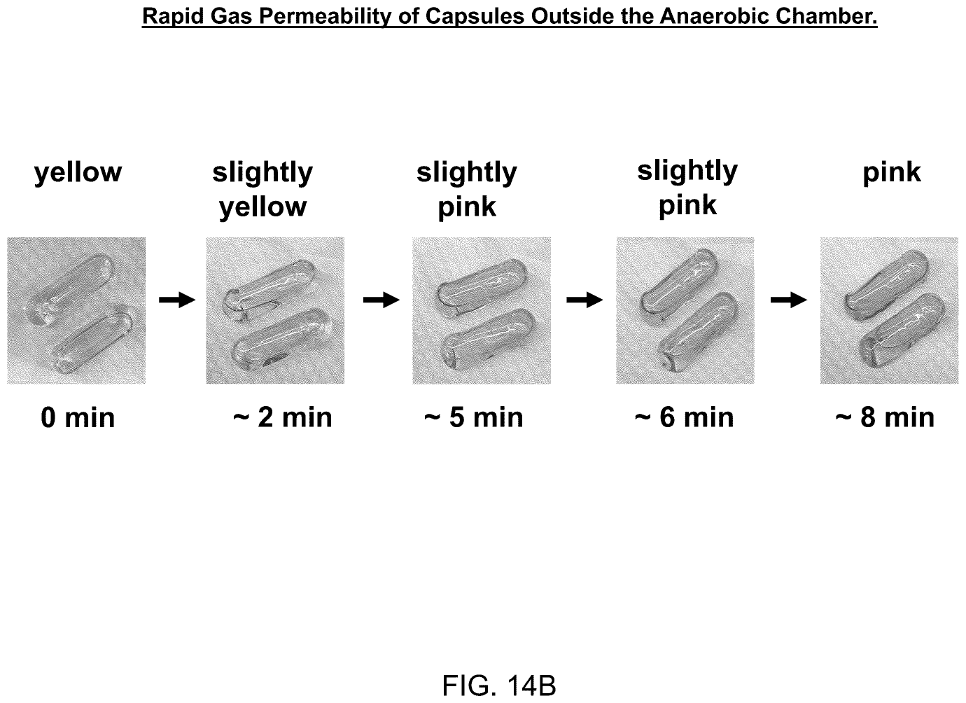

To determine the gas permeability of the V-caps capsule used to contain the drug product, the capsule was moved into the anaerobic chamber (AS-150) chamber and filled with the YFAP broth and 0.12% Rezasurin. The filled capsule was incubated in AS-150. Rezasurin appeared pink in the presence of oxygen and colorless (or yellow) in the absence of oxygen. As shown in FIG. 14A, it took about 60 minutes for the inside of the capsule to achieve anaerobic environment.

In contrast, when the capsule incubated in AS-150 overnight was filled with the YFAP broth and 0.12% Rezasurin and moved out of AS-150 to be exposed to air, it took less than 10 minutes for the anaerobic environment inside the capsule to become aerobic, as depicted in FIG. 14B.

### Example 11—Testing the Stability of a Drug Product

Provided herein are methods for assaying the stability of the drug product stored in capsule packed in aluminum blister packs in freeze-thaw cycles and transportation. The drug product may be the pharmaceutical compositions described in this disclosure. The bacterial strains or drug substances may comprise the bacterial populations described in this disclosure.

The drug product was packed in the aluminum blister according to the methods described in EXAMPLE 9. The blister was stored at −20° C.

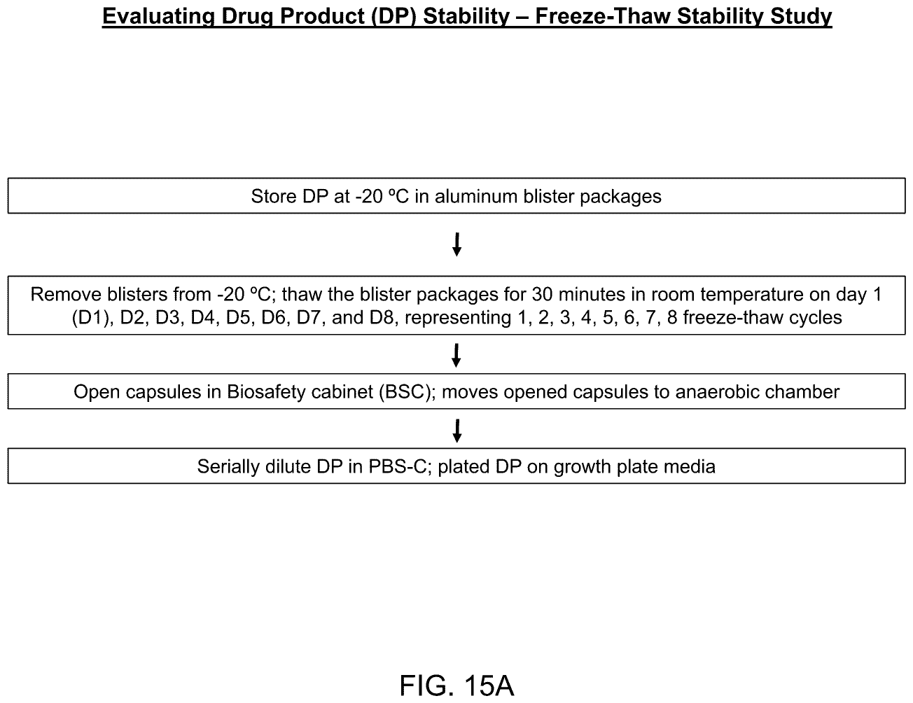

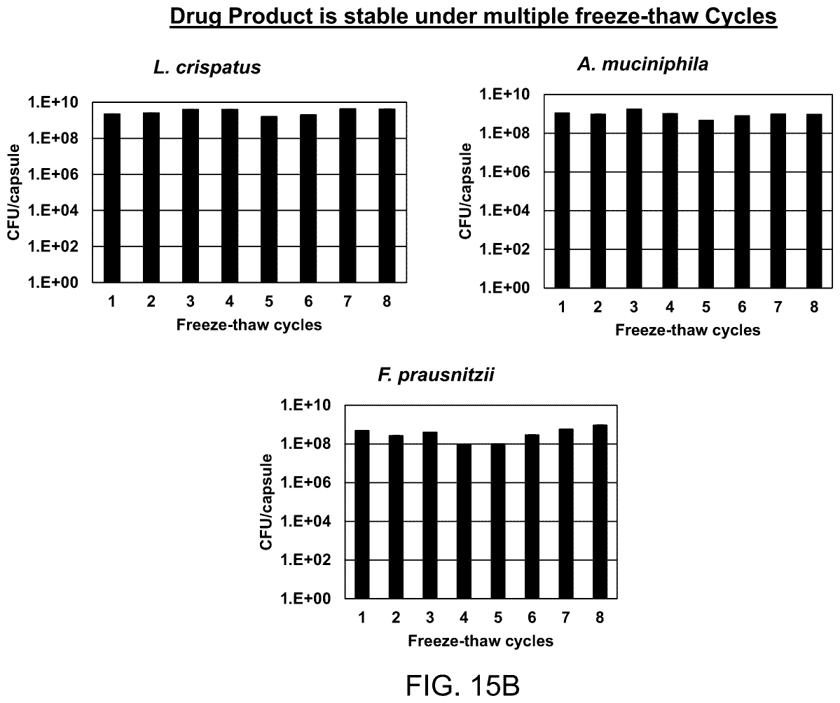

As depicted in FIG. 15A, to assay for the freeze-thaw stability of the drug product in the aluminum blister, the blister was removed from −20° C. and thawed for 30 minutes in room temperature on day 1 (D1), D2, D3, D4, D5, D6, D7, and D8, representing 1, 2, 3, 4, 5, 6, 7, 8 freeze-thaw cycles. The capsules of the thawed blister were opened in Biosafety cabinet (BSC). The opened capsules were moved to anaerobic chamber. The drug product was serially diluted in the standard phosphate buffered saline (PBS, 137 mM NaCl, 2.7 mM KCl, 10 mM Na₂HPO₄, and 1.8 mM KH₂PO₄) and 0.1% w/w cysteine as an antioxidant and plated on growth plate media. As depicted in FIG. 15B F. prausnitzii (DSM 33185), A. muciniphila (DSM 33213), and L. crispatus (DSM 33187) were stable under every freeze-thaw cycle.

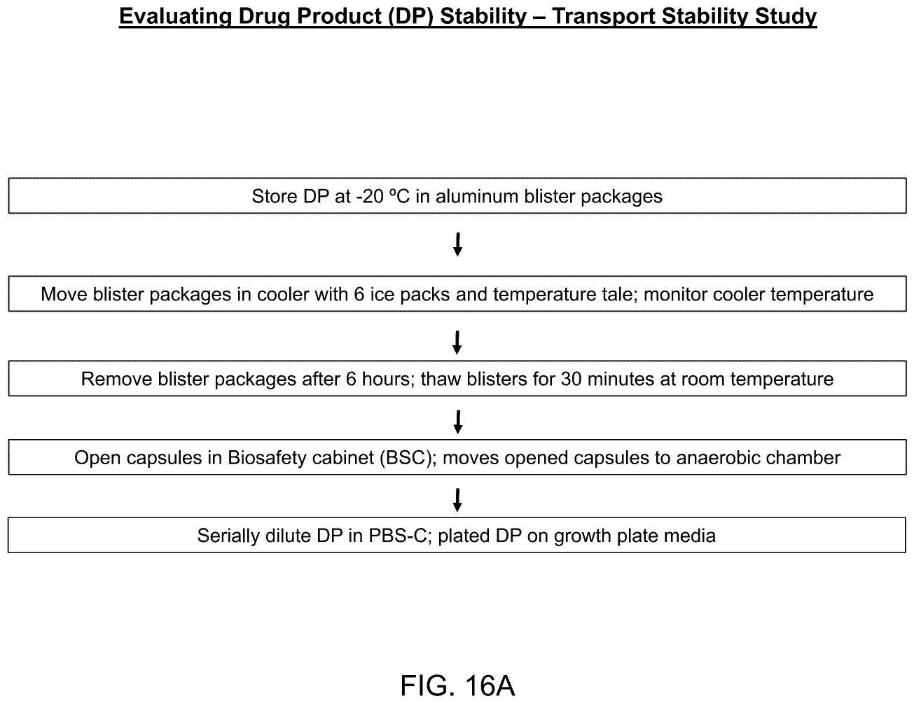

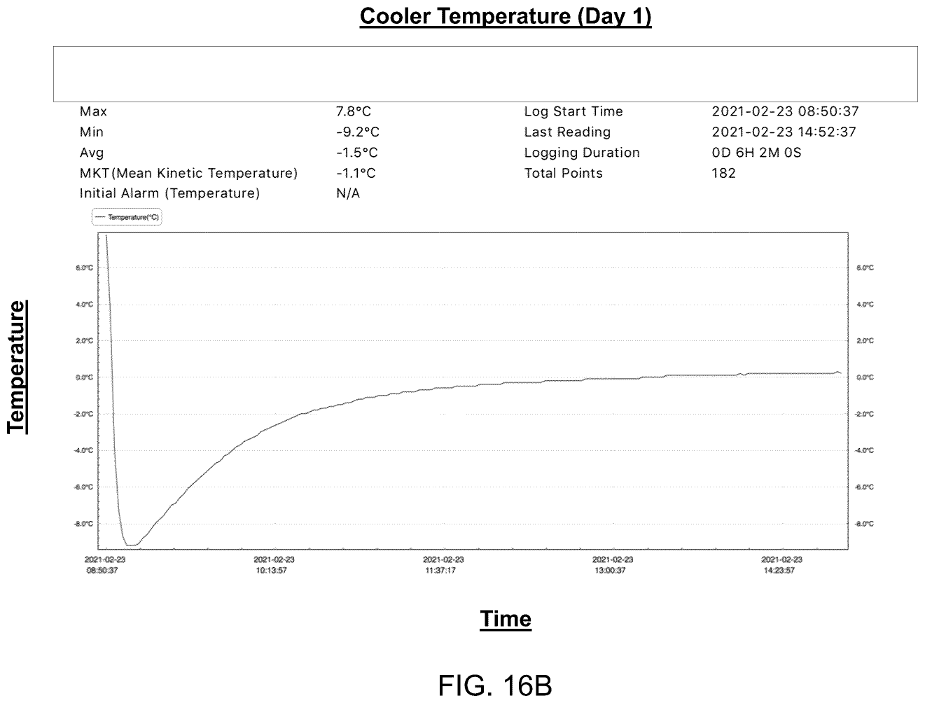

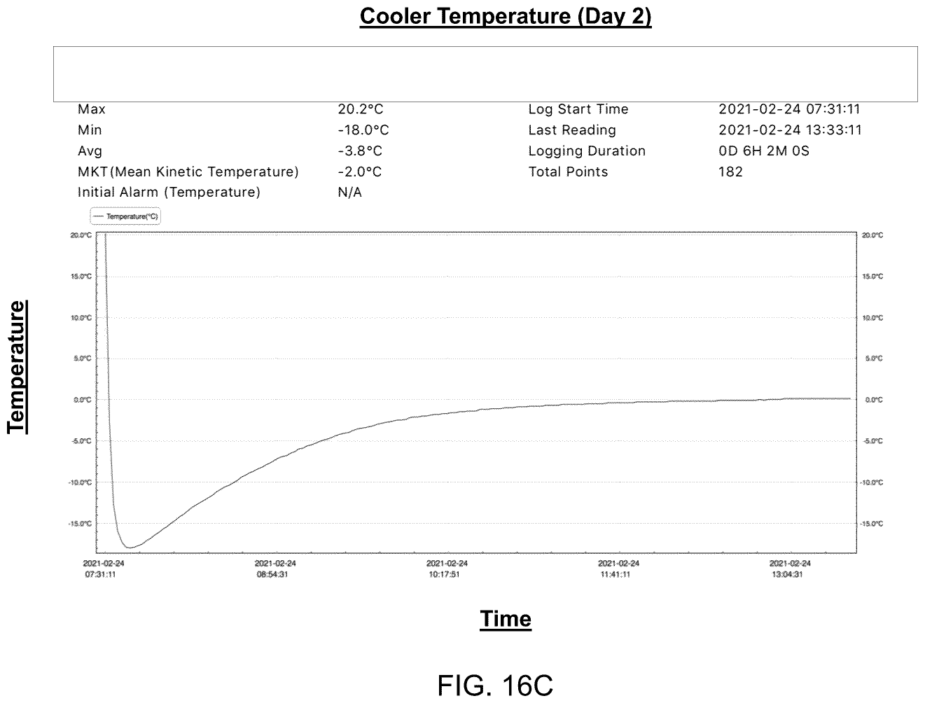

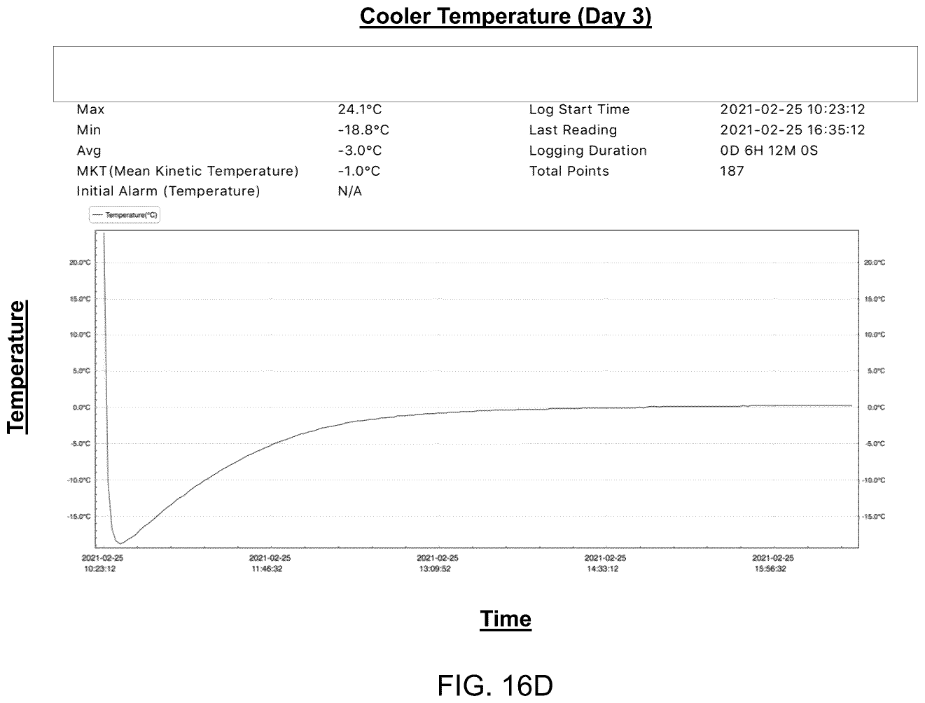

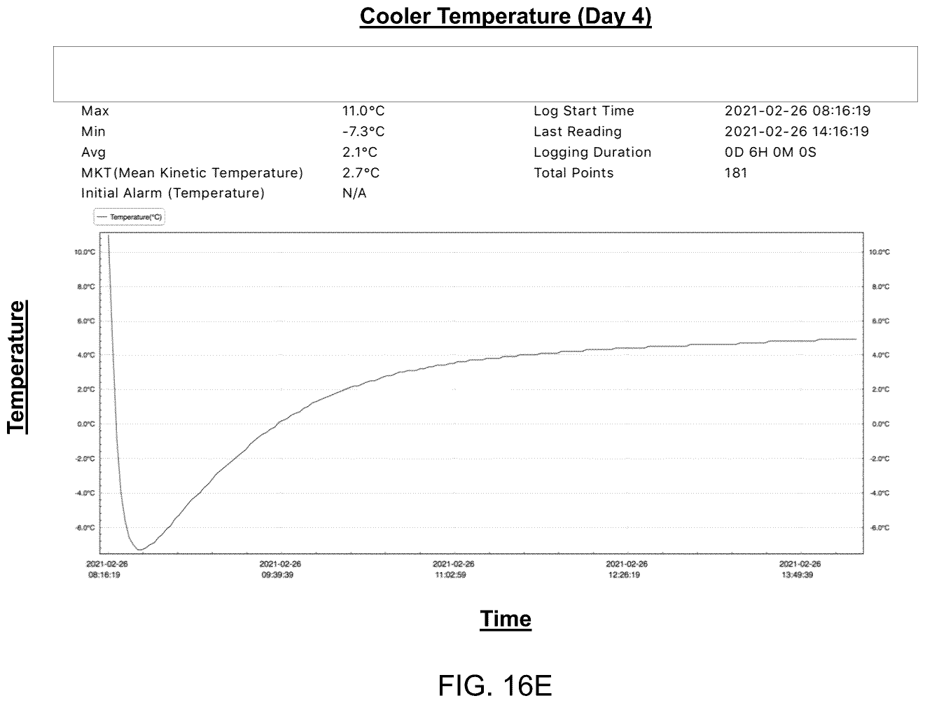

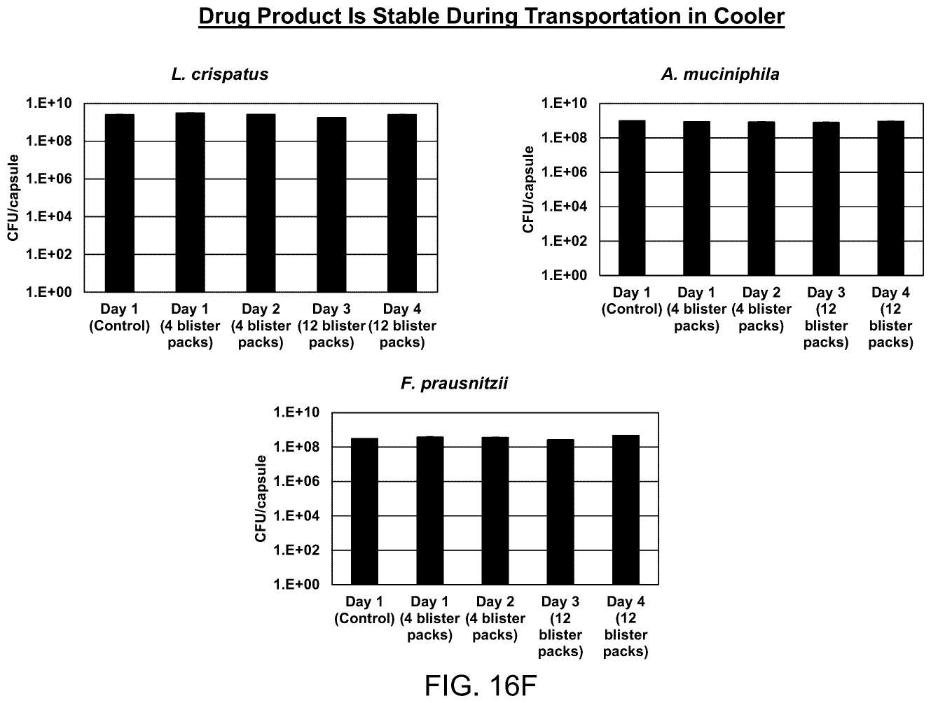

As depicted in FIG. 15B, to assay for the stability of the drug product in the aluminum blister during transportation, the blister was removed from −20° C. and moved to the cooler with ice packs and temperature tale monitor. In different day, different numbers of blisters were stored in the cooler. The temperature inside the cooler was monitored, as shown in FIGS. 16B-16E. The blister was removed from the cooler after six hours and thawed for 30 minutes at room temperature. Once thawed, the capsules were opened in the biosafety cabinet and moved into an anaerobic chamber. The drug products were serially diluted with PBS-C and plated on growth plate media. The colony-forming unit per capsule (CFU/capsule) was scored. On different days, different numbers of blisters were tested in the cooler. The DP without being moved in the cooler was used as a control. As shown in FIG. 16G, F. prausnitzii (DSM 33185), A. muciniphila (DSM 33213), and L. crispatus (DSM 33187) were stable during transportation inside the cooler.

### Example 12—Growth Media Recipes

Provided herein are growth media recipes. Other growth media recipes may also be found elsewhere in this disclosure (e.g., TABLES 3 & 4 in EXAMPLE 1).

The components for YFAP media are listed in TABLE 36.

TABLE 36
Recipe for the 100 L YFAP Media.

| | Weight (g, unless |
|---|---|
| | specified otherwise) |
| Components | for 100 L YFAP media |
| Pea peptone | 200.0 |
| Yeast extract | 500.0 |
| Sodium chloride | 100.0 |
| Sodium bicarbonate | 100.0 |
| Dibasic potassium phosphate | 250.0 |
| Magnesium sulfate heptahydrate | 20.0 |
| Sodium acetate | 500.0 |
| L-cysteine HCl | 100.0 |
| Vitamin Mix solution | 20 mL |

The components for NAGT media are listed in TABLE 37.

TABLE 37
Recipe for the 100 L NAGT Media.

| | Weight (g) for |
|---|---|
| Components | 100 L NAGT media |
| Pea peptone | 2003.2 |
| Yeast extract | 500.0 |
| Sodium chloride | 30.0 |
| Sodium bicarbonate | 100.0 |
| Dibasic potassium phosphate | 250.3 |
| Magnesium sulfate heptahydrate | 10.0 |
| Calcium chloride | 10.0 |
| L-cysteine HCl | 100.0 |
| L-threonine | 401.9 |

### Example 13—Identification of the Genetic and Phenotypic Signatures of a

Bacterial Population, Strain, or Cell

Provided herein are methods for identifying genetic or phenotypic signatures of a bacterial population, strain, or bacterial cell of a pharmaceutical composition described in this disclosure.

A bacterial population, strain, or bacterial cell of the pharmaceutical composition is subjected to genome sequencing. The genomic DNA of the bacterial population, strain, or bacterial cell is isolated. A genome library is constructed. The genome library is subjected to genome sequencing to identify the genome sequence and copy number of the bacterial population, strain, or bacterial cell. The genome sequence information is compared to that of a comparative bacterial population, strain, or bacterial cell (e.g., a frozen stock of the comparative bacterial population, strain, or bacterial cell or a wildtype strain of the comparative bacterial population, strain, or bacterial cell). The difference of the genomes between them are identified.

A bacterial population, strain, or bacterial cell of the pharmaceutical composition is subjected to RNA sequencing. The mRNA of the bacterial population, strain, or bacterial cell is isolated. A transcriptome library is constructed. The transcriptome library is subjected to RNA sequencing to identify the mRNA sequence and expression level of the mRNA of the bacterial population, strain, or bacterial cell. The mRNA sequence information is compared to that of a comparative bacterial population, strain, or bacterial cell (e.g., a frozen stock of the comparative bacterial population, strain, or bacterial cell or a wildtype strain of the comparative bacterial population, strain, or bacterial cell). The difference of the transcriptomes between them are identified.

A bacterial population, strain, or bacterial cell of the pharmaceutical composition is subjected to mass spectroscopy. The proteome of the bacterial population, strain, or bacterial cell is isolated and subjected to mass spectroscopy to identify the proteome makeup of the bacterial population, strain, or bacterial cell. The proteome information is compared to that of a comparative bacterial population, strain, or bacterial cell (e.g., a frozen stock of the comparative bacterial population, strain, or bacterial cell or a wildtype strain of the comparative bacterial population, strain, or bacterial cell). The difference of the proteomes between them are identified.

A bacterial population, strain, or bacterial cell of the pharmaceutical composition is subjected to NMR spectroscopy or mass spectroscopy. The metabolome of the bacterial population, strain, or bacterial cell is isolated and subjected to NMR spectroscopy or mass spectroscopy to identify the metabolome makeup of the bacterial population, strain, or bacterial cell. The metabolome information is compared to that of a comparative bacterial population, strain, or bacterial cell (e.g., a frozen stock of the comparative bacterial population, strain, or bacterial cell or a wildtype strain of the comparative bacterial population, strain, or bacterial cell). The difference of the metabolomes between them are identified.

### Example 14—Testing the Makeup of a Pharmaceutical Composition

Provided herein are methods for testing whether a pharmaceutical composition is a plant-based composition.

A real-time PCR assay is used to determine in which organism species the materials in the pharmaceutical composition are derived from. Species-specific primer pairs are used to identify in which species a material is derived from. One such pair targets mitochondrial cytochrome b locus. DNA is extracted from the pharmaceutical composition and subjected to the PCR amplification using the species-specific primer pairs. The amplification product or reaction may be quantified by the real-time PCR assay. A real-time PCR test may also be Vegan Control™ from Minerva biolabs (Cat. Nr. 370-2025 or Cat. Nr. 370-2100) or mericon MeatTracker Kit from Qiagen (Cat. No./ID: 290145). Alternatively, the amplification product may be measured by gel electrophoresis, sequencing, or DNA-blotting. Other species-specific primers or probes may also be used.

Other assays can comprise ELISA or by quantification of Glycerol Tri-Heptanoate (GTH).

### Example 15—Testing Cryoprotectants Combinations with Drug Product

Provided herein are methods for testing combinations of cryoprotectants and/or pharmaceutically acceptable excipients to increase viability of the bacterial cells in the drug product during manufacture.

To increase the viability of a bacteria stain and/or species during manufacturing, various combinations and ratios of cryoprotectants are tested. A late-log bacteria stain and/or species culture is harvested by centrifugation and resuspended in a growth media. The resuspended bacteria strain and/or species culture is mixed with different combinations of cryoprotectants at two different ratios: 1 biomass (bacteria stain and/or species) to 1 cryoprotectant mixture (1:1) and 1 biomass to 2 cryoprotectant mixture (1:2). The resulting cultures are exposed to oxygen (02) in a biosafety cabinet for up to 4 hours without shaking. The cultures are tested at the following time points: 0 hours, 0.5 hours, 1 hour, 2 hours, and 4 hours of 02 exposure. In some instances, the cultures from the different time points are serially diluted in 96-well plates and the bacterial growth of each well is measured by OD600. In some instances, the cultures from the different time points are plated on solid agar plates to determine the potency of the bacteria stain and/or species. The potency is determined by calculating the colony-forming units per milliliter (CFU/mL) of the bacteria stain and/or species mixed with the different cryoprotectant combinations. In some instances, only the bacterial growth of the cultures from all the time points are measured. In some instances, only the bacterial growth of the culture from the 4-hour time point is measured. In some instances, only the bacterial potency of the cultures from all the time points are measured. In some instances, only the bacterial potency of the cultures from the 4-hour time point is measured. In some instances, both the bacterial growth and the bacterial potency of the cultures from all the time points are measured. In some instances, both the bacterial growth and the bacterial potency of the cultures from only the 4-hour time point are measured.

In some variations of the method, after exposure to 02 for 4 hours, the different cultures are lyophilized. The lyophilized cultures are serially diluted in 96-well plates and the bacterial growth of each well is measured by OD600. In some instances, the lyophilized cultures from the various time points are also plated on solid agar plates to determine the potency of bacteria stain and/or species. In some instances, only the bacterial growth of the lyophilized cultures from the 4-hour time point is measured. In some instances, only the bacterial potency of the lyophilized cultures from the 4-hour time point is measured. In some instances, both the bacterial growth and the bacterial potency of the lyophilized cultures from only the 4-hour time point are measured.

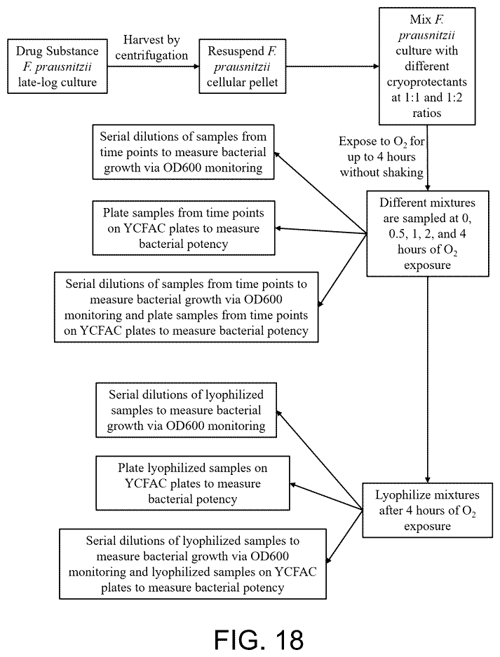

In some variations of the method, the different cultures are tested in either pre-lyophilized or post-lyophilized forms. The different cultures are tested at the following time points: 0 hours, 1 hour, and 4 hours. At each time point, the cultures are tested. The tested cultures from each time point are serially diluted in 96 well plates and bacterial growth is measured by OD600. After the 4-hour 02 exposure, the different cultures are lyophilized. The lyophilized cultures are serially diluted in 96-well plates and the bacterial growth is measured by OD600. In addition, the lyophilized samples are plated on solid plates to measure bacterial potency. In some instances, only the bacterial growth of the cultures from the time points are measured. In some instances, only the bacterial growth of the culture from the 4-hour time point is measured. In some instances, only the bacterial potency of the cultures from the time points are measured. In some instances, only the bacterial potency of the cultures from the 4-hour time point is measured. In some instances, both the bacterial growth and the bacterial potency of the cultures from the time points are measured. In some instances, both the bacterial growth and the bacterial potency of the cultures from only the 4-hour time point are measured. For example, the method described in EXAMPLE 15 may comprise the workflow of FIG. 18 to test the cryoprotectants for F. prausnitzii.

The above disclosed methods can be used to test combinations and ratios of cryoprotectants to increase the viability of any bacterial strain, species, genus, or family disclosed in the present disclosure.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiments

1. A pharmaceutical composition, comprising:
i. a purified bacterial population comprising at least one strain of Akkermansia sp., at least one strain of Faecalibacterium sp., or at least one strain of Lactobacillus sp., and
ii. a pharmaceutically acceptable excipient, wherein the pharmaceutically acceptable excipient comprises a moisture absorbent material.

2. The pharmaceutical composition of embodiment 1, wherein the moisture absorbent material is selected from the group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose, and dicalcium phosphate (DCP).

3. The pharmaceutical composition of any one of embodiments 1 or 2, wherein the moisture absorbent material comprises SiO₂.

4. The pharmaceutical composition of embodiment 3, wherein the SiO₂ is present in an amount of about 0.1% to about 10% by weight.

5. The pharmaceutical composition of embodiment 3, wherein the SiO₂ is present in an amount of about 1% by weight.

6. The pharmaceutical composition of any one of embodiments 1 to 5, wherein the moisture absorbent material comprises mannitol.

7. The pharmaceutical composition of embodiment 6, wherein the mannitol is present in an amount of about 1% to about 90% by weight.

8. The pharmaceutical composition of embodiment 7, wherein the mannitol is present in an amount of about 5% to about 81.5% by weight.

9. The pharmaceutical composition of any one of embodiments 1 to 8, wherein the moisture absorbent material comprises anhydrous lactose.

10. The pharmaceutical composition of embodiment 9, wherein the anhydrous lactose is present in an amount of about 1% to about 90% by weight.

11. The pharmaceutical composition of any one of embodiments 1 to 10, wherein the anhydrous lactose is present in an amount of about 5% to about 81.5% by weight.

12. The pharmaceutical composition of any one of embodiments 1-11, wherein the pharmaceutically acceptable excipient further comprises magnesium stearate.

13. The pharmaceutical composition of embodiment 12, wherein the magnesium stearate is present in an amount of about 0.1% to about 10% by weight.

14. The pharmaceutical composition of embodiment 13, wherein the magnesium stearate is present in an amount of about 1.5% by weight.

15. The pharmaceutical composition of any one of embodiments 1 to 14, wherein the pharmaceutically acceptable excipient further comprises microcrystalline cellulose.

16. The pharmaceutical composition of any one of embodiments 1 to 15, wherein the pharmaceutical composition is formulated into a suspension.

17. The pharmaceutical composition of any one of embodiments 1 to 16, wherein the pharmaceutical composition is formulated as an oral dosage form.

18. The pharmaceutical composition of embodiment 17, wherein the oral dosage form is a capsule, a tablet, an emulsion, a suspension, a syrup, a gel, a gum, a paste, a herbal tea, drops, dissolving granules, powders, tablets, a lyophilizate, a popsicle, a foam, or an ice cream.

19. The pharmaceutical composition of any one of embodiments 1 to 18, wherein the purified bacterial population comprises at least two of: the at least one strain of Akkermansia sp., the at least one strain of Faecalibacterium sp., and the at least one strain of Lactobacillus sp.

20. The pharmaceutical composition of any one of embodiments 1 to 20, wherein the purified bacterial population comprises the at least one strain of Akkermansia sp., the at least one strain of Faecalibacterium sp., and the at least one strain of Lactobacillus sp.

21. The pharmaceutical composition of any one of embodiments 1 to 21, wherein the purified bacterial population is lyophilized.

22. A pharmaceutical composition, comprising:
i. a purified bacterial population comprising at least one strain of Akkermansia sp., at least one strain of Lactobacillus sp., or at least one strain of Faecalibacterium sp., and
ii. a pharmaceutically acceptable excipient,
wherein the pharmaceutical composition is encompassed by a plant-based capsule.

23. The pharmaceutical composition of embodiment 22, wherein the plant-based capsule is encompassed by a blister pack.

24. The pharmaceutical composition of embodiment 22 or 23, wherein the plant-based capsule is a hypromellose capsule.

25. The pharmaceutical composition of embodiment 23 or 24, wherein the blister pack is selected from the group consisting of polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, and plastic film.

26. The pharmaceutical composition of any one of embodiments 23 to 25, wherein the blister pack is an aluminum blister pack.

27. The pharmaceutical composition of any one of embodiments 23 or 26, wherein the blister pack is filled with nitrogen gas.

28. The pharmaceutical composition of any one of embodiments 22 to 27, wherein the pharmaceutically acceptable excipient comprises a moisture absorbent material.

29. The pharmaceutical composition of embodiment 28, wherein the moisture absorbent material is selected from a group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose, and dicalcium phosphate (DCP).

30. The pharmaceutical composition of embodiment 28 or 29, wherein the moisture absorbent material comprises SiO₂.

31. The pharmaceutical composition of embodiment 30, wherein the SiO₂ is present in an amount of about 0.1% to about 10% by weight.

32. The pharmaceutical composition of embodiment 30, wherein the SiO₂ is present in an amount of about 1% by weight.

33. The pharmaceutical composition of any one of embodiments 28 to 32, wherein the moisture absorbent material comprises mannitol.

34. The pharmaceutical composition of embodiment 32, wherein the mannitol is present in an amount of about 1% to about 90% by weight.

35. The pharmaceutical composition of embodiment 33, wherein the mannitol is present in an amount of about 5% to about 81.5% by weight.

36. The pharmaceutical composition of any one of embodiments 28 to 35, wherein the moisture absorbent material comprises anhydrous lactose.

37. The pharmaceutical composition of embodiment 36, wherein the anhydrous lactose is present in an amount of about 1% to about 90% by weight.

38. The pharmaceutical composition of embodiment 37, wherein the anhydrous lactose is present in an amount of about 5% to about 81.5% by weight.

39. The pharmaceutical composition of any one of embodiments 22 to 38, wherein the pharmaceutically acceptable excipient further comprises magnesium stearate.

40. The pharmaceutical composition of embodiment 39, wherein the magnesium stearate is present in an amount of about 0.1% to about 10% by weight.

41. The pharmaceutical composition of embodiment 40, wherein the magnesium stearate is present in an amount of about 1.5% by weight.

42. The pharmaceutical composition of any one of embodiments 22 to 41, wherein the pharmaceutically acceptable excipient further comprises microcrystalline cellulose.

43. The pharmaceutical composition of any one of embodiments 22 to 42, wherein the purified bacterial population comprises at least two of: the at least one strain of Akkermansia sp., the at least one strain of Faecalibacterium sp., and the at least one strain of Lactobacillus sp.

44. The pharmaceutical composition of any one of embodiments 22 to 43, wherein the purified bacterial population comprises the at least one strain of Akkermansia sp., the at least one strain of Faecalibacterium sp., and the at least one strain of Lactobacillus sp.

45. A pharmaceutical composition, comprising:
i. a purified bacterial population comprising at least one strain of Akkermansia sp., at least one strain of Lactobacillus sp., or at least one strain of Faecalibacterium sp., and
ii. a pharmaceutically acceptable excipient,
wherein the pharmaceutical composition is encompassed by a blister pack.

46. The pharmaceutical composition of embodiment 45, wherein the blister pack is selected from the group consisting of polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, and plastic film.

47. The pharmaceutical composition of embodiment 46, wherein the blister pack is an aluminum blister pack.

48. The pharmaceutical composition of any one of embodiments 45 to 47, wherein the blister pack is filled with nitrogen gas.

49. The pharmaceutical composition of any one of embodiments 45 to 48, wherein the pharmaceutically acceptable excipient comprises a moisture absorbent material.

50. The pharmaceutical composition of embodiment 49, wherein the moisture absorbent material is selected from a group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose, and dicalcium phosphate (DCP).

51. The pharmaceutical composition of embodiment 49 or 50, wherein the moisture absorbent material comprises SiO₂.

52. The pharmaceutical composition of embodiment 51, wherein the SiO₂ is present in an amount of about 0.1% to about 10% by weight.

53. The pharmaceutical composition of embodiment 51, wherein the SiO₂ is present in an amount of about 1% by weight.

54. The pharmaceutical composition of any one of embodiments 49 to 53, wherein the moisture absorbent material comprises mannitol.

55. The pharmaceutical composition of embodiment 53, wherein the mannitol is present in an amount of about 1% to about 90% by weight.

56. The pharmaceutical composition of embodiment 54, wherein the mannitol is present in an amount of about 5% to about 81.5% by weight.

57. The pharmaceutical composition of any one of embodiments 49 to 56, wherein the moisture absorbent material comprises anhydrous lactose.

58. The pharmaceutical composition of embodiment 56, wherein the anhydrous lactose is present in an amount of about 1% to about 90% by weight.

59. The pharmaceutical composition of embodiment 57, wherein the anhydrous lactose is present in an amount of about 5% to about 81.5% by weight.

60. The pharmaceutical composition of any one of embodiments 45 to 59, wherein the pharmaceutically acceptable excipient further comprises magnesium stearate.

61. The pharmaceutical composition of embodiment 59, wherein the magnesium stearate is present in an amount of about 0.1% to about 10% by weight.

62. The pharmaceutical composition of embodiment 60, wherein the magnesium stearate is present in an amount of about 1.5% by weight.

63. The pharmaceutical composition of any one of embodiments 45 to 62, wherein the pharmaceutically acceptable excipient further comprises microcrystalline cellulose.

64. The pharmaceutical composition of any one of embodiments 45 to 63, wherein the purified bacterial population comprises at least two of: the at least one strain of Akkermansia sp., the at least one strain of Faecalibacterium sp., and the at least one strain of Lactobacillus sp.

65. The pharmaceutical composition of any one of embodiments 45 to 64, wherein the purified bacterial population comprises at least one strain of Akkermansia sp., at least one strain of Faecalibacterium sp., and at least one strain of Lactobacillus sp.

66. The pharmaceutical composition of any one of embodiments 45 to 65, wherein the purified bacterial population is lyophilized.

67. The pharmaceutical composition of any one of embodiments 1 to 66, wherein the at least one strain of Akkermansia sp., the at least one strain of Faecalibacterium sp., and the at least one strain of Lactobacillus sp. are selected from the strains listed in Table 1.

68. The pharmaceutical composition of any one of embodiments 1 to 67, wherein the bacterial population comprises A. muciniphila (DSM 33213), F. prausnitzii (DSM 33185), or L. crispatus (DSM 33187).

69. The pharmaceutical composition of any one of embodiments 1 to 68, wherein the bacterial population comprises at least two of the bacterial strains A. muciniphila (DSM 33213), F. prausnitzii (DSM 33185), and L. crispatus (DSM 33187).

70. The pharmaceutical composition of any one of embodiments 1 to 69, wherein the bacterial population comprises the bacterial strains A. muciniphila (DSM 33213), F. prausnitzii (DSM 33185), and L. crispatus (DSM 33187).

71. The pharmaceutical composition of any one of embodiments 1 to 70, wherein each bacterial strain is present in an amount from about 10 \hat{ }3 CFU/dose to about 10\hat{ }12 CFU/dose.

72. The pharmaceutical composition of any one of embodiments 1 to 70, wherein each bacterial strain is present in an amount from about 10\hat{ }7 CFU/dose to about 10\hat{ }10 CFU/dose.

73. The pharmaceutical composition of any one of embodiments 1 to 70, wherein the at least one strain of Akkermansia sp. is present in an amount of about 10\hat{ }6 CFU/dose to about 2×10\hat{ }9 CFU/dose.

74. The pharmaceutical composition of any one of embodiments 1 to 70, wherein the at least one strain of Faecalibacterium sp. is present in an amount of about 10\hat{ }6 CFU/dose to about 2×10\hat{ }9 CFU/dose.

75. The pharmaceutical composition of any one of embodiments 1 to 70, wherein the at least one strain of Lactobacillus sp. is present in an amount of about 10\hat{ }7 CFU/dose to about 5×10\hat{ }9 CFU/dose.

76. The pharmaceutical composition of any one of embodiments 1 to 70, wherein the bacterial population is present in a total amount of about 10 \hat{ }3 CFU/dose to about 10\hat{ }12 CFU/dose.

77. The pharmaceutical composition of any one of embodiments 1 to 70, wherein the bacterial population is present in a total amount of about 10\hat{ }7 CFU/dose to about 10\hat{ }10 CFU/dose.

78. A method for treating a subject having or suspected of having a disease, the method comprising administering to the subject a pharmaceutical composition of any one of embodiments 1-77.

79. The method of embodiment 78, wherein the disease is an inflammatory disease.

80. The method of embodiment 79, wherein the inflammatory disease is an allergy or dermatitis.

81. The method of embodiment 80, wherein the allergy is allergic asthma, allergic pediatric asthma, or food allergy.

82. The method of embodiment 78, wherein the disease is a metabolic disease.

83. The method of embodiment 82, wherein the metabolic disease is obesity, diabetes, or a metabolic syndrome.

84. A method for a large-scale growth of Lactobacillus sp. comprising performing a plurality of inoculation rounds with an increasing amount of growth media, wherein an inoculation round comprises at least about 0.5% by volume of a total batch material of a preceding inoculation round, wherein a growth media of the inoculation round is at least about 50 L.

85. The method of embodiment 84, wherein the Lactobacillus sp. comprises Lactobacillus crispatus.

86. The method of embodiment 84 or 85, wherein the Lactobacillus sp. comprises Lactobacillus crispatus (DSM 33187).

87. The method of any one of embodiments 84-86, wherein the growth media of the inoculation round is from about 50 L to about 4,000 L.

88. The method of any one of embodiments 84-87, further comprising an initial inoculation round of about 500 mL growth media.

89. The method of any one of embodiments 84-88, wherein at least one of the inoculation rounds is in a volume of about 3500 L growth media.

90. The method of embodiment 88 or 89, wherein the initial inoculation round comprises a frozen stock of Lactobacillus crispatus of about 0.4% of an initial inoculation round growth media.

91. The method of any one of embodiments 88-90, wherein the initial inoculation round comprises growing Lactobacillus crispatus in anaerobic condition.

92. The method of any one of embodiment 84-91, further comprising performing a plurality of sterilization and degassing rounds for the growth media.

93. The method of any one of embodiments 84-92, further comprising lyophilizing the batch.

94. The method of any one of embodiments 84-93, further comprising centrifuging the batch before the lyophilizing.

95. The method of any one of embodiments 84-94, further comprising grinding the batch after the lyophilizing.

96. A method for producing a pharmaceutical composition comprising:
1) providing a mixture comprising a purified bacterial population comprising at least one strain of Akkermansia sp., at least one strain of Faecalibacterium sp., or at least one strain of Lactobacillus sp.;
2) filling capsules with the mixture; and
3) packing the capsules into a blister pack; wherein the providing and the packing are under an oxygen-free atmosphere.

97. The method of embodiment 96, further comprising storing the capsules in a container before the packing.

98. The method of embodiment 97, wherein the storing is under an oxygen-free atmosphere.

99. The method of embodiment 98, wherein the oxygen-free atmosphere is accomplished by injecting nitrogen gas or an oxygen scrubber.

100. The method of any one of embodiments 96-99, wherein the filling is accomplished by a capsule filler.

101. The method of any one of embodiments 96-99, wherein the filling is accomplished in an anaerobic chamber.

102. The method of any one of embodiments 96-101, wherein the capsule is a plant-based capsule.

103. The method of embodiment 102, wherein the plant-based capsule is a hypromellose capsule.

104. The method of any one of embodiments 96-103, wherein the blister pack is selected from polyethylene terephthalate (PET), polyvinyl chloride (PVC), polyvinylidene chloride (PVDC), polychlorotrifluoroethylene (PCTFE), cyclic olefin polymers (COP), oriented polyamide (OPA), aluminum foil, plastic film.

105. The method of embodiment 104, wherein the blister pack is an aluminum blister pack.

106. The method of any one of embodiments 96-105, wherein the blister pack is filled with nitrogen gas.

107. The method of any one of embodiments 96-106, wherein the pharmaceutical composition further comprises a pharmaceutically acceptable excipient.

108. The method of embodiment 107, wherein the pharmaceutically acceptable excipient comprises a moisture absorbent material.

109. The method of embodiment 108, wherein the moisture absorbent material is selected from the group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose, and dicalcium phosphate (DCP).

110. The method of embodiment 108, wherein the moisture absorbent material comprises SiO₂.

111. The method of embodiment 110, wherein the SiO₂ is present in an amount of about 0.1% to about 10% by weight.

112. The method of embodiment 111, wherein the SiO₂ is present in an amount of about 1% by weight.

113. The method of any one of embodiments 108-112, wherein the moisture absorbent material comprises mannitol.

114. The method of embodiment 113, wherein the mannitol is present in an amount of about 1% to about 90% by weight.

115. The method of embodiment 114, wherein the mannitol is present in an amount of about 5% to about 81.5% by weight.

116. The method of any one of embodiments 108-115, wherein the moisture absorbent material comprises anhydrous lactose.

117. The method of embodiment 116, wherein the anhydrous lactose is present in an amount of about 1% to about 90% by weight.

118. The method of embodiment 117, wherein the anhydrous lactose is present in an amount of about 5% to 81.5% by weight.

119. The method of any one of embodiments 108-118, wherein the pharmaceutically acceptable excipient further comprises magnesium stearate.

120. The method of embodiment 119, wherein the magnesium stearate is present in an amount of about 0.1% to about 10% by weight.

121. The method of embodiment 120, wherein the magnesium stearate is present in an amount of about 1.5% by weight.

122. The method of embodiment any one of embodiments 108-121, wherein the pharmaceutically acceptable excipient further comprises microcrystalline cellulose.

123. The method of any one of embodiments 96-122, wherein the purified bacterial population comprises at least two of: the at least one strain of Akkermansia sp., the at least one strain of Faecalibacterium sp., or the at least one strain of Lactobacillus sp.

124. The method of embodiment 123, wherein the purified bacterial population comprises the at least one strain of Akkermansia sp., the at least one strain of Faecalibacterium sp., and the at least one strain of Lactobacillus sp.

125. The method of any one of embodiments 96-124, wherein the bacterial population is lyophilized.

126. A pharmaceutical composition, comprising: a purified bacterial population comprising at least one strain of Akkermansia sp., at least one strain of Faecalibacterium sp., or at least one strain of Lactobacillus sp.,
wherein the pharmaceutical composition has a shelf stability subsequent to being stored in anaerobic environment for a period of at least 3 days at a temperature of at least 15° C.

127. The pharmaceutical composition of embodiment 126, wherein the shelf stability of the pharmaceutical composition comprises a ratio of a viability of the at least one strain of the purified bacterial population of the pharmaceutical composition relative to a viability of the at least one strain of the purified bacterial population of a comparable pharmaceutical composition prior to or without being stored in the anaerobic environment for the period of at least 3 days at the temperature of at least 15° C.

128. The pharmaceutical composition of embodiment 127, wherein the viability of the at least one strain of the purified bacterial population of the pharmaceutical composition or the viability of the at least one strain of the purified bacterial population of the comparable pharmaceutical composition is measured in colony forming unit (CFU). 129. The pharmaceutical composition of embodiment 127 or 128, wherein the ratio is at least about 1×10\hat{ }-5%.

130. The pharmaceutical composition of any one of embodiments 127-129, wherein the ratio is at least about 1×10\hat{ }-4%.

131. The pharmaceutical composition of any one of embodiments 127-130, wherein the ratio is at least about 1×10\hat{ }-2%.

132. The pharmaceutical composition of any one of embodiments 127-121, wherein the ratio is at least about 1%.

133. The pharmaceutical composition of any one of embodiments 127-132, wherein the ratio is at least about 10%.

134. The pharmaceutical composition of any one of embodiments 127-133, wherein the ratio is at least about 20%.

135. The pharmaceutical composition of any one of embodiments 127-134, wherein the ratio is at least about 50%.

136. The pharmaceutical composition of any one of embodiments 127-135, wherein the pharmaceutical composition is stored in the anaerobic environment for a period of at least 3 days at a temperature of about 25° C.

137. The pharmaceutical composition of any one of embodiments 127-135, wherein the pharmaceutical composition is stored in the anaerobic environment for a period of at least 10 days at a temperature of at least 15° C.

138. The pharmaceutical composition of embodiment 137, wherein the pharmaceutical composition is stored in the anaerobic environment for a period of at least 100 days at a temperature of at least 15° C.

139. The pharmaceutical composition of any one of embodiments 126-138, wherein the pharmaceutical composition comprises a cryoprotectant.

140. The pharmaceutical composition of embodiment 139, wherein the pharmaceutical composition is lyophilized.

141. The pharmaceutical composition of any one of embodiments 126-140, wherein the pharmaceutical composition comprises a pharmaceutically acceptable excipient.

142. The pharmaceutical composition of embodiment 141, wherein the pharmaceutically acceptable excipient comprises maltodextrin, cellulose, methionine, ascorbic acid, magnesium stearate, beta-cyclodextrin, dextrin, 2-Hydroxypropyl-β-cyclodextrin cysteine, riboflavin, starch, Glucidex, mannitol, saccharose, trehalose, anhydrous lactose, or a combination thereof.

143. The pharmaceutical composition of any one of embodiments 126-142, wherein the pharmaceutical composition is encapsulated in a capsule.

144. The pharmaceutical composition of embodiment 143, wherein the capsule is a plant-based capsule.

145. The pharmaceutical composition of any one of embodiments 126-144, wherein the pharmaceutical composition is encompassed by a blister pack, a sachet pack, a vial, a bottle, an ampoule, or a combination thereof.

146. The pharmaceutical composition of embodiment 145, wherein the pharmaceutical composition is encompassed by the blister pack.

147. The pharmaceutical composition of embodiment 145, wherein the pharmaceutical composition is encompassed by the sachet pack.

148. The pharmaceutical composition of any one of embodiments 126-147, wherein the pharmaceutical composition is formulated into a suspension.

149. The pharmaceutical composition of any one of embodiments 126-148, wherein the pharmaceutical composition is formulated as an oral dosage form.

150. The pharmaceutical composition of embodiment 149, wherein the oral dosage form is a capsule, a tablet, an emulsion, a suspension, a syrup, a gel, a gum, a paste, a herbal tea, drops, dissolving granules, powders, tablets, a lyophilizate, a popsicle, a foam, or an ice cream.

151. The pharmaceutical composition of any one of embodiments 126-150, wherein the purified bacterial population comprises at least two of: the at least one strain of Akkermansia sp., the at least one strain of Faecalibacterium sp., and the at least one strain of Lactobacillus sp.

152. The pharmaceutical composition of embodiment 151, wherein the purified bacterial population comprises the at least one strain of Akkermansia sp., the at least one strain of Faecalibacterium sp., and the at least one strain of Lactobacillus sp.

1.-152. (canceled)

153. A pharmaceutical composition, comprising:
i. a purified bacterial population comprising at least one strain of Akkermansia sp., at least one strain of Faecalibacterium sp., or at least one strain of Lactobacillus sp., and
ii. a pharmaceutically acceptable excipient, wherein the pharmaceutically acceptable excipient comprises a moisture absorbent material.

154. The pharmaceutical composition of claim 153, wherein the moisture absorbent material is selected from the group consisting of microcrystalline cellulose (MCC), hydroxypropyl methylcellulose, silicon dioxide (SiO₂), polyethylene glycol 8000, lactose, D-trehalose dihydrate, mannitol, calcium phosphate tribasic, calcium sulfate, corn starch, fructose, xylitol, maltitol, anhydrous lactose, and dicalcium phosphate (DCP).

155. The pharmaceutical composition of claim 153, wherein the moisture absorbent material comprises SiO₂.

156. The pharmaceutical composition of claim 155, wherein the SiO₂ is present in an amount of about 0.1% to about 10% by weight of the pharmaceutical composition.

157. The pharmaceutical composition of claim 155, wherein the SiO₂ is present in an amount of about 1% by weight of the pharmaceutical composition.

158. The pharmaceutical composition of claim 153, wherein the moisture absorbent material comprises mannitol.

159. The pharmaceutical composition of claim 158, wherein the mannitol is present in an amount of about 1% to about 90% by weight of the pharmaceutical composition.

160. The pharmaceutical composition of claim 159, wherein the mannitol is present in an amount of about 5% to about 81.5% by weight of the pharmaceutical composition.

161. The pharmaceutical composition of claim 153, wherein the moisture absorbent material comprises anhydrous lactose.

162. The pharmaceutical composition of claim 161, wherein the anhydrous lactose is present in an amount of about 1% to about 90% by weight of the pharmaceutical composition.

163. The pharmaceutical composition of claim 162, wherein the anhydrous lactose is present in an amount of about 5% to about 81.5% by weight of the pharmaceutical composition.

164. The pharmaceutical composition of claim 153, wherein the pharmaceutically acceptable excipient further comprises magnesium stearate.

165. The pharmaceutical composition of claim 164, wherein the magnesium stearate is present in an amount of about 0.1% to about 10% by weight of the pharmaceutical composition.

166. The pharmaceutical composition of claim 165, wherein the magnesium stearate is present in an amount of about 1.5% by weight of the pharmaceutical composition.

167. The pharmaceutical composition of claim 153, wherein the pharmaceutically acceptable excipient further comprises microcrystalline cellulose.

168. The pharmaceutical composition of claim 153, wherein the pharmaceutical composition is formulated as an oral dosage form.

169. The pharmaceutical composition of claim 168, wherein the oral dosage form is a capsule, a tablet, an emulsion, a suspension, a syrup, a gel, a gum, a paste, a herbal tea, drops, dissolving granules, powders, tablets, a lyophilizate, a popsicle, a foam, or an ice cream.

170. The pharmaceutical composition of claim 153, wherein the purified bacterial population comprises at least two of: the at least one strain of Akkermansia sp., the at least one strain of Faecalibacterium sp., and the at least one strain of Lactobacillus sp.

171. The pharmaceutical composition of claim 153, wherein the purified bacterial population comprises the at least one strain of Akkermansia sp., the at least one strain of Faecalibacterium sp., and the at least one strain of Lactobacillus sp.

172. The pharmaceutical composition of claim 153, wherein the purified bacterial population is lyophilized.
